(19) United States
(12) Patent Application Publication
(10) Pub. No.: US 2023/0045048 A1
(43) Pub. Date: Feb. 9, 2023

(54) IL-15 COMPOSITIONS AND METHODS OF USE THEREOF

(71) Applicant: PROVIVA THERAPEUTICS (HONG KONG) LIMITED, Hong Kong (CN)

(72) Inventor: Zijuan LI, Shanghai (CN)

(21) Appl. No.: 17/413,786

(22) PCT Filed: Dec. 13, 2019
(86) PCT No.: PCT/US2019/066287

§ 371 Date: Jun. 14, 2021

Related U.S. Application Data
(60) Provisional application No. 62/779,793, filed on Dec. 14, 2018.

(51) Int. Cl. C07K 14/54; C07K 14/715

(52) U.S. Cl. CPC C07K 14/5443; C07K 14/7155; A61K 38/00; C07K 2319/50; C07K 2319/30

(57) ABSTRACT

Provided are compositions comprising an activatable proprotein comprising a first IL-15 or a variant thereof and a second IL-15Rα or variant thereof fused to a masking moiety comprising an antibody Fc region, and methods of using the same for cancer immunotherapy and other therapies.

### CROSS-REFERENCE TO RELATED APPLICATIONS

This application claims priority under 35 U.S.C. § 119(e) to U.S. Application No. 62/779,793, filed Dec. 14, 2018, which is incorporated by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is VIVA_001_01WO_ST25.txt. The text file is about 1,733 KB, created on Dec. 14, 2019, and is being submitted electronically via EFS-Web.

### BACKGROUND OF THE INVENTION

IL-15 is a pleiotropic cytokine that has been demonstrated to induce and regulate a myriad of immune functions. In particular, IL-15 is critical for lymphoid development and peripheral maintenance of innate immune cells and immunological memory of T cells, specifically natural killer (NK) and CD8+ T cell populations. However, while IL-15 does not promote the maintenance of CD4+CD25+FOXP3+ regulatory T cells (Tregs), IL-2 has been demonstrated to induce their development and IL-15 has been shown to protect effector T cells from IL-2-mediated activation-induced cell death (AICD). For these reasons, IL-15 has long been speculated to have high therapeutic potential for long-term anti-tumor immunity. IL-15 is a four-α-helix protein belonging to a family of cytokines consisting of interleukins IL-2, IL-4, IL-7, IL-9, and IL-21. IL-15 signals through a receptor complex composed of the IL-2/IL-15 receptor β (IL-15Rβ) (CD122) subunit, which is shared with IL-2 and the common gamma chain (γC) (CD132) receptor subunit, which is also utilized by all of the additional family members. While it does not have a crucial direct role in IL-15 signaling per se, IL-15Rα is a critical component of the IL-15 cytokine-receptor complex. IL-15Rα is a transmembrane protein with very high affinity for IL-15 that facilitates IL-15 trafficking from the endoplasmic reticulum (ER) through the cytoplasm and presentation of IL-15/IL-15Rα complexes on the cell surface. In addition to remaining associated throughout cytoplasmic and cell surface expression, IL-15/IL-15Rα can also be cleaved as a complex into the extracellular space.

Once expressed on the cell surface, IL-15/IL-15Rα complexes can stimulate neighboring or opposing cells in trans through IL-15Rβ/γC. This type of cytokine stimulation requires cell-cell contact and is referred to as transpresentation. Under steady-state conditions, trans-presentation is believed to be the primary mode of action for IL-15-mediated development and homeostasis of CD8 T cells, NK cells, NKT cells and intraepithelial lymphocytes. Trans-presentation offers a tighter regulation than that of a secreted cytokine by providing cell-directed delivery of IL-15 to specific cell types. Nevertheless, soluble (s) IL-15/IL-15Rα complexes are also cleaved from the cell surface in response to a variety of inflammatory signals, such as TLR ligation, type I Interferons, and CD40 ligation. This production of sIL-15 complexes is generally transient in nature and may provide a short-lived, but strong burst of IL-15 activity to support early immune responses. In both of these circumstances, IL-15Rα binding of IL-15 is not only a platform for IL-15 delivery but also increases the half-life of IL-15 and may also increase the affinity of IL-15 for IL-15Rβ/γC. Indeed, sIL-15 complexes have been found to be superior in their ability to stimulate IL-15 responses compared to unassociated rIL-15. This garnered a lot of interest in IL-15 and sparked the generation of multiple formulations of sIL-15 complexes that are now being examined for therapeutic efficacy.

The immune effect of IL-15 in cancer patients has been examined and has been demonstrated to induce the expansion of NK cells and significantly increased the proliferation of γδT cells and CD8+ T cells following administration in humans.

Despite its promising anti-tumor immune capacity, IL-15 has been shown to exhibit a short half-life and high doses were required to achieve biological responses in vivo, thereby resulting in clinical toxicities and limited anti-tumor responses in patients. To increase the therapeutic effectiveness and facilitate the use of IL-15 in the immunotherapy of cancer and chronic infection, several companies are currently developing IL-15 and IL-15 derivatives, However, major drawbacks of the mentioned drug candidates exist: (1) high serum Cmax initially causing over-activation of immune system; (2) short PK due to either small molecular size for IL-15 (13-14 kD) or catabolism by the large number of immune cells expressing IL-15 receptors for IL-15 or IL-15 Fc fusion proteins; (3) poor accumulation in the target tumor due to short PK, lack of or ineffective tumor targeting; and (4) undesirable accumulation and immune activation activities in normal tissues.

The present invention solves all of the problems mentioned above by providing an activatable proprotein comprising interleukin 15 (IL-15) that is activated within a cancer tissue or tumor. This platform has the potential to address the drawbacks of IL-15 and its derivatives currently being developed for cancer immunotherapy.

### SUMMARY OF THE INVENTION

In one aspect, the invention relates to an activatable proprotein comprising a first polypeptide and a second polypeptide, wherein the first and second polypeptide each comprise a masking moiety operably linked via a first linker on the C-terminus to an IL-15 or variant thereof, wherein the IL-15 or variant thereof is linked via a second linker on the C-terminus to an IL-15Rα or variant thereof, and wherein the masking moiety masks the active portion of the proprotein.

In one aspect, the invention relates to an activatable proprotein comprising a first polypeptide and a second polypeptide, wherein the first and second polypeptide each comprise an IL-15 or variant thereof operably linked via a first linker on the C-terminus to an IL-15Rα or variant thereof, wherein the IL-15Rα or variant thereof is linked via a second linker on the C-terminus to a masking moiety present on each of the first and second polypeptides, and wherein the masking moiety masks the active portion of the proprotein.

In a related aspect, the first linker on the first or second polypeptide, or on both the first and the second polypeptide is a cleavable linker. In another related aspect, the second linker on the first or second polypeptide, or on both the first and the second polypeptide is a cleavable linker.

In a related aspect, the IL-15 or variant thereof and the IL-15Rα or variant thereof in the first polypeptide and the IL-15 or variant thereof and the IL-15Rα or variant thereof in the second polypeptide comprise one or more Cys substitution mutations.

In another related aspect, the IL-15 or variant thereof in the first polypeptide comprising one or more Cys substitution mutations forms a disulfide bond with the IL-15Rα or variant thereof comprising one or more Cys substitution mutations in the second polypeptide. In another embodiment, the IL-15 or variant thereof in the second polypeptide comprising one or more Cys substitution mutations forms a disulfide bond with the IL-15Rα or variant thereof comprising one or more Cys substitution mutations in the first polypeptide.

In a related aspect, the proprotein is a dimeric proprotein. In another related aspect, the dimeric proprotein comprises a first polypeptide and a second polypeptide each comprising from N-terminus to C-terminus the masking moiety linked via the first linker to the IL-15 or variant thereof, wherein the IL-15 or variant thereof is linked via the second linker to the IL-15Rα or variant thereof, and wherein the masking moiety of the first polypeptide forms covalent disulfide bond or non-covalent bond is fused to the masking moiety of the second polypeptide. In another related aspect, the dimeric proprotein comprises a first polypeptide and a second polypeptide each comprising from N-terminus to C-terminus the masking moiety linked via the first linker to the IL-15 or variant thereof, wherein the IL-15 or variant thereof is linked via the second linker to the IL-15Rα or variant thereof, and wherein the masking moiety of the first polypeptide forms one or more disulfide bonds or non-covalent bonds with the masking moiety of the second polypeptide. In one embodiment, the dimeric proprotein comprises significantly reduced biological activity in comparison to native IL-15. In another embodiment, the dimeric protein is activatable through protease cleavage to restore the activity of the IL-15 or variant thereof present in the dimeric proprotein.

In a related aspect, provided is a recombinant nucleic acid molecule encoding an activatable proprotein disclosed herein. In another related aspect, provided is a vector comprising the recombinant nucleic acid molecule encoding an activatable proprotein.

In a related aspect, provided is a pharmaceutical composition comprising the activatable proprotein disclosed herein, and a pharmaceutically acceptable carrier.

In another aspect, the invention relates to a method comprising the step of administering the activatable proprotein or the pharmaceutical composition comprising the activatable proprotein disclosed herein to a subject having a cancer to treat the cancer in the subject, wherein following administration, the activatable proprotein is activated through protease cleavage in a cancerous tissue. In another embodiment, the activatable proprotein is activated following or after protease cleavage in a cancerous tissue.

In a related aspect, the invention relates to a method comprising the step of administering the activatable proprotein or the pharmaceutical composition comprising the activatable proprotein disclosed herein to a subject in need thereof, to elicit or enhance an anti-tumor immune response in the subject, wherein following administration, the activatable proprotein is activated through protease cleavage in the tumor. In another embodiment, the activatable proprotein is activated following or after protease cleavage in the tumor.

In another aspect, the invention relates to a use of the activatable proprotein or the pharmaceutical composition comprising the activatable proprotein disclosed herein for treating a cancer in a subject, comprising the step of administering the activatable proprotein or pharmaceutical composition comprising the same, and wherein following administration, the activatable proprotein is activated through protease cleavage in a cancerous tissue.

In another aspect, the invention relates to a use of the activatable proprotein or the pharmaceutical composition comprising the activatable proprotein disclosed herein for eliciting or enhancing an anti-tumor immune response in a subject, comprising the step of administering the activatable proprotein or pharmaceutical composition, and wherein following administration, the activatable proprotein is activated through protease cleavage in a tumor.

In another embodiment of the methods or uses disclosed herein, the protease cleavage partially or completely removes the masking moiety in the activatable proprotein such that the IL-15/IL-IL-15Rα complex in the first and the second polypeptide can bind IL-15Rβ/γC present on the surface of a lymphocyte of blood cell in vitro or in vivo.

Other features and advantages of the present invention will become apparent from the following detailed description examples and figures. It should be understood, however, that the detailed description and the specific examples while indicating embodiments of the invention are given by way of illustration only, since various changes and modifications within the spirit and scope of the invention will become apparent to those skilled in the art from this detailed description.

### BRIEF DESCRIPTION OF THE DRAWINGS

Various objects and advantages and a more complete understanding of the present invention are apparent and more readily appreciated by reference to the following Detailed Description and to the appended claims when taken in conjunction with the accompanying Drawing wherein:

FIG. 1A shows the protein topology of human interleukin 15 (IL-15) and human interleukin 15 receptor alpha chain (IL-15Rα)

FIG. 1B shows human_IL-15_LSP: Amino acid sequences of IL-15 with long signal peptide (1-162); human_IL-15_mature: Amino acid sequences of IL-15 without signal peptide (49-162); human_IL-15Rα_FL: full length sequence of IL-15Rα with signal peptide underlined (1-267); human_IL-15Rα-ECD: extracellular domain sequence of IL-15Rα (31-205); human_IL-15Rα-sushi+: extracellular sushi domain sequence plus the first 13 amino acids from the linker region (31-98); human_IL-15Rα-sushi: extracellular sushi domain sequence (31-85).

FIG. 1C shows quaternary structure of IL-15 in complex with its receptors IL-15Rα (CD215), IL-15Rβ (CD122) and the common gamma chain (CD132) (PDB:4GS7) with schematic drawing of cell membrane and transmembrane and signaling domains of the receptors.

FIG. 1D illustrates a schematic diagram of IL-15 and cell surface IL-15 receptor complex.

FIG. 2A illustrates the IL-15 complex with its receptor IL-15Rα-sushi (PDB:2Z3Q) with an artificial flexible peptide linker between the C-terminus of IL-15 and N-terminus of IL-15Rα-sushi (to form an ILR)). The interaction site on IL-15 in ILR with its signaling IL-15Rβ/γc receptors is indicated at IL-15Rβ/γc Interaction interface.

FIG. 2B illustrates a homodimeric association of ILR fusion proteins described in FIG. 2A. The interaction sites on IL-15 in the homodimeric ILR with its signaling IL-15Rβ/γc receptors is indicated at IL-15Rβ/γc Interaction interface. Unlike an RLI fusion protein, ILR fusion protein favors homodimer formation.

FIG. 2C illustrates a schematic diagram of ILR structure described in FIG. 2A. IL-15 in the ILR is able to bind to and signal through IL-15Rβ/γc receptors.

FIG. 2D illustrates a schematic diagram of a dimeric ILR structure described in FIG. 2B. IL-15 in the dimeric ILR is able to bind to and signal through IL-15Rβ/γc receptors.

FIG. 3A illustrates a fusion of the C-terminus of an ILR (described in FIG. 2B) to the N-terminus of a fusion moiety. Similar to the structure described in FIG. 2B, this fusion also favors a dimer formation. The interaction sites on IL-15 in the homodimeric ILR with its signaling IL-15Rβ/γc receptors is indicated at IL-15Rβ/γc Interaction interface.

FIG. 3B illustrates a schematic diagram of a dimeric fusion structure described in FIG. 3A. IL-15 in the dimeric ILR is able to bind to and signal through IL-15Rβ/γc receptors. IL-15 in this dimeric fusion is able to bind to and signal through IL-15Rβ/γc receptors.

FIG. 3C illustrates a diagram of the protein sequence motifs and configurations for proteins described in FIGS. 3A and 3B.

FIG. 3D illustrates a fusion of the N-terminus of an ILR (described in FIG. 2B) to the C-terminus of a fusion moiety (a masking moiety). Similar to the structure described in FIG. 2B, this fusion also favors a dimer formation. The interaction sites on IL-15 in the homodimeric ILR with its signaling IL-15Rβ/γc receptors is indicated at IL-15Rβ/γc Interaction interface.

FIG. 3E illustrates a schematic diagram of a dimeric fusion structure described in FIG. 3D. IL-15 in the dimeric ILR is able to bind to and signal through IL-15Rβ/γc receptors. IL-15 in this dimeric fusion is not able to bind to and signal through IL-15Rβ/γc receptors because of the steric hindrance resulted from the masking moiety and the linkers between the ILR and the masking moiety.

FIG. 3F illustrates a diagram of the protein sequence motifs and configurations for proteins described in FIGS. 3D and 3E.

FIG. 3G illustrates a schematic diagram of a dimeric fusion structure with a protein domain at the C-terminal of IL-15Rα on the first polypeptide.

FIG. 3H illustrates a diagram of the protein sequence motifs and configurations for proteins described in FIG. 3G.

FIG. 3I illustrates a schematic diagram of a dimeric fusion structure with a protein domain at the C-terminal of IL-15Rα on the second polypeptide.

FIG. 3J illustrates a diagram of the protein sequence motifs and configurations for proteins described in FIG. 3I.

FIG. 3K illustrates a schematic diagram of a dimeric fusion structure with a protein domain at the C-terminal of IL-15Rα on the first and second polypeptides.

FIG. 3L illustrates a diagram of the protein sequence motifs and configurations for proteins described in FIG. 3K.

FIG. 4A illustrates a diagram of the protein sequence motifs and configurations similar but not limited to what was described in FIG. 3F. Any one of more of the protein sequence motifs in the first polypeptide diagram can be different from the corresponding motifs in the second polypeptide as indicated by the double headed arrows. The linker sequence indicated in the diagram can be a flexible and protease cleavable or a flexible and protease non-cleavable or combination of them.

FIG. 4B illustrates examples of masking motifs in the first and second polypeptides. The masking motif in the first polypeptide can be the same or different than the masking motif in the second polypeptide. The masking motif from the first polypeptide can either form non-covalent interaction or covalent interaction or both covalent and non-covalent interaction with the masking motif in the second polypeptide.

FIGS. 4C-4D illustrate examples of Fab-CH2CH3 (4C) and Fab-Cys-CH2CH3 (4D) as masking moieties in polypeptide fusion with ILR.

FIGS. 4E-4F illustrate examples of CH2CH3 (4E) and Cys-CH2CH3 (4F) as masking moieties in polypeptide fusion with ILR.

FIGS. 4G-4H show examples of CH3 (4G) and Cys-CH3 (4H) as masking moieties in polypeptide fusion with ILR.

FIGS. 4I-4J show examples of VH-CH1 and VL-CL (4I) and VH-CH1-Cys and VL-CL-Cys (4J) as masking moieties in polypeptide fusion with ILR

FIGS. 4K-4L show examples of heterodimeric CH2CH3 (4K) and heterodimeric Cys-CH2CH3 (4L) as masking moieties in polypeptide fusion with ILR

FIGS. 4M-4N show examples of Leucine zipper (Coiled coil) (4M) and Cys-leucine zipper (Coiled coil) (4N) as masking moieties in polypeptide fusion with ILR

FIG. 4O shows examples of Cys containing flexible peptide (4O) as masking moieties in polypeptide fusion with ILR.

FIG. 5 shows schematic diagram of dimeric Cys-CH2-CH3-ILR. Lack of IL15 signaling activity through IL15Rβ/γc receptors suggests that inter-chain association of IL15 and IL15R is more favorable than the intra-chain association of IL15 and IL15R resulting in the masking of the IL15Rβ/γc interaction site on IL15.

FIG. 6 shows a schematic diagram of activation of dimeric Cys-CH2-CH3-ILR through protease cleavage of the substrate linker sequences in the fusion polypeptide. Example of protease substrate sequence for linker 1 and linker 3 is shown. Digestion of one of the protease substrate sequences (partial digestion) in the linkers releases the steric hindrance imposed by the masking moiety, therefore allow the IL-15 in the fusion to bind to and signal through IL15Rβ/γc receptors. Digestion of the both cleavable linkers releases the ILR from the Fc fusion resulting in full activity of the ILR towards the IL15Rβ/γc receptors.

FIG. 7 shows a schematic diagram of activation of dimeric Cys-CH2-CH3-ILR through protease cleavage of the substrate linker sequences in the fusion polypeptide. Example of protease substrate sequence for linker 2 and linker 4 is shown. Digestion of one of the two protease substrate sequences in the linkers does not effectively release the steric hindrance imposed by the masking moiety. Therefore full digestion of both protease linkers is necessary to activate the fusion polypeptide signaling through IL15Rβ/γc receptors.

FIG. 8 shows a sequence identifier for exemplary fusion proteins.

FIGS. 9A-9F show representative SDS-PAGE results of purified proteins. “M” on the figures represents a protein standard marker.

FIGS. 10A-10G illustrate representative HPLC analysis results of purified proteins.

FIGS. 11A-11B illustrate representative fluorescence assisted cell sorting (FACS) gating and dot plot of pSTAT5 cell signaling activity of IL-15 fusion protein P1185.

FIGS. 11C-11D illustrate cell signaling activity of IL-15 fusion proteins, as measured by pSTAT5 FACS assay.

FIGS. 12A-12C illustrate representative FACS gating of IL-15 stimulated sample in cell signaling assay using human whole blood.

FIGS. 12D-12F illustrate cell signaling activity of IL-15 fusion proteins using human whole blood, as measured by pSTAT5 FACS assay.

FIGS. 13A-13H illustrate activity of IL15 fusion proteins on M-07e proliferation determined by a colorimetric assay (Cell Counting Kit-8 (CCK-8)).

FIG. 14A illustrates representative gating of lymphocytes on side scatter (SSC) and forward scatter (FSC) for IL-15 stimulated samples.

FIGS. 14B-14F illustrate human PBMC proliferation assay results for IL-15 fusion proteins.

FIGS. 15A-15I illustrate protease (TEV) cleavage of IL-15 fusion proteins. “M” on the figures represents a protein standard maker.

FIGS. 16A-16G illustrate cleavage of IL-15 fusion proteins with uPA, matriptase, legumain, MMP-2 and MMP-9. M represents the protein standard marker; B represents the sample before protease cleavage; uPA represents for uPA protease; Mat represents matriptase protease; L represents legumain protease; M-2 represents MMP-2 protease; M-9 represents MMP-9 protease. Red-arrowed band: uncleaved protein; pink-arrowed band: Fc/Fc-L15R; yellow arrowed band: Fc dimer; green arrowed band: L15R.

FIGS. 17A-17B illustrate cell signaling activity of IL15 fusion proteins, as measured by a pSTAT5 FACS assay.

FIGS. 18A-18I show activity of IL15 fusion proteins on M-07e proliferation determined by a colorimetric assay (Cell Counting Kit-8 (CCK-8)).

FIGS. 19A-19D show human PBMC proliferation assay results of IL-15 fusion protein P1256 with and without TEV protease cleavage. P1185 was used as positive control. P1256 digested represents for P1256 protein digested by TEV protease at TEV to protein ratio of 1 to 2 at 37° C. for overnight. P1256+TEV 1-2 represents for P1256 mixed with TEV at TEV to protein ratio of 1 to 2 for proliferation assay. Cell culture medium was used in TEV digestion reactions.

FIGS. 20A-20F show protease (TEV) cleavage of IL-15 fusion proteins. “M” represents a protein standard marker; B represents the protein sample prior to TEV cleavage; A represents the protein sample after TEV cleavage.

FIG. 21 shows cell signaling activity of IL15 fusion proteins, as measured by a pSTAT5 FACS assay.

FIGS. 22A-22F show the activity of IL15 fusion proteins on M-07e proliferation, as determined by a colorimetric assay (Cell Counting Kit-8 (CCK-8)).

FIGS. 23A-23F show reduced SDS-PAGE results of purified proteins. “M” on the figures represents the protein standard marker.

FIGS. 24A-24F show non-reduced SDS-PAGE results of purified proteins. “M” on the figures represents the protein standard marker.

FIGS. 25A-25X illustrate representative HPLC analysis results of purified proteins.

FIGS. 26A-26P show SDS-PAGE results of cleavage of IL-15 fusion proteins. “M” on the figures represents the protein standard marker. “B” represents for protein before protease cleavage. “B-4” represents for protein before cleavage and store at 4 degree. “B-37” represents for protein before cleavage and store at 37 degree. “uPA” represents for uPA protease. “Mat” represents for matriptase protease. “L” represents for legumain protease. “M-2” represents for MMP-2 protease. “M-9” represents for MMP-9 protease. “K5” represents for KLK-5 protease. “K7” represents for KLK-7 protease.

FIGS. 27A-27N and FIGS. 28A-28W show activity of IL-15 fusion proteins on M-07e proliferation determined by a colorimetric assay (Cell Counting Kit-8 (CCK-8)).

FIGS. 29A-29F illustrate representative results of in vitro serum stability. Western blot was probed by biotinylated anti-IL-15 antibody and subsequently with streptavidin-HRP. FIG. 29A: in vitro stability of P1482 in human serum. FIG. 29B: in vitro stability of P1482 in mouse serum. FIG. 29C: in vitro protein stability in human serum at the time point of 164 h. FIG. 29D: in vitro protein stability in mouse serum at the time point of 96 h. FIG. 29E: in vitro protein stability in rat serum at the time point of 96 h. FIG. 29F: in vitro protein stability in cyno serum at the time point of 96 h.

FIGS. 30A-30B show SDS-PAGE results of purified proteins. FIG. 30A shows reduced SDS-PAGE results and 30B shows non-reduced SDS-PAGE results. “M” on the figures represents the protein standard marker.

FIGS. 31A-31F illustrate representative HPLC analysis results of purified proteins.

FIG. 32 shows SDS-PAGE results of cleavage of IL-15 fusion proteins. “M” on the figures represents the protein standard marker. “B” represents for protein before protease cleavage. “uPA” represents for uPA protease. “Mat” represents for matriptase protease. “L” represents for legumain protease. “M-2” represents for MMP-2 protease. “K5” represents for KLK-5 protease. “K7” represents for KLK-7 protease.

FIGS. 33A-33J show activity of IL-15 fusion proteins on M-07e proliferation determined by a colorimetric assay (Cell Counting Kit-8 (CCK-8)).

FIGS. 34A-34B illustrates representative results of in vitro serum stability. Western blot was probed using HRP-anti-human IgG antibody. FIG. 34A: in vitro stability of P18121450 in mouse serum. FIG. 34B: in vitro stability of P18121450 in rat serum.

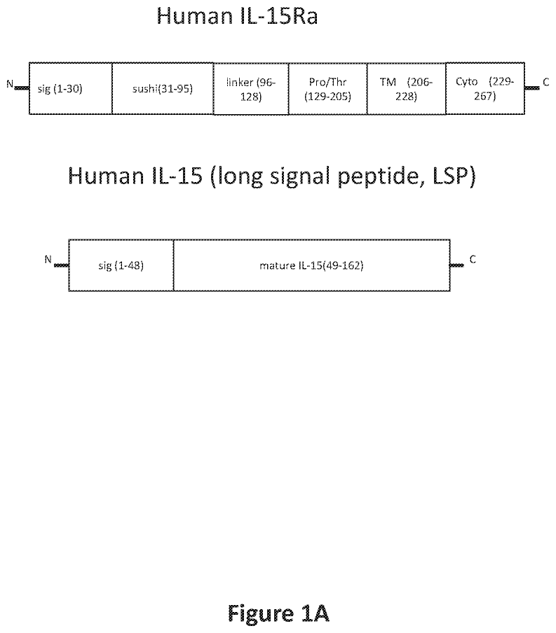

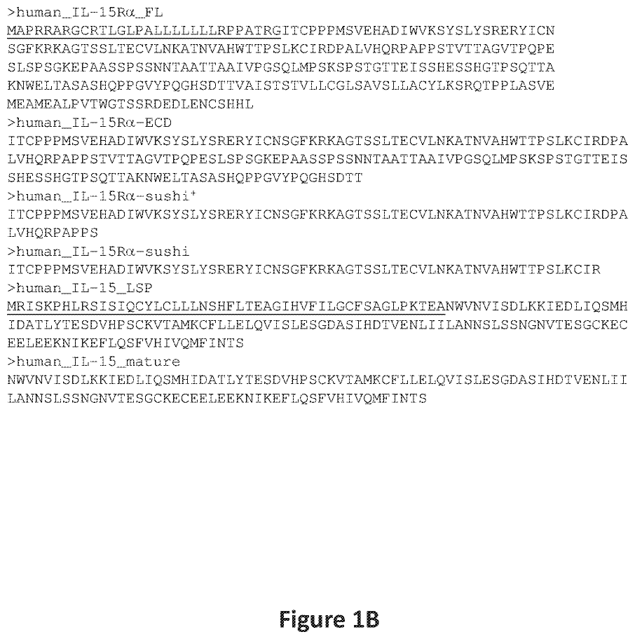

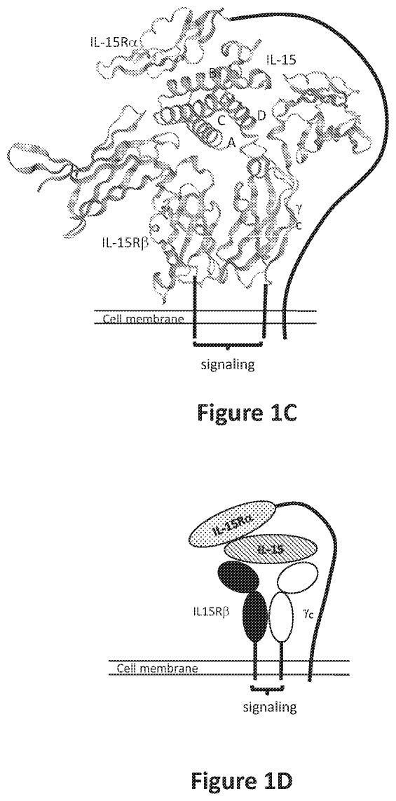

### DETAILED DESCRIPTION

The current invention provides activatable proproteins comprising a masking moiety and an active portion, which is also interchangeably referred to herein as an “active domain”, wherein the active domain comprises a complex of IL-15/IL-15Rα. The invention further provides compositions comprising the same and methods of administering or using the same for preventing or treating a tumor or cancer.

### Definitions

Unless defined otherwise, technical and scientific terms used herein have the same meaning as commonly understood by one of ordinary skill in the art to which this invention belongs.

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

The term “active,” as used herein, refers to a portion, domain or fragment having a biological activity or biological function. In one embodiment, the term “active domain” or “active portion” are used interchangeably herein within the context of an activatable protein disclosed herein. In some embodiments, the activity is equal to or approximates the activity of the wild-type protein. Hence, an active domain of an activatable proprotein, comprising an IL-15/IL-15Rα complex as described herein, is expected to have equal activity or approximates the activity of the wild-type IL-15/IL-15Rα complex whether in soluble form or when bound to a cell surface.

The term “subject” as used herein includes, but is not limited to, a mammal, including, e.g., a human, non-human primate (e.g., monkey), mouse, pig, cow, goat, rabbit, rat, guinea pig, hamster, horse, monkey, sheep, or other non-human mammal, a non-mammal, including, e.g., a non-mammalian vertebrate, such as a bird (e.g., a chicken or duck) or a fish; and a non-mammalian invertebrate. In some embodiments, the methods and compositions of the invention are used to treat (both prophylactically and/or therapeutically) non-human animals.

The term “pharmaceutical composition” herein means a composition suitable for pharmaceutical use in a subject, including an animal or human. A pharmaceutical composition generally comprises an effective amount of an active agent (e.g., the activatable proprotein provided herein) and a pharmaceutically acceptable carrier (e.g., a buffer, adjuvant, or the like).

The term “effective amount” means a dosage or amount sufficient to produce a desired result. The desired result may comprise an objective or subjective improvement in the recipient of the dosage or amount (e.g., long-term survival, decrease in number and/or size of tumors, effective prevention of a disease state, etc.).

A “prophylactic treatment” is a treatment administered to a subject who does not display signs or symptoms of a disease, pathology, or medical disorder, or displays only early signs or symptoms of a disease, pathology, or disorder, such that treatment is administered for the purpose of diminishing, preventing, or decreasing the risk of developing the disease, pathology, or medical disorder. A prophylactic treatment functions as a preventative treatment against a disease or disorder. A “prophylactic activity” is an activity of an agent, such as the activatable proprotein disclosed herein, or composition thereof, that, when administered to a subject who does not display signs or symptoms of a pathology, disease or disorder (or who displays only early signs or symptoms of a pathology, disease, or disorder) diminishes, prevents, or decreases the risk of the subject developing the pathology, disease, or disorder. A “prophylactically useful” agent or compound (e.g., an activatable proprotein provided herein) refers to an agent or compound that is useful in diminishing, preventing, treating, or decreasing development of a pathology, disease or disorder.

A “therapeutic treatment” is a treatment administered to a subject who displays symptoms or signs of pathology, disease, or disorder, in which treatment is administered to the subject for the purpose of diminishing or eliminating those signs or symptoms of pathology, disease, or disorder. A “therapeutic activity” is an activity of an agent, such an activatable proprotein provided herein, or a composition thereof, that eliminates or diminishes signs or symptoms of a pathology, disease or disorder, when administered to a subject suffering from such signs or symptoms. A “therapeutically useful” agent or compound (e.g., an activatable proprotein provided herein) indicates that an agent or compound is useful in diminishing, treating, or eliminating such signs or symptoms of the pathology, disease or disorder.

The term “treating” as used herein, unless otherwise indicated, means reversing, alleviating, or reducing the reoccurrence of, either partially or completely, the growth of tumors, tumor metastases, or other cancer-causing or neoplastic cells in a patient. The term “treatment” as used herein, unless otherwise indicated, refers to the act of treating a disease, such as a cancer or tumor.

The term “preventing” as used herein, unless otherwise indicated, means inhibiting the progress of, delaying the onset of, or reducing the occurrence of, either partially or completely, the growth of tumors, tumor metastases, or other cancer-causing or neoplastic cells in a patient. The term “preventing” as used herein, unless otherwise indicated, refers to the act of preventing a disease, such as a cancer or tumor.

The terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence. To determine the percent identity, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In some embodiments, the two sequences are the same length.

The term “substantially identical,” in the context of two nucleic acids or polypeptides, refers to two or more sequences or subsequences that have at least 90%, at least 91%, at least 92%, at least 93%, at least 94%, at least 95%, at least 96%, at least 97%, at least 98% identity, or at least 99% identity (e.g., as determined using one of the methods set forth infra).

As used herein, the term “binds,” “specifically binds to,” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, for example, by a radioimmunoassay (RIA). In certain embodiments, an antibody that specifically binds to a target has a dissociation constant (Kd) of <1 μM, <100 nM, <10 nM, <1 nM, or <0.1 nM.

As used in this specification, the singular forms “a”, “an”, and “the” include plural references unless the context clearly dictates otherwise. Reference to “the formulation” or “the method” includes one or more formulations, methods, and/or steps of the type described herein and/or which will become apparent to those persons skilled in the art upon reading this disclosure.

The terms “activatable proprotein,” “activatable prodrug”, “prodrug” or “proprotein” are used interchangeably herein and refer to an activatable proprotein comprising at least a masking moiety and an active domain, or derivatives/variants therefrom, as described herein. In one embodiment, the proprotein may also comprise one or more protein domains.

The term “polypeptide” refers, in one embodiment, to a polymer of amino acids and its equivalent and does not refer to a specific length of a product; thus, “peptides” and “proteins” are included within the definition of a polypeptide. Also included within the definition of polypeptides are “masking moieties” as defined herein. A “polypeptide region” refers to a segment of a polypeptide, which segment may contain, for example, one or more domains or motifs (e.g., a polypeptide region of an antibody can contain, for example, one or more constant domains). The term “fragment” refers to a portion of a polypeptide preferably having at least 10 contiguous amino acids of the polypeptide. In another embodiment, the term refers to a portion of a polypeptide having least 20, 30, 40, 50, 60, or 70 contiguous amino acids of the polypeptide.

Unless otherwise indicated by context, a “derivative” is a polypeptide or fragment thereof having one or more non-conservative or conservative amino acid substitutions relative to another polypeptide (also referred to as a “variant”); or a polypeptide or fragment thereof that is modified by covalent attachment of another molecule such as, e.g., by attachment of a heterologous polypeptide, or by glycosylation, acetylation, phosphorylation, and the like. Further included within the definition of “derivative” are, for example, polypeptides containing one or more analogs of an amino acid (e.g., unnatural amino acids and the like), polypeptides with unsubstituted linkages, as well as other modifications known in the art, both naturally and non-naturally occurring.

An “isolated” polypeptide is one which has been identified and separated and/or recovered from a component of its natural environment. Contaminant components of its natural environment are materials which would interfere with diagnostic or therapeutic uses for the polypeptide, and may include enzymes, hormones, and other proteinaceous or nonproteinaceous solutes. An isolated polypeptide includes an isolated antibody, or a fragment or derivative thereof.

The term “antibody” refers to a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (e.g., antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains, respectively.

Antibodies exist as intact immunoglobulins or as a number of well-characterized fragments produced by digestion with various peptidases. Thus, for example, pepsin digests an antibody below the disulfide linkages in the hinge region to produce F(ab′)2, a dimer of Fab which itself is a light chain joined to VH-CH1 by a disulfide bond. The F(ab′)2 may be reduced under mild conditions to break the disulfide linkage in the hinge region thereby converting the F(ab′)2dimer into an Fab′ monomer. The Fab′ monomer is essentially an Fab with part of the hinge region (see, Fundamental Immunology, W. E. Paul, ed., Raven Press, New York (1999), for a more detailed description of other antibody fragments). While various antibody fragments are defined in terms of the digestion of an intact antibody, one of skill will appreciate that such Fab′ fragments, etc. may be synthesized de novo either chemically or by utilizing recombinant DNA methodology. Thus, the term antibody, as used herein also includes antibody fragments either produced by the modification of whole antibodies or synthesized de novo using recombinant DNA methodologies. Antibodies include single chain antibodies, including single chain Fv (sFv or scFv) antibodies in which a variable heavy and a variable light chain are joined together (directly or through a peptide linker) to form a continuous polypeptide.

In one embodiment, the terms “antibody portion” and “antibody domain” are used interchangeably herein and refer to a portion/domain of an antibody used to create a masking moiety described herein.

The term “about” as used herein means in quantitative terms plus or minus 5%, or in another embodiment plus or minus 10%, or in another embodiment plus or minus 15%, or in another embodiment plus or minus 20%.

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned herein are incorporated herein by reference for the purpose of disclosing and describing material for which the reference was cited in connection with.

### Activatable Proproteins

In one embodiment, an activatable proprotein provided herein comprises a masking moiety linked to an active domain. In another embodiment, the activatable proprotein is a dimer. In one embodiment, the active domain comprises a dimer complex of IL-15/IL-15Rα. It is to be understood that the terms “complex of IL-15/IL-15Rα”, and “IL-15/IL-15Rα complex” are used interchangeably herein and refer to a monomer that joins to another monomer complex of IL-15/IL-15Rα via one or more linkers and/or one or more disulfide bonds and/or one or more non-covalent bonds, to form a dimer that makes up the active domain of an activatable proprotein described herein.

In one embodiment, an activatable proprotein provided herein comprises a first polypeptide and a second polypeptide, wherein the first and second polypeptide each comprise a masking moiety operably linked via a first linker on the C-terminus to an IL-15 or variant thereof, wherein the IL-15 or variant thereof is linked via a second linker on the C-terminus to an IL-15Rα or variant thereof, and wherein the masking moiety masks the active portion of the proprotein. In one embodiment, such an activatable proprotein comprises the format of Fc-IL-15-linker-IL-15Rα (Fc-ILR), where “Fc” corresponds to the masking domain. In another embodiment, a homodimer of an activatable proprotein comprises an amino acid sequence set forth in SEQ ID NO: 26.

Fc-GS-ENLYFQG-GS-IL-15-(GGS)5-IL-15Rα (homodimer)
(SEQ ID NO: 26)
EPKSSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK [symbol] NW
VNVISDLKKIEDLIQSMHIDATLYTESDVHPSCKVTAMKCFLLELQVIS
LESGDASIHDTVENLIILANNSLSSNGNVTESGCKECEELEEKNIKEFL
QSFVHIVQMFINTS [symbol] ITCPPPMSVEHADIWVKSYS
LYSRERYICNSGFKRKAGTSSLTECVLNKATNVAHWTTPSLKCIRDPAL
VHQRPAPPS.

In one embodiment, an activatable proprotein disclosed herein comprises SEQ ID NO: 26. In another embodiment, an activatable proprotein disclosed herein consists of SEQ ID NO: 26. In another embodiment, an activatable proprotein disclosed herein comprises a variant or homolog of SEQ ID NO: 26.

In one embodiment, provided is a cleavable linker comprising a protease cleavage site. An exemplary cleavable linker comprises a protease (TEV) cleavage site, which is demonstrated in bold and italics in SEQ ID NO: 26 above and is represented by SEQ ID NO: 344; the italicized and underlined sequences in SEQ ID NO: 26 above represent linker sequences surrounding the TEV cleavage site. It is to be understood that any cleavage site from any protease disclosed herein or available in the art may be inserted in a cleavable linker that links any of the components of the activatable proprotein, as further described herein. In one embodiment, the sequence of the proprotein, without a protease cleavage site comprises P1187 (SEQ ID NO: 18), P1188 (SEQ ID NO: 19), P1250 (SEQ ID NO: 20), P1251 (SEQ ID NO: 21), and P1252 (SEQ ID NO: 22), P1254 (SEQ ID NO: 24), P1255 (SEQ ID NO: 25), P1279 (SEQ ID NO: 41) and P1280 (SEQ ID NO: 42), see Example 1 and sequences that follow herein.

In a related embodiment, the activatable proprotein is a dimeric proprotein, where, in another embodiment, the dimeric proprotein comprises a first and second polypeptide each comprising an IL-15 or variant thereof operably linked via a first linker on the C-terminus to an IL-15Rα or variant thereof, wherein the IL-15Rα or variant thereof is linked via a second linker on the C-terminus to a masking moiety present on each of the first and second polypeptides, and wherein the masking moiety masks the active portion of the proprotein, and wherein the masking moiety of the first polypeptide forms one or more covalent disulfide bonds or one or more non-covalent bonds to the masking moiety of the second polypeptide. In one embodiment, such an activatable proprotein comprises the format of IL-15-linker-IL-15Rα-Fc (ILR-Fc), where “Fc” corresponds to the masking domain.

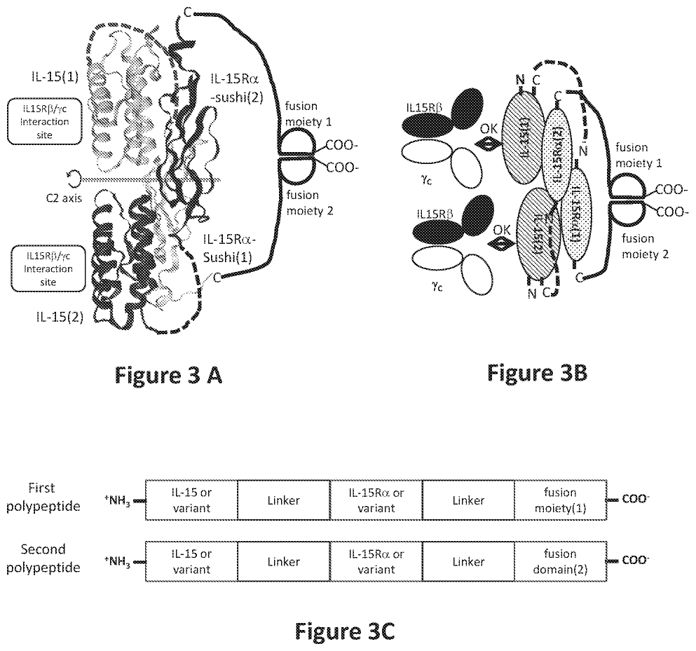

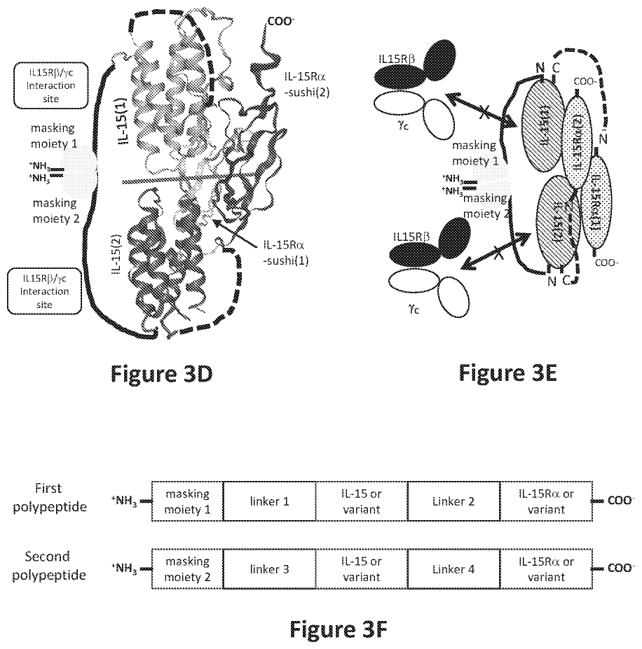

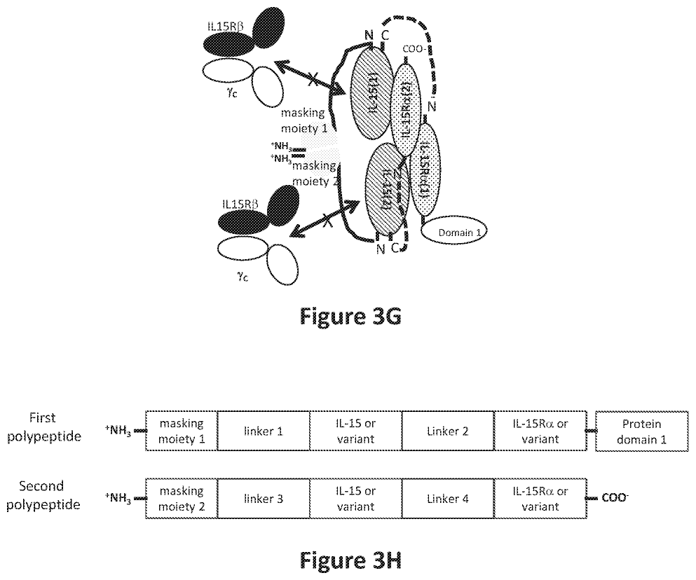

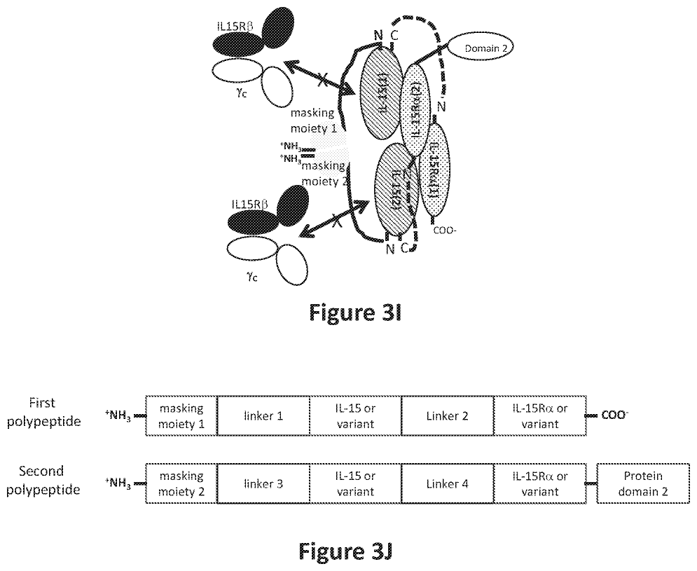

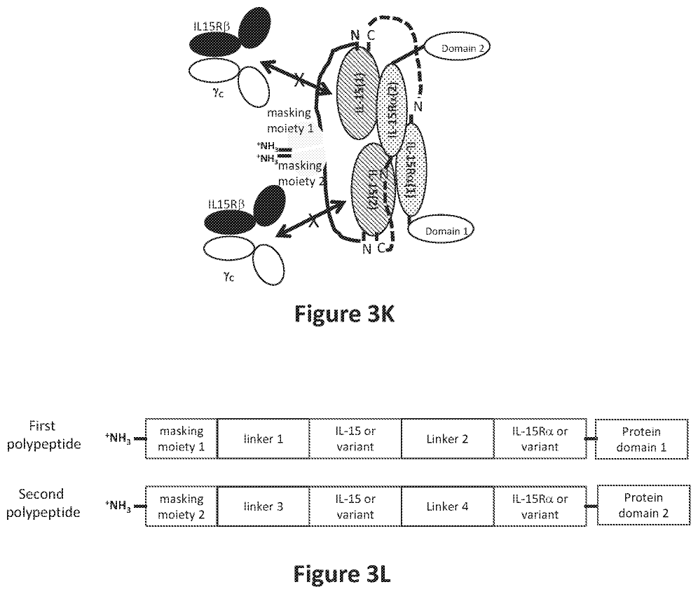

In one embodiment, the Fc-ILR form of the activatable proprotein comprises one or more protein domains attached to the C-terminal of IL-15Rα on the first polypeptide, the second polypeptide, or both the first and second polypeptides of the activatable proprotein (see for e.g. FIG. 3G-FIG. 3L).

In one embodiment, an activatable proprotein provided herein is designed to be activated preferentially inside tumor or cancer tissue (not in normal tissues).

In one embodiment, provided herein is an activatable proprotein comprising a first polypeptide and a second polypeptide, wherein the first and second polypeptide each comprise an IL-15 or variant thereof operably linked via a first linker on the C-terminus to an IL-15Rα or variant thereof, wherein the IL-15Rα or variant thereof is linked via a second linker on the C-terminus to a masking moiety present on each of the first and second polypeptides, and wherein the masking moiety masks the active portion of the proprotein. In another embodiment, such an activatable proprotein comprises SEQ ID NO: 17 or SEQ ID NO: 73 (see also FIG. 3B herein).

In another embodiment, an activatable proprotein disclosed herein is a dimeric proprotein. In another embodiment, an activatable proprotein disclosed herein is a dimeric fusion protein. In another embodiment, the dimeric proprotein comprises a first polypeptide and a second polypeptide each comprising from N-terminus to C-terminus the IL-15 or variant thereof linked via the first linker on the C-terminus to the IL-15Rα or variant thereof, wherein the IL-15Rα or variant thereof is linked via the second linker on the C-terminus to the masking moiety, and wherein the masking moiety of the first polypeptide forms one or more covalent disulfide bonds or non-covalent bonds with the masking moiety of the second polypeptide.

In another embodiment an activatable proprotein is a heterodimeric protein comprising heterodimeric masking moieties. In another embodiment, an activatable proprotein is a heterodimeric protein comprising heterodimeric active domain moieties. In another embodiment, an activatable proprotein provided herein is a heterodimeric protein comprising heterodimeric masking moieties and active domain moieties (see Figures herein).

### Activatable Proprotein—Active Domain

In one embodiment, the active domain of the activatable proprotein comprises a IL-15/IL-15Rα complex or variant thereof. In one embodiment, upon protease cleavage and exposure of the active domain in an activatable proprotein disclosed herein, IL-15 on the IL-15/IL-15Rα complex binds IL-15Rβ/γC on a cell surface of a lymphocyte of blood cell to activate a series of downstream pathways that lead to increased cellular growth, decreased apoptosis, enhanced immune cell activation and migration. Indeed, upon ligand binding, the IL-2/15Rβ and γC subunits to stimulate Janus kinase (Jak)1, Jak3, and signal transducer and activator of transcription (STAT)-5 pathway. After phosphorylation, STAT5 homo-dimerizes, translocates to the nucleus, and promotes the transcription of target genes. Additionally, IL-15 stimulates both the PI3K-AKT and RAS-MAPK pathways. Altogether, IL-15 signaling stimulates an array of downstream pathways leading to responses that have a crucial role in the development, function, and survival of CD8 T cells, NK cells, NKT cells and intestinal intraepithelial lymphocytes.

In some embodiments, the IL-15 or variant thereof is a human IL-15 or an amino acid mutant derived therefrom. In another embodiment, the IL-15Rα or variant thereof comprises a human IL-15Rα, a truncated human IL-15Rα or an amino acid mutant derived from human IL-15Rα. In another embodiment, a IL-15/IL-15Rα complex or variant thereof disclosed herein as part of the activatable proprotein comprises a human IL-15 or an amino acid mutant derived therefrom, and a human IL-15Rα, a truncated human IL-15Rα or an amino acid mutant derived from human IL-15Rα.

In one embodiment, the precursor amino acid sequence of human IL-15 comprises or consists of SEQ ID NO: 1, (See examples below; underlined sequence corresponds to the signal sequence or signal peptide). In another embodiment, the underlined sequence in SEQ ID NO: 1 corresponds to the signal sequence or signal peptide. In one embodiment, the mature amino acid sequence of human IL-15 with a point mutation of S162A comprises or consists of SEQ ID NO: 3.

In another embodiment, a human IL-15 amino acid sequence comprises a variant or homolog of SEQ ID NO: 1, SEQ ID NO: 2 or SEQ ID NO: 3.

In one embodiment, the full-length amino acid sequence of IL-15Rα comprises or consists of SEQ ID NO: 4 (See Examples below; underlined sequence corresponds to the signal sequence or signal peptide). In another embodiment, the IL-15Rα extracellular domain (ECD) comprises or consists of SEQ ID NO: 5. In another embodiment, the IL-15Rα sushi+ domain comprises or consists of SEQ ID NO: 6. In another embodiment, the IL-15Rα, sushi domain comprises or consists of SEQ ID NO: 7.

In one embodiment, the IL-15 or variant thereof and the IL-15Rα or variant thereof in the first polypeptide and the IL-15 or variant thereof and the IL-15Rα or variant thereof in the second polypeptide comprise one or more Cys substitution mutations. Having these mutations facilitates binding of the IL-15/IL-15 Rα complex on the first polypeptide with the IL-15/IL-15 Rα complex on the second polypeptide through disulfide bonds.

In another embodiment, the IL-15 or variant thereof in the first polypeptide comprising one or more Cys substitution mutations forms a disulfide bond with the IL-15Rα or variant thereof comprising one or more Cys substitution mutations in the second polypeptide. In another embodiment, the IL-15 or variant thereof in the second polypeptide comprising one or more Cys substitution mutations forms a disulfide bond with the IL-15Rα or variant thereof comprising one or more Cys substitution mutations in the first polypeptide.

### Activatable Proprotein—Linkers

In one embodiment, the first linker on the first or second polypeptide of an activatable proprotein disclosed herein, or on both the first and the second polypeptide of the same is a cleavable linker. In another embodiment, the second linker on the first or second polypeptide of an activatable proprotein disclosed herein, or on both the first and the second polypeptide of the same is a cleavable linker.

The term “linker” is art-recognized and refers to a molecule (including but not limited to unmodified or modified nucleic acids or amino acids) or group of molecules (for example, 2 or more, e.g., 2, 3, 4, 5, 6, 7, 8, 9, 10, 15, 20, 25, 30, 35, 40, 45, 50, 55, 60, 65, 70, 75, 80, 85, 90, 95, 100 or more) connecting two compounds, such as two polypeptides. The linker may be comprised of a single linking molecule or may comprise a linking molecule and at least one spacer molecule, intended to separate the linking molecule and a compound by a specific distance.

In one embodiment, the linkers include cleavable or non-cleavable linkers. Examples of a cleavable linker includes a linker have a cleavage site for a protease, for e.g. a TEV protease. Examples of a non-cleavable linker include a linker that is removed or degraded intracellularly or not cleaved.

In one embodiment, the activatable proprotein described herein and pharmaceutical compositions comprising the same can be used to directly target cancer cells so that the activatable proprotein is essentially activated when the a protease cleavable linker is cleaved so as to provide the active payload or when the non-cleavable linker is degraded or removed intracellularly by the targeted cell type.

In one embodiment, a non-cleavable linker is used to link any of the components of an activatable proprotein described herein. A non-limiting example of a non-cleavable linker comprises or consists of an amino acid sequence of GGSGGSGGSGGSGGS (SEQ ID NO: 345), or a homolog or variant thereof. It is to be understood that any non-cleavable linker known in the art may be used and is not limited to those disclosed herein.

In one embodiment, a linker used in the activatable proprotein described herein comprises an immunoglobulin (Ig)/antibody hinge region. In one embodiment, the hinge region is obtained from an IgG1 antibody. In one embodiment, the term Ig “hinge” region refers to a polypeptide comprising an amino acid sequence that shares sequence identity, or similarity, with a portion of a naturally-occurring Ig hinge region sequence, which includes the cysteine residues at which the disulfide bonds link the two heavy chains of the immunoglobulin. Sequence similarity of the hinge region linkers of the present invention with naturally-occurring immunoglobulin hinge region amino acid sequences can range from at least 50% to about 75-80%, and typically greater than about 90%.

Derivatives and analogs of the hinge region can be obtained by mutations. A derivative or analog, as referred to herein, is a polypeptide comprising an amino acid sequence that shares sequence identity, or similarity, with the full-length sequence of the wild type (or naturally occurring protein), except that it has one or more amino acid sequence differences attributable to a deletion, insertion and/or substitution.

The present invention also encompasses fragments of the hinge region for use as linkers. Such a fragment need only be long enough to allow the proteins attached by the hinge region fragment to attain a biologically active conformation. In one embodiment, the heterodimeric masking moiety of the activatable proprotein provided herein is joined to the heterodimeric active domain through fusion using an Ig hinge region. In one embodiment, the masking moiety and the active domain are homodimeric.

In one embodiment, a cleavable linker comprises or consists of a protease-cleavage site, flanked by linker sequences. In one embodiment, non-limiting example of a cleavable linker comprises or consists of an amino acid sequence of GSENLYFQGGS (SEQ ID NO: 346), or a homolog or variant thereof. It is to be understood that any cleavable linker known in the art may be used and is not limited to those disclosed herein. Cleavage sites for proteases disclosed herein are known in the art and are contemplated for use with any of the proteases disclosed herein or proteases available in the art.

In one embodiment, non-cleavable or cleavable linkers are inserted in an activatable proprotein (in the order of C-terminus to N-terminus) between the masking moiety and IL-15 and between IL-15 and IL-15Rα of an activatable proprotein disclosed herein. In another embodiment, non-cleavable or cleavable linkers are inserted in an activatable proprotein (in the order of C-terminus to N-terminus) between IL-15 and IL-15Rα and between IL-15Rα and a masking moiety of an activatable proprotein disclosed herein. In one embodiment, the cleavable linker(s) within the activatable proprotein comprises a cleavage site for a protease. In another embodiment, a first linker and/or a second linker of an activatable proprotein disclosed herein is a cleavable linker that comprises a cleavage site for a protease. In another embodiment, the protease is selected from a metalloprotease, a serine protease, a cysteine protease, an aspartic acid protease or any combination thereof. In another embodiment, the protease cleavage site is cleavable by a MMP1, MMP2, MMP3, MMP4, MMP5, MMP6, MMP7, MMP8, MMP9, MMP10, MMP11, MMP12, MMP13, MMP14, TEV, matriptase, uPA, FAP, Legumain, PSA, Kallikrein, Cathepsin A, or a Cathepsin B protease, or any protease available in the art. In one embodiment, the protease is any protease known in the art and is not limited to one disclosed herein.

In one embodiment, following a complete protease (e.g.—TEV protease) cleavage of a homodimer of an activatable proprotein disclosed herein, the activatable proprotein is cleaved into two cleaved products each comprising two chains:

Cleaved Product 1) Fc-GSENLYFQ Homodimer (Product from Complete Cleavage) (SEO ID NO: 27)

Chains 1 and 2 (Each Having the Same Sequence)

(SEQ ID NO: 27)
EPKSSDKTHTCPPCPAPELLGGPSVFLFPPKPKDTLMISRTPEVTC
VVVDVSHEDPEVKFNWYVDGVEVHNAKTKPREEQYNSTYRVVSVLT
VLHQDWLNGKEYKCKVSNKALPAPIEKTISKAKGQPREPQVYTLPP
SREEMTKNQVSLTCLVKGFYPSDIAVEWESNGQPENNYKTTPPVLD
SDGSFFLYSKLTVDKSRWQQGNVFSCSVMHEALHNHYTQKSLSLSP
GK [symbol] .

In one embodiment, SEQ ID NO: 27 comprises the masking moiety following protease cleavage of the activatable proprotein.

Cleaved Product 2) GGS-IL-15-(GGS)5-IL-15Rα Homodimer (Product from Complete Cleavage, Active Domain) (SEO ID NO: 28)

Chains 1 and 2 (Each Having the Same Sequence)

(SEQ ID NO: 28) [symbol]
NWVNVISDLKKIEDLIQSMHIDATLYTESDVHPSCKVTAMKCF
LLELQVISLESGDASIHDTVENLIILANNSLSSNGNVTESGCKECE
ELEEKNIKEFLQSFVHIVQMFINTS [symbol] [symbol] ITCPPP
MSVEHADIWVKSYSLYSRERYICNSGFKRKAGTSSLTECVLNKATN
VAHWTTPSLKCIRDPALVHQRPAPPS.

In one embodiment, SEQ ID NO: 28 comprises the active domain of the activatable proprotein following protease cleavage.

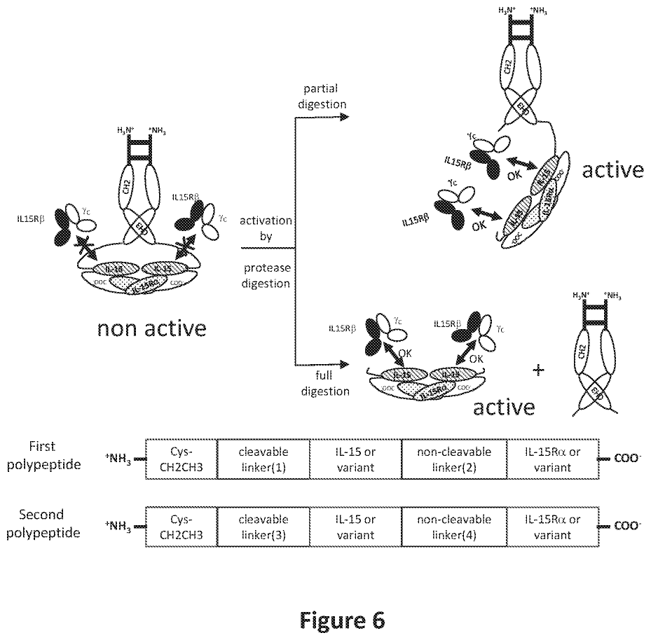

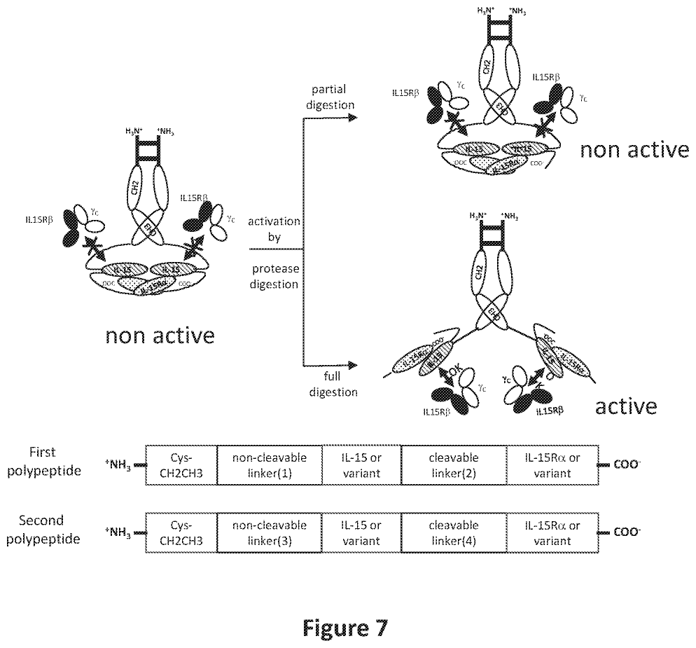

In one embodiment, following a partial protease cleavage (e.g.—using TEV protease) of a homodimer of an activatable proprotein disclosed herein, the masking moiety in the activatable proprotein is partially removed allowing access of the active domain (IL-15/IL-15Rα complex) to binding by IL-15Rβ/γC (see FIG. 6). In another embodiment, the partially cleaved activatable proprotein comprises SEQ ID NO: 29, 30 and 31.

Cleaved Product 3) Fc-GS-ENLYFQG-GS-IL-15-(GGS)5-IL-15Rα/Fc-GSENLYFQ/GGS-IL-15-(GGS)5-IL-15Rα (Product from Partial Cleavage. Active)(SEO ID NO:29, 30 and 31).

Chain 1: Fc-GS-ENLYFQG-GS-IL-15-(GGS)5-IL-15Rα
(SEQ ID NO: 29)
EPKSSDKTHTCPPCPAPELLGGPSVFLFPPKPKDTLMISRTPEVTC
VVVDVSHEDPEVKFNWYVDGVEVHNAKTKPREEQYNSTYRVVSVLT
VLHQDWLNGKEYKCKVSNKALPAPIEKTISKAKGQPREPQVYTLPP
SREEMTKNQVSLTCLVKGFYPSDIAVEWESNGQPENNYKTTPPVLD
SDGSFFLYSKLTVDKSRWQQGNVFSCSVMHEALHNHYTQKSLSLSP
GK [symbol] NWVNVISDLKKIEDLIQSMHIDATLYTESDVHP
SCKVTAMKCFLLELQVISLESGDASIHDTVENLIILANNSLSSNGN
VTESGCKECEELEEKNIKEFLQSFVHIVQMFINTS [symbol] [symbol]
ITCPPPMSVEHADIWVKSYSLYSRERYICNSGFKRKA
GTSSLTECVLNKATNVAHWTTPSLKCIRDPALVHQRPAPPS
Chain 2: Fc-GSENLYFQ
(SEQ ID NO: 30)
EPKSSDKTHTCPPCPAPELLGGPSVFLFPPKPKDTLMISRTPEVTC
VVVDVSHEDPEVKFNWYVDGVEVHNAKTKPREEQYNSTYRVVSVLT
VLHQDWLNGKEYKCKVSNKALPAPIEKTISKAKGQPREPQVYTLPP
SREEMTKNQVSLTCLVKGFYPSDIAVEWESNGQPENNYKTTPPVLD
SDGSFELYSKLTVDKSRWQQGNVFSCSVMHEALHNHYTQKSLSLSP
GK [symbol] .
Chain 3: GGS-IL-15-(GGS)5-IL-15Rα
(SEQ ID NO: 31) [symbol]
NWVNVISDLKKIEDLIQSMHIDATLYTESDVHPSCKVTAMKCF
LLELQVISLESGDASIHDTVENLIILANNSLSSNGNVTESGCKECE
ELEEKNIKEFLQSFVHIVQMFINTS [symbol] [symbol] ITCPPP
MSVEHADIWVKSYSLYSRERYICNSGFKRKAGTSSLTECVLNKATN
VAHWTTPSLKCIRDPALVHQRPAPPS.

Linkers suitable for use in compositions described herein are generally ones that provide flexibility of the masking moiety or activatable proprotein to facilitate the inhibition of the binding active portion of the activatable proprotein, namely the IL-15/IL-15Rα complex. Such linkers are generally referred to as flexible linkers. Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids. In one embodiment, the linker is 26 amino acids long. In another embodiment, the linker is 5-9, 10-15, 16-20, 21-25, 26-30, 31-35, 36-40, 41-45, or 46-50 amino acids long.

Exemplary flexible linkers include glycine polymers (G)n, glycine-serine polymers (including, for example, (GS)n, (GSGGS)n, and (GGGS)n, where n is an integer of at least one), glycine-alanine polymers, alanine-serine polymers, and other flexible linkers known in the art, see e.g. U.S. Pat. No. 10,059,762 B2, which is incorporated by reference herein in its entirety. Glycine and glycine-serine polymers are relatively unstructured, and therefore may be able to serve as a neutral tether between components. Glycine accesses significantly more phi-psi space than even alanine, and is much less restricted than residues with longer side chains (see Scheraga, Rev. Computational Chem. 11173-142 (1992)). The ordinarily skilled artisan will recognize that design of an activatable proprotein can include linkers that are all or partially flexible, such that the linker can include a flexible linker as well as one or more portions that confer less flexible structure to provide for a desired activatable proprotein structure.

In one embodiment, protease cleavage of a cleavable linker in a first or second polypeptide partially removes the masking moiety in the activatable proprotein such that the IL-15/IL-15Rα complex in the first or second polypeptide can bind IL-15Rβ/γC present on the surface of a lymphocyte or a blood cell in vivo. In one embodiment, protease cleavage of a cleavable linker in a first and second polypeptide completely removes the masking moiety in the activatable proprotein such that the IL-15/IL-IL-15Rα complex in the first and the second polypeptide can bind IL-15Rβ/γC present on the surface of a lymphocyte or a blood cell in vivo.

In one embodiment, the linker may comprise a spacer element and a cleavable element so as to make the cleavable element more accessible to the enzyme responsible for cleavage.

### Activatable Proprotein—Masking Moiety

In one embodiment, an activatable proprotein of the invention comprises a masking moiety dimer that masks the active domain. Hence, within the context of an activatable proprotein provided herein, when the active domain comprising an IL-15/IL-IL-15Rα complex is modified by the addition of a masking moiety (via one or more cleavable linkers) and is in the presence of the target (IL-15Rβ/γC), specific binding of the active domain to its target is blocked, reduced or inhibited, as compared to the specific binding of the active domain not modified by the addition of a masking moiety. In another embodiment, when the active domain comprising an IL-15/IL-IL-15Rα complex is modified by the addition of a masking moiety (for example, via one or more cleavable linkers) and is in the presence of the target (IL-15Rβ/γC), specific binding of the active domain to its target is blocked, reduced or inhibited, as compared to the specific binding of the active proprotein in the presence of sufficient protease enzyme or protease enzyme activity to partially or completely cleave the masking moiety from the activatable proprotein. In some embodiments, protease cleavage of linkers between the masking moiety and the active domain in the activatable proprotein leads to activation (“frees up”) the active domain. This activation preferably occurs at a tumor site or in a cancer tissue.

In one embodiment, the masking moiety is fused to an active domain to form a fusion protein. In one embodiment, the fusion protein is recombinantly generated. In another embodiment, the fusion protein is chemically generated (i.e.—chemically fused).

In one embodiment, the masking moiety specific binds to the active domain. In another embodiment, the masking moiety lacks the ability to specifically bind to the active domain. In one embodiment, the masking moiety and the linkers between the ILR and the masking moiety sterically hinder the ability of IL-15 in the active domain to bind to and signal through IL-15Rβ/γc.

In one embodiment, a masking moiety of an activatable proprotein disclosed herein comprises the amino acid sequence set forth in SEQ ID NO: 8, SEQ ID NO: 9, SEQ ID NO: 10 or SEQ ID NO: 11. In another embodiment, a masking moiety of an activatable proprotein disclosed herein comprises an amino acid sequence selected from the group consisting of SEQ ID NO: 8, SEQ ID NO: 9, SEQ ID NO: 10 and SEQ ID NO: 11.

In one embodiment, the masking moiety can sterically inhibit the binding of the active domain of the activatable proprotein to a target, wherein in another embodiment, the target is IL-15Rβ/γC. In another embodiment, the masking moiety can allosterically inhibit the binding of the activatable proprotein to its target. In these embodiments when the active domain is modified by coupling to a masking moiety (to form an activatable proprotein as described herein) and in the presence of target, there is no binding or substantially no binding of the active domain to the target, or no more than 0.001%, 0.01%, 0.1%, 1%, 2%, 3%, 4%, 5%, 6%, 7%, 8%, 9%, 10%, 15%, 20%, 25%, 30%, 35%, 40%, or 50% binding of the active domain to the target, as compared to the binding of the active domain not modified with a masking moiety or the binding of the active domain not coupled to a masking moiety for at least 2, 4, 6, 8, 12, 28, 24, 30, 36, 48, 60, 72, 84, 96 hours, or 5, 10, 15, 30, 45, 60, 90, 120, 150, 180 days, or 1, 2, 3, 4, 5, 6, 7, 8, 9, 10, 11, 12 months or greater when measured in vivo or in a Target Displacement in vitro assay available in the art.

In one embodiment, when the active domain is in a ‘masked’ state, even in the presence of a target the masking moiety interferes with or inhibits the binding of the active domain to the target. However, in the unmasked state of the active domain, the masking moiety's interference with target binding to the active domain is reduced, thereby allowing greater access of the active domain to the target and providing for target binding.

In one embodiment, the active domain in the activatable proprotein can be unmasked upon cleavage, in the presence of enzyme, where in a preferred embodiment, the enzyme is a disease-specific enzyme, such as a cancer-specific or a tumor-specific protease. Thus, the masking moiety is one that when the active domain within the activatable proprotein is uncleaved provides for masking of the active domain from target binding, but does not substantially or significantly interfere or compete for binding of the target to the active domain when the activatable proprotein has been cleaved. Thus, the activatable proprotein facilitates the switchable/activatable phenotype, with the masking moiety decreasing binding of target when the activatable proprotein is uncleaved, and with cleavage of the masking moiety by protease providing for increased binding of target by the active domain.

In one embodiment, the structural properties of the masking moiety will vary according to a variety of factors such as the minimum amino acid sequence required for interference with active domain binding to target, the length of the linker between the masking moiety and the active domain, the presence or absence of a cysteine within or flanking the active domain that is suitable for providing dissociation of a cysteine-cysteine disulfide bond, and the like.

In one embodiment, the masking moiety in the first polypeptide and/or in the second polypeptide comprises one or more protein domains. In another embodiment, the masking moiety in the first polypeptide and/or in the second polypeptide comprises a CH3 variant domain of an antibody constant (Fc) region. In another embodiment, the CH3 variant domain of an antibody constant (Fc) region comprises a CH1, CH2, CH3, CH2CH3, or CH1CH2CH3 domain. In another embodiment, the masking moiety in the first polypeptide and/or in the second polypeptide comprises a CH1, CH2, CH3, CH2CH3, or CH1CH2CH3 domain of an antibody constant (Fc) region. In another embodiment, the masking moiety in the first polypeptide comprises one or more heterodimers of at least two different CH3 variant domains of an antibody constant (Fc) region. In another embodiment, the masking moiety in the first polypeptide and/or in the second polypeptide comprises a CH3 variant domain of an antibody constant (Fc) region, wherein the CH3 variant domain comprises one or more mutations.

In one embodiment, the masking moiety in the first polypeptide and/or in the second polypeptide comprises a fusion of an antigen binding domain with a CH3 variant domain of an antibody constant (Fc) region. In another embodiment, the masking moiety in the first polypeptide and/or in the second polypeptide comprises a fusion of an antigen binding domain with a CH1, CH2, CH3, CH2CH3, or CH1CH2CH3 domain of an antibody constant (Fc) region.

In one embodiment, the masking moiety in the first polypeptide and/or in the second polypeptide does not bind an antigen. In another embodiment, the masking moiety in the first polypeptide and/or in the second polypeptide binds to an antigen. In another embodiment, the masking moiety in the first polypeptide and/or in the second polypeptide comprises an antigen binding domain. In another embodiment, the antigen binding domain is a variable heavy chain domain (VH), a variable domain from a heavy chain antibody (VHH), or an antigen specific peptide. In another embodiment, the antigen binding domain comprises an antibody light chain (LC) and an antibody heavy chain (HC). In another embodiment, the masking moiety comprises a constant domain of an antibody light chain (CL), wherein the light chain is a lambda or kappa chain.

The antibody domain of the fusion protein disclosed herein optionally comprises all or part of an immunoglobin molecule and optionally contains all or part of an immunoglobin constant domain region (Fc region).

The antibodies used herein optionally comprise IgG domains. However, other embodiments comprise alternate immunoglobins such as IgM, IgA, IgD, and IgE. Furthermore, all possible isotypes of the various immunoglobins are also encompassed within the current embodiments. Thus, IgG1, IgG2, IgG3, etc. are all possible molecules in the antibody domains of the antibody-immunostimulant fusion proteins used in the invention. In addition to choice in selection of the type of immunoglobin and isotype, different embodiments of the invention comprise various hinge regions (or functional equivalents thereof). Such hinge regions provide flexibility between the different domains of the antibody-immunostimulant fusion proteins. See, e.g., Penichet, et al. 2001 “Antibody-cytokine fusion proteins for the therapy of cancer” J Immunol Methods 248:91-101. In some embodiments, the antibody portion used in the compositions and methods provided herein is from an immunoglobulin class selected from IgG1, IgG2, IgG3, IgG4, IgD, IgA, or IgM.

In one embodiment, the masking moiety can be selected through a screening procedure that determines the optimal combination of masking moiety with cleavable linker that yields optimal inhibition of an active domain within the context of an activatable proprotein.

In one embodiment, the masking moiety in the first and/or second polypeptide prevents binding of a complex of IL-15/IL-15Rα in the first and/or second polypeptide to IL-15Rβ/γC present on the surface of a lymphocyte or a blood cell in vitro or in vivo.

In one embodiment, the cleavage of the masking moieties in any one of the first and/or second polypeptides in the activatable proprotein leads to partial activation of the activatable proprotein. In one embodiment, the masking moiety can interact or not interact with the complex of IL-15/IL-15Rα following partial activation (partial cleavage) of the activatable proprotein. In another embodiment, the cleavage of the masking moiety in both the first and second polypeptides in the activatable proprotein leads to complete activation of the activatable proprotein into an active complex of IL-15/IL-15Rα or a variant thereof.

In one embodiment, a humanized antibody may be used in the compositions and methods provided herein. In another embodiment, a humanized antibody may be used to generate a masking moiety provided herein. In another embodiment, a humanized antibody may be used to generate a masking moiety comprising an antigen binding domain provided herein. In another embodiment, a humanized antibody may be used to generate an antigen binding domain provided herein. In some embodiments, the term “humanized antibody” or “humanized version of an antibody” refers to antibodies in which the framework or “complementarity determining regions” (CDR) have been modified to comprise the CDR of an immunoglobulin of different specificity as compared to that of the parent immunoglobulin. In other embodiments, the CDRs of the VH and VL are grafted into the framework region of a human antibody to prepare the “humanized antibody.” See e.g. Riechmann, L., et al, Nature 332 (1988) 323-327; and Neuberger, M. S., et al, Nature 314 (1985) 268-270. The heavy and light chain variable framework regions can be derived from the same or different human antibody sequences. The human antibody sequences can be the sequences of naturally occurring human antibodies. Human heavy and light chain variable framework regions are listed e.g. in Lefranc, M.-P., Current Protocols in Immunology (2000)—Appendix IP A.1P.1-A.1P.37 and are accessible via IMGT, the international ImMunoGeneTics information System® (http://imgt.cines.fr) or via http://vbase.mrc-cpe.cam.ac.uk. Optionally the framework region can be modified by further mutations. The term “humanized antibody” as used herein also comprises such antibodies which are modified in the constant region to generate the properties according to the invention, especially in regard to Clq binding and/or FcR binding, e.g. by “class switching” i.e. change or mutation of Fc parts (e.g. from IgGl to IgG4 and/or IgGl/IgG4 mutation). The term “human antibody”, as used herein, is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al, Proc. Natl. Acad. Sci. USA 90 (1993) 2551-2555; Jakobovits, A., et al, Nature 362 (1993) 255-258; Brueggemann, M. D., et al., Year Immunol. 7 (1993) 33-40). Human antibodies can also be produced in phage display libraries (Hoogenboom, H. R., and Winter, G., J. Mol. Biol. 227 (1992) 381-388; Marks, J. D., et al, J. Mol. Biol. 222 (1991) 581-597). The techniques of Cole, A., et al. and Boerner, P., et al. are also available for the preparation of human monoclonal antibodies (Cole, A., et al., Monoclonal Antibodies and Cancer Therapy, Liss, A. L., p. 77 (1985); and Boerner, P., et al, J. Immunol. 147 (1991) 86-95). As already mentioned for humanized antibodies according to the invention the term “human antibody” as used herein also comprises such antibodies or portions thereof which are modified in the constant region to generate the properties according to the invention.

In one particular embodiment, the mAb comprised by the activatable proprotein provided herein comprises at least one or more domains from the Fc region and/or one or more domains from the Variable regions.

In one embodiment, a recombinant human antibody may be the source of the masking moiety of the disclosed activatable proprotein. The term “recombinant human antibody”, as used herein, is intended to include all human antibodies that are prepared, expressed, created or isolated by recombinant means, such as antibodies isolated from a host cell, for example a NS0 or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. The recombinant human antibodies according to the invention have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

In some embodiments, a masking moiety comprising an antigen binding domain that binds to an antigen as disclosed herein binds to the antigen with an optimal binding affinity. In other embodiments, a masking moiety comprising an antigen binding domain that binds to an antigen as disclosed herein binds to the antigen with a binding affinity within a range of KD 1.0×10⁻⁸ mol/l-1.0×10⁻¹ mol/l) at 25° C. The binding affinity is determined with a standard binding assay at 25° C., such as surface plasmon resonance technique (BIAcore®, GE-Healthcare Uppsala, Sweden). In one embodiment, a masking moiety comprised by an activatable proprotein disclosed herein comprises a variable region heavy (VH) chain and/or a variable region light (VL) chain.

In one embodiment of the invention, the antibody constant domains present in a masking moiety of an activatable proprotein provided herein is glycosylated. In some embodiments, the glycosylation is N-glycosylation. In other embodiments, the glycosylation is O-glycosylation.

### Constructs Encoding Activatable Proproteins

In one embodiment, provided herein is a vector comprising a recombinant nucleic acid molecule or polynucleotide encoding the activatable proprotein disclosed herein.

In one embodiment, provided herein is a recombinant nucleic acid molecule encoding the activatable proprotein of the invention. In one embodiment, one or more native or recombinant nucleic acid molecules are used to arrive at any of the amino acid sequences disclosed in the Sequence Listing, including SEQ ID NO: 1-SEQ ID NO: 346.

The disclosure further provides vectors and nucleic acid constructs which include sequences coding for a proprotein disclosed herein. Suitable nucleic acid constructs include, but are not limited to, constructs which are capable of expression in prokaryotic or eukaryotic cells. Expression constructs are generally selected so as to be compatible with the host cell in which they are to be used. In certain embodiments, the vector encodes an activatable proprotein (masking moiety, IL-15/IL-15Rα complex and cleavable linkers).

For example, non-viral and/or viral constructs vectors may be prepared and used, including plasmids, which provide for replication of a proprotein—encoding DNA and/or expression in a host cell. The choice of vector will depend on the type of cell in which propagation is desired and the purpose of propagation. Certain constructs are useful for amplifying and making large amounts of the desired DNA sequence. Other vectors are suitable for expression in cells in culture. The choice of appropriate vector is well within the skill of the art. Many such vectors are available commercially. Methods for generating constructs can be accomplished using methods well known in the art.

In order to effect expression in a host cell, a polynucleotide encoding an activatable proprotein disclosed herein or component thereof is operably linked to a regulatory sequence as appropriate to facilitate the desired expression properties. These regulatory sequences can include promoters, enhancers, terminators, operators, repressors, and inducers. Expression constructs generally also provide a transcriptional and translational initiation region as may be needed or desired, which may be inducible or constitutive, where the coding region is operably linked under the transcriptional control of the transcriptional initiation region, and a transcriptional and translational termination region. These control regions may be native to the species from which the nucleic acid is obtained, or may be derived from exogenous sources. In the context of an activatable proprotein provided herein, the masking moiety may be produced via recombinant means along with the additional components of a first polypeptide and a second polypeptide of the activatable proprotein (i.e.—IL-15/IL-15Rα and linkers), where the first and second polypeptide are joined via disulfide bonds and/or non-covalent bonds to form a dimer. Such methods are widely known in the state of the art and comprise protein expression in prokaryotic and eukaryotic cells with subsequent isolation of the activatable proprotein and usually purification to a pharmaceutically acceptable purity. For the protein expression nucleic acids encoding the components of the activatable proprotein (including light and heavy chains or fragments thereof of the masking moiety) are inserted into expression vectors by standard methods. Expression is performed in appropriate prokaryotic or eukaryotic host cells, such as CHO cells, NS0 cells, SP2/0 cells, HEK293 cells, COS cells, yeast, or E. coli cells, and the activatable proprotein is recovered from the cells (from the supernatant or after cells lysis).

A nucleic acid sequence is “operably linked” when it is placed into a functional relationship with another nucleic acid sequence. For example, a nucleic acid presequence or secretory leader is operably linked to a nucleic acid encoding a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the nucleic acid sequences being linked are contiguous, and, in the case of a secretory leader, contiguous and in reading frame. However, enhancers are optionally contiguous. Linking can be accomplished, for example, by ligation at convenient restriction sites. If such sites do not exist, synthetic oligonucleotide adaptors, linkers or other methods known in the art can be used. In another embodiment, the “operably linked” also refers to the functional pairing of distinct amino acid sequences, peptides or proteins, as in the pairing of the masking moiety and IL-15 described herein via a linker sequence also described herein.

Constructs, including expression constructs, can also include a selectable marker operative in the host to facilitate, for example, growth of host cells containing the construct of interest. Such selectable marker genes can provide a phenotypic trait for selection of transformed host cells such as dihydrofolate reductase or neomycin resistance for eukaryotic cell culture.

In one embodiment, the heavy and light chain constant domains in a masking moiety according to the invention are combined with sequences of promoter, translation initiation, constant region, 3′ untranslated region, polyadenylation, and transcription termination to form expression vector constructs. The heavy and light chain expression constructs can be combined into a single vector, co-transfected, serially transfected, or separately transfected into host cells which are then fused to form a single host cell expressing both chains. In one embodiment, the heavy and light chains are recombinantly constructed together with the other components of an activatable proprotein disclosed herein.

In some embodiments, the activatable proprotein utilized in the current invention may be optionally obtained or created by any method known in the art. For example, nucleic acid sequences encoding the appropriate components are optionally cloned and ligated into appropriate vectors (e.g., expression vectors for, e.g., prokaryotic or eukaryotic organisms). Additionally, nucleic acid sequences encoding the appropriate components are optionally cloned into the same vector in the appropriate orientation and location so that expression from the vector produces an activatable proprotein of the invention. Some optional embodiments also require post-expression modification, e.g., assembly of antibody subunits, etc. The techniques and art for the above (and similar) manipulations are well known to those skilled in the art. Pertinent instructions are found in, e.g., Sambrook et al., Molecular Cloning-A Laboratory Manual (2nd Ed.), Vols. 1-3, Cold Spring Harbor Laboratory, Cold Spring Harbor, N.Y., 1989 and Current Protocols in Molecular Biology, F. M. Ausubel et al., eds., Current Protocols, a joint venture between Greene Publishing Associates, Inc. and John Wiley & Sons, Inc. (supplemented through 1999). In some alternate embodiments, the components of the activatable proprotein are assembled post-expression through, e.g., chemical means.

In some embodiments, an activatable proprotein provided herein is administered to a patient in therapeutically effective amount which is the amount of the subject compound or combination that will elicit the biological or medical response of a tissue, system, animal or human that is being sought by a researcher, veterinarian, medical doctor or other clinician.

### Activatable Proprotein—Compositions

In one embodiment, the present invention provides a composition, e.g. a pharmaceutical composition comprising the activatable proprotein of the invention. In another embodiment, provided herein is a pharmaceutical composition comprising the activatable proprotein, and a pharmaceutically acceptable carrier. In some embodiments, provided are pharmaceutical compositions comprising the activatable proprotein disclosed herein formulated together with a pharmaceutical carrier.

In one embodiment, the activatable proprotein amino acid sequence is homologous to any amino acid sequence encoding an activatable proprotein disclosed herein. The term “homology” may refer to identity to an a sequence disclosed herein of greater than 70%. In another embodiment, “homology” refers to identity to any sequence disclosed herein of greater than 72%. In another embodiment, “homology” refers to identity to any sequence disclosed herein of greater than 75%. In another embodiment, “homology” refers to identity to any sequence disclosed herein of greater than 78%. In another embodiment, “homology” refers to identity to any of SEQ ID SEQ ID NO: X-Y of greater than 80%. In another embodiment, “homology” refers to identity to any sequence disclosed herein of greater than 82%. In another embodiment, “homology” refers to identity to any sequence disclosed herein of greater than 83%. In another embodiment, “homology” refers to identity to any sequence disclosed herein of greater than 85%. In another embodiment, “homology” refers to identity to any sequence disclosed herein of greater than 87%. In another embodiment, “homology” refers to identity to any sequence disclosed herein of greater than 88%. In another embodiment, “homology” refers to identity to any sequence disclosed herein of greater than 90%. In another embodiment, “homology” refers to identity to any sequence disclosed herein of greater than 92%. In another embodiment, “homology” refers to identity to any sequence disclosed herein of greater than 93%. In another embodiment, “homology” refers to identity to any sequence disclosed herein of greater than 95%. In another embodiment, “homology” refers to identity to any sequence disclosed herein of greater than 96%. In another embodiment, “homology” refers to identity to any sequence disclosed herein of greater than 97%. In another embodiment, “homology” refers to identity to any sequence disclosed herein of greater than 98%. In another embodiment, “homology” refers to identity to any sequence disclosed herein of greater than 99%. In another embodiment, “homology” refers to identity to any sequence disclosed herein of 100%.

The determination of percent identity between two sequences can be accomplished using a mathematical algorithm. A non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403-410. BLAST nucleotide searches can be performed with the NBLAST program, score=100, wordlength=12, to obtain nucleotide sequences homologous to a nucleic acid encoding a protein of interest. BLAST protein searches can be performed with the XBLAST program, score=50, wordlength=3, to obtain amino acid sequences homologous to a protein of interest. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (id.). When utilizing BLAST, Gapped BLAST, and PSI-BLAST programs, the default parameters of the respective programs (e.g., XBLAST and NBLAST) can be used. Another non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Natl. Acad. Sci. USA 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. Alternatively, protein sequence alignment may be carried out using the CLUSTAL W algorithm, as described by Higgins et al., 1996, Methods Enzymol. 266:383-402.

In some embodiments, polynucleotides of the present invention are prepared using PCR techniques using procedures and methods known to one skilled in the art. In some embodiments, the procedure involves the ligation of two different DNA sequences (See, for example, “Current Protocols in Molecular Biology”, eds. Ausubel et al., John Wiley & Sons, 1992).

In one embodiment, an affinity tag be included in an activatable proprotein disclosed herein. Affinity tags are well known in the art and are attached to a target and used to detect or isolate the target using a molecule that binds the affinity tag. In principal, any peptide or protein for which an antibody or other specific binding agent is available can be used as an affinity tag. Exemplary affinity tags suitable for use include, but are not limited to, a monocytic adaptor protein (MONA) binding peptide, a T7 binding peptide, a streptavidin binding peptide, a polyhistidine tract, protein A (Nilsson et al., EMBO J. 4:1075 (1985); Nilsson et al., Methods Enzymol. 198:3 (1991)), glutathione S transferase (Smith and Johnson, Gene 67:31 (1988)), Glu-Glu affinity tag (Grussenmeyer et al., Proc. Natl. Acad. Sci. USA 82:7952 (1985)), substance P, FLAG peptide (Hopp et al., Biotechnology 6:1204 (1988)), or other antigenic epitope or binding domain. See, in general, Ford et al., Protein Expression and Purification 2:95 (1991). DNA molecules encoding affinity tags are available from commercial suppliers (e.g., Pharmacia Biotech, Piscataway, N.J.). In one embodiment, a His6 tag is included in an activatable proprotein disclosed herein.

In one embodiment, polynucleotides of the present invention are inserted into expression vectors (i.e., a nucleic acid construct) to enable expression of the polypeptides described herein. In one embodiment, the expression vector of the present invention includes additional sequences which render this vector suitable for replication and integration in prokaryotes. In one embodiment, the expression vector of the present invention includes additional sequences which render this vector suitable for replication and integration in eukaryotes. In one embodiment, the expression vector of the present invention includes a shuttle vector which renders this vector suitable for replication and integration in both prokaryotes and eukaryotes. In some embodiments, cloning vectors comprise transcription and translation initiation sequences (e.g., promoters, enhancer) and transcription and translation terminators (e.g., polyadenylation signals).

In one embodiment, a variety of prokaryotic or eukaryotic cells can be used as host-expression systems to express the polypeptides of the present invention. In some embodiments, these include, but are not limited to, microorganisms, such as bacteria transformed with a recombinant bacteriophage DNA, plasmid DNA or cosmid DNA expression vector containing the polypeptide coding sequence; yeast transformed with recombinant yeast expression vectors containing the polypeptide coding sequence.

In some embodiments, non-bacterial expression systems are used (e.g., mammalian expression systems such as CHO cells) to express the polypeptide of the present invention. In one embodiment, the expression vector used to express polynucleotides of the present invention in mammalian cells is pCI-DHFR vector comprising a CMV promoter and a neomycin resistance gene.

In some embodiments, in bacterial systems of the present invention, a number of expression vectors can be advantageously selected depending upon the use intended for the polypeptide expressed. In one embodiment, large quantities of polypeptide are desired. In one embodiment, vectors that direct the expression of high levels of the protein product, possibly as a fusion with a hydrophobic signal sequence, which directs the expressed product into the periplasm of the bacteria or the culture medium where the protein product is readily purified are desired. In one embodiment, vectors adaptable to such manipulation include, but are not limited to, the pET series of E. coli expression vectors [Studier et al., Methods in Enzymol. 185:60-89 (1990)].

In one embodiment, yeast expression systems are used. In one embodiment, a number of vectors containing constitutive or inducible promoters can be used in yeast as disclosed in U.S. Pat. No. 5,932,447. In another embodiment, vectors which promote integration of foreign DNA sequences into the yeast chromosome are used.

In one embodiment, the expression vector of the present invention can further include additional polynucleotide sequences that allow, for example, the translation of several proteins from a single mRNA such as an internal ribosome entry site (IRES) and sequences for genomic integration of the promoter-chimeric polypeptide.

In some embodiments, mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p205. Other exemplary vectors include pMSG, pAV009/A⁺, pMTO01/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors are useful for in vivo expression of the polypeptides of the present invention since they offer advantages such as lateral infection and targeting specificity. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

In one embodiment, various methods can be used to introduce the expression vector encoding the polypeptides of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In some embodiments, introduction of nucleic acid by viral infection offers several advantages over other methods such as lipofection and electroporation, since higher transfection efficiency can be obtained due to the infectious nature of viruses.

It will be appreciated that the activatable proproteins or polypeptides comprised thereby of the present invention can also be expressed from a nucleic acid construct administered to the individual employing any suitable mode of administration, described hereinabove (i.e., in-vivo gene therapy). In one embodiment, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex-vivo gene therapy).

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

In some embodiments, transformed cells are cultured under effective conditions, which allow for the expression of high amounts of an activatable proprotein or polypeptides that make up the same (i.e.—a first and a second polypeptide as further described herein). In some embodiments, effective culture conditions include, but are not limited to, effective media, bioreactor, temperature, pH and oxygen conditions that permit protein production. In one embodiment, an effective medium refers to any medium in which a cell is cultured to produce an activatable proprotein provided herein. In some embodiments, a medium typically includes an aqueous solution having assimilable carbon, nitrogen and phosphate sources, and appropriate salts, minerals, metals and other nutrients, such as vitamins. In some embodiments, cells of the present invention can be cultured in conventional fermentation bioreactors, shake flasks, test tubes, microtiter dishes and petri plates. In some embodiments, culturing is carried out at a temperature, pH and oxygen content appropriate for a recombinant cell. In some embodiments, culturing conditions are within the expertise of one of ordinary skill in the art.

In some embodiments, depending on the vector and host system used for production, the resultant polypeptides of the activatable proprotein provided herein either remain within a recombinant cell, are secreted into the fermentation medium, are secreted into a space between two cellular membranes, such as the periplasmic space in E. coli, or are retained on the outer surface of a cell or viral membrane. In another embodiment, following a predetermined time in culture, recovery of the activatable proprotein or polypeptides that make up the same (including any precursors) is effected.

In one embodiment, the phrase “recovering the activatable proprotein or polypeptides comprised by or that make up the same” used herein refers to collecting the whole fermentation medium containing the activatable proprotein/polypeptides and need not imply additional steps of separation or purification.

In one embodiment, an activatable proprotein or polypeptides that make up the same of the present invention are purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

In one embodiment, to facilitate recovery, the expressed coding sequence can be engineered to encode an activatable proprotein of the present invention. In one embodiment, the activatable proprotein can be designed so that it can be readily isolated by affinity chromatography; e.g., by immobilization on a column specific for the cleavable moiety.

In one embodiment, an activatable proprotein of the present invention is retrieved in “substantially pure” form.

In one embodiment, the phrase “substantially pure” refers to a purity that allows for the effective use of an activatable proprotein in the applications described herein.

In one embodiment, an activatable proprotein provided herein can also be synthesized using in vitro expression systems. In one embodiment, in vitro synthesis methods are well known in the art and the components of the system are commercially available.

In some embodiments, once an activatable proprotein disclosed herein is synthesized and purified, their therapeutic efficacy can be assayed in vivo or in vitro.

In one embodiment, the pharmaceutical composition provided herein comprising an activatable proprotein of the invention is further formulated with a pharmaceutical carrier. As used herein, “pharmaceutical carrier” includes any and all solvents, dispersion media, coatings, antibacterial and antifungal agents, isotonic and absorption delaying agents, and the like that are physiologically compatible.

### Activatable Proproteins—Therapeutic Methods

Activatable proproteins described herein can be selected for use in methods of treatment of suitable subjects in need thereof. The activatable proproteins or pharmaceutical compositions provided herein can be administered by any suitable means, including oral, parenteral, subcutaneous, intraperitoneal, intrapulmonary, intranasal, spinal or epidermal administration (e.g. by injection or infusion), and, if desired for local injection (e.g., at the site of a solid tumor). Parenteral administration routes include intramuscular, intravenous, intraarterial, intraperitoneal, or subcutaneous administration.

In one embodiment, provided herein is a method comprising administering the activatable proprotein or the pharmaceutical composition disclosed herein to a subject having a cancer to prevent or treat the cancer in the subject, wherein following administration, the activatable proprotein is activated through protease cleavage in a cancerous tissue.

In another embodiment, provided herein is a method comprising administering the activatable proprotein or the pharmaceutical composition disclosed herein to a subject in need thereof, to elicit or enhance an anti-tumor immune response in the subject, wherein following administration, the activatable proprotein is activated through protease cleavage in the tumor.

In one embodiment, provided herein is a use of the activatable proprotein or the pharmaceutical composition disclosed herein for preventing or treating a cancer in a subject, comprising the step of administering the activatable proprotein or pharmaceutical composition, and wherein following administration, the activatable proprotein is activated through protease cleavage in a cancerous tissue.

In another embodiment, provided herein is a use of the activatable proprotein or the pharmaceutical composition disclosed herein for eliciting or enhancing an anti-tumor immune response in a subject, comprising the step of administering the activatable proprotein or pharmaceutical composition, and wherein following administration, the activatable proprotein is activated through protease cleavage in a tumor.

The term treatment site or disease site is meant to refer to a site at which an activatable proprotein is designed to be switchable, as described herein, e.g., a site at which a masking moiety is cleaved from the active domain. Treatment sites include tissues that can be accessed by local administration (e.g., injection, infusion (e.g., by catheter), etc.) or by systemic administration (e.g., administration to a site remote from a treatment site). Treatment sites include those that are relatively biologically confined (e.g., an organ, sac, tumor site, and the like).

In one embodiment, a disease disclosed herein is a cancer. In another embodiment, a cancer disclosed herein is selected from adrenocortical carcinoma, AIDS-related cancers, AIDS-related lymphoma, lymphoma, thyroid cancer, pancreatic cancer, anal cancer, anorectal cancer, cancer of the anal canal, appendix cancer, childhood cerebellar astrocytoma, childhood cerebral astrocytoma, basal cell carcinoma, biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, neuroblastoma, cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, leukemia, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, ocular cancer, islet cell tumors (endocrine pancreas), Kaposi Sarcoma, kidney cancer, renal cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, lung cancer, non-small cell lung cancer, small cell lung cancer, AIDS-related lymphoma, non-Hodgkin lymphoma, primary central nervous system lymphoma, Waldenstroem macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, Ewing family of sarcoma tumors, Kaposi Sarcoma, soft tissue sarcoma, epithelioid sarcoma, synovial sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, or Wilm's Tumor, or any tumor associated with, but not limited to, the type of cancer mentioned above, or any metastases following, but not limited to, any of the cancers mentioned above.

In one embodiment, the present invention provides a method of treating a disease or condition in a subject in need thereof, the method comprising administering a therapeutically effective amount of the activatable proprotein or the pharmaceutical composition disclosed herein.

In one embodiment, the present invention provides a method of treating a cancer or tumor disclosed herein in a subject in need thereof, the method comprising administering a therapeutically effective amount of the activatable proprotein or a pharmaceutical composition disclosed herein.

In one embodiment, the present invention provides a method of preventing, inhibiting, suppressing or delaying the onset of a cancer or tumor in a subject, the method comprising administering an effective amount of the activatable proprotein or the pharmaceutical composition described herein.

In some embodiments, treating a patient with a pharmaceutical composition comprising the activatable proprotein provided herein can result in increase in average survival time of a population of treated subjects in comparison to a population receiving monotherapy with a drug that is not a compound of the disclosure, or a pharmaceutically acceptable salt, solvate, analog or derivative thereof. Preferably, after treatment with the strategies, treatment modalities, methods, combinations, and compositions provided herein, the average survival time is increased by more than 30 days; more preferably, by more than 60 days; more preferably, by more than 90, 120, or 365 days; more preferably, by more than 365 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with the pharmaceutical composition disclosed herein.

In some embodiments, treating a patient with a pharmaceutical composition comprising the activatable proprotein provided herein can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the disclosure, or a pharmaceutically acceptable salt, solvate, analog or derivative thereof. Preferably, after treatment with the strategies, treatment modalities, methods, combinations, and compositions provided herein, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with the pharmaceutical composition disclosed herein.

### Activatable Proprotein—Administration, Dosing

An activatable proprotein or pharmaceutical composition comprising the same can be parenterally administered to a subject in need thereof, or can be administered by a variety of methods known in the art. As will be appreciated by the skilled artisan, the route and/or mode of administration will vary depending upon the desired results. To administer a compound of the invention by certain routes of administration, it may be necessary to coat the compound with, or co-administer the compound with, a material to prevent its inactivation. For example, the compound may be administered to a subject in an appropriate carrier, for example, liposomes, or a diluent. Pharmaceutically acceptable diluents include saline and aqueous buffer solutions. Pharmaceutical carriers include sterile aqueous solutions or dispersions and sterile powders for the extemporaneous preparation of sterile injectable solutions or dispersion. The use of such media and agents for pharmaceutically active substances is known in the art.

In typical embodiments, preparations for administration to subjects include sterile aqueous or non-aqueous solutions, suspensions, and emulsions. Some embodiments include non-aqueous solvents such as propylene glycol, polyethylene glycol, vegetable oils (e.g., olive oils), organic esters (e.g., ethyl oleate) and other solvents known to those of skill in the art. Physiologically acceptable carriers (or excipients) are optionally used in certain embodiments of the invention. Examples of such include, e.g., saline, PBS, Ringer's solution, lactated Ringer's solution, etc. Additionally, preservatives and additives are optionally added to the compositions to help ensure stability and sterility. For example, antibiotics and other bactericides, antioxidants, chelating agents, and the like are all optionally present in various embodiments of the compositions herein.

The phrases “parenteral administration” and “administered parenterally” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intra-articular, subcapsular, subarachnoid, intraspinal, epidural and intrasternal injection and infusion.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

The activatable proprotein, or pharmaceutical composition comprising the same are optionally administered to subjects in need of treatment (either therapeutically or prophylactically) in any appropriate sterile pharmaceutical carrier. Such pharmaceutical carrier acts to maintain the solubility and action of then activatable proprotein. In some embodiments, it may be desired to administer additional components in conjunction with the activatable proprotein. For example, in some treatment regimes, chemotherapeutic agents, antibiotics, additional formulations comprising the activatable proprotein provided herein and one or more standard of care agents, etc. are all optionally included with the compositions of the invention.

As used herein, the terms “combination treatment,” “combination therapy,” and “co-therapy” are used interchangeably and generally refer to treatment modalities featuring an activatable proprotein or pharmaceutical composition comprising the same as provided herein and an additional therapeutic agent. Typically, combination treatment modalities are part of a specific treatment regimen intended to provide a beneficial effect from the concurrent action of the therapeutic agent combination. The beneficial effect of the combination may include, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). In some embodiments, combination treatment comprises administration of two or more therapeutic agents in a sequential manner, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single dosage form having a fixed ratio of each therapeutic agent or in multiple, separate dosage forms for the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. The therapeutic agents can be administered according to the same or to a different administration interval. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection.

In some embodiments, combination therapy also embraces the administration of the therapeutic agents as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

In some embodiments, the additional therapeutic agent is a chemotherapeutic agent (also referred to as an anti-neoplastic agent or anti-proliferative agent), e.g., an alkylating agent; an antibiotic; an anti-metabolite; a detoxifying agent; an interferon; a polyclonal or monoclonal antibody; an EGFR inhibitor; a HER2 inhibitor; a histone deacetylase inhibitor; a hormone; a mitotic inhibitor; an MTOR inhibitor; a multi-kinase inhibitor; a serine/threonine kinase inhibitor; a tyrosine kinase inhibitors; a VEGF/VEGFR inhibitor; a taxane or taxane derivative, an aromatase inhibitor, an anthracycline, a microtubule targeting drug, a topoisomerase poison drug, an inhibitor of a molecular target or enzyme (e.g., a kinase or a protein methyltransferase), a cytidine analogue drug or any chemotherapeutic, an immune checkpoint inhibitor, or any anti-neoplastic or anti-proliferative agent known to those of skill in the art.

Exemplary alkylating agents suitable for use according to the combination treatment modalities provided herein include, but are not limited to, cyclophosphamide (Cytoxan; Neosar); chlorambucil (Leukeran); melphalan (Alkeran); carmustine (BiCNU); busulfan (Busulfex); lomustine (CeeNU); dacarbazine (DTIC-Dome); oxaliplatin (Eloxatin); carmustine (Gliadel); ifosfamide (Ifex); mechlorethamine (Mustargen); busulfan (Myleran); carboplatin (Paraplatin); cisplatin (CDDP; Platinol); temozolomide (Temodar); thiotepa (Thioplex); bendamustine (Treanda); or streptozocin (Zanosar).

Exemplary suitable anthracyclines include, but are not limited to, doxorubicin (Adriamycin); doxorubicin liposomal (Doxil); mitoxantrone (Novantrone); bleomycin (Blenoxane); daunorubicin (Cerubidine); daunorubicin liposomal (DaunoXome); dactinomycin (Cosmegen); epirubicin (Ellence); idarubicin (Idamycin); plicamycin (Mithracin); mitomycin (Mutamycin); pentostatin (Nipent); or valrubicin (Valstar).

Exemplary anti-metabolites include, but are not limited to, fluorouracil (Adrucil); capecitabine (Xeloda); hydroxyurea (Hydrea); mercaptopurine (Purinethol); pemetrexed (Alimta); fludarabine (Fludara); nelarabine (Arranon); cladribine (Cladribine Novaplus); clofarabine (Clolar); cytarabine (Cytosar-U); decitabine (Dacogen); cytarabine liposomal (DepoCyt); hydroxyurea (Droxia); pralatrexate (Folotyn); floxuridine (FUDR); gemcitabine (Gemzar); cladribine (Leustatin); fludarabine (Oforta); methotrexate (MTX; Rheumatrex); methotrexate (Trexall); thioguanine (Tabloid); TS-1 or cytarabine (Tarabine PFS).

Exemplary detoxifying agents include, but are not limited to, amifostine (Ethyol) or mesna (Mesnex).

Exemplary interferons include, but are not limited to, interferon alfa-2b (Intron A) or interferon alfa-2a (Roferon-A).

Exemplary polyclonal or monoclonal antibodies include, but are not limited to, trastuzumab (Herceptin); ofatumumab (Arzerra); bevacizumab (Avastin); rituximab (Rituxan); cetuximab (Erbitux); panitumumab (Vectibix); tositumomab/iodine-131 tositumomab (Bexxar); alemtuzumab (Campath); ibritumomab (Zevalin; In-111; Y-90 Zevalin); gemtuzumab (Mylotarg); eculizumab (Soliris) or denosumab.

Exemplary EGFR inhibitors include, but are not limited to, gefitinib (Iressa); lapatinib (Tykerb); cetuximab (Erbitux); erlotinib (Tarceva); panitumumab (Vectibix); PKI-166; canertinib (CI-1033); matuzumab (EMD 72000) or EKB-569.

Exemplary HER2 inhibitors include, but are not limited to, trastuzumab (Herceptin); lapatinib (Tykerb) or AC-480.

Histone Deacetylase Inhibitors include, but are not limited to, vorinostat (Zolinza).

Exemplary hormones include, but are not limited to, tamoxifen (Soltamox; Nolvadex); raloxifene (Evista); megestrol (Megace); leuprolide (Lupron; Lupron Depot; Eligard; Viadur); fulvestrant (Faslodex); letrozole (Femara); triptorelin (Trelstar LA; Trelstar Depot); exemestane (Aromasin); goserelin (Zoladex); bicalutamide (Casodex); anastrozole (Arimidex); fluoxymesterone (Androxy; Halotestin); medroxyprogesterone (Provera; Depo-Provera); estramustine (Emcyt); flutamide (Eulexin); toremifene (Fareston); degarelix (Firmagon); nilutamide (Nilandron); abarelix (Plenaxis); or testolactone (Teslac).

Exemplary mitotic inhibitors include, but are not limited to, paclitaxel (Taxol; Onxol; Abraxane); docetaxel (Taxotere); vincristine (Oncovin; Vincasar PFS); vinblastine (Velban); etoposide (Toposar; Etopophos; VePesid); teniposide (Vumon); ixabepilone (Ixempra); nocodazole; epothilone; vinorelbine (Navelbine); camptothecin (CPT); irinotecan (Camptosar); topotecan (Hycamtin); amsacrine or lamellarin D (LAM-D).

Exemplary MTOR inhibitors include, but are not limited to, everolimus (Afinitor) or temsirolimus (Torisel); rapamune, ridaforolimus; or AP23573.

Exemplary multi-kinase inhibitors include, but are not limited to, sorafenib (Nexavar); sunitinib (Sutent); BIBW 2992; E7080; Zd6474; PKC-412; motesanib; or AP24534.

Exemplary serine/threonine kinase inhibitors include, but are not limited to, ruboxistaurin; eril/fasudil hydrochloride; flavopiridol; seliciclib (CYC202; Roscovitine); SNS-032 (BMS-387032); Pkc412; bryostatin; KAI-9803; SF1126; VX-680; Azd1152; Arry-142886 (AZD-6244); SCIO-469; GW681323; CC-401; CEP-1347 or PD 332991.

Exemplary tyrosine kinase inhibitors include, but are not limited to, erlotinib (Tarceva); gefitinib (Iressa); imatinib (Gleevec); sorafenib (Nexavar); sunitinib (Sutent); trastuzumab (Herceptin); bevacizumab (Avastin); rituximab (Rituxan); lapatinib (Tykerb); cetuximab (Erbitux); panitumumab (Vectibix); everolimus (Afinitor); alemtuzumab (Campath); gemtuzumab (Mylotarg); temsirolimus (Torisel); pazopanib (Votrient); dasatinib (Sprycel); nilotinib (Tasigna); vatalanib (Ptk787; ZK222584); CEP-701; SU5614; MLN518; XL999; VX-322; Azd0530; BMS-354825; SKI-606 CP-690; AG-490; WHI-P154; WHI-P131; AC-220; or AMG888.

Exemplary VEGF/VEGFR inhibitors include, but are not limited to, bevacizumab (Avastin); sorafenib (Nexavar); sunitinib (Sutent); ranibizumab; pegaptanib; or vandetinib.

Exemplary microtubule targeting drugs include, but are not limited to, paclitaxel, docetaxel, vincristin, vinblastin, nocodazole, epothilones and navelbine.

Exemplary topoisomerase poison drugs include, but are not limited to, teniposide, etoposide, adriamycin, camptothecin, daunorubicin, dactinomycin, mitoxantrone, amsacrine, epirubicin and idarubicin.

Exemplary taxanes or taxane derivatives include, but are not limited to, paclitaxel and docetaxol.

Exemplary general chemotherapeutic, anti-neoplastic, anti-proliferative agents include, but are not limited to, altretamine (Hexalen); isotretinoin (Accutane; Amnesteem; Claravis; Sotret); tretinoin (Vesanoid); azacitidine (Vidaza); bortezomib (Velcade) asparaginase (Elspar); levamisole (Ergamisol); mitotane (Lysodren); procarbazine (Matulane); pegaspargase (Oncaspar); denileukin diftitox (Ontak); porfimer (Photofrin); aldesleukin (Proleukin); lenalidomide (Revlimid); bexarotene (Targretin); thalidomide (Thalomid); temsirolimus (Torisel); arsenic trioxide (Trisenox); verteporfin (Visudyne); mimosine (Leucenol); (1M tegafur—0.4 M 5-chloro-2,4-dihydroxypyrimidine—1 M potassium oxonate) or lovastatin.

In some embodiments, combination treatment modalities are provided in which the additional therapeutic agent is a cytokine, e.g., G-CSF (granulocyte colony stimulating factor). In another aspect, a pharmaceutical composition provided herein may be administered in combination with radiation therapy. Radiation therapy can also be administered in combination with a pharmaceutical composition provided herein and another chemotherapeutic agent described herein as part of a multi-agent therapy. In yet another aspect, a pharmaceutical composition provided herein may be administered in combination with standard chemotherapy combinations such as, but not restricted to, CMF (cyclophosphamide, methotrexate and 5-fluorouracil), CAF (cyclophosphamide, adriamycin and 5-fluorouracil), AC (adriamycin and cyclophosphamide), FEC (5-fluorouracil, epirubicin, and cyclophosphamide), ACT or ATC (adriamycin, cyclophosphamide, and paclitaxel), rituximab, Xeloda (capecitabine), Cisplatin (CDDP), Carboplatin, TS-1 (tegafur, gimestat and otastat potassium at a molar ratio of 1:0.4:1), Camptothecin-11 (CPT-11, Irinotecan or Camptosar™), CHOP (cyclophosphamide, hydroxydaunorubicin, oncovin, and prednisone or prednisolone), R-CHOP (rituximab, cyclophosphamide, hydroxydaunorubicin, oncovin, prednisone or prednisolone), or CMFP (cyclophosphamide, methotrexate, 5-fluorouracil and prednisone).

In some embodiments, a pharmaceutical composition provided herein may be administered with an inhibitor of an enzyme, such as a receptor or non-receptor kinase. Receptor and non-receptor kinases are, for example, tyrosine kinases or serine/threonine kinases. Kinase inhibitors described herein are small molecules, polynucleic acids, polypeptides, or antibodies.

Exemplary kinase inhibitors include, but are not limited to, Bevacizumab (targets VEGF), BIBW 2992 (targets EGFR and Erb2), Cetuximab/Erbitux (targets Erb1), Imatinib/Gleevec (targets Bcr-Abl), Trastuzumab (targets Erb2), Gefitinib/Iressa (targets EGFR), Ranibizumab (targets VEGF), Pegaptanib (targets VEGF), Erlotinib/Tarceva (targets Erb1), Nilotinib (targets Bcr-Abl), Lapatinib (targets Erb1 and Erb2/Her2), GW-572016/lapatinib ditosylate (targets HER2/Erb2), Panitumumab/Vectibix (targets EGFR), Vandetinib (targets RET/VEGFR), E7080 (multiple targets including RET and VEGFR), Herceptin (targets HER2/Erb2), PKI-166 (targets EGFR), Canertinib/CI-1033 (targets EGFR), Sunitinib/SU-11464/Sutent (targets EGFR and FLT3), Matuzumab/Emd7200 (targets EGFR), EKB-569 (targets EGFR), Zd6474 (targets EGFR and VEGFR), PKC-412 (targets VEGR and FLT3), Vatalanib/Ptk787/ZK222584 (targets VEGR), CEP-701 (targets FLT3), SU5614 (targets FLT3), MLN518 (targets FLT3), XL999 (targets FLT3), VX-322 (targets FLT3), Azd0530 (targets SRC), BMS-354825 (targets SRC), SKI-606 (targets SRC), CP-690 (targets JAK), AG-490 (targets JAK), WHI-P154 (targets JAK), WHI-P131 (targets JAK), sorafenib/Nexavar (targets RAF kinase, VEGFR-1, VEGFR-2, VEGFR-3, PDGFR- 3, KIT, FLT-3, and RET), Dasatinib/Sprycel (BCR/ABL and Src), AC-220 (targets Flt3), AC-480 (targets all HER proteins, “panHER”), Motesanib diphosphate (targets VEGF1-3, PDGFR, and c-kit), Denosumab (targets RANKL, inhibits SRC), AMG888 (targets HER3), and AP24534 (multiple targets including Flt3).

In another embodiment, an activatable proprotein or pharmaceutical composition comprising the same polypeptide disclosed herein is administered to a subject once a day. In some embodiments, the activatable proprotein or pharmaceutical composition comprising the same is administered to a subject once every two days. In another embodiment, the activatable proprotein or pharmaceutical composition comprising the same is administered to a subject once every three days. In another embodiment, the activatable proprotein or pharmaceutical composition comprising the same is administered to a subject once every four days. In another embodiment, the activatable proprotein or pharmaceutical composition comprising the same is administered to a subject once every five days. In another embodiment, the activatable proprotein or pharmaceutical composition comprising the same polypeptide is administered to a subject once every six days. In another embodiment, the activatable proprotein or pharmaceutical composition comprising the same is administered to a subject once every week. In another embodiment, the activatable proprotein or pharmaceutical composition comprising the same is administered to a subject once every 7-14 days. In another embodiment, the activatable proprotein or pharmaceutical composition comprising the same is administered to a subject once every 10-20 days. In another embodiment, the activatable proprotein or pharmaceutical composition comprising the same is administered to a subject once every 5-15 days. In another embodiment, the activatable proprotein or pharmaceutical composition comprising the same is administered to a subject once every 15-30 days.

In one embodiment, a dose of an activatable proprotein of the present invention comprises from 0.005 to 0.1 milligrams/kg in an injectable solution. In another embodiment, the dose comprises from 0.005 to 0.5 milligrams/kg of the activatable proprotein. In another embodiment, the dose comprises from 0.05 to 0.1 micrograms of the activatable proprotein. In another embodiment, the dose comprises from 0.005 to 0.1 milligrams/kg of the activatable proprotein in an injectable solution.

In another embodiment, an activatable proprotein or pharmaceutical composition comprising the same is administered to a subject in a dose ranging from 0.0001 mg to 0.6 mg. In another embodiment, the activatable proprotein or pharmaceutical composition comprising the same is administered to a subject in a dose ranging from 0.001 mg to 0.005 mg. In another embodiment, the activatable proprotein or pharmaceutical composition comprising the same is administered to a subject in a dose ranging from 0.005 mg to 0.01 mg. In another embodiment, the activatable proprotein or pharmaceutical composition comprising the same is administered to a subject in a dose ranging from 0.01 mg to 0.3 mg. In another embodiment, the activatable proprotein or pharmaceutical composition comprising the same is administered to a subject in a dose ranging from 0.2 mg to 0.6 mg.

In another embodiment, an activatable proprotein or pharmaceutical composition comprising the same is administered to a subject in a dose ranging from 1-100 mcg/kg. In another embodiment, the activatable proprotein or pharmaceutical composition comprising the same is administered to a subject in a dose ranging from 10-80 mcg/kg. In another embodiment, the activatable proprotein or pharmaceutical composition comprising the same is administered to a subject in a dose ranging from 20-60 mcg/kg. In another embodiment, the activatable proprotein or pharmaceutical composition comprising the same is administered to a subject in a dose ranging from 10-50 mcg/kg. In another embodiment, the activatable proprotein or pharmaceutical composition comprising the same is administered to a subject in a dose ranging from 40-80 mcg/kg. In another embodiment, the activatable proprotein or pharmaceutical composition comprising the same is administered to a subject in a dose ranging from 10-30 mcg/kg. In another embodiment, the activatable proprotein or pharmaceutical composition comprising the same is administered to a subject in a dose ranging from 30-60 mcg/kg.

In another embodiment, an activatable proprotein or pharmaceutical composition comprising the same is administered to a subject in a dose ranging from 0.1 mcg/kg to 100 mg/kg. In another embodiment, the activatable proprotein or pharmaceutical composition comprising the same is administered to a subject in a dose ranging from 0.1 mcg/kg to 50 mg/kg. In another embodiment, the activatable proprotein or pharmaceutical composition comprising the same is administered to a subject in a dose ranging from 0.1 mcg/kg to 25 mg/kg. In another embodiment, the activatable proprotein or pharmaceutical composition comprising the same is administered to a subject in a dose ranging from 0.1 mcg/kg to 10 mg/kg. In another embodiment, the activatable proprotein or pharmaceutical composition comprising the same is administered to a subject in a dose ranging from 0.1 mcg/kg to 5 mg/kg. In another embodiment, the activatable proprotein or pharmaceutical composition comprising the same is administered to a subject in a dose ranging from 0.1 mcg/kg to 1 mg/kg. In another embodiment, the activatable proprotein or pharmaceutical composition comprising the same is administered to a subject in a dose ranging from 0.1 mcg/kg to 0.1 mg/kg.

In another embodiment, an activatable proprotein or pharmaceutical composition comprising the same is administered to a subject in a dose ranging from 0.2 mg to 2 mg. In another embodiment, the activatable proprotein or pharmaceutical composition comprising the same is administered to a subject in a dose ranging from 2 mg to 6 mg. In another embodiment, the activatable proprotein or pharmaceutical composition comprising the same is administered to a subject in a dose ranging from 4 mg to 10 mg. In another embodiment, the activatable proprotein or pharmaceutical composition comprising the same is administered to a subject in a dose ranging from 5 mg and 15 mg.

In one embodiment, an activatable proprotein or pharmaceutical composition comprising the same is administered to a subject in a dose ranging from 10 μg/kg-1000 μg/kg. In another embodiment, an activatable proprotein or pharmaceutical composition comprising the same is administered to a subject in a dose ranging from 25 μg/kg-600 μg/kg. In another embodiment, an activatable proprotein or pharmaceutical composition comprising the same is administered to a subject in a dose ranging from 50 μg/kg-400 μg/kg. In another embodiment, an activatable proprotein or pharmaceutical composition comprising the same is administered to a subject in a dose of about 25 μg/kg. In another embodiment, an activatable proprotein or pharmaceutical composition comprising the same is administered to a subject in a dose of about 50 μg/kg. In another embodiment, an activatable proprotein or pharmaceutical composition comprising the same is administered to a subject in a dose of about 100 μg/kg. In another embodiment, an activatable proprotein or pharmaceutical composition comprising the same is administered to a subject in a dose of about 200 μg/kg. In another embodiment, an activatable proprotein or pharmaceutical composition comprising the same is administered to a subject in a dose of about 300 μg/kg. In another embodiment, an activatable proprotein or pharmaceutical composition comprising the same is administered to a subject in a dose of about 400 μg/kg. In another embodiment, an activatable proprotein or pharmaceutical composition comprising the same is administered to a subject in a dose of about 500 μg/kg. In another embodiment, an activatable proprotein or pharmaceutical composition comprising the same is administered to a subject in a dose of about 600 μg/kg.

In one embodiment, a single one time dose of the activatable proprotein or pharmaceutical composition comprising the same is administered to a subject. In another embodiment, a total of two doses are administered to the subject. In another embodiment, a total of two or more doses are administered to the subject.

In another embodiment, a dose of an activatable proprotein or pharmaceutical composition comprising the same is administered to a subject at least once a day. In another embodiment, a dose of the activatable proprotein or pharmaceutical composition comprising the same is administered to a subject at least once every two days. In another embodiment, a dose of the activatable proprotein or pharmaceutical composition comprising the same is administered to a subject at least once a every two or more days. In another embodiment, a dose of the activatable proprotein or pharmaceutical composition comprising the same is administered to a subject every week, biweekly or every three weeks. In another embodiment, a dose of the activatable proprotein or pharmaceutical composition comprising the same is administered to a subject at least once a week. In another embodiment, a dose of the activatable proprotein or pharmaceutical composition comprising the same is administered to a subject at least once every two weeks. In another embodiment, a dose of the activatable proprotein or pharmaceutical composition comprising the same is administered to a subject at least once every three weeks. In another embodiment, a dose of the activatable proprotein or pharmaceutical composition comprising the same is administered to a subject at least once every three or more weeks. In another embodiment, a dose of the activatable proprotein or pharmaceutical composition comprising the same is administered to a subject two or more times a week. In another embodiment, a dose of the activatable proprotein or pharmaceutical composition comprising the same is administered to a subject two or more times a month. In another embodiment, a dose of the activatable proprotein or pharmaceutical composition comprising the same is administered to a subject two or more times a year. In another embodiment, a dose of the activatable proprotein or pharmaceutical composition comprising the same is administered to a subject two or more times every two years. In another embodiment, a dose of the activatable proprotein or pharmaceutical composition comprising the same is administered to a subject two or more times every two or more years.

In another embodiment, a dose of an activatable proprotein or pharmaceutical composition comprising the same is administered at least once every 36 hours. In another embodiment, a dose of the activatable proprotein or pharmaceutical composition comprising the same is administered at least once every 48 hours. In another embodiment, a dose of the activatable proprotein or pharmaceutical composition comprising the same is administered at least once every 60 hours. In another embodiment, a dose of the activatable proprotein or pharmaceutical composition comprising the same is administered at least once every 72 hours. In another embodiment, a dose of the activatable proprotein or pharmaceutical composition comprising the same is administered at least once every 84 hours. In another embodiment, a dose of the activatable proprotein or pharmaceutical composition comprising the same is administered at least once every 96 hours. In another embodiment, a dose of the activatable proprotein or pharmaceutical composition comprising the same is administered at least once every 5 days. In another embodiment, a dose of the activatable proprotein or pharmaceutical composition comprising the same is administered at least once every 6 days. In another embodiment, a dose of the activatable proprotein or pharmaceutical composition comprising the same is administered at least once every 7 days. In another embodiment, a dose of the activatable proprotein or pharmaceutical composition comprising the same is administered at least once every 8-10 days. In another embodiment, a dose of the activatable proprotein or pharmaceutical composition comprising the same is administered at least once every 10-12 days. In another embodiment, a dose of the activatable proprotein or pharmaceutical composition comprising the same is administered at least once every 12-15 days. In another embodiment, a dose of the activatable proprotein or pharmaceutical composition comprising the same is administered at least once every 15-25 days. In another embodiment, a dose of the activatable proprotein or pharmaceutical composition comprising the same is administered at least once every 20-30 days.

In one embodiment, a dose of an activatable proprotein or pharmaceutical composition comprising the same is administered to a subject at least once every 1 month. In one embodiment, a dose of the activatable proprotein or pharmaceutical composition comprising the same is administered at least once every 2 months. In one embodiment, a dose of the activatable proprotein or pharmaceutical composition comprising the same is administered at least once every 3 months. In one embodiment, a dose of the activatable proprotein or pharmaceutical composition comprising the same is administered at least once every 4 months. In one embodiment, a dose of the activatable proprotein or pharmaceutical composition comprising the same is administered at least once every 5 months. In one embodiment, a dose of the activatable proprotein or pharmaceutical composition comprising the same is administered at least once every 6 months. In one embodiment, a dose of the activatable proprotein or pharmaceutical composition comprising the same is administered at least once every 6-12 months. In another embodiment, a dose of the activatable proprotein or pharmaceutical composition comprising the same is administered quarterly. In another embodiment, the dose is administered daily, weekly, biweekly, monthly or annually. In another embodiment, the dose is administered once, twice, or two or more times a day, a week, a month or a year. In another embodiment, the dose is administered every two, three, four, or at least five years.

In one embodiment, repeat administrations (doses) of compositions of this invention may be undertaken immediately following the first course of treatment or after an interval of days, weeks, or years to achieve the desired effect as further provided herein (e.g. to prevent or treat cardiovascular disease or condition, or a CNS-related disease or condition).

In one embodiment, the pharmaceutical compositions are administered by intravenous, intra-arterial, subcutaneous or intramuscular injection of a liquid preparation. In another embodiment, liquid formulations include solutions, suspensions, dispersions, emulsions, oils and the like. In one embodiment, the pharmaceutical compositions are administered intravenously, and are thus formulated in a form suitable for intravenous administration. In another embodiment, the pharmaceutical compositions are administered intra-arterially, and are thus formulated in a form suitable for intra-arterial administration.

In some embodiments, compositions for use in the methods disclosed herein comprise solutions or emulsions, which in some embodiments are aqueous solutions or emulsions comprising a safe and effective amount of the compounds disclosed herein and optionally, other compounds, intended for intravenous or subcutaneous administration.

In some embodiments, the various constituents of the compositions come pre-measured and/or prepackaged and/or ready for use without additional measurement, etc. The present invention also optionally comprises kits for conducting/using the methods and/or the compositions of the invention. Additionally, such kits can also comprise appropriate excipients (e.g., pharmaceutically acceptable excipients) for performing therapeutic and/or prophylactic treatments of the invention. Such kits optionally contain additional components for the assembly and/or use of the compositions of the invention including, but not limited to, e.g., diluents, etc.

The compositions described herein are optionally packaged to include all (or almost all) necessary components for performing the methods of the invention or for using the compositions of the invention (optionally including, e.g., written instructions for the use of the methods/compositions of the invention). For example, the kits can optionally include such components as, e.g., buffers, reagents, serum proteins, antibodies, substrates, etc. In the case of prepackaged reagents, the kits optionally include pre-measured or pre-dosed amounts that are ready to incorporate into the methods without measurement, e.g., pre-measured fluid aliquots, or pre-weighed or pre-measured solid reagents that can be easily reconstituted by the end-user of the kit.

Such kits also typically include appropriate instructions for performing the methods of the invention and/or using the compositions of the invention. In some embodiments, the components of the kits/packages are provided in a stabilized form, so as to prevent degradation or other loss during prolonged storage, e.g., from leakage. A number of stabilizing processes/agents are widely used for reagents, etc. that are to be stored, such as the inclusion of chemical stabilizers (i.e., enzymatic inhibitors, microbicides/bacteriostats, anticoagulants), etc. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The composition must be sterile and fluid to the extent that the composition is deliverable by syringe. In addition to water, the carrier preferably is an isotonic buffered saline solution. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements 5 other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The present invention further provides a kit for preventing, treating or delaying a cardiovascular disease or condition in a human, wherein the kit comprises one or more doses of pharmaceutical composition comprising an activatable proprotein disclosed herein used for preventing, treating or delaying a cardiovascular disease or condition, and instructions on how to use the pharmaceutical preparation or composition.

The present invention further provides a kit for preventing, treating or delaying a CNS-related disease or condition in a human, wherein the kit comprises one or more doses of pharmaceutical composition comprising an activatable proprotein disclosed herein used for preventing, treating or delaying a cardiovascular disease or condition, and instructions on how to use the pharmaceutical preparation or composition.

The present invention further provides a kit for preventing, treating or delaying heart failure with preserved ejection fraction in a human, wherein the kit comprises one or more doses of pharmaceutical composition comprising an activatable protein disclosed herein used for preventing, treating or delaying heart failure with preserved ejection fraction, and instructions on how to use the pharmaceutical preparation or composition.

The following examples are presented in order to more fully illustrate the preferred embodiments of the invention. They should in no way be construed, however, as limiting the broad scope of the invention.

### EXAMPLES

### Example 1: Engineering of Fc-IL-15-Linker-IL-15Rα Fusion Proteins

In order to reduce the toxicity of IL-15 related therapeutic drugs, Fc-IL-15-linker-IL-15Rα fusion proteins (hereon referred to as Fc-ILR fusion proteins) were generated as prodrugs (i.e.—activatable proprotein as described elsewhere herein). The prodrugs have very low activities. Full activities can be restored upon protease cleavage of the designed protease specific linker sequence within the prodrugs.

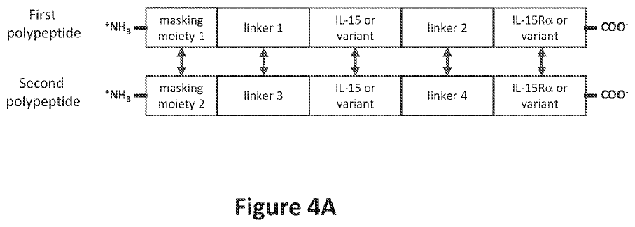

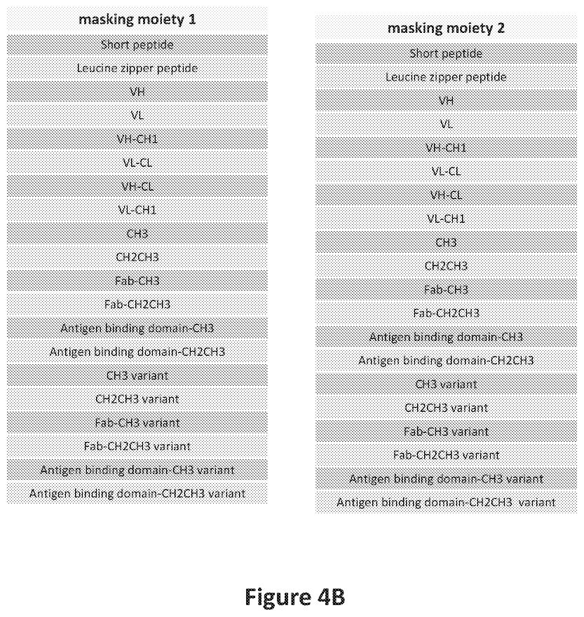

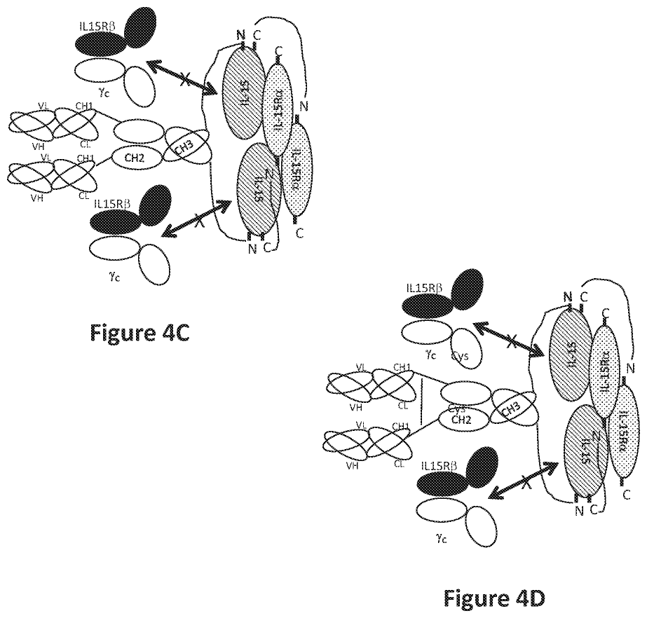

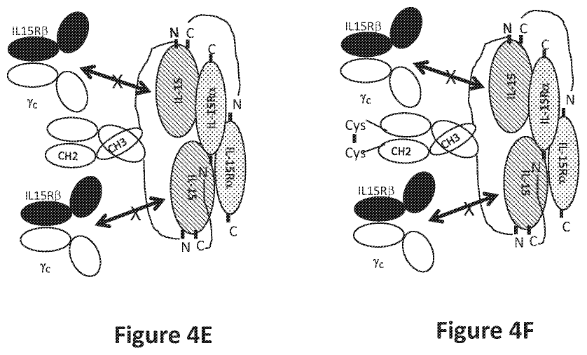

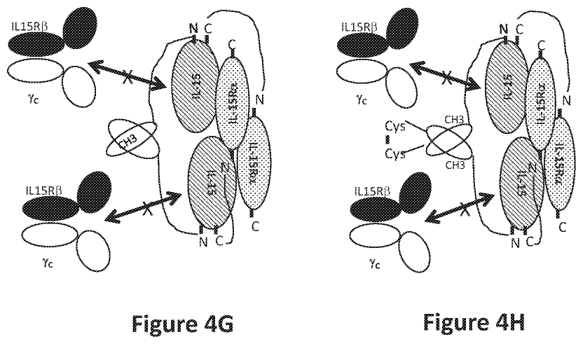

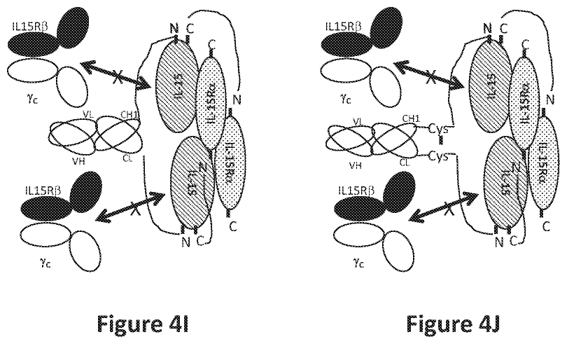

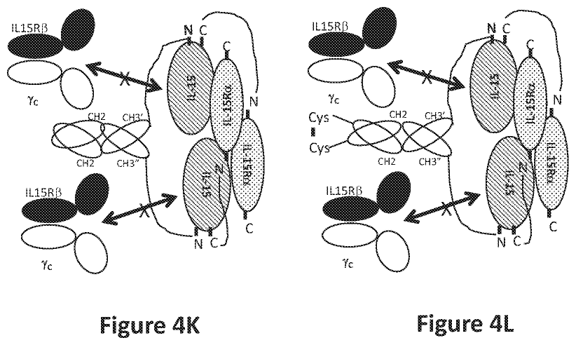

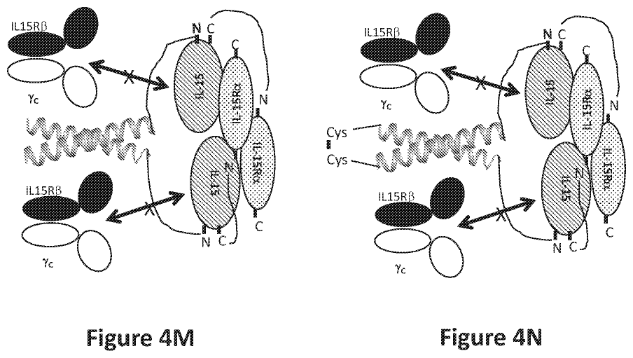

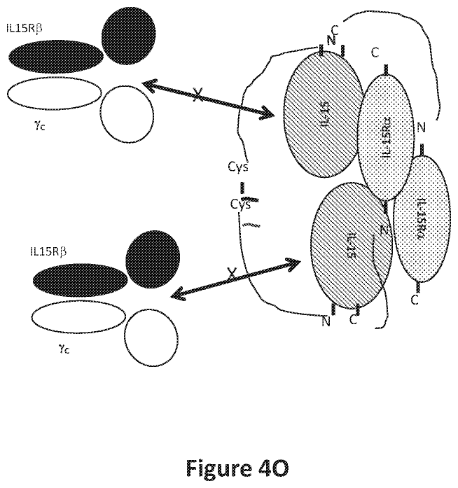

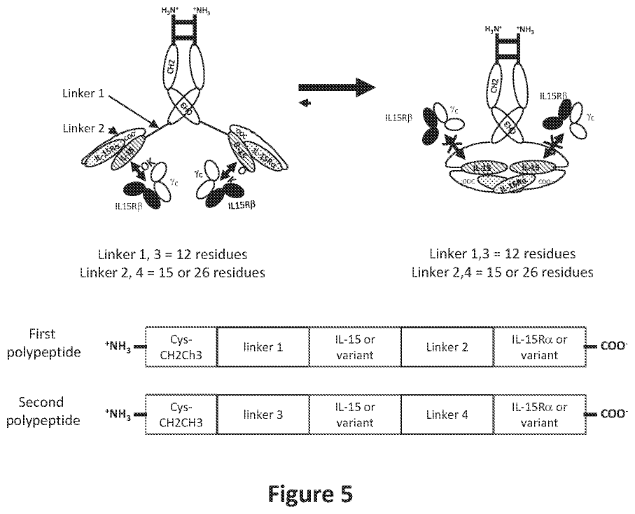

Plasmids coding for single chain IL-15-linker-IL-15Rα (ILR) with or without Fc fusion were constructed by standard gene synthesis, followed by sub-cloning into pTT5 expression vector. Schematics of illustrative Fc-ILR fusion protein formats are depicted in FIGS. 3E, 3F, 4A, and 4F.

Illustrative proteins of Fc-ILR format without protease cleavage site include P1187 (SEQ ID NO: 18), P1188 (SEQ ID NO: 19), P1250 (SEQ ID NO: 20), P1251 (SEQ ID NO: 21), and P1252 (SEQ ID NO: 22).

Illustrative proteins of IL-15-linker-IL-15Rα-Fc (ILR-Fc) format (FIG. 3B) are P1186 (SEQ ID NO: 17) and P1331 (SEQ ID NO: 73).

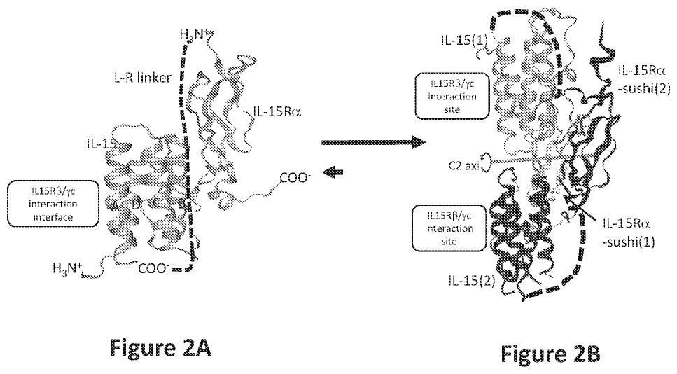

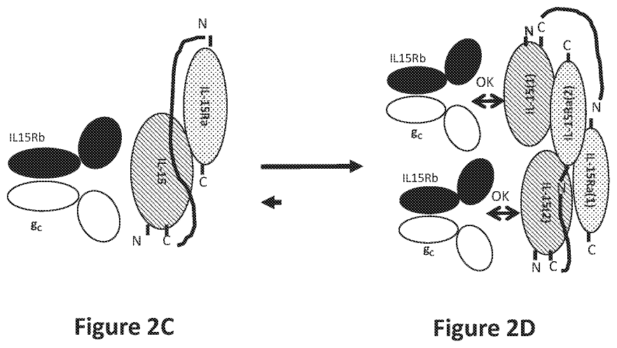

Illustrative proteins of IL-15-linker-IL-15Rα format (FIGS. 2C and 2D) with His6-tag at the C-terminal of IL-15Rα are P1185 (SEQ ID NO: 16) and P1275 (SEQ ID NO: 39).

Illustrative proteins of ILR-Fc-a format (FIG. 3B) are P1332 (SEQ ID NO: 74) and P1333 (SEQ ID NO: 75).

C-terminal serine (S162) of IL-15 in IL15 fusion proteins is a potential glycosylation site in IL-15 fusion protein and glycosylation of S162 may affect the potency of the fusion proteins. A point mutation was introduced by standard mutagenesis techniques into IL-15-linker-IL-15Rα-Fc (ILR-Fc), Fc- IL-15-linker-IL-15Rα (Fc-ILR) and IL-15-linker-IL-15Rα (ILR). An illustrative protein of ILR-Fc with S162A is P1253 (SEQ ID NO: 23). Illustrative proteins of Fc-ILR with S162A are P1254 (SEQ ID NO: 24), P1255 (SEQ ID NO: 25), P1279 (SEQ ID NO: 41) and P1280 (SEQ ID NO: 42). Illustrative proteins of ILR-His6 with S162A are P1274 (SEQ ID NO: 38) and P1276 (SEQ ID NO: 40).

### Production, Purification and Characterization

Fc fusion proteins were produced by transient transfection in Expi293 cells and purified by a two-step purification process comprising MabSelect SuRe chromatography (GE Healthcare) and size exclusion chromatography (Superdex 200, GE Healthcare).

His-tagged protein were produced by transient transfection in Expi293 cells and purified by a two-step purification process comprising nickel affinity chromatography (GE Healthcare) and size exclusion chromatography (Superdex 75 or Superdex 200, GE Healthcare).

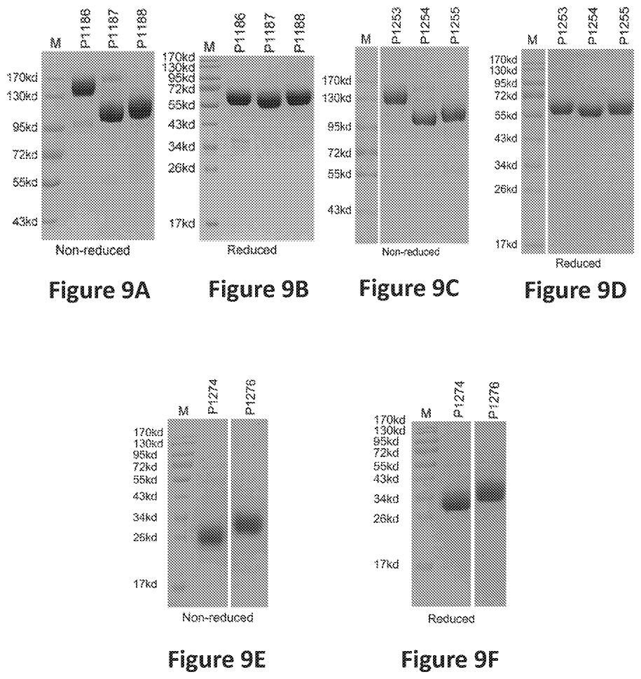

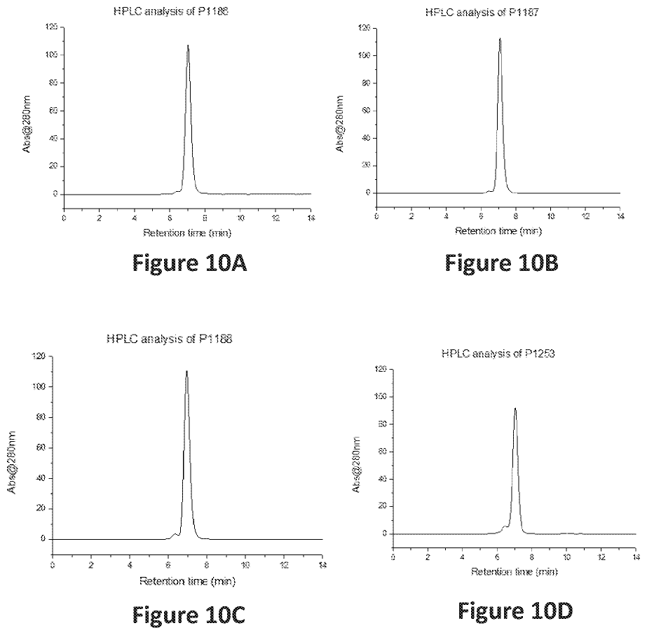

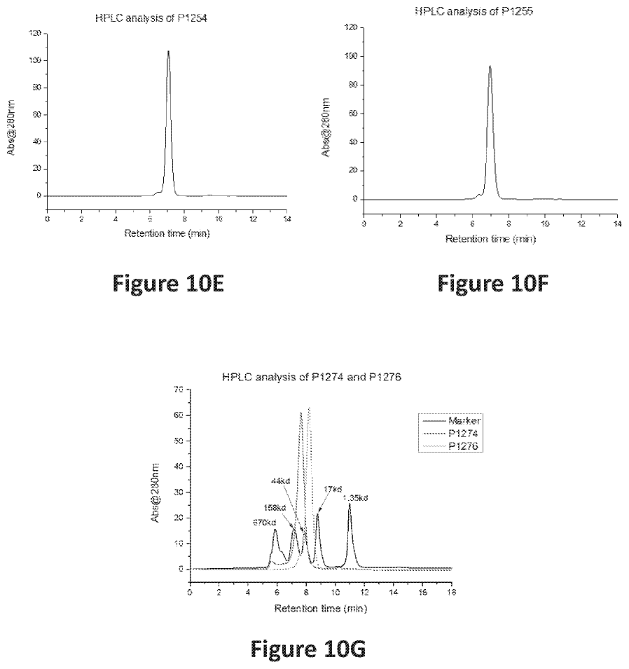

Purified proteins were characterized by SDS-PAGE and high performance liquid chromatography (HPLC) for purity and homogeneity assessment. HPLC analysis was performed using Nanofilm SEC-250 column (Sepax) and Agilent 1260 according to the manufacturer's instructions. Representative SDS-PAGE and HPLC results were shown in FIGS. 9A-9F and FIGS. 10A-10G. Purified protein showed good purity and homogeneity. For ILR-His format, P1274 (SEQ ID NO: 38) with 15 amino acid linker and P1276 (SEQ ID NO: 40) with 26 amino acid linker, the theoretic MW of monomer is around 24KD and dimer is around 50KD. Based on the HPLC result shown in FIG. 10G, it seems that P1274 (SEQ ID NO: 38) existed as homodimer in solution, indicating interchain IL-15/IL-15Rα complex formation, and P1276 (SEQ ID NO: 40) existed as monomer, indicating intrachain IL-15/IL-15Rα complex formation.

### Functional Assays—Cell Signaling

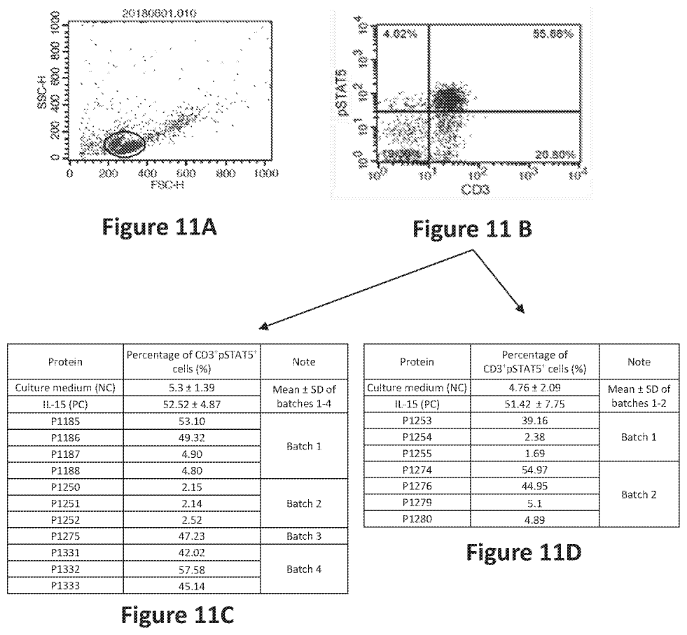

The proteins were then tested in in vitro functional assays. In cell signaling assay (phospho-STAT5) using PBMC, purified fusion proteins (with or without Fc) were incubated with PBMCs at 37° C. for 15 minutes. Following incubation, cellular stimulation was terminated by the addition of a fixation buffer to fix the cells for cellular staining. Cells were subsequently permeabilized by the addition of a permeabilization buffer (BD Perm III buffer, Cat #558050) following manufacturer protocols. PBMCs were then stained with anti-CD3-FITC (UCHT1, BD, Cat #555916) and a phospho-STAT5 antibody (pY694, Alexa647 conjugated, BD, Cat #612599). FIGS. 11A and 11B depict selection of cell population following incubation with P1185 (SEQ ID NO: 16). Lymphocytes were first gated on the basis of side scatter (SSC) and forward scatter (FSC) (FIG. 11A). Lymphocytes were then gated based on CD3 expression and STAT5 phosphorylation (FIG. 11B). The signaling assay results are summarized in FIG. 11C and FIG. 11D. ILR-His (P1185 with SEQ ID NO: 16, P1275 with SEQ ID NO: 39, P1274 with SEQ ID NO: 38 and P1276 with SEQ ID NO: 40) and ILR-Fc (P1186 with SEQ ID NO: 17, P1331 with SEQ ID NO: 73, P1253 with SEQ ID NO: 23, P1332 with SEQ ID NO: 74 and P1333 with SEQ ID NO: 75) showed cell signaling activity (phospho-STAT5) while Fc-ILR (P1187 with SEQ ID NO: 18, P1188 with SEQ ID NO: 19, P1250 with SEQ ID NO: 20, P1251 with SEQ ID NO: 21, P1252 with SEQ ID NO: 22, P1254 with SEQ ID NO: 24, P1255 with SEQ ID NO: 25, P1279 with SEQ ID NO: 41 and P1280 with SEQ ID NO: 42) are not active at stimulating STAT5 phosphorylation.

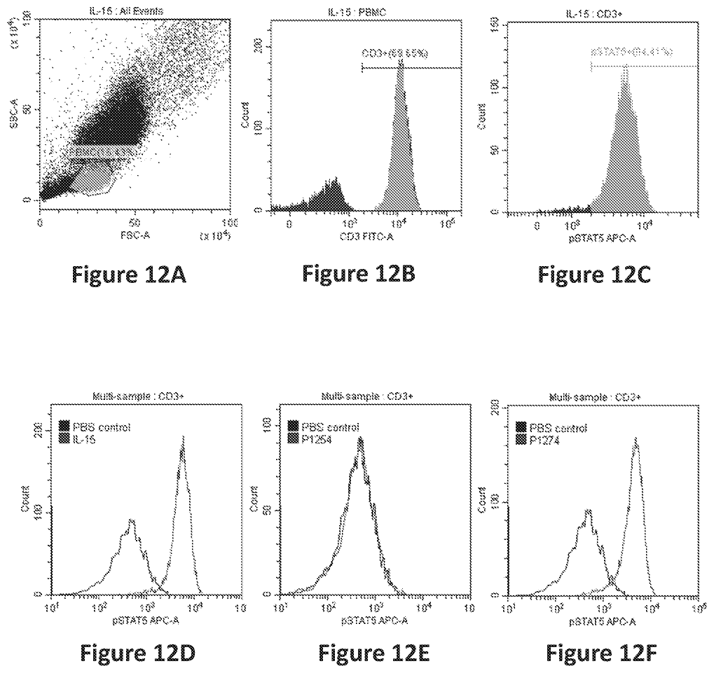

Cell signaling assay was also performed using human whole blood for a few purified fusion proteins. Human whole blood samples were collected from healthy individuals and used following the guidelines according to signed consent agreement. Purified fusion proteins were incubated with fresh human whole blood at 37° C. for 15 minutes. Following incubation, cellular stimulation was terminated by the addition of a lysis and fixation buffer (BD, Cat #558049) to remove red blood cells and fix the remaining cells for cellular staining. Anti-CD3-FITC (UCHT1, BD, Cat #555916) was added to the cell suspensions and incubated for 1 h at 37° C. for staining before being removed by two washing steps using phosphate buffered saline (PBS). Cells were subsequently permeabilized by the addition of a permeabilization buffer (BD Perm III buffer, Cat #558050) following manufacturer protocols and stained for 1 h using a phospho-STAT5 antibody (pY694, Alexa647 conjugated, BD, Cat #612599). The results are summarized in FIGS. 12A-12F and are consistent with cell signaling assay results using PBMC. ILR-His (P1274 with SEQ ID NO: 38) stimulated the proliferation of PBMCs compared to the control group while Fc-ILR (P1254 with SEQ ID NO: 24) did not do so.

### Functional Assays—Proliferation

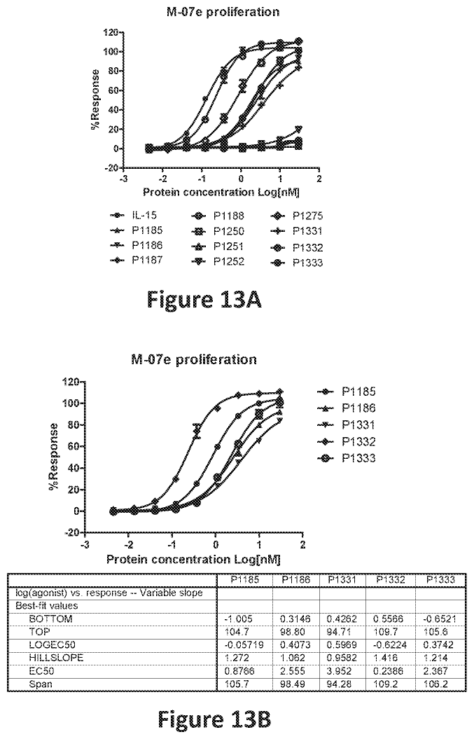

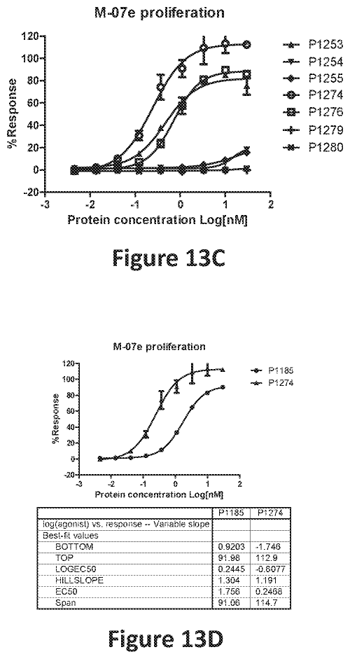

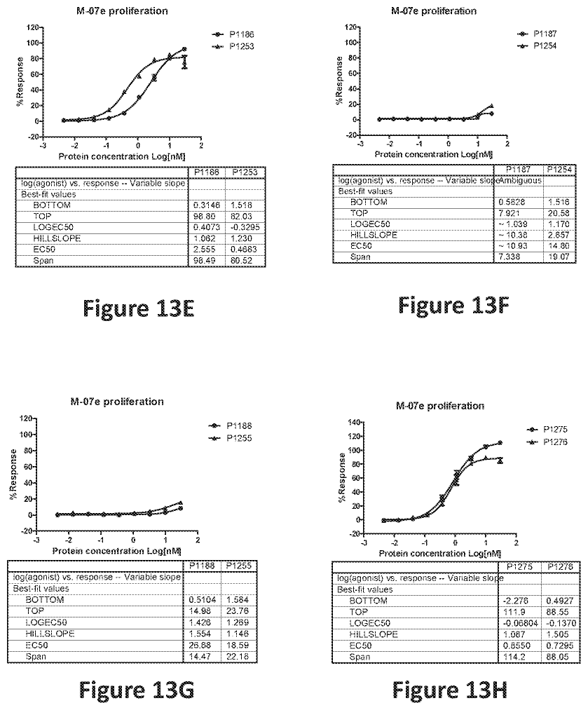

Purified proteins were also tested in proliferation assay. M-07e (IL-2R□/□c) cells were cultured in RPMI 1640 supplemented with 20% fetal bovine serum (FBS), 1% non-essential amino acids (NEAA) and 10% of 5637 cell culture supernatant. To measure cytokine-dependent cell proliferation, M-07e cells were harvested in their logarithmic growth phase and washed twice with PBS. 90 μl of cell suspension (2×10⁴ cells/well) was seeded into 96-well plate and incubated for 4 hours in assay medium (RPMI 1640 supplemented with 10% FBS and 1% NEAA) for cytokine starvation at 37° C. and 5% C02. IL-15 and purified proteins samples used in assays were prepared in assay medium to an initial concentration of 300 nM, followed by ⅓ serial dilutions. 10 μl diluted protein was added into corresponding wells and incubated at 37° C. and 5% C02 for 72 hours. Colorimetric assays using a Cell Counting Kit-8 (CCK-8, Dojindo, CK04) were performed to measure the amount of live cells and the results are shown in FIGS. 13A and 13C. For ILR-Fc format (FIG. 13B), P1186 (SEQ ID NO 17), P1331 (SEQ ID NO: 73) and P1333 (SEQ ID NO: 75) showed 3-4 fold lower activity compared with P1185 (SEQ ID NO: 16).

Constructs with S162A mutation were compared with the corresponding constructs with S162 for potency (FIGS. 13D-13H). For Fc-ILR format (P1254 with SEQ ID NO: 24 and P1255 with SEQ ID NO: 25), activity was not improved with S162A mutation (FIGS. 13F-13G). For ILR-Fc (P1253 with SEQ ID NO: 23) and ILR-His with 15 amino acid linker between IL-15 and IL-15Rα (P1274 with SEQ ID NO: 38) with S162A mutation, potency of the fusion protein was improved by 5-7 fold (FIGS. 13D-13E). For ILR-His with 26 amino acid linker between IL-15 and IL-15Rα (P1276 with SEQ ID NO: 40), potency was not improved with S162A mutation (FIG. 13H), it seems that the linker between IL-15 and IL15-Ra may interfere with receptor P7 binding to IL-15 due to intrachain LR dimer formation.

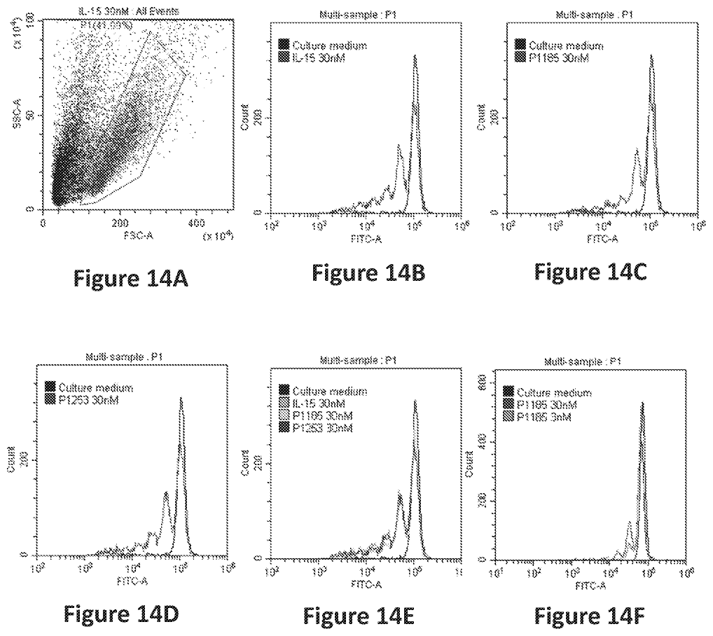

Proliferation assay was also performed using human peripheral blood mononuclear cells (PBMCs) for a few purified fusion proteins. Human PBMCs were prepared by Ficoll centrifugation and stained with 5-(6)-carboxy-fluorescein succinimidyl ester (CFSE, Thermofisher, Cat #C34554) according to the manufacturer's instructions. Stained human PBMCs were seeded into 96-well plate (5×10⁴ cells per well) and incubated with purified fusion proteins at 37° C. and 5% C02 for 5-6 days. Human PBMC proliferation was assessed by flow cytometry and the results are summarized in FIGS. 14A-14F. Human PBMC proliferation results were consistent with other functional assay results, ILR-His (P1185 with SEQ ID NO: 16) and ILR-Fc (P1253 with SEQ ID NO: 23) could stimulate the proliferation of PBMCs.

### Example 2: Engineering of Fc-IL-15-Linker-IL-15Rα Fusion Proteins with Protease Cleavage Site in the Linker Between Fc and IL-15

In order to restore the activity of IL-15, TEV protease cleavage site was used to prove the concept and introduced into the linker between Fc and IL-15 of Fc-ILR constructs.

Plasmids coding for Fc-TEV-ILR were constructed by standard gene synthesis and sub-cloned into pTT5 expression vector with different lengths of linker flanking TEV cleavage site. Cartoon schematics of illustrative Fc-TEV-ILR fusion protein format are depicted in FIG. 6. Illustrative proteins of Fc-TEV-ILR format include P1256 (SEQ ID NO: 26), P1261 (SEQ ID NO: 32), P1281 (SEQ ID NO: 43), P1282 (SEQ ID NO: 49), P1283 (SEQ ID NO: 55), P1284 (SEQ ID NO: 61) and P1285 (SEQ ID NO: 67).

To form tighter IL-15/IL-15Rα complex, a disulfide bond was introduced between IL-15 and IL-15Rα with L52C mutation on IL-15 and S40C mutation on IL-15Rα. Illustrative proteins of this format include P1378 (SEQ ID NO: 226) and P1379 (SEQ ID NO: 232).

For activatable prodrug design, real protease cleavage sites (PSs) were introduced into the linker between Fc and IL-15 of Fc-ILR constructs. Plasmids coding for Fc-PSs-ILR were constructed by standard gene synthesis and sub-cloned into pTT5 expression vector with linker flanking protease cleavage sites. Cartoon schematics of illustrative Fc-PSs-ILR fusion protein format are depicted in FIG. 6. Illustrative proteins of Fc-PSs-ILR format include P1334 (SEQ ID NO: 76), P1335 (SEQ ID NO: 87), P1336 (SEQ ID NO: 98), P1337 (SEQ ID NO: 114), P1338 (SEQ ID NO: 130), P1339 (SEQ ID NO: 146), P1340 (SEQ ID NO: 162), P1341 (SEQ ID NO: 178), P1342 (SEQ ID NO: 194), P1343 (SEQ ID NO: 210), P1380 (SEQ ID NO: 238), P1381 (SEQ ID NO: 254), P1382 (SEQ ID NO: 270), P1383 (SEQ ID NO: 286), P1423 (SEQ ID NO: 338), P1424 (SEQ ID NO: 339), P1425 (SEQ ID NO: 340), P1426 (SEQ ID NO: 341), P1427 (SEQ ID NO: 342) and P1428 (SEQ ID NO: 343).

Fc fusion proteins were produced, purified and characterized as described in Example 1.

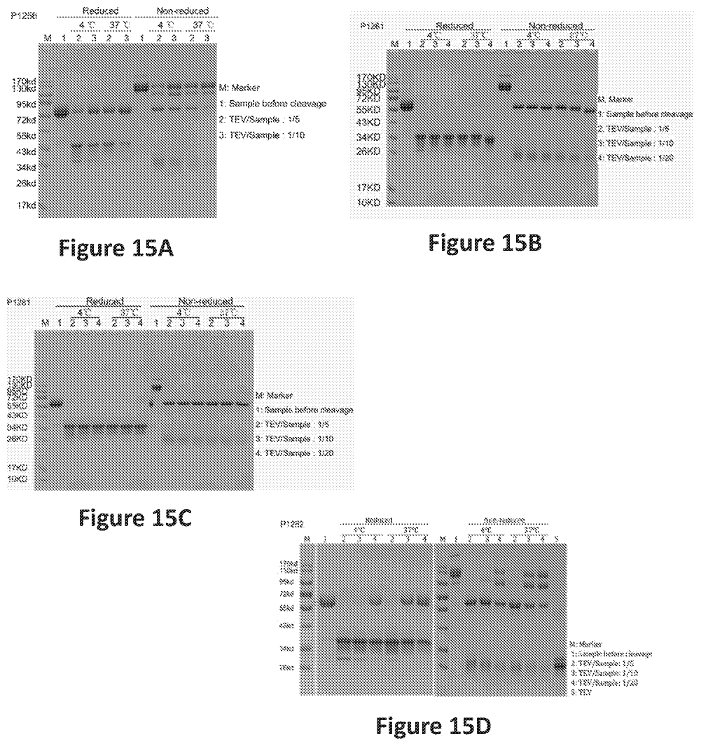

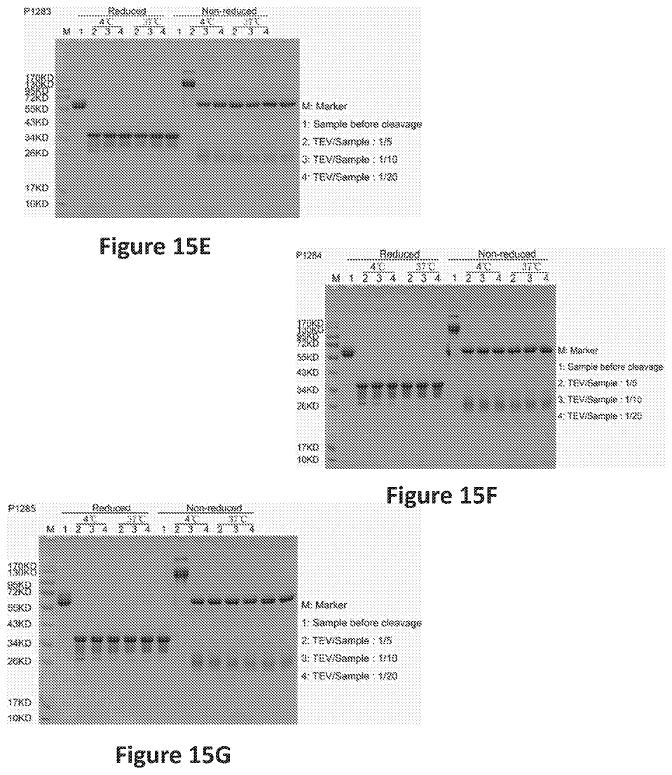

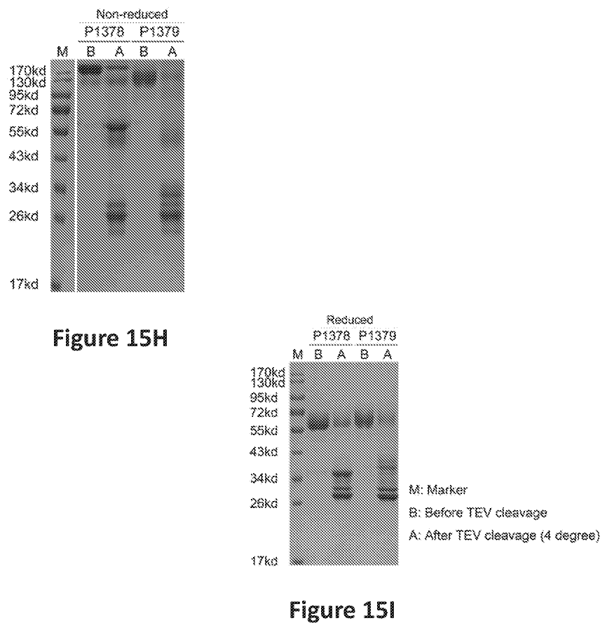

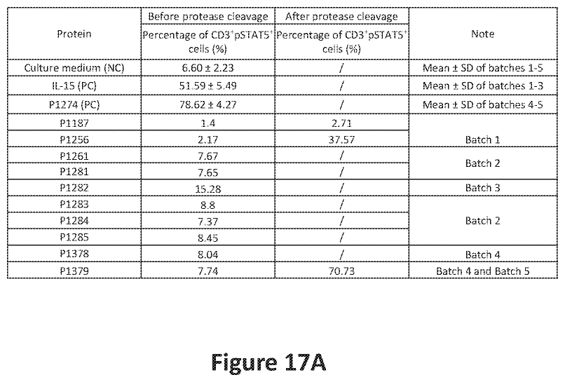

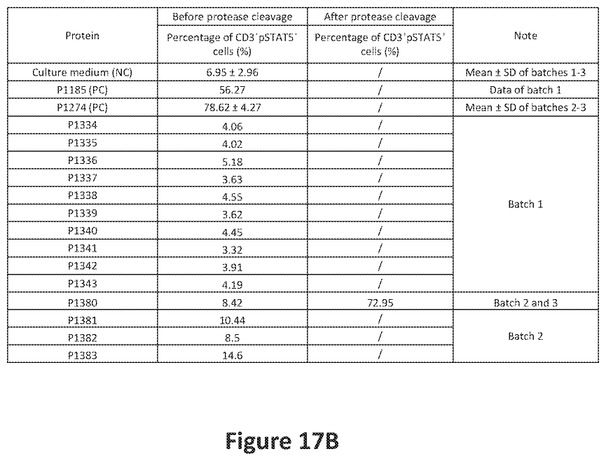

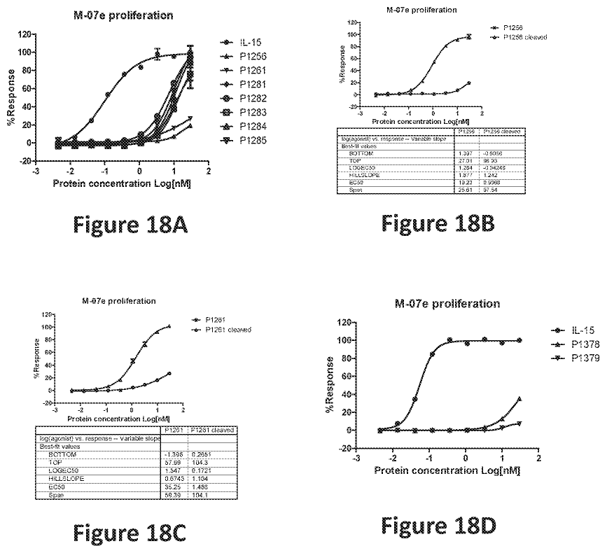

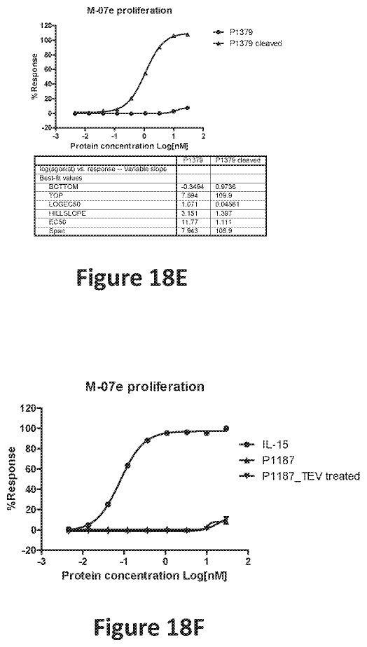

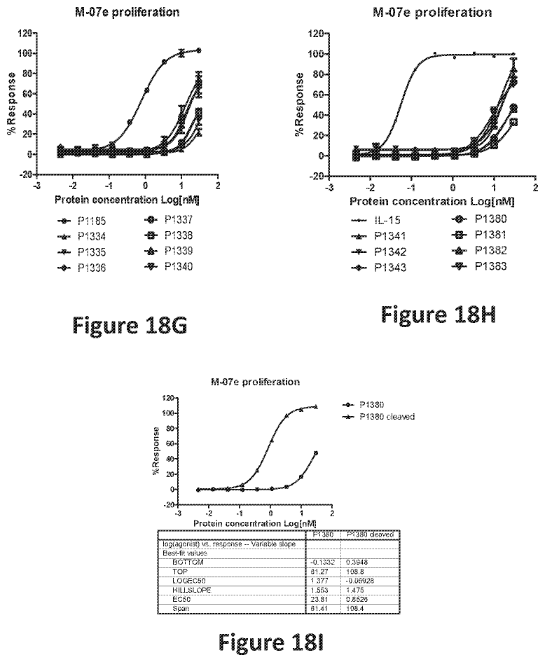

Purified proteins with TEV cleavage site were tested by TEV cleavage (FIGS. 15A-15I). P1256 (SEQ ID NO: 26), with shortest linker flanking TEV cleavage site, could be partially cleaved by TEV at both 4° C. and 37° C. at TEV to protein ratio of 1 to 5 and 1:10. For proteins (P1261 with SEQ ID NO 32, P2181 with SEQ ID NO 43, P1282 with SEQ ID NO 49, P1283 with SEQ ID NO 55, P1284 with SEQ ID NO 61 and P1285 with SEQ ID NO 67) with longer linker flanking TEV cleavage site than P1256 (SEQ ID NO: 26), most of the target protein could be cleaved by TEV at both 4° C. and 37° C. at a TEV to protein ratio of 1 to 20. P1378 (SEQ ID NO 226) and P1379 (SEQ ID NO 232) could be partially cleaved by TEV at 4° C. at a TEV to protein ratio of 1 to 1. Uncleaved and TEV cleaved proteins were then tested in in vitro functional assays using the same method as described in Example 1. Cell signaling assay (phospho-STAT5) results are summarized in FIG. 17A and M-07e proliferation assay results are summarized in FIGS. 18A-18F. Based on functional assay results, IL-15 activity was restored after TEV cleavage between Fc and IL-15 on Fc-TEV-ILR fusion proteins while TEV treated P1187 (SEQ ID NO: 18) did not show function.

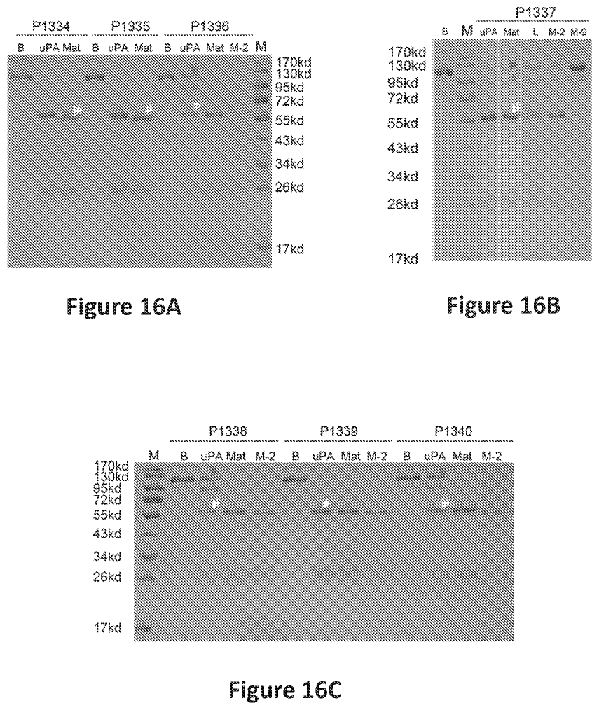

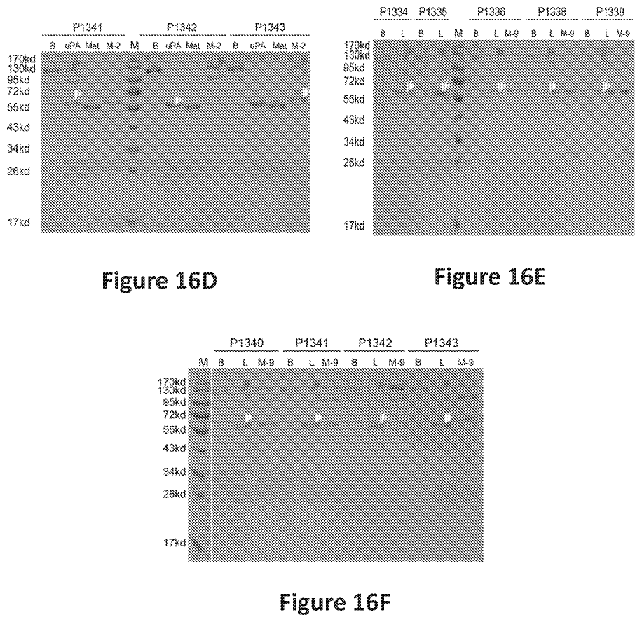

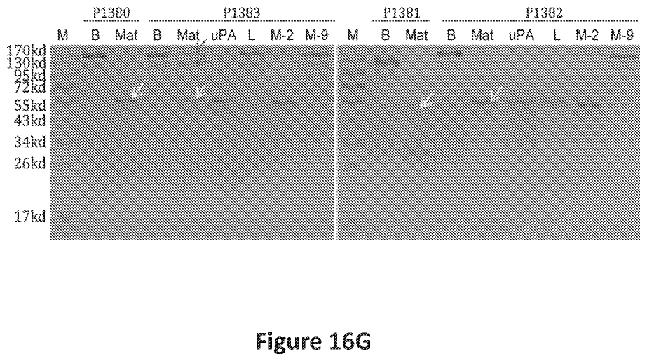

Purified Fc-PSs-ILR proteins were tested for protease cleavage assays by protease uPA (R&D, Cat #1310-SE-010), matriptase (R&D, Cat #3946-SEB-010), legumain (R&D, Cat #2199-CY-010), MMP-2 (R&D, Cat #902-MP-010) and MMP-9 (R&D, Cat #911-MP-010) respectively and the results are shown in FIGS. 16A-16G. The purified proteins could be cleaved completely or partially by the above proteases.

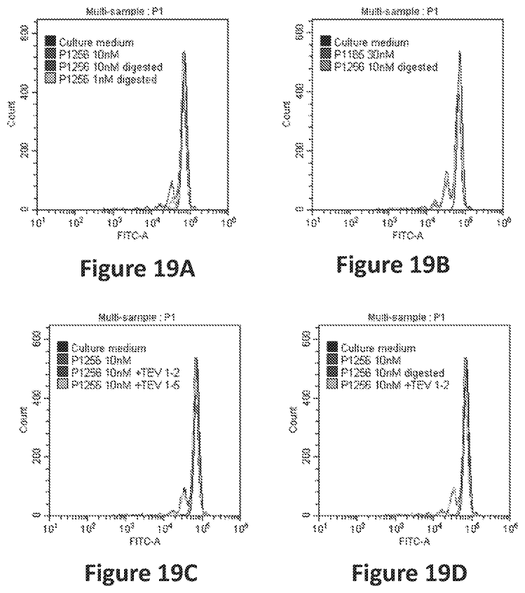

Uncleaved and protease cleaved proteins were then tested in in vitro functional assays using the same methods as described in Example 1. Cell signaling assay (phospho-STAT5) results are summarized in FIG. 17B, M-07e proliferation assay results are summarized in FIGS. 18G-18I and PBMC proliferation assay results are summarized in FIG. 19A-19D. IL-15 activity was restored after protease cleavage between Fc and IL-15.

### Example 3: Engineering of Fc-IL-15-Linker-IL-15Rα Fusion Proteins with Protease Cleavage Site in the Linker Between IL-15 and IL-15Rα

In order to restore the activity of IL-15, TEV protease cleavage site was used to prove the concept and introduced into the linker between IL-15 and IL-15Rα of Fc-ILR constructs.

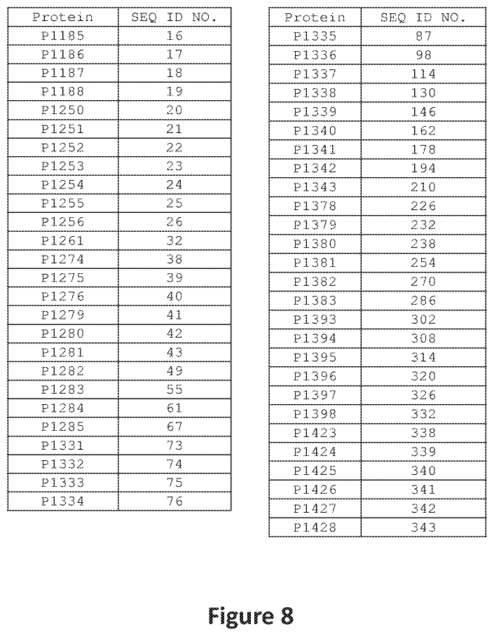

Plasmids were constructed using the same method as described in Example 2. Cartoon schematics of illustrative Fc-ILR fusion protein format are depicted in FIG. 8.

Illustrative proteins of Fc-IL-15 (S162A)-TEV-IL-15Rα format include P1393 (SEQ ID NO: 302), P1394 (SEQ ID NO: 308) and P1395 (SEQ ID NO: 314).

Illustrative proteins of Fc-IL-15 (S162A, L52C)-TEV-IL-15Rα (S40C) format include P1396 (SEQ ID NO: 320), P1397 (SEQ ID NO: 326) and P1398 (SEQ ID NO: 332).

Fc fusion proteins were produced, purified and characterized as described in Example 1.

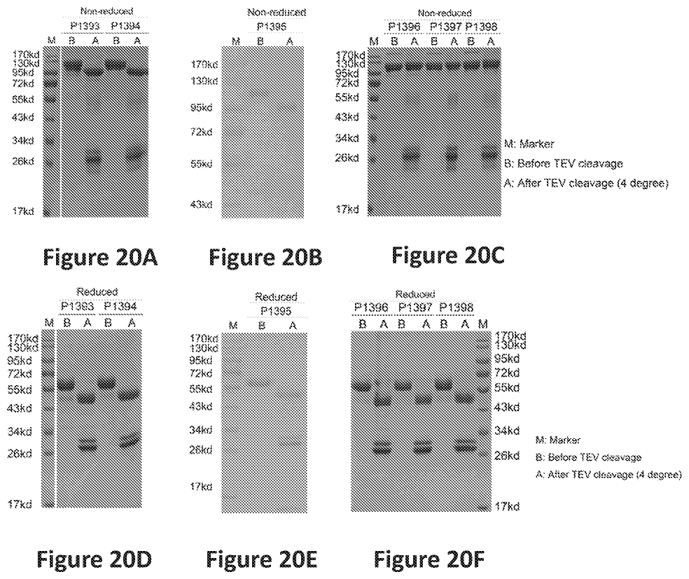

Purified proteins were tested by TEV cleavage at TEV to protein ratio of 1 to 1 and the results are shown in FIGS. 20A-20F. The results showed that all of the six proteins could be cleaved by TEV completely at TEV to protein ratio of 1 to 1 at 4° C.

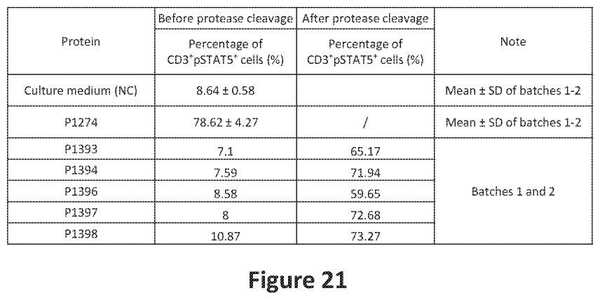

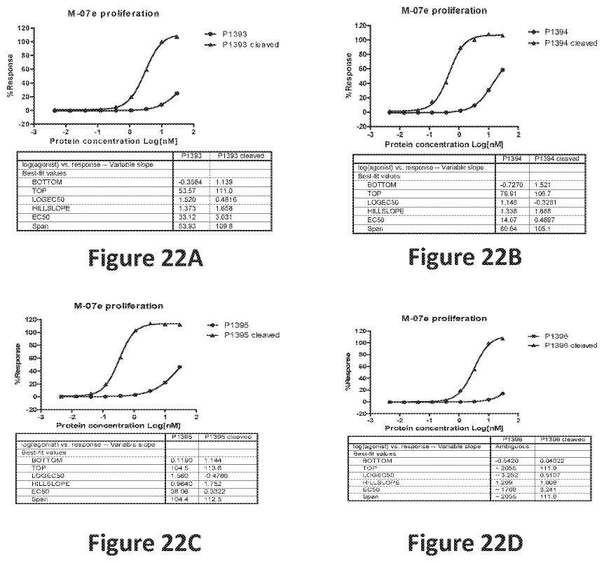

Uncleaved and TEV cleaved proteins were then tested in in vitro functional assays using the same methods as described in Example 1. Cell signaling assay (phospho-STAT5) results are summarized in FIG. 21 and M-07e proliferation assay results are summarized in FIGS. 22A-22F. IL-15 activity was restored after protease cleavage of the linker sequence between IL-15 and IL-15Rα.

### Example 4: Optimization of Cleavable Linker Sequence

For activatable prodrug design, different protease cleavage sequences were introduced into the linker between Fc and IL-15 for Fc-IL-15-PS-IL-15Rα format. Plasmids coding for Fc-IL-15-PS-IL-15Rα were synthesized by standard gene synthesis and sub-cloned into pTT5 vector.

Illustrative proteins of Fc-PS-IL-15-stable linker-IL-15Rα include P1423, P1424, P1425, P1426, P1427, P1428, P1471, P1472, P1473, P1474, P1475, P1476, P1477, P1478, P1479, P1480, P1481, P1482, P1483, P1484, P1485, P1486, P1487, P1488, P1489, P1490, P1491, P1492, P1493, P1494, P1495, P1496, P1497, P1498, P1499, P1500, P1501, P1502, P1503, P1504, P1505, P1506, P1511, P1540, P1541, P1637, P1638, P1639, P1658 and P1659.

Illustrative proteins of Fc-PS-IL-15mut-stable linker-IL-15Rα include P1652, P1653, P1654, P1655, P1656 and P1657.

Illustrative proteins of Fc-PS-IL-15-stable linker-IL-15Rα with optimized codon for IL-15 and IL-15Rα include P1660, P1661 and P1663.

Illustrative proteins of Fc-PS-IL-15-stable linker-IL-15Rα with different stable linker length include P2162, P2163, P2164, P2165, P2166, P2167, P2168 and P2169.

Illustrative proteins of Fc-stable linker-IL-15-PS-IL-15Rα include P1542, P1636, P1696, P1697, P1698, P1699, P1700, P1701, P1702, P1703, P1704, P1705, P1706 and P1707.

Illustrative proteins of Fc-stable linker-IL-15-PS-IL-15Rα with disulfide bond between two IL-15Rα molecules include P1973, P1974 and P1975.

Illustrative proteins of Fc-PS-IL-15-stable linker-IL-15Rα-sushi include P1682 and P1683.

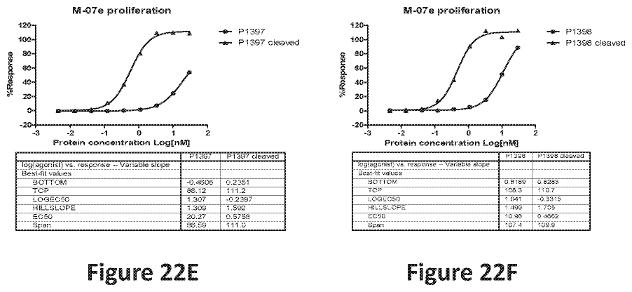

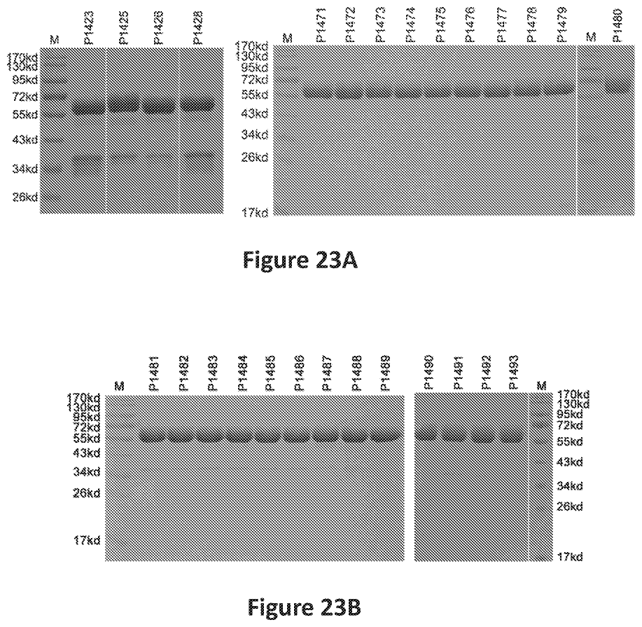

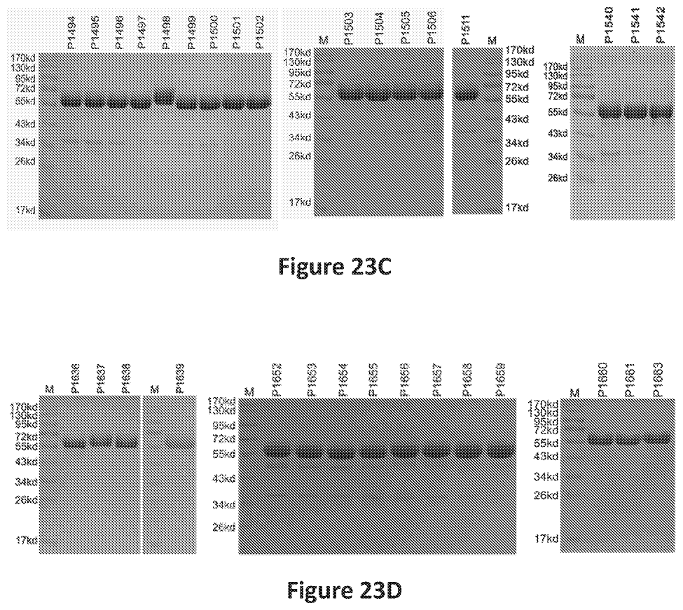

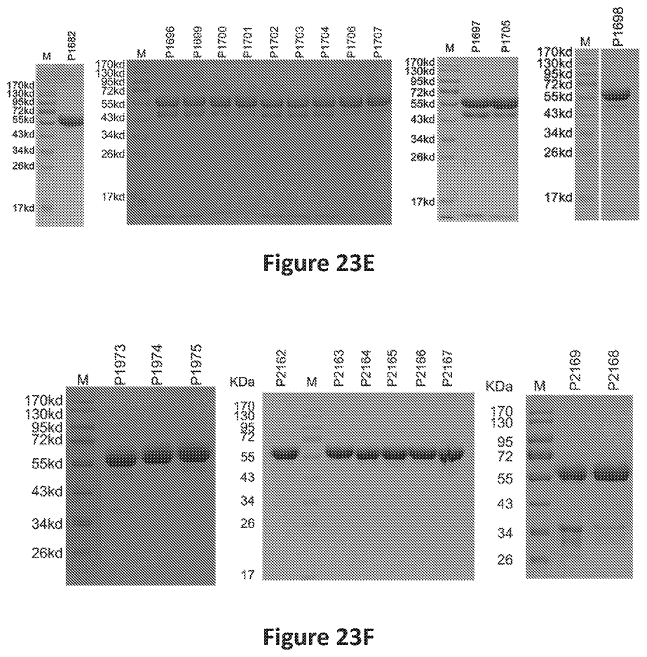

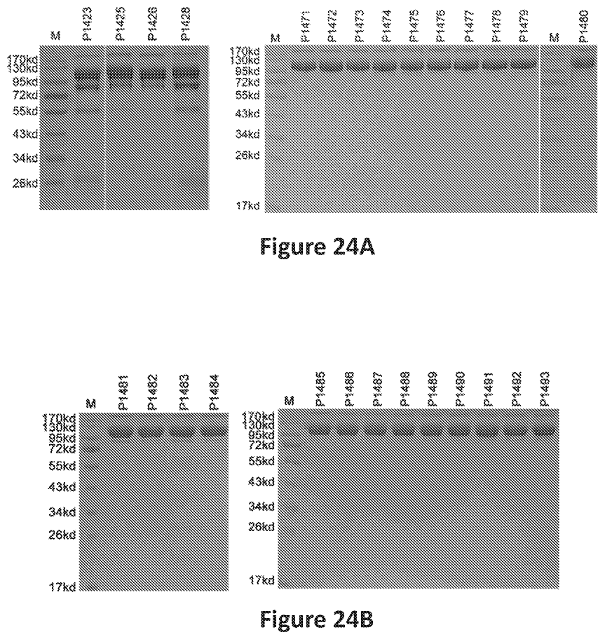

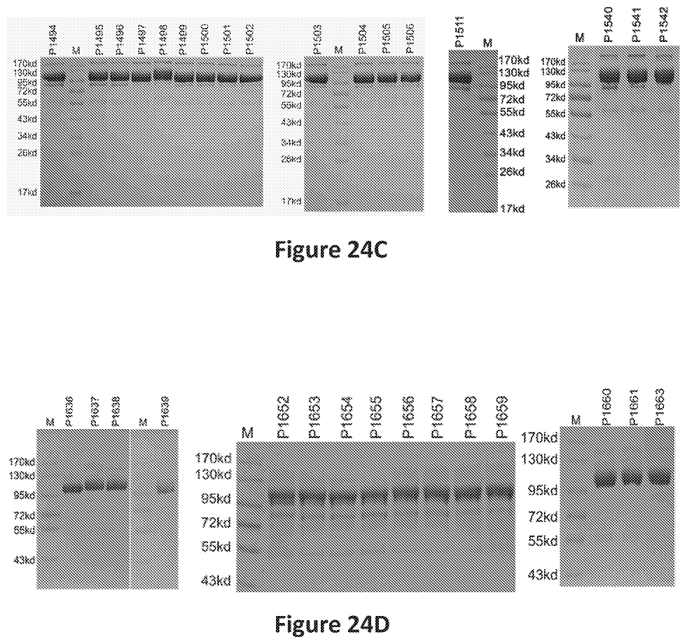

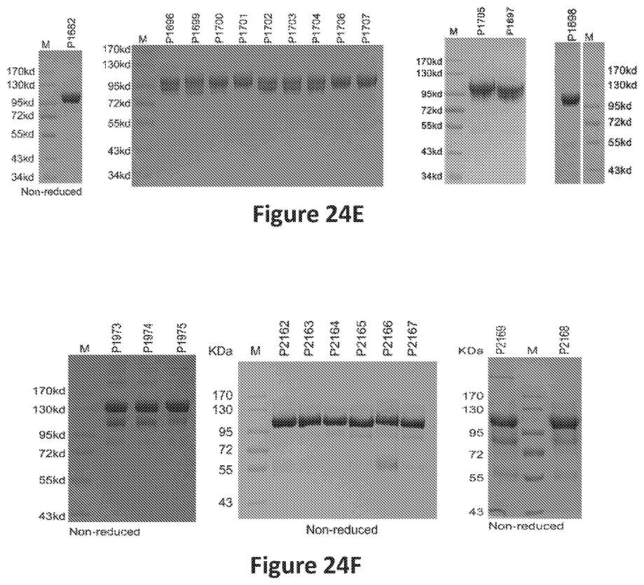

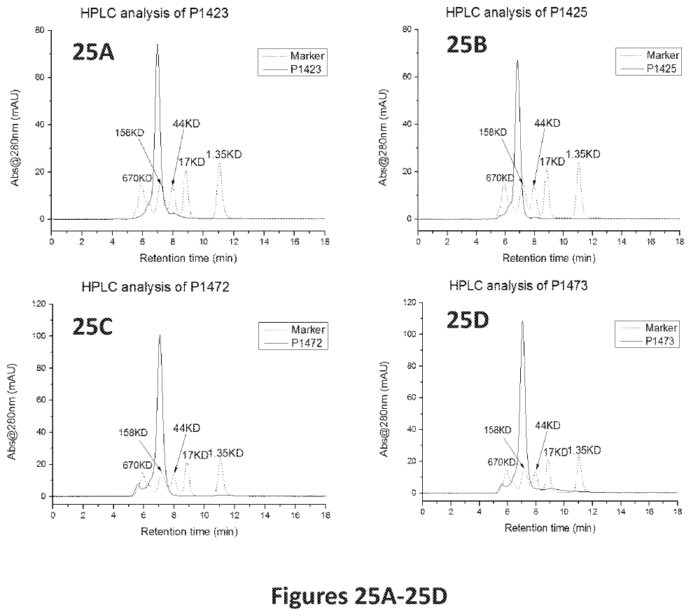

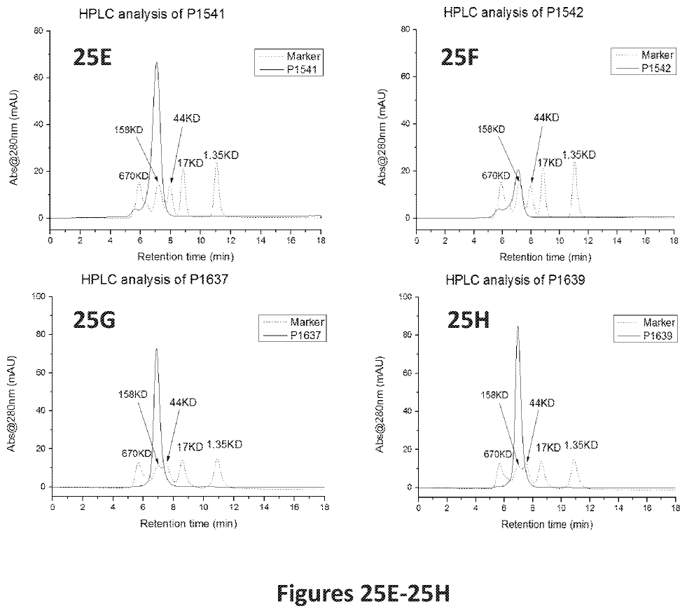

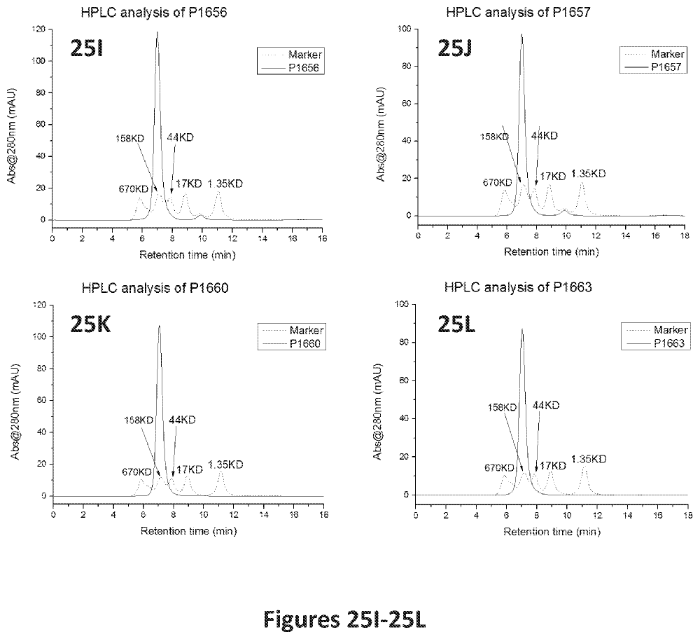

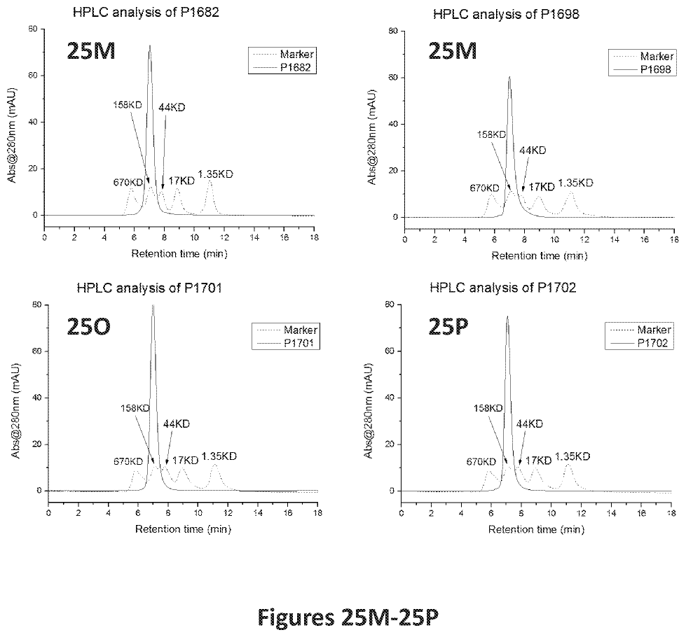

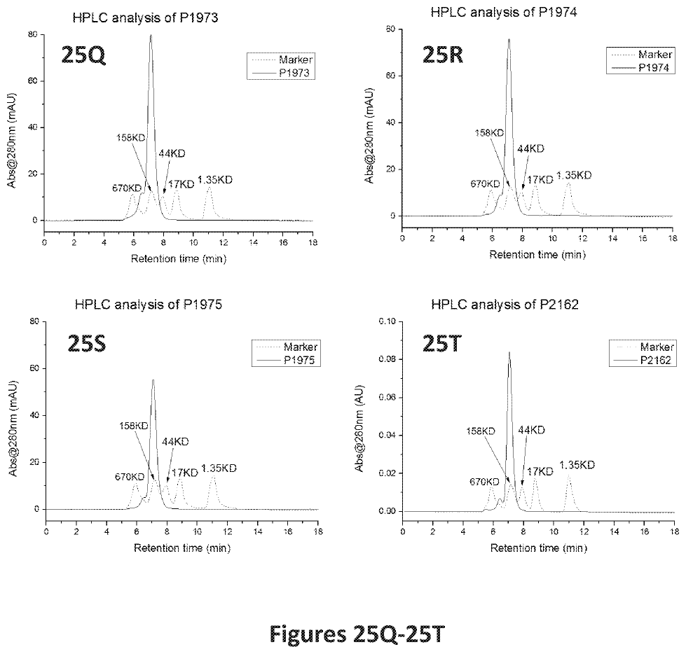

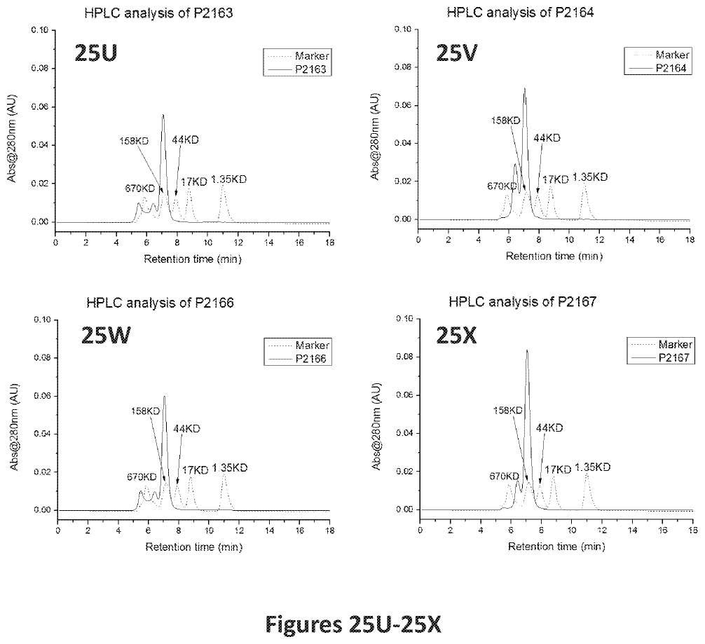

Fc fusion proteins were produced, purified and characterized as described in Example 1. Representative SDS-PAGE and HPLC results are summarized in FIGS. 23A-23F, FIGS. 24A-24F, and FIGS. 25A-25X. Purified proteins showed good purity based on the SDS-PAGE results. Most of the purified proteins showed good homogeneity based on the HPLC results. Dimer and/or polymer were observed for the fusion proteins with shorter linker between IL-15 and IL-15Rα, e.g. P2163, P2164, P2166 and P2167.

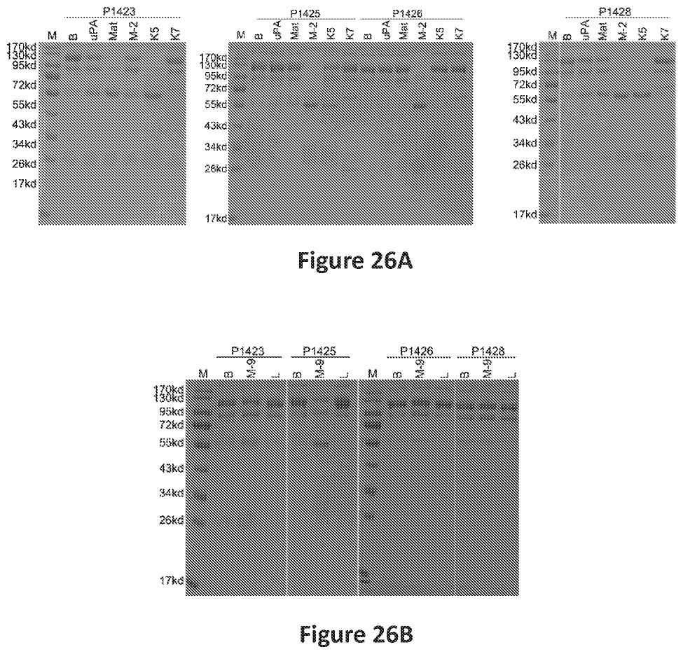

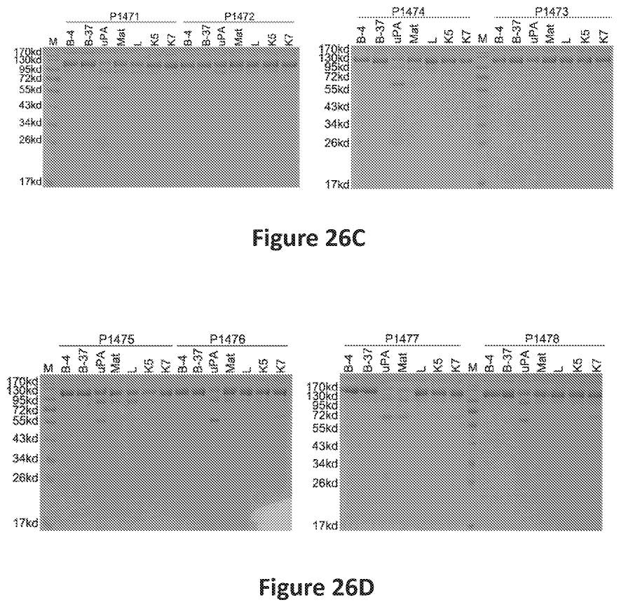

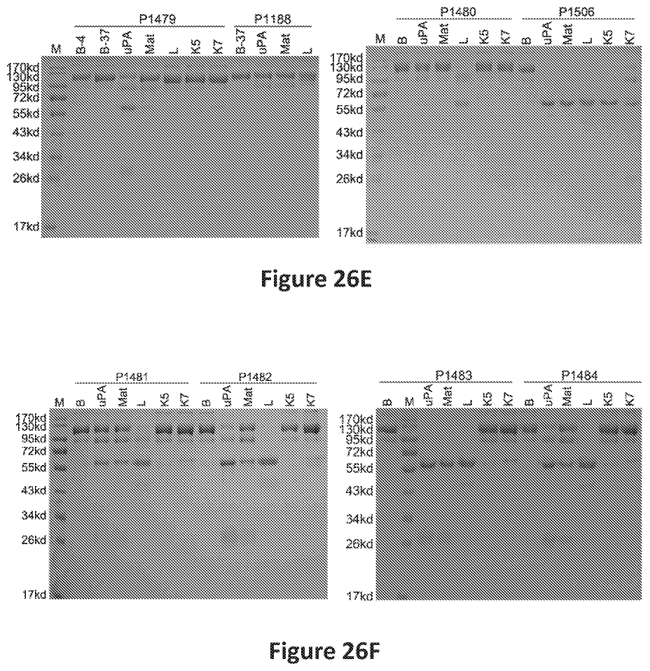

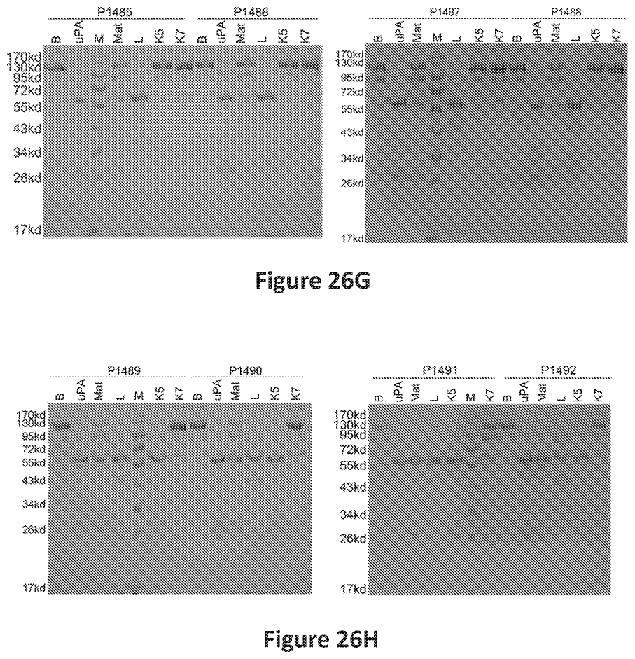

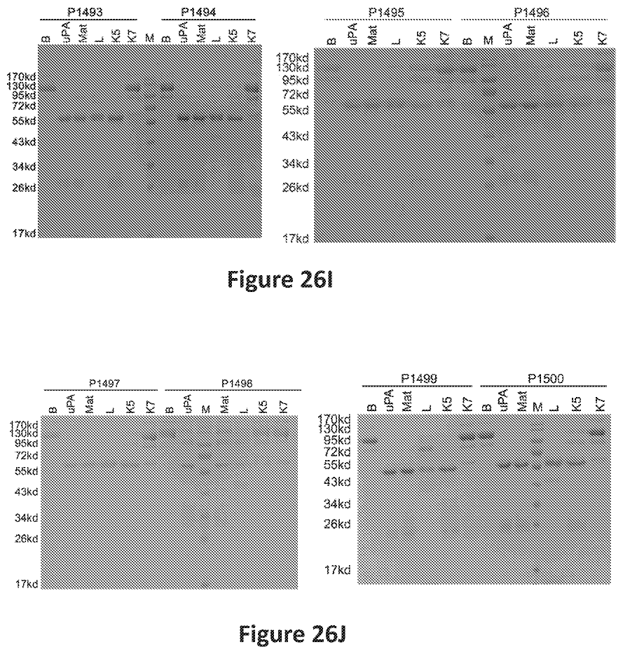

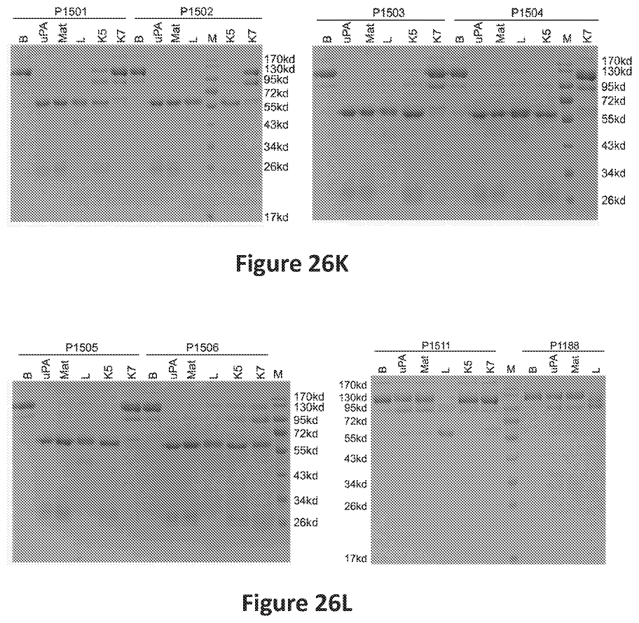

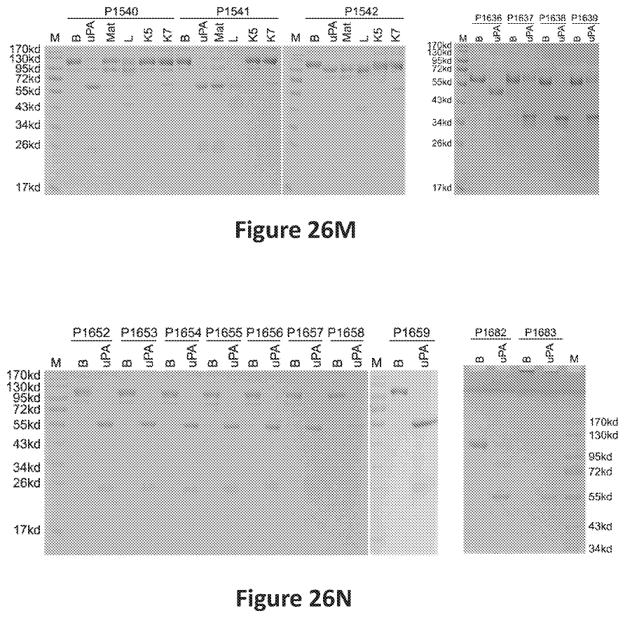

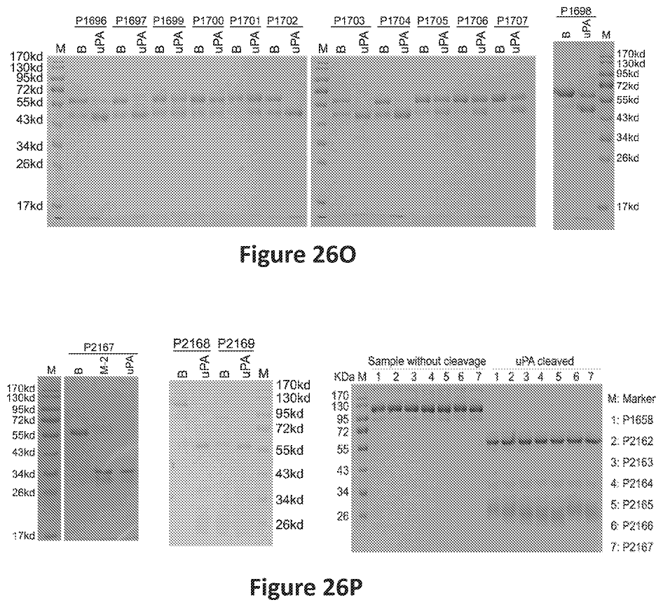

Purified proteins were tested for protease cleavage assays by protease uPA (R&D, Cat #1310-SE-010), matriptase (R&D, Cat #3946-SEB-010), legumain (R&D, Cat #2199-CY-010), MMP-2 (R&D, Cat #902-MP-010), MMP-9 (R&D, Cat #911-MP-010), KLK5 (R&D, Cat #1108-SE) or/and KLK7 (R&D, Cat #2624-SE) respectively and representative results are shown in FIGS. 26A-26P.

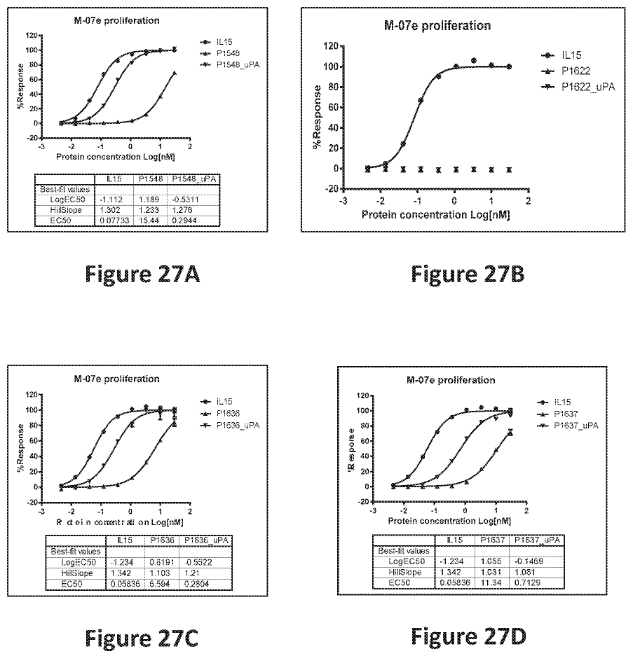

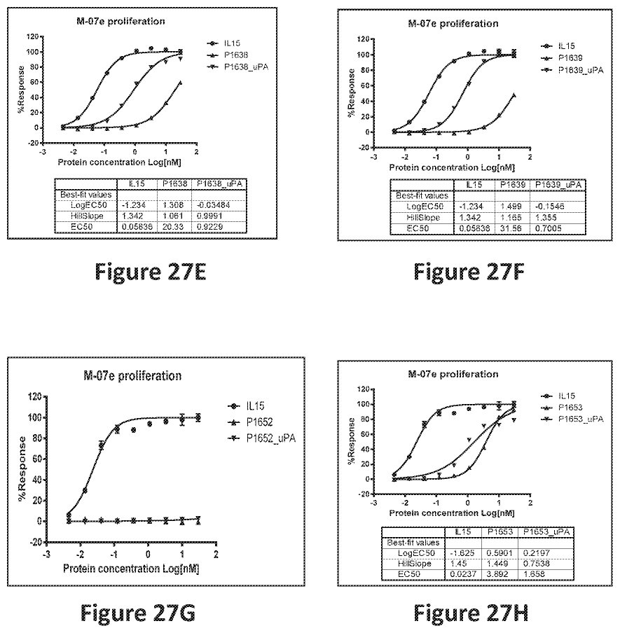

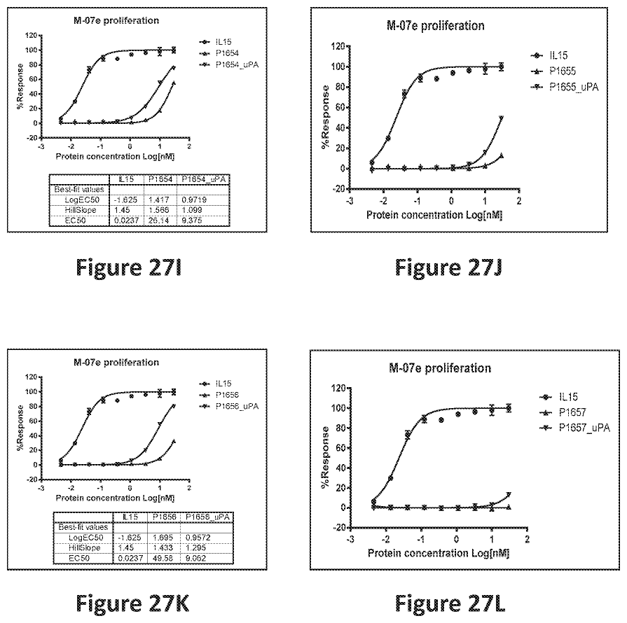

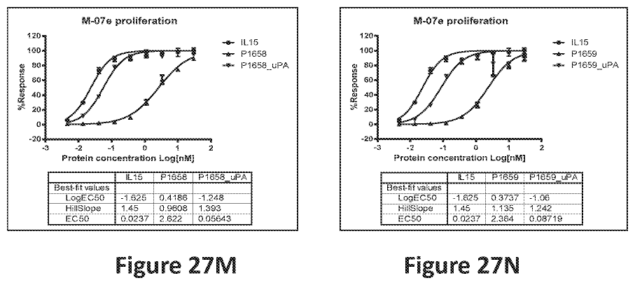

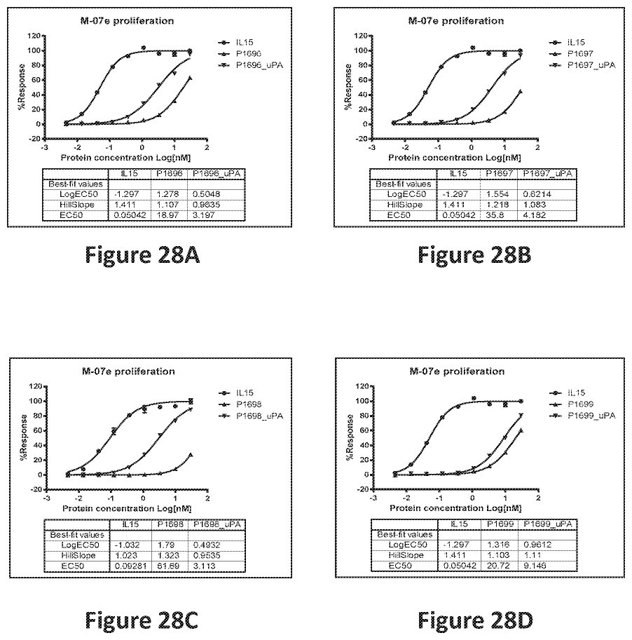

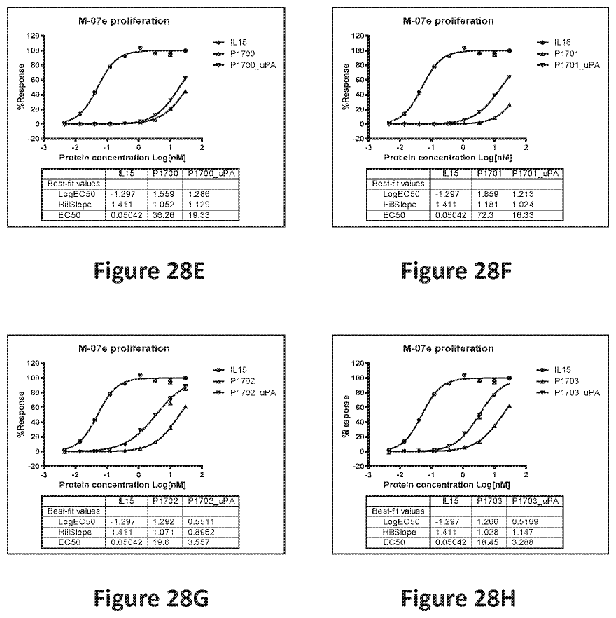

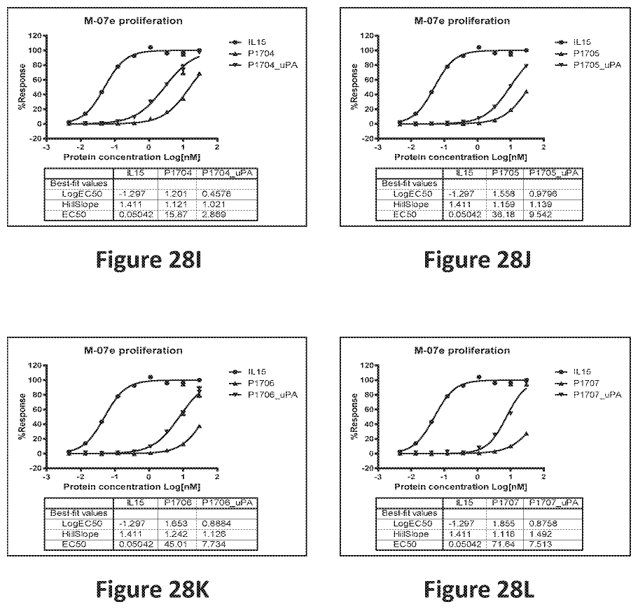

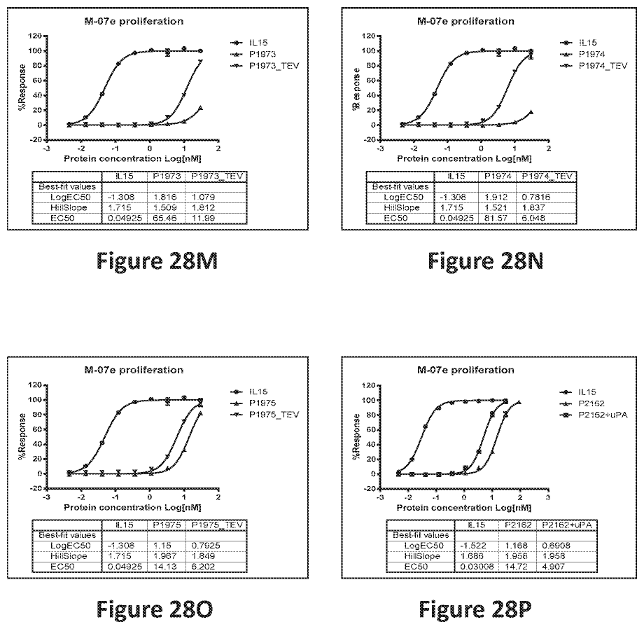

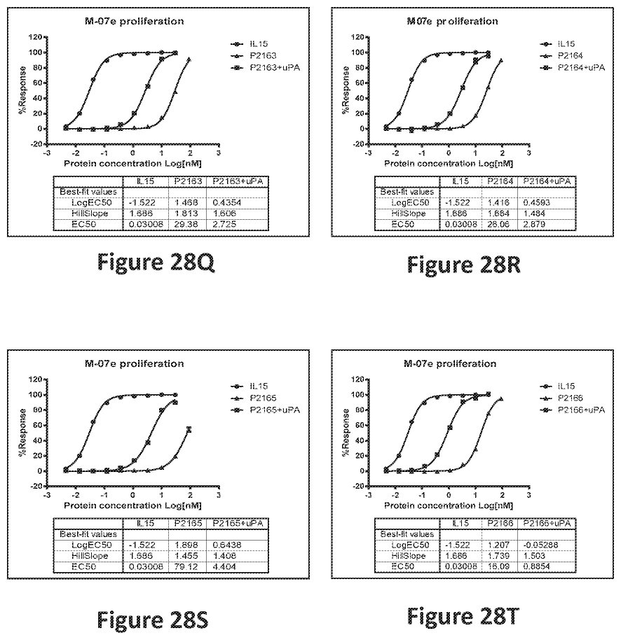

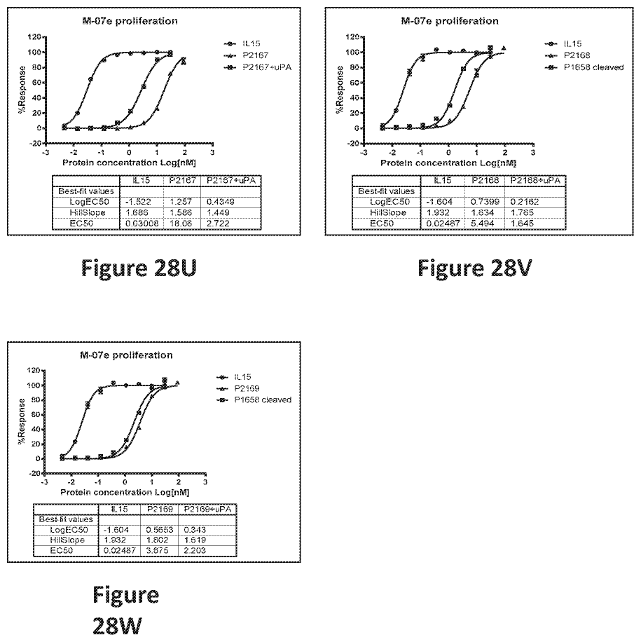

Uncleaved and protease cleaved proteins were then tested in M-07e proliferation assay using the same method as described in Example 1. The results are summarized in FIGS. 27A-27N and FIGS. 28A-28W.

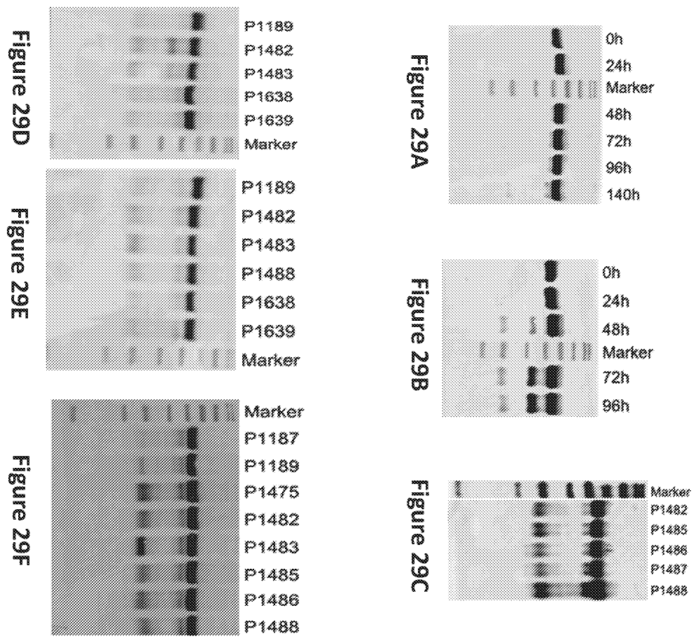

Protein in vitro serum stability was tested in serum from human, cyno, mouse and rat. Equivalent amounts of protein were incubated with fresh serum at 37° C. for different times. Protein was run on 12% SDS-PAGE gel and transferred to nitrocellulose membrane. Membranes were blocked with non-fat dry milk or BSA in TBST, probed with biotinylated anti-IL-15 antibody (R&D, Cat #BAF247), and subsequently probed with streptavidin-HRP. Alternatively, membranes were probed with HRP-anti-human IgG antibody. Representative western blot results are shown in FIGS. 29A-29F.

### Example 5: Engineering of Anti-FAP-IL-15 Prodrug

IL-15 prodrug was fused onto the C-terminal of anti-FAP antibody with protease cleavable sequence between Fc and IL-15 or/and with cleavable protease sequence between IL-15 and IL-15Rα. Plasmids coding anti-FAP-IL-15 prodrug was synthesized by standard gene synthesis and sub-cloned into pTT5 vector.

Illustrative proteins with protease cleavage site between Fc and IL-15 include P15431450 and P16401450.

Illustrative proteins with protease cleavage site between IL-15 and IL-15Rα include P18121450, P18131453, P18141563, P18151450, P18161453, P18171563, P19681450 and P19691450.

Illustrative proteins with protease cleavage site between Fc and IL-15 and with protease cleavage site between IL-15 and IL-15Rα include P24872158, P24882158, P24892158, P24902158, P24912158, P25162158 and P25172158.

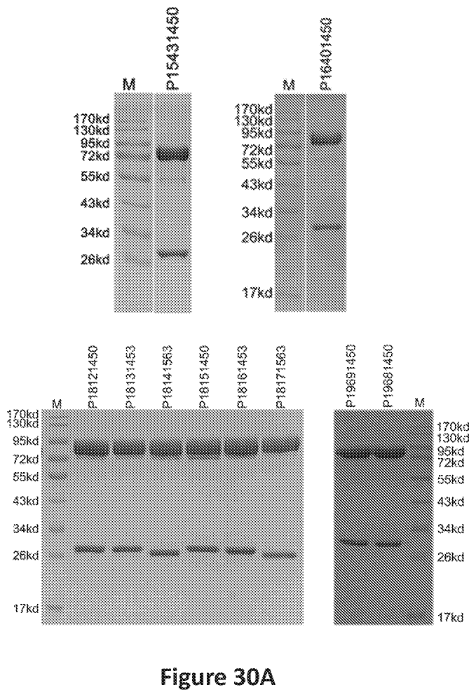

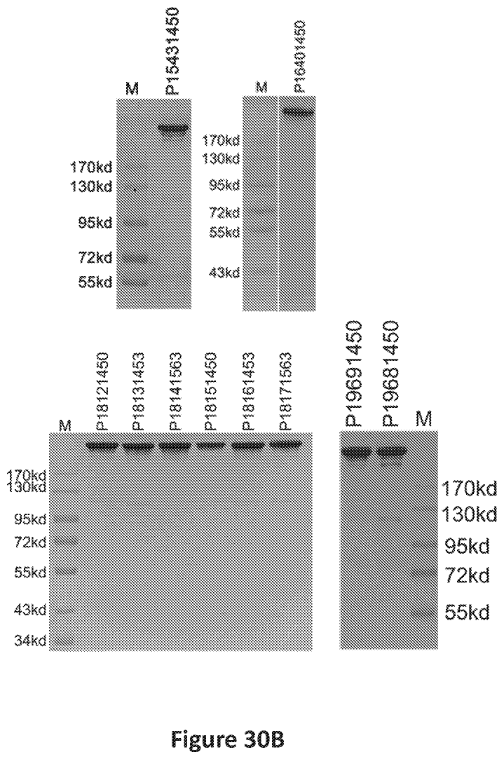

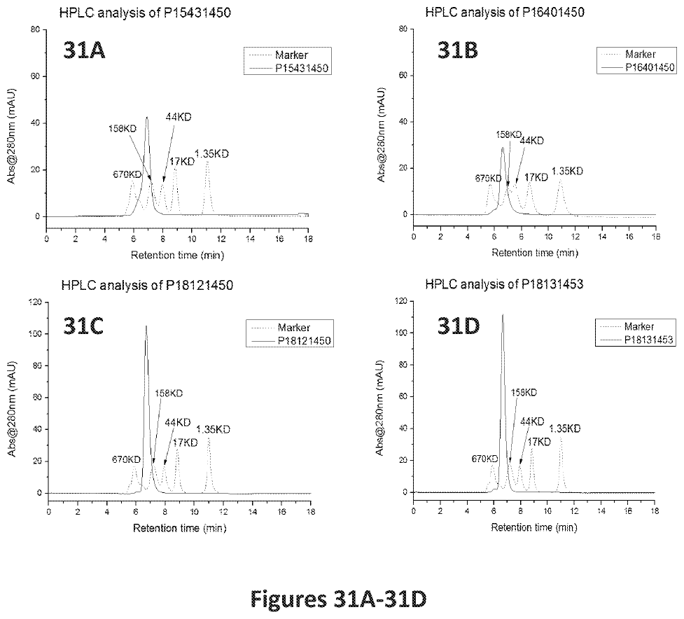

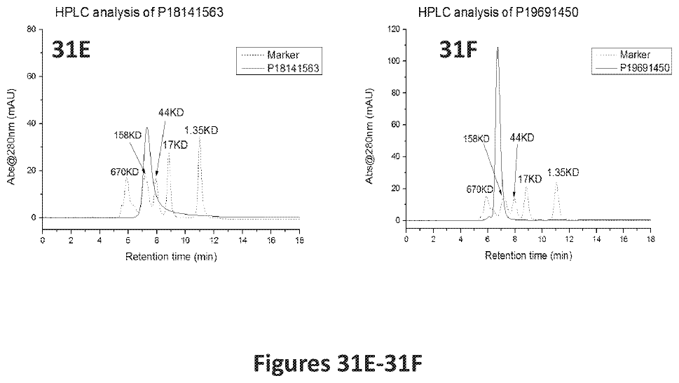

Anti-FAP-IL-15 fusion proteins were produced, purified and characterized as described in Example 1. Representative SDS-PAGE and HPLC results were summarized in FIGS. 30A-30B and FIGS. 31A-31F. Purified proteins showed good purity based on the SDS-PAGE results. Most of the purified proteins showed good homogeneity based on the HPLC results.

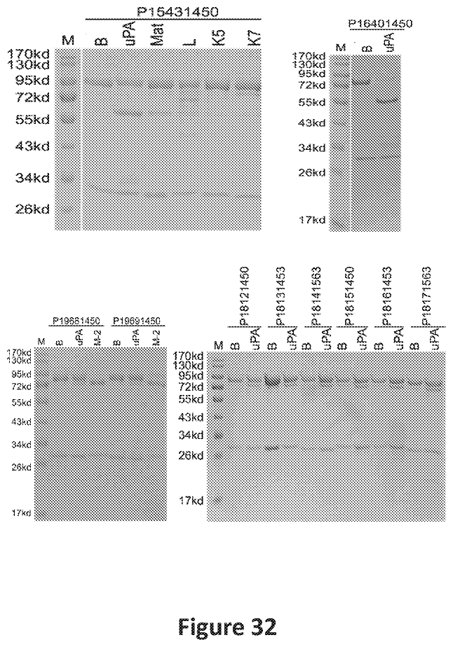

Purified proteins were tested for protease cleavage assays by protease uPA (R&D, Cat #1310-SE-010), matriptase (R&D, Cat #3946-SEB-010), legumain (R&D, Cat #2199-CY-010), MMP-2 (R&D, Cat #902-MP-010), MMP-9 (R&D, Cat #911-MP-010), KLK5 (R&D, Cat #1108-SE) or/and KLK7 (R&D, Cat #2624-SE) respectively and the results are shown in FIG. 32.

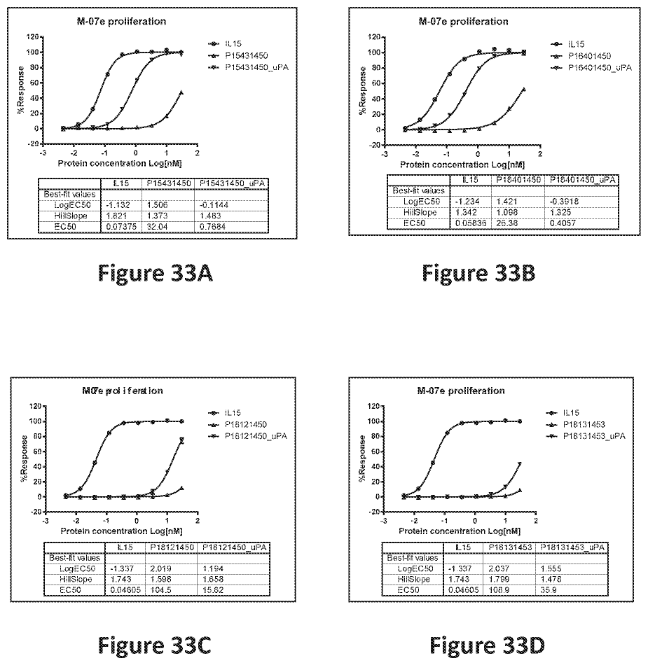

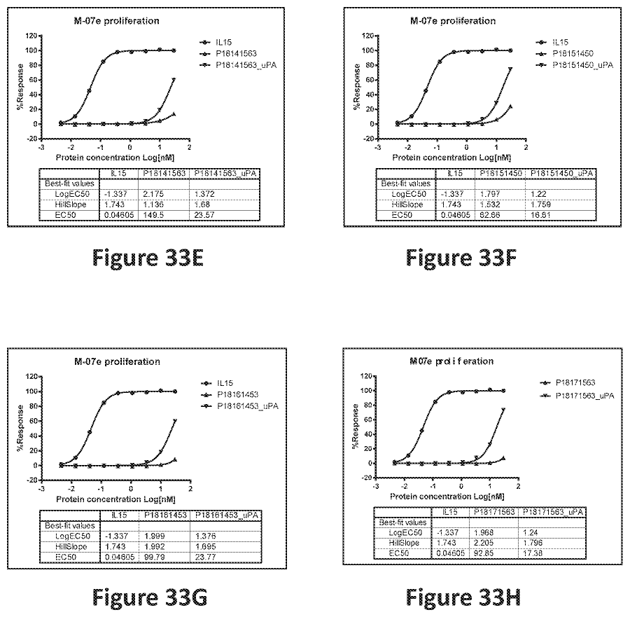

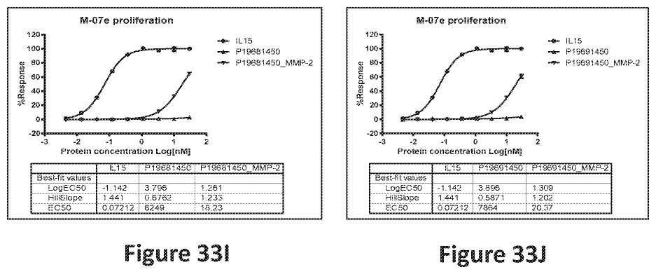

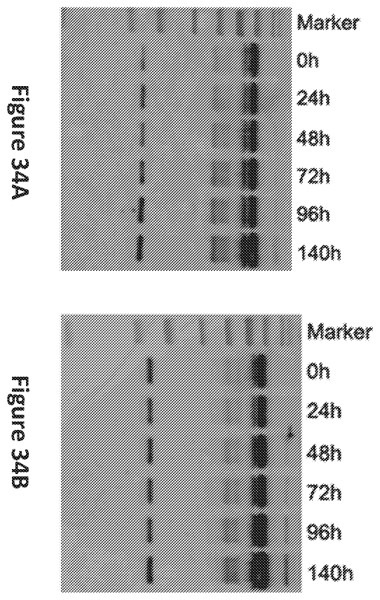

Uncleaved and protease cleaved proteins were then tested in M-07e proliferation assay using the same method as described in Example 1. The results are summarized in FIGS. 33A-33J.

Protein in vitro serum stability was tested in serum from human, cyno, mouse and rat, as described in Example 4. Representative results are shown in FIGS. 34A-34B.

### EXEMPLARY SEQUENCES

Human IL-15 precursor sequence
>human_IL-15_LSP (P40933)
(SEQ ID NO: 1)
MRISKPHLRSISIQCYLCLLLNSHFLTEAGIHVFILGCFSAGLPKTEANWVNVISDLKKIEDLIQSMHIDATLYT
ESDVHPSCKVTAMKCFLLELQVISLESGDASIHDTVENLIILANNSLSSNGNVTESGCKECEELEEKNIKEFLQS
FVHIVQMFINTS
Human IL-15 mature form sequence
>P40933 (N49-S162) (L)
(SEQ ID NO: 2)
NWVNVISDLKKIEDLIQSMHIDATLYTESDVHPSCKVTAMKCFLLELQVISLESGDASIHDTVENLIILANNSLS
SNGNVTESGCKECEELEEKNIKEFLQSFVHIVQMFINTS
Human IL-15 mature form sequence with point mutation of S162A
>IL-15_v1 (49-162 with S162A) (L)
(SEQ ID NO: 3)
NWVNVISDLKKIEDLIQSMHIDATLYTESDVHPSCKVTAMKCFLLELQVISLESGDASIHDTVENLIILANNSLS
SNGNVTESGCKECEELEEKNIKEFLQSFVHIVQMFINTA
Human IL-15Rα full length sequence
>human_IL-15Rα_FL (Q13261)
(SEQ ID NO: 4)
MAPRRARGCRTLGLPALLLLLLLRPPATRGITCPPPMSVEHADIWVKSYSLYSRERYICNSGFKRKAGTSSLTEC
VLNKATNVAHWTTPSLKCIRDPALVHQRPAPPSTVTTAGVTPQPESLSPSGKEPAASSPSSNNTAATTAAIVPGS
QLMPSKSPSTGTTEISSHESSHGTPSQTTAKNWELTASASHQPPGVYPQGHSDTTVAISTSTVLLCGLSAVSLLA
CYLKSRQTPPLASVEMEAMEALPVTWGTSSRDEDLENCSHHL
Human IL-15Rα extracellular domain
>human_IL-15Rα-ECD
(SEQ ID NO: 5)
ITCPPPMSVEHADIWVKSYSLYSRERYICNSGFKRKAGTSSLTECVLNKATNVAHWTTPSLKCIRDPALVHQRPA
PPSTVTTAGVTPQPESLSPSGKEPAASSPSSNNTAATTAAIVPGSQLMPSKSPSTGTTEISSHESSHGTPSQTTA
KNWELTASASHQPPGVYPQGHSDTT
Human IL-15Rα, sushi+
>Q13261 31-108 (R)
(SEQ ID NO: 6)
ITCPPPMSVEHADIWVKSYSLYSRERYICNSGFKRKAGTSSLTECVLNKATNVAHWTTPSLKCIRDPALVHQRPA
PPS
Human IL-15Rα, sushi
>human_IL-15Rα-sushi
(SEQ ID NO: 7)
ITCPPPMSVEHADIWVKSYSLYSRERYICNSGFKRKAGTSSLTECVLNKATNVAHWTTPSLKCIR
Fc-IL-15-IL-15Rα backbone 1
>Human IgG1 Fc
(SEQ ID NO: 8)
EPKSSDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK
Fc-IL-15-IL-15Rα backbone 2
>Human IgG1 Fc-a
(SEQ ID NO: 9)
KTHTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALHNHYTQKSLSLSPG
K
Fc-IL-15-IL-15Rα backbone 3
>Human IgG1 Fc-b_L234A/L235A/P329A/M252Y/S254T/T256E
(SEQ ID NO: 10)
EPKSSDKTHTCPPCPAPEAAGGPSVFLFPPKPKDTLYITREPEVTCVVVDVSHEDPEVKFNWYVDGVEVHNAKTK
PREEQYNSTYRVVSVLTVLHQDWLNGKEYKCKVSNKALAAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK
Fc-IL-15-IL-15Rα backbone 4
>Human IgG1 Fc-c
(SEQ ID NO: 11)
EPKSSDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK
IL-15-(GGS)5-IL-15Rα
>L15R
(SEQ ID NO: 12)
NWVNVISDLKKIEDLIQSMHIDATLYTESDVHPSCKVTAMKCFLLELQVISLESGDASIHDTVENLIILANNSLS
SNGNVTESGCKECEELEEKNIKEFLQSFVHIVQMFINTSGGSGGSGGSGGSGGSITCPPPMSVEHADIWVKSYSL
YSRERYICNSGFKRKAGTSSLTECVLNKATNVAHWTTPSLKCIRDPALVHQRPAPPS
IL-15 (L52C)-(GGS)5-IL-15Rα (S40C)
>L15R_(L:L52C)_(R:S40C)
(SEQ ID NO: 13)
NWVNVISDLKKIEDLIQSMHIDATLYTESDVHPSCKVTAMKCFLLELQVISCESGDASIHDTVENLIILANNSLS
SNGNVTESGCKECEELEEKNIKEFLQSFVHIVQMFINTSGGSGGSGGSGGSGGSITCPPPMSVEHADIWVKSYSL
YSRERYICNSGFKRKAGTCSLTECVLNKATNVAHWTTPSLKCIRDPALVHQRPAPPS
IL-15-(GGS)2-EPKSSDKTHT-(GGS)2-IL-15Rα
>L22R
(SEQ ID NO: 14)
NWVNVISDLKKIEDLIQSMHIDATLYTESDVHPSCKVTAMKCFLLELQVISLESGDASIHDTVENLIILANNSLS
SNGNVTESGCKECEELEEKNIKEFLQSFVHIVQMFINTSGGSGGSEPKSSDKTHTGGSGGSITCPPPMSVEHADI
WVKSYSLYSRERYICNSGFKRKAGTSSLTECVLNKATNVAHWTTPSLKCIRDPALVHQRPAPPS
IL-15-(GGS)2-EPKSSDKTHT-(GGS)2GGGS-IL-15Rα
>L26R
(SEQ ID NO: 15)
NWVNVISDLKKIEDLIQSMHIDATLYTESDVHPSCKVTAMKCFLLELQVISLESGDASIHDTVENLIILANNSLS
SNGNVTESGCKECEELEEKNIKEFLQSFVHIVQMFINTS [symbol] [symbol] ITCPPPMSVE
HADIWVKSYSLYSRERYICNSGFKRKAGTSSLTECVLNKATNVAHWTTPSLKCIRDPALVHQRPAPPS
IL-15-(GGS)5-IL-15Rα-G4S-HHHHHH homodimer
>P1185_L15R-G4S-his6_chains 1 and 2
(SEQ ID NO: 16)
NWVNVISDLKKIEDLIQSMHIDATLYTESDVHPSCKVTAMKCFLLELQVISLESGDASIHDTVENLIILANNSLS
SNGNVTESGCKECEELEEKNIKEFLQSFVHIVQMFINTS [symbol] [symbol] ITCPPPMSVEHADIWVKSYSL
YSRERYICNSGFKRKAGTSSLTECVLNKATNVAHWTTPSLKCIRDPALVHQRPAPPSGGGGSHHHHHH
IL-15-(GGS)5-IL-15Rα-G4S-Fc homodimer
>P1186_L15R-stable-Fc_chains 1 and 2
(SEQ ID NO: 17)
NWVNVISDLKKIEDLIQSMHIDATLYTESDVHPSCKVTAMKCFLLELQVISLESGDASIHDTVENLIILANNSLS
SNGNVTESGCKECEELEEKNIKEFLQSFVHIVQMFINTS [symbol] ITCPPPMSVEHADIWVKSYSL
YSRERYICNSGFKRKAGTSSLTECVLNKATNVAHWTTPSLKCIRDPALVHQRPAPPS [symbol] EPKSSDKTHTCPP
CPAPELLGGPSVFLFPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVMHEALHNHYTQKSLSLSPGK
Fc-GGGSKTHTGGGS-IL-15-(GGS)5-IL-15Rα homodimer
>P1187_Fc-stableS-L15R_chains 1 and 2
(SEQ ID NO: 18)
EPKSSDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] [symbol] NWVNVISDLKKIEDLIQSMHIDATLYTESDVHPSCKVTAMKCFLLELQVISLESGD
ASIHDTVENLIILANNSLSSNGNVTESGCKECEELEEKNIKEFLQSFVHIVQMFINTS [symbol] IT
CPPPMSVEHADIWVKSYSLYSRERYICNSGFKRKAGTSSLTECVLNKATNVAHWTTPSLKCIRDPALVHQRPAPP
S
Fc-GGGSKTHTGGGS-IL-15-(GGS)2-EPKSSDKTHT-(GGS)2-IL-15Rα homodimer
>P1188_Fc-stable-L22R_chains 1 and 2
(SEQ ID NO: 19)
EPKSSDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] NWVNVISDLKKIEDLIQSMHIDATLYTESDVHPSCKVTAMKCFLLELQVISLESGD
ASIHDTVENLIILANNSLSSNGNVTESGCKECEELEEKNIKEFLQSFVHIVQMFINTS [symbol] [symbol] [symbol]
ITCPPPMSVEHADIWVKSYSLYSRERYICNSGFKRKAGTSSLTECVLNKATNVAHWTTPSLKCIRDPALV
HQRPAPPS
Fc-GGGS-IL-15-(GGS)5-IL-15Rα homodimer
>P1250_Fc-4-L15R_chains 1 and 2
(SEQ ID NO: 20)
EPKSSDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] NWVNVISDLKKIEDLIQSMHIDATLYTESDVHPSCKVTAMKCFLLELQVISLESGDASIHDTVE
NLIILANNSLSSNGNVTESGCKECEELEEKNIKEFLQSFVHIVQMFINTS [symbol] [symbol] ITCPPPMSVE
HADIWVKSYSLYSRERYICNSGFKRKAGTSSLTECVLNKATNVAHWTTPSLKCIRDPALVHQRPAPPS
Fc-GGGS-IL-15-(GGS)2-EPKSSDKTHT-(GGS)2-IL-15Rα homodimer
>P1251_Fc-4-L22R_chains 1 and 2
(SEQ ID NO: 21)
EPKSSDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] NWVNVISDLKKIEDLIQSMHIDATLYTESDVHPSCKVTAMKCFLLELQVISLESGDASIHDTVE
NLIILANNSLSSNGNVTESGCKECEELEEKNIKEFLQSFVHIVQMFINTS [symbol] [symbol] ITC
PPPMSVEHADIWVKSYSLYSRERYICNSGFKRKAGTSSLTECVLNKATNVAHWTTPSLKCIRDPALVHQRPAPPS
Fc-GGGSKTHTGGGS-IL-15-(GGS)2-EPKSSDKTHT-(GGS)2-GGGS-IL-15Rα homodimer
>P1252_Fc-stable-L26R_chains 1 and 2
(SEQ ID NO: 22)
EPKSSDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] [symbol] NWVNVISDLKKIEDLIQSMHIDATLYTESDVHPSCKVTAMKCFLLELQVISLESGD
ASIHDTVENLIILANNSLSSNGNVTESGCKECEELEEKNIKEFLQSFVHIVQMFINTS [symbol] [symbol] [symbol]
ITCPPPMSVEHADIWVKSYSLYSRERYICNSGFKRKAGTSSLTECVLNKATNVAHWTTPSLKCIRD
PALVHQRPAPPS
IL-15 (S162A)-(GGS)5-IL-15Rα-GGGGS-Fc homodimer
>P1253_L15R(IL15-S162A)-stable-Fc)_chains 1 and 2
(SEQ ID NO: 23)
NWVNVISDLKKIEDLIQSMHIDATLYTESDVHPSCKVTAMKCFLLELQVISLESGDASIHDTVENLIILANNSLS
SNGNVTESGCKECEELEEKNIKEFLQSFVHIVQMFINT [symbol] [symbol] ITCPPPMSVEHADIWVKSYSL
YSRERYICNSGFKRKAGTSSLTECVLNKATNVAHWTTPSLKCIRDPALVHQRPAPPS [symbol] EPKSSDKTHTCPP
CPAPELLGGPSVFLFPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVMHEALHNHYTQKSLSLSPGK
Fc-GGGSKTHTGGGS-IL-15 (S162A)-(GGS)5-IL-15Rα homodimer
>P1254_Fc-stableS-L15R(IL15-S162A)_chains 1 and 2
(SEQ ID NO: 24)
EPKSSDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] [symbol] NWVNVISDLKKIEDLIQSMHIDATLYTESDVHPSCKVTAMKCFLLELQVISLESGD
ASIHDTVENLIILANNSLSSNGNVTESGCKECEELEEKNIKEFLQSFVHIVQMFINTA [symbol] IT
CPPPMSVEHADIWVKSYSLYSRERYICNSGFKRKAGTSSLTECVLNKATNVAHWTTPSLKCIRDPALVHQRPAPP
S
Fc-GGGSKTHTGGGS-IL-15 (S162A)-(GGS)2-EPKSSDKTHT-(GGS)2-IL15Rα homodimer
>P1255_Fc-stable-L22R(IL15-S162A)_chains 1 and 2
(SEQ ID NO: 25)
EPKSSDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] [symbol] NWVNVISDLKKIEDLIQSMHIDATLYTESDVHPSCKVTAMKCFLLELQVISLESGD
ASIHDTVENLIILANNSLSSNGNVTESGCKECEELEEKNIKEFLQSFVHIVQMFINT [symbol] [symbol] [symbol]
ITCPPPMSVEHADIWVKSYSLYSRERYICNSGFKRKAGTSSLTECVLNKATNVAHWTTPSLKCIRDPALV
HQRPAPPS
Fc-GS-ENLYFQG-GS-IL-15-(GGS)5-IL-15Rα homodimer
>P1256_Fc-TEV-L15R_chains 1 and 2
Chains 1 and 2
(SEQ ID NO: 26)
EPKSSDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] [symbol] NWVNVISDLKKIEDLIQSMHIDATLYTESDVHPSCKVTAMKCFLLELQVISLESGDA
SIHDTVENLIILANNSLSSNGNVTESGCKECEELEEKNIKEFLQSFVHIVQMFINTS [symbol] [symbol] ITC
PPPMSVEHADIWVKSYSLYSRERYICNSGFKRKAGTSSLTECVLNKATNVAHWTTPSLKCIRDPALVHQRPAPPS

Expected Cleaved Products from TEV Cleavage of P1256

1) Fc-GSENLYFQ homodimer (product from complete
cleavage)
Chains 1 and 2
(SEQ ID NO: 27)
EPKSSDKTHTCPPCPAPELLGGPSVFLFPPKPKDTLMISRTPEVTC
VVVDVSHEDPEVKFNWYVDGVEVHNAKTKPREEQYNSTYRVVSVLI
VLHQDWLNGKEYKCKVSNKALPAPIEKTISKAKGQPREPQVYTLPP
SREEMTKNQVSLICLVKGFYPSDIAVEWESNGQPENNYKTTPPVLD
SDGSFFLYSKLTVDKSRWQQGNVFSCSVMHEALHNHYTQKSLSLSP
GK [symbol]
2) GGS-IL-15-(GGS)5-IL-15Rα homodimer(product
from complete cleavage, active)
Chains 1 and 2
(SEQ ID NO: 28) [symbol]
NWVNVISDLKKIEDLIQSMHIDATLYTESDVHPSCKVTAMKCF
LLELQVISLESGDASIHDTVENLIILANNSLSSNGNVTESGCKECE
ELEEKNIKEFLQSFVHIVQMFINTS [symbol] [symbol] ITCPPP
MSVEHADIWVKSYSLYSRERYICNSGFKRKAGTSSLTECVLNKATN
VAHWTTPSLKCIRDPALVHQRPAPPS
3) Fc-GS-ENLYFQG-GS-IL-15-(GGS)5-IL-15Rα/Fc-
GSENLYFQ/GGS-IL-15-(GGS)5-IL-15Rα (product
from partial cleavage, active)
Chain 1: Fc-GS-ENLYFQG-GS-IL-15-(GGS)5-IL-15Rα
(SEQ ID NO: 29)
EPKSSDKTHTCPPCPAPELLGGPSVFLFPPKPKDTLMISRTPEVTC
VVVDVSHEDPEVKFNWYVDGVEVHNAKTKPREEQYNSTYRVVSVLT
VLHQDWLNGKEYKCKVSNKALPAPIEKTISKAKGQPREPQVYTLPP
SREEMTKNQVSLTCLVKGFYPSDIAVEWESNGQPENNYKTTPPVLD
SDGSFFLYSKLTVDKSRWQQGNVFSCSVMHEALHNHYTQKSLSLSP
GK [symbol] [symbol] NWVNVISDLKKIEDLIQSMHIDATLYTESDVHP
SCKVTAMKCFLLELQVISLESGDASIHDTVENLIILANNSLSSNGN
VTESGCKECEELEEKNIKEFLQSFVHIVQMFINTS [symbol] [symbol]
ITCPPPMSVEHADIWVKSYSLYSRERYICNSGFKRKA
GTSSLTECVLNKATNVAHWTTPSLKCIRDPALVHQRPAPPS
Chain 2: Fc-GSENLYFQ
(SEQ ID NO: 30)
EPKSSDKTHTCPPCPAPELLGGPSVFLFPPKPKDTLMISRTPEVTC
VVVDVSHEDPEVKFNWYVDGVEVHNAKTKPREEQYNSTYRVVSVLT
VLHQDWLNGKEYKCKVSNKALPAPIEKTISKAKGQPREPQVYTLPP
SREEMTKNQVSLTCLVKGFYPSDIAVEWESNGQPENNYKTTPPVLD
SDGSFFLYSKLTVDKSRWQQGNVFSCSVMHEALHNHYTQKSLSLSP
GK [symbol]
Chain 3: GGS-IL-15-(GGS)5-IL-15Rα
(SEQ ID NO: 31) [symbol]
NWVNVISDLKKIEDLIQSMHIDATLYTESDVHPSCKVTAMKCF
LLELQVISLESGDASIHDTVENLIILANNSLSSNGNVTESGCKECE
ELEEKNIKEFLQSFVHIVQMFINTS [symbol] [symbol] ITCPPP
MSVEHADIWVKSYSLYSRERYICNSGFKRKAGTSSLTECVLNKATN
VAHWTTPSLKCIRDPALVHQRPAPPS
Fc-GGGGSENLYFQGGGGGS-IL-15-(GGS)5-IL-15Rα
homodimer
>P1261_15LR-13_Fc-linker-TEV-linker-L15R
chains 1 and 2
(SEQ ID NO: 32)
EPKSSDKTHTCPPCPAPELLGGPSVFLFPPKPKDTLMISRTPEVTC
VVVDVSHEDPEVKFNWYVDGVEVHNAKTKPREEQYNSTYRVVSVLT
VLHQDWLNGKEYKCKVSNKALPAPIEKTISKAKGQPREPQVYTLPP
SREEMTKNQVSLTCLVKGFYPSDIAVEWESNGQPENNYKTTPPVLD
SDGSFFLYSKLTVDKSRWQQGNVFSCSVMHEALHNHYTQKSLSLSP
GK [symbol] [symbol] NWVNVISDLKKIEDLIQSMHIDATLYT
ESDVHPSCKVTAMKCFLLELQVISLESGDASIHDTVENLIILANNS
LSSNGNVTESGCKECEELEEKNIKEFLQSFVHIVQMFINTS [symbol] [symbol] [symbol]
ITCPPPMSVEHADIWVKSYSLYSRERYICNSGFKRK
AGTSSLTECVLNKATNVAHWTTPSLKCIRDPALVHQRPAPPS

Expected Cleaved Products from TEV Cleavage of P1261:

1) Fc-GGGGSENLYFQ homodimer (product from complete
cleavage)
Chains 1 and 2
(SEQ ID NO: 33)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTOKSLSLSPGK [symbol]
2) GGGGGS-IL-15-(GGS)5-IL-15Rα homodimer(product from complete
cleavage, active)
Chains 1 and 2
(SEQ ID NO: 34) [symbol]
NWVNVISDLKKIEDLIQSMHIDATLYTESDVHPSCKVTAMKCFLLELQVISLESGDA
SIHDTVENLIILANNSLSSNGNVTESGCKECEELEEKNIKEFLQSFVHIVQMFINTS [symbol] [symbol] ITC
PPPMSVEHADIWVKSYSLYSRERYICNSGFKRKAGISSLTECVLNKATNVAHWTTPSLKCIRDPALVHQRPAPPS
3) Fc-GGGGS-ENLYFQG-GGGGS-IL-15-(GGS)5-IL-15Rα/Fc-GGGGSENLYFQ/
GGGGGS-IL-15-(GGS)5-IL-15Rα (product from partial cleavage, active)
Chain 1: Fc-GGGGS-ENLYFQG-GGGGS-IL-15-(GGS)5-IL-15Rα
(SEQ ID NO: 35)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKVTA
MKCFLLELQVISLESGDASIHDTVENLIILANNSLSSNGNVTESGCKECEELEEKNIKEFLQSFVHIVQMFINTS [symbol] [symbol]
ITCPPPMSVEHADIWVKSYSLYSRERYICNSGFKRKAGTSSLTECVLNKATNVAHWTTPS
LKCIRDPALVHQRPAPPS
Chain 2: Fc-GGGGSENLYFQ
(SEQ ID NO: 36)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: GGGGGS-IL-15-(GGS)5-IL-15Rα
(SEQ ID NO: 37) [symbol]
NWVNVISDLKKIEDLIQSMHIDATLYTESDVHPSCKVTAMKCFLLELQVISLESGDA
SIHDTVENLIILANNSLSSNGNVTESGCKECEELEEKNIKEFLQSFVHIVQMFINTS [symbol] [symbol] ITC
PPPMSVEHADIWVKSYSLYSRERYICNSGFKRKAGTSSLTECVLNKATNVAHWTTPSLKCIRDPALVHQRPAPPS
IL-15(S162A)-(GGS)5-IL-15Rα-GGGGSHHHHHH
>P1274_L15R(IL15-S162A)-G4S-his6 chains 1 and 2
(SEQ ID NO: 38)
NWVNVISDLKKIEDLIQSMHIDATLYTESDVHPSCKVTAMKCFLLELQVISLESGDASIHDTVENLI
ILANNSLSSNGNVTESGCKECEELEEKNIKEFLQSFVHIVQMFINT [symbol] [symbol] ITCPPPMSVEHAD
IWVKSYSLYSRERYICNSGFKRKAGTSSLTECVLNKATNVAHWTTPSLKCIRDPALVHQRPAPPSGGGGSHHHHH
H
IL-15-(GGS)2-EPKSSDKTHT-(GGS)2-GGGS-IL-15Rα-GGGGSHHHHHH
>P1275_L26R-G4S-his6
(SEQ ID NO: 39)
NWVNVISDLKKIEDLIQSMHIDATLYTESDVHPSCKVTAMKCFLLELQVISLESGDASIHDTVENLIILANNSLS
SNGNVTESGCKECEELEEKNIKEFLQSFVHIVQMFINTS [symbol] [symbol] ITCPPPMSVE
HADIWVKSYSLYSRERYICNSGFKRKAGTSSLTECVLNKATNVAHWTTPSLKCIRDPALVHQRPAPPSGGGGSHH
HHHH
IL-15(S162A)-(GGS)2-EPKSSDKTHT-(GGS)2-GGGS-IL-15Rα-GGGGSHHHHHH
>P1276_L26R(IL15-S162A)-G4S-his6
(SEQ ID NO: 40)
NWVNVISDLKKIEDLIQSMHIDATLYTESDVHPSCKVTAMKCFLLELQVISLESGDASIHDTVENLIILANNSLS
SNGNVTESGCKECEELEEKNIKEFLQSFVHIVQMFINT [symbol] [symbol] ITCPPPMSVE
HADIWVKSYSLYSRERYICNSGFKRKAGTSSLTECVLNKATNVAHWTTPSLKCIRDPALVHQRPAPPSGGGGSHH
HHHH
Fc-G-IL-15(S162A)-(GGS)5-IL-15Rα
>F1279__Fc-1-L15R (IL15-S162A) chains 1 and 2
(SEQ ID NO: 41)
EPKSSDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] NWVNVISDLKKIEDLIQSMHIDATLYTESDVHPSCKVTAMKCFLLELQVISLESGDASIHDTVENLI
ILANNSLSSNGNVTESGCKECEELEEKNIKEFLQSFVHIVQMFINT [symbol] [symbol] ITCPPPMSVEHAD
IWVKSYSLYSRERYICNSGFKRKAGTSSLTECVLNKATNVAHWTTPSLKCIRDPALVHQRPAPPS
Fc-G-IL-15(S162A)-(GGS)2-EPKSSDKTHT-(GGS)2GGGS-IL-15Rα
>F1280__Fc-1-L26R (IL15_S162A) chains 1 and 2
(SEQ ID NO: 42)
EPKSSDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGKGNWVNVISDLKKIEDLIQSMHIDATLYTESDVHPSCKVTAMKCFLLELQVISLESGDASIHDTVENLI
ILANNSLSSNGNVTESGCKECEELEEKNIKEFLQSFVHIVQMFINT [symbol] [symbol] IT
CPPPMSVEHADIWVKSYSLYSRERYICNSGFKRKAGTSSLTECVLNKATNVAHWTTPSLKCIRDPALVHQRPAPP
S
Fc-GSGGGGSENLYFQGGGGGSGS-IL-15-(GGS)5-IL-15Rα
>F1281__Fc-7-TEV-7-L15R chains 1 and 2
(SEQ ID NO: 43)
EPKSSDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] [symbol] NWVNVISDLKKIEDLIQSMHIDATLYTESDVHPSCKVTAMKCFLLEL
QVISLESGDASIHDTVENLIILANNSLSSNGNVTESGCKECEELEEKNIKEFLQSFVHIVQMFINTS [symbol] [symbol]
ITCPPPMSVEHADIWVKSYSLYSRERYICNSGFKRKAGTSSLTECVLNKATNVAHWTTPSLKCIRDPA
LVHQRPAPPS

Expected Cleaved Products from TEV Cleavage of P1281:

1) Fc-GSGGGGSENLYFQ homodimer (product from complete cleavage)
Chains 1 and 2
(SEQ ID NO: 44)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) GGGGGSGS-IL-15-(GGS)5-IL-15Rα homodimer(product from
complete cleavage, active)
Chains 1 and 2
(SEQ ID NO: 45) [symbol]
NWVNVISDLKKIEDLIQSMHIDATLYTESDVHPSCKVTAMKCFLLELQVISLESG
DASIHDTVENLIILANNSLSSNGNVTESGCKECEELEEKNIKEFLQSFVHIVQMFINTS [symbol] [symbol]
TCPPPMSVEHADIWVKSYSLYSRERYICNSGFKRKAGTSSLTECVLNKATNVAHWTTPSLKCIRDPALVHQRPAP
PS
3) Fc-GSGGGGS-ENLYFQG-GGGGSGS-IL-15-(GGS)5-IL-15Rα/Fc-
GSGGGGSENLYFQ/GGGGGSGS-IL-15-(GGS)5-IL-15Rα (product from partial
cleavage, active)
Chain 1: Fc-GSGGGGS-ENLYFQG-GGGGSGS-IL-15-(GGS)5-IL-15Rα
(SEQ ID NO: 46)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSC
KVTAMKCFLLELQVISLESGDASIHDTVENLIILANNSLSSNGNVTESGCKECEELEEKNIKEFLQSFVHIVQMF
INTS [symbol] [symbol] ITCPPPMSVEHADIWVKSYSLYSRERYICNSGFKRKAGTSSLTECVLNKATNVAHW
TTPSLKCIRDPALVHQRPAPPS
Chain 2: Fc-GSGGGGSENLYFQ
(SEQ ID NO: 47)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: GGGGGSGS-IL-15-(GGS)5-IL-15Rα
(SEQ ID NO: 48) [symbol]
NWVNVISDLKKIEDLIQSMHIDATLYTESDVHPSCKVTAMKCFLLELQVISLESG
DASIHDTVENLIILANNSLSSNGNVTESGCKECEELEEKNIKEFLQSFVHIVQMFINTS [symbol] I
TCPPPMSVEHADIWVKSYSLYSRERYICNSGFKRKAGTSSLTECVLNKATNVAHWTTPSLKCIRDPALVHQRPAP
PS
Fc-GGGSGGGGSENLYFQGGGGGSGGGS-IL-15-(GGS)5-IL-15Rα
>F1282_Fc-9-TEV-9-L15R chains 1 and 2
(SEQ ID NO: 49)
EPKSSDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] [symbol] NWVNVISDLKKIEDLIQSMHIDATLYTESDVHPSCKVTAMKCF
LLELQVISLESGDASIHDTVENLIILANNSLSSNGNVTESGCKECEELEEKNIKEFLQSFVHIVQMFINTS [symbol] [symbol]
ITCPPPMSVEHADIWVKSYSLYSRERYICNSGFKRKAGTSSLTECVLNKATNVAHWTTPSLKCI
RDPALVHQRPAPPS

Expected Cleaved Products from TEV Cleavage of P1282:

1) Fc-GGGSGGGGSENLYFQ homodimer (product from complete cleavage)
Chains 1 and 2
(SEQ ID NO: 50)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol]
2) GGGGGSGGGS-IL-15-(GGS)5-IL-15Rα homodimer(product from
complete cleavage, active)
Chains 1 and 2
(SEQ ID NO: 51) [symbol]
NWVNVISDLKKIEDLIQSMHIDATLYTESDVHPSCKVTAMKCFLLELQVISLE
SGDASIHDTVENLIILANNSLSSNGNVTESGCKECEELEEKNIKEFLQSFVHIVQMFINTS [symbol] [symbol] [symbol]
ITCPPPMSVEHADIWVKSYSLYSRERYICNSGFKRKAGTSSLTECVLNKATNVAHWTTPSLKCIRDPALVHQRP
APPS
3) Fc-GGGSGGGGS-ENLYFQG-GGGGSGGGS-IL-15-(GGS)5-IL-15Rα/Fc-
GGGSGGGGSENLYFQ/GGGGGSGGGS-IL-15-(GGS)5-IL-15Rα (product from partial
cleavage, active)
Chain 1: Fc-GGGSGGGGS-ENLYFQG-GGGGSGGGS-IL-15-(GGS)5-IL-15Rα
(SEQ ID NO: 52)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDV
HPSCKVTAMKCFLLELQVISLESGDASIHDTVENLIILANNSLSSNGNVTESGCKECEELEEKNIKEFLQSFVHI
VQMFINTS [symbol] [symbol] ITCPPPMSVEHADIWVKSYSLYSRERYICNSGFKRKAGTSSLTECVLNKATN
VAHWTTPSLKCIRDPALVHQRPAPPS
Chain 2: Fc-GGGSGGGGSENLYFQ
(SEQ ID NO: 53)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: GGGGGSGGGS-IL-15-(GGS)5-IL-15Rα
(SEQ ID NO: 54) [symbol]
NWVNVISDLKKIEDLIQSMHIDATLYTESDVHPSCKVTAMKCFLLELQVISLE
SGDASIHDTVENLIILANNSLSSNGNVTESGCKECEELEEKNIKEFLQSFVHIVQMFINTS [symbol] [symbol]
ITCPPPMSVEHADIWVKSYSLYSRERYICNSGFKRKAGTSSLTECVLNKATNVAHWTTPSLKCIRDPALVHQRP
APPS
Fc-GSGGGSGGGGSENLYFQGGGGGSGGGSGS-IL-15-(GGS)5-IL-15Rα
>F1283_Fc-11-TEV-11-L15R chains 1 and 2
(SEQ ID NO: 55)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] [symbol] NWVNVISDLKKIEDLIQSMHIDATLYT
ESDVHPSCKVTAMKCFLLELQVISLESGDASIHDTVENLIILANNSLSSNGNVTESGCKECEELEEKNIKEFLQS
FVHIVQMFINTS [symbol] ITCPPPMSVEHADIWVKSYSLYSRERYICNSGFKRKAGTSSLTECVLN
KATNVAHWTTPSLKCIRDPALVHQRPAPPS

Expected Cleaved Products from TEV Cleavage of P1283:

1) Fc-GSGGGSGGGGSENLYFQ homodimer (product from complete cleavage)
Chains 1 and 2
(SEQ ID NO: 56)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol]
2) GGGGGSGGGSGS-IL-15-(GGS)5-IL-15Rα homodimer(product from
complete cleavage, active)
Chains 1 and 2
(SEQ ID NO: 57) [symbol]
NWVNVISDLKKIEDLIQSMHIDATLYTESDVHPSCKVTAMKCFLLELQVIS
LESGDASIHDTVENLIILANNSLSSNGNVTESGCKECEELEEKNIKEFLQSFVHIVQMFINTS [symbol] [symbol]
ITCPPPMSVEHADIWVKSYSLYSRERYICNSGFKRKAGTSSLTECVLNKATNVAHWTTPSLKCIRDPALVHQ
RPAPPS
3) Fc-GSGGGSGGGGS-ENLYFQG-GGGGSGGGSGS-IL-15-(GGS)5-IL-15Rα/Fc-
GSGGGSGGGGSENLYFQ/GGGGGSGGGSGS-IL-15-(GGS)5-IL-15Rα (product from partial
cleavage, active)
Chain 1: Fc-GSGGGSGGGGS-ENLYFQG-GGGGSGGGSGS-IL-15-(GGS)5-IL-15Rα
(SEQ ID NO: 58)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] [symbol] NWVNVISDLKKIEDLIQSMHIDATLYT
ESDVHPSCKVTAMKCFLLELQVISLESGDASIHDTVENLIILANNSLSSNGNVTESGCKECEELEEKNIKEFLQS
FVHIVQMFINTS [symbol] [symbol] ITCPPPMSVEHADIWVKSYSLYSRERYICNSGFKRKAGTSSLTECVLN
KATNVAHWTTPSLKCIRDPALVHQRPAPPS
Chain 2: Fc-GSGGGSGGGGSENLYFQ
(SEQ ID NO: 59)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol]
Chain 3: GGGGGSGGGSGS-IL-15-(GGS)5-IL-15Rα
(SEQ ID NO: 60) [symbol] [symbol]
NWVNVISDLKKIEDLIQSMHIDATLYTESDVHPSCKVTAMKCFLLELQVIS
LESGDASIHDTVENLIILANNSLSSNGNVTESGCKECEELEEKNIKEFLQSFVHIVQMFINTS [symbol] [symbol]
ITCPPPMSVEHADIWVKSYSLYSRERYICNSGFKRKAGTSSLTECVLNKATNVAHWTTPSLKCIRDPALVHQ
RPAPPS
Fc-GGGSGGGSGGGGSENLYFQGGGGGSGGGSGGGS-IL-15-(GGS)5-IL-15Rα
>F1284_Fc-13-TEV-13-L15R chains 1 and 2
(SEQ ID NO: 61)
EPKSSDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] [symbol] [symbol] NWVNVISDLKKIEDLIQSMHIDATLYTESDVHPSC
KVTAMKCFLLELQVISLESGDASIHDTVENLIILANNSLSSNGNVTESGCKECEELEEKNIKEFLQSFVHIVQMF
INTS [symbol] ITCPPPMSVEHADIWVKSYSLYSRERYICNSGFKRKAGTSSLTECVLNKATNVAHW
TTPSLKCIRDPALVHQRPAPPS

Expected Cleaved Products from TEV Cleavage of P1284:

1) Fc-GGGSGGGSGGGGSENLYFQ homodimer (product from complete cleavage)
Chains 1 and 2
(SEQ ID NO: 62)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol]
2) GGGGGSGGGSGGGS-IL-15-(GGS)5-IL-15Rα homodimer(product from
complete cleavage, active)
Chains 1 and 2
(SEQ ID NO: 63) [symbol]
NWVNVISDLKKIEDLIQSMHIDATLYTESDVHPSCKVTAMKCFLLELQV
ISLESGDASIHDTVENLIILANNSLSSNGNVTESGCKECEELEEKNIKEFLQSFVHIVQMFINTS [symbol] [symbol]
ITCPPPMSVEHADIWVKSYSLYSRERYICNSGFKRKAGTSSLTECVLNKATNVAHWTTPSLKCIRDPALV
HQRPAPPS
3) Fc-GGGSGGGSGGGGS-ENLYFQG-GGGGSGGGSGGGS-IL-15-(GGS)5-IL-
15Rα/Fc-GGGSGGGSGGGGSENLYFQ/GGGGGSGGGSGGGS-IL-15-(GGS)5-IL-15Rα (product
from partial cleavage, active)
Chain 1: Fc-GGGSGGGSGGGGS-ENLYFQG-GGGGSGGGSGGGS-IL-15-(GGS)5-IL-15Rα
(SEQ ID NO: 64)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] [symbol] NWVNVISDLKKIEDLIQSMHIDA
TLYTESDVHPSCKVTAMKCFLLELQVISLESGDASIHDTVENLIILANNSLSSNGNVTESGCKECEELEEKNIKE
FLQSFVHIVQMFINTS [symbol] ITCPPPMSVEHADIWVKSYSLYSRERYICNSGFKRKAGTSSLTE
CVLNKATNVAHWTTPSLKCIRDPALVHQRPAPPS
Chain 2: Fc-GGGSGGGSGGGGSENLYFQ
(SEQ ID NO: 65)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol]
Chain 3: GGGGGSGGGSGGGS-IL-15-(GGS)5-IL-15Rα
(SEQ ID NO: 66) [symbol] [symbol]
NWVNVISDLKKIEDLIQSMHIDATLYTESDVHPSCKVTAMKCFLLELQV
ISLESGDASIHDTVENLIILANNSLSSNGNVTESGCKECEELEEKNIKEFLQSFVHIVQMFINTS [symbol] [symbol]
ITCPPPMSVEHADIWVKSYSLYSRERYICNSGFKRKAGTSSLTECVLNKATNVAHWTTPSLKCIRDPALV
HQRPAPPS
Fc-GSGGGSGGGSGGGGSENLYFQGGGGGSGGGSGGGSGS-IL-15-(GGS)5-IL-15Rα
>F1285_Fc-15-TEV-15-L15R chains 1 and 2
(SEQ ID NO: 67)
EPKSSDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] [symbol] [symbol] [symbol] NWVNVISDLKKIEDLIQSMHIDATLYTESDV
HPSCKVTAMKCFLLELQVISLESGDASIHDTVENLIILANNSLSSNGNVTESGCKECEELEEKNIKEFLQSFVHI
VQMFINTS [symbol] ITCPPPMSVEHADIWVKSYSLYSRERYICNSGFKRKAGTSSLTECVLNKATN
VAHWTTPSLKCIRDPALVHQRPAPPS

Expected Cleaved Products from TEV Cleavage of P1285:

1) Fc-GSGGGSGGGSGGGGSENLYFQ homodimer (product from complete cleavage)
Chains 1 and 2
(SEQ ID NO: 68)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol]
2) GGGGGSGGGSGGGSGS-IL-15-(GGS)5-IL-15Rα homodimer(product from
complete cleavage, active)
Chains 1 and 2
(SEQ ID NO: 69) [symbol] [symbol]
NWVNVISDLKKIEDLIQSMHIDATLYTESDVHPSCKVTAMKCFLLEL
QVISLESGDASIHDTVENLIILANNSLSSNGNVTESGCKECEELEEKNIKEFLQSFVHIVQMFINTS [symbol] [symbol]
ITCPPPMSVEHADIWVKSYSLYSRERYICNSGFKRKAGTSSLTECVLNKATNVAHWTTPSLKCIRDPA
LVHQRPAPPS
3) Fc-GSGGGSGGGSGGGGS-ENLYFQG-GGGGSGGGSGGGSGS-IL-15-(GGS)5-IL-
15Rα/Fc-GSGGGSGGGSGGGGSENLYFQ/GGGGGSGGGSGGGSGS-IL-15-(GGS)5-IL-15Rα
(product from partial cleavage, active)
Chain 1: Fc-GSGGGSGGGSGGGGS-ENLYFQG-GGGGSGGGSGGGSGS-IL-15-(GGS)5-IL-15Rα
(SEQ ID NO: 70)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] [symbol] NWVNVISDLKKIEDLIQSM
HIDATLYTESDVHPSCKVTAMKCFLLELQVISLESGDASIHDTVENLIILANNSLSSNGNVTESGCKECEELEEK
NIKEFLQSFVHIVQMFINTS [symbol] ITCPPPMSVEHADIWVKSYSLYSRERYICNSGFKRKAGTS
SLTECVLNKATNVAHWTTPSLKCIRDPALVHQRPAPPS
Chain 2: Fc-GSGGGSGGGSGGGGSENLYFQ
(SEQ ID NO: 71)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol]
Chain 3: GGGGGSGGGSGGGSGS-IL-15-(GGS)5-IL-15Rα
(SEQ ID NO: 72) [symbol] [symbol]
NWVNVISDLKKIEDLIQSMHIDATLYTESDVHPSCKVTAMKCFLLEL
QVISLESGDASIHDTVENLIILANNSLSSNGNVTESGCKECEELEEKNIKEFLQSFVHIVQMFINTS [symbol] [symbol]
ITCPPPMSVEHADIWVKSYSLYSRERYICNSGFKRKAGTSSLTECVLNKATNVAHWTTPSLKCIRDPA
LVHQRPAPPS
IL-15-GGSGGSEPKSSDKTHTGGSGGSGGGS-IL-15Rα-G45-Fc
>P1331_L26R-stable-Fc chains 1 and 2
(SEQ ID NO: 73)
NWVNVISDLKKIEDLIQSMHIDATLYTESDVHPSCKVTAMKCFLLELQVISLESGDASIHDTVENLIILANNSLS
SNGNVTESGCKECEELEEKNIKEFLQSFVHIVQMFINTS [symbol] [symbol] ITCPPPMSVE
HADIWVKSYSLYSRERYICNSGFKRKAGTSSLTECVLNKATNVAHWTTPSLKCIRDPALVHQRPAPPS [symbol] EP
KSSDKTHTCPPCPAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMHEALHNHYTQKSLS
LSPGK
IL-15-(GGS)5-IL-15Rα-Fc-a
>P1332_L15R-Fc-a chains 1 and 2
(SEQ ID NO: 74)
NWVNVISDLKKIEDLIQSMHIDATLYTESDVHPSCKVTAMKCFLLELQVISLESGDASIHDTVENLI
ILANNSLSSNGNVTESGCKECEELEEKNIKEFLQSFVHIVQMFINTS [symbol] ITCPPPMSVEHAD
IWVKSYSLYSRERYICNSGFKRKAGTSSLTECVLNKATNVAHWTTPSLKCIRDPALVHQRPAPPS

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

IL-15-GGSGGSEPKSSDKTHTGGSGGSGGGS-IL-15Rα-Fc-a
>P1333_L26R-Fc-a chains 1 and 2
(SEQ ID NO: 75)
NWVNVISDLKKIEDLIQSMHIDATLYTESDVHPSCKVTAMKCFLLELQVISLESGDASIHDTVENLI
ILANNSLSSNGNVTESGCKECEELEEKNIKEFLQSFVHIVQMFINTS [symbol] [symbol] IT
CPPPMSVEHADIWVKSYSLYSRERYICNSGFKRKAGTSSLTECVLNKATNVAHWTTPSLKCIRDPALVHQRPAPP
S

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

Fc-b-GSLGGSGRSANAGS-IL-15(S162A)-(GGS)5-IL-15Rα
>P1334_Fc-b-PS1-L15R-S162A chains 1 and 2
(SEQ ID NO: 76)
EPKSSDKTHTCPPCPAPEAAGGPSVFLFPPKPKDTLYITREPEVTCVVVDVSHEDPEVKFNWYVDGVEVHNAKTK
PREEQYNSTYRVVSVLTVLHQDWLNGKEYKCKVSNKALAAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] [symbol] NWVNVISDLKKIEDLIQSMHIDATLYTESDVHPSCKVTAMKCFLLELQVISLES
GDASIHDTVENLIILANNSLSSNGNVTESGCKECEELEEKNIKEFLQSFVHIVQMFINT [symbol] [symbol]
ITCPPPMSVEHADIWVKSYSLYSRERYICNSGFKRKAGTSSLTECVLNKATNVAHWTTPSLKCIRDPALVHQRPA
PPS

Expected Cleavage Products from uPA or Matriptase Cleavage of P1334:

1) Fc-b-GSLGGSGR homodimer (product from complete cleavage)
Chains 1 and 2
(SEQ ID NO: 77)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) SANAGS-IL-15-(GGS)5-IL-15Rα homodimer(product from complete
cleavage, active)
Chains 1 and 2
(SEQ ID NO: 78) [symbol]
NWVNVISDLKKIEDLIQSMHIDATLYTESDVHPSCKVTAMKCFLLELQVISLESGDA
SIHDTVENLIILANNSLSSNGNVTESGCKECEELEEKNIKEFLQSFVHIVQMFINT [symbol] [symbol] ITC
PPPMSVEHADIWVKSYSLYSRERYICNSGFKRKAGTSSLTECVLNKATNVAHWTTPSLKCIRDPALVHQRPAPPS
3)Fc-b-GSLGGSGRSANAGS-IL-15(S162A)-(GGS)5-IL-15Rα/Fc-b-GSLGGSGR/
SANAGS-IL-15-(GGS)5-IL-15Rα (product from partial cleavage, active)
Chain 1: Fc-b-GSLGGSGRSANAGS-IL-15(S162A)-(GGS)5-IL-15Rα
(SEQ ID NO: 79)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKVTAMKC
FLLELQVISLESGDASIHDTVENLIILANNSLSSNGNVTESGCKECEELEEKNIKEFLQSFVHIVQMFINT [symbol] [symbol]
ITCPPPMSVEHADIWVKSYSLYSRERYICNSGFKRKAGTSSLTECVLNKATNVAHWTTPSLKC
IRDPALVHQRPAPPS
Chain 2: Fc-b-GSLGGSGR
(SEQ ID NO: 80)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: SANAGS-IL-15-(GGS)5-IL-15Rα
(SEQ ID NO: 81) [symbol]
NWVNVISDLKKIEDLIQSMHIDATLYTESDVHPSCKVTAMKCFLLELQVISLESGDA
SIHDTVENLIILANNSLSSNGNVTESGCKECEELEEKNIKEFLQSFVHIVQMFINTA [symbol] [symbol] TC
PPPMSVEHADIWVKSYSLYSRERYICNSGFKRKAGTSSLTECVLNKATNVAHWTTPSLKCIRDPALVHQRPAPPS

Expected Cleavage Products from Legumain Cleavage of P1334:

1) Fc-b-GSLGGSGRSAN homodimer (product from
complete cleavage)
Chains 1 and 2
(SEQ ID NO: 82)
EPKSSDKTHTCPPCPAPEAAGGPSVFLFPPKPKDTLYITREPEVTCV
VVDVSHEDPEVKFNWYVDGVEVHNAKTKPREEQYNSTYRVVSVLTVL
HQDWLNGKEYKCKVSNKALAAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVMHEALHNHYTQKSLSLSPGK

[symbol]

2) AGS-IL-15-(GGS)5-IL-15Rα (homodimer(product
from complete cleavage, active)
Chains 1 and 2
(SEQ ID NO: 83) [symbol]
NWVNVISDLKKIEDLIQSMHIDATLYTESDVHPSCKVTAMKCFL
LELQVISLESGDASIHDTVENLIILANNSLSSNGNVTESGCKECEEL
EEKNIKEFLQSFVHIVQMFINTA [symbol] [symbol] ITCPPPMSV
EHADIWVKSYSLYSRERYICNSGFKRKAGTSSLTECVLNKATNVAHW
TTPSLKCIRDPALVHQRPAPPS
3) Fc-b-GSLGGSGRSANAGS-IL-15(S162A)-(GGS)5-IL-
15Rα/Fc-b-GSLGGSGRSAN/AGS-IL-15-(GGS)5-IL-15Rα
(product from partial cleavage, active
Chain 1: Fc-b-GSLGGSGRSANAGS-IL-15(S162A)-
(GGS)5-IL-15Rα
(SEQ ID NO: 84)
EPKSSDKTHTCPPCPAPEAAGGPSVFLFPPKPKDTLYITREPEVTCV
VVDVSHEDPEVKFNWYVDGVEVHNAKTKPREEQYNSTYRVVSVLTVL
HQDWLNGKEYKCKVSNKALAAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVMHEALHNHYTQKSLSLSPGK [symbol] [symbol]
NWVNVISDLKKIEDLIQSMHIDATLYTESDVHPSCK
VTAMKCFLLELQVISLESGDASIHDTVENLIILANNSLSSNGNVTES
GCKECEELEEKNIKEFLQSFVHIVQMFINTA [symbol] [symbol] I
TCPPPMSVEHADIWVKSYSLYSRERYICNSGFKRKAGTSSLTECVLN
KATNVAHWTTPSLKCIRDPALVHQRPAPPS
Chain 2: Fc-b-GSLGGSGRSAN
(SEQ ID NO: 85)
EPKSSDKTHTCPPCPAPEAAGGPSVFLFPPKPKDTLYITREPEVTCV
VVDVSHEDPEVKFNWYVDGVEVHNAKTKPREEQYNSTYRVVSVLTVL
HQDWLNGKEYKCKVSNKALAAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVMHEALHNHYTQKSLSLSPGK

[symbol]

Chain 3: AGS-IL-15-(GGS)5-IL-15Rα
(SEQ ID NO: 86) [symbol]
NWVNVISDLKKIEDLIQSMHIDATLYTESDVHPSCKVTAMKCFL
LELQVISLESGDASIHDTVENLIILANNSLSSNGNVTESGCKECEEL
EEKNIKEFLQSFVHIVQMFINTA [symbol] [symbol] ITCPPPMSV
EHADIWVKSYSLYSRERYICNSGFKRKAGTSSLTECVLNKATNVAHW
TTPSLKCIRDPALVHQRPAPPS
Fc-b-GGSLSGRSANAGGS-IL-15(S162A)-(GGS)5-IL-15Rα
>P1335_Fc-b-PS2-L15R-S162A chains 1 and 2
(SEQ ID NO: 87)
EPKSSDKTHTCPPCPAPEAAGGPSVFLFPPKPKDTLYITREPEVTCV
VVDVSHEDPEVKFNWYVDGVEVHNAKTKPREEQYNSTYRVVSVLTVL
HQDWLNGKEYKCKVSNKALAAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVMHEALHNHYTQKSLSLSPGK [symbol] [symbol]
NWVNVISDLKKIEDLIQSMHIDATLYTESDVHP
SCKVTAMKCFLLELQVISLESGDASIHDTVENLIILANNSLSSNGNV
TESGCKECEELEEKNIKEFLQSFVHIVQMFINTA [symbol] [symbol]
ITCPPPMSVEHADIWVKSYSLYSRERYICNSGFKR
KAGTSSLTECVLNKATNVAHWTTPSLKCIRDPALVHQRPAPPS

Expected Cleavage Products from uPA or Matriptase Cleavage of P1335:

1) Fc-b-GGSLSGR homodimer (product from
complete cleavage)
Chains 1 and 2
(SEQ ID NO: 88)
EPKSSDKTHTCPPCPAPEAAGGPSVFLFPPKPKDTLYITREPEVTCV
VVDVSHEDPEVKFNWYVDGVEVHNAKTKPREEQYNSTYRVVSVLTVL
HQDWLNGKEYKCKVSNKALAAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVMHEALHNHYTQKSLSLSPGK

[symbol]

2) SANAGGS-IL-15-(GGS)5-IL-15Rα homodimer
(product from complete cleavage, active)
Chains 1 and 2
(SEQ ID NO: 89) [symbol]
NWVNVISDLKKIEDLIQSMHIDATLYTESDVHPSCKVTAM
KCFLLELQVISLESGDASIHDTVENLIILANNSLSSNGNVTESGCKECEE
LEEKNIKEFLQSFVHIVQMFINTA [symbol] [symbol] ITCPP
PMSVEHADIWVKSYSLYSRERYICNSGFKRKAGTSSLTECVLNKATN
VAHWTTPSLKCIRDPALVHQRPAPPS
3) Fc-b-GGSLSGRSANAGGS-IL-15(S162A)-(GGS)5-IL-
15Rα/Fc-b-GGSLSGR/SANAGGS-IL-15-(GGS)5-IL-15Rα
(product from partial cleavage, active)
Chain 1: Fc-b-GGSLSGRSANAGGS-IL-15(S162A)-
(GGS)5-IL-15Rα
(SEQ ID NO: 90)
EPKSSDKTHTCPPCPAPEAAGGPSVFLFPPKPKDTLYITREPEVTCV
VVDVSHEDPEVKFNWYVDGVEVHNAKTKPREEQYNSTYRVVSVLTVL
HQDWLNGKEYKCKVSNKALAAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVMHEALHNHYTQKSLSLSPGK [symbol] [symbol]
NWVNVISDLKKIEDLIQSMHIDATLYTESDV
HPSCKVTAMKCFLLELQVISLESGDASIHDTVENLIILANNSLSSNGNV
TESGCKECEELEEKNIKEFLQSFVHIVQMFINTA [symbol] [symbol] [symbol]
ITCPPPMSVEHADIWVKSYSLYSRERYICNSGFKRKAGTS
SLTECVLNKATNVAHWTTPSLKCIRDPALVHQRPAPPS
Chain 2: Fc-b-GGSLSGR
(SEQ ID NO: 91)
EPKSSDKTHTCPPCPAPEAAGGPSVFLFPPKPKDTLYITREPEVTCV
VVDVSHEDPEVKFNWYVDGVEVHNAKTKPREEQYNSTYRVVSVLTVL
HQDWLNGKEYKCKVSNKALAAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVMHEALHNHYTQKSLSLSPGK

[symbol]

Chain 3: SANAGGS-IL-15-(GGS)5-IL-15Rα
(SEQ ID NO: 92) [symbol]
NWVNVISDLKKIEDLIQSMHIDATLYTESDVHPSCKVTAM
KCFLLELQVISLESGDASIHDTVENLIILANNSLSSNGNVTESGCKECEEL
EEKNIKEFLQSFVHIVQMFINTA [symbol] [symbol] ITCPP
PMSVEHADIWVKSYSLYSRERYICNSGFKRKAGTSSLTECVLNKATN
VAHWTTPSLKCIRDPALVHQRPAPPS

Expected Cleavage Products from Legumain Cleavage of P1335:

1) Fc-b-GGSLSGRSAN homodimer (product from
complete cleavage)
Chains 1 and 2
(SEQ ID NO: 93)
EPKSSDKTHTCPPCPAPEAAGGPSVFLFPPKPKDTLYITREPEVTCV
VVDVSHEDPEVKFNWYVDGVEVHNAKTKPREEQYNSTYRVVSVLTVL
HQDWLNGKEYKCKVSNKALAAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVMHEALHNHYTQKSLSLSPGK

[symbol]

2) AGGS-IL-15-(GGS)5-IL-15Rα homodimer (product
from complete cleavage, active)
Chains 1 and 2
(SEQ ID NO: 94) [symbol]
NWVNVISDLKKIEDLIQSMHIDATLYTESDVHPSCKVTAMKCF
LLELQVISLESGDASIHDTVENLIILANNSLSSNGNVTESGCKECEE
LEEKNIKEFLQSFVHIVQMFINTA [symbol] [symbol] ITCPPPMS
VEHADIWVKSYSLYSRERYICNSGFKRKAGTSSLTECVLNKATNVAH
WTTPSLKCIRDPALVHQRPAPPS
3) Fc-b-GGSLSGRSANAGGS-IL-15(S162A)-(GGS)5-IL-
15Rα/Fc-b-GGSLSGRSAN/AGGS-IL-15-(GGS)5-IL-15Rα
(product from partial cleavage, active)
Chain 1: Fc-b-GGSLSGRSANAGGS-IL-15(S162A)-
(GGS)5-IL-15Rα
(SEQ ID NO: 95)
EPKSSDKTHTCPPCPAPEAAGGPSVFLFPPKPKDTLYITREPEVTCV
VVDVSHEDPEVKFNWYVDGVEVHNAKTKPREEQYNSTYRVVSVLTVL
HQDWLNGKEYKCKVSNKALAAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVMHEALHNHYTQKSLSLSPGK [symbol] [symbol]
NWVNVISDLKKIEDLIQSMHIDATLYTESDVHP
SCKVTAMKCFLLELQVISLESGDASIHDTVENLIILANNSLSSNGNV
TESGCKECEELEEKNIKEFLQSFVHIVQMFINTA [symbol] [symbol]
ITCPPPMSVEHADIWVKSYSLYSRERYICNSGFKRKAGTSSLTECVL
NKATNVAHWTTPSLKCIRDPALVHQRPAPPS
Chain 2: Fc-b-GGSLSGRSAN
(SEQ ID NO: 96)
EPKSSDKTHTCPPCPAPEAAGGPSVFLFPPKPKDTLYITREPEVTCV
VVDVSHEDPEVKFNWYVDGVEVHNAKTKPREEQYNSTYRVVSVLTVL
HQDWLNGKEYKCKVSNKALAAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVMHEALHNHYTQKSLSLSPGK

[symbol]

Chain 3: AGGS-IL-15-(GGS)5-IL-15Rα
(SEQ ID NO: 97) [symbol]
NWVNVISDLKKIEDLIQSMHIDATLYTESDVHPSCKVTAMKCF
LLELQVISLESGDASIHDTVENLIILANNSLSSNGNVTESGCKECEE
LEEKNIKEFLQSFVHIVQMFINTA [symbol] [symbol] ITCPPPMS
VEHADIWVKSYSLYSRERYICNSGFKRKAGTSSLTECVLNKATNVAH
WTTPSLKCIRDPALVHQRPAPPS
Fc-b-GPLGLAGRSANAGS-IL-15(S162A)-(GGS)5-IL-15Rα
>P1336_Fc-b-PS3-L15R-S162A chains 1 and 2
(SEQ ID NO: 98)
EPKSSDKTHTCPPCPAPEAAGGPSVFLFPPKPKDTLYITREPEVTCV
VVDVSHEDPEVKFNWYVDGVEVHNAKTKPREEQYNSTYRVVSVLTVL
HQDWLNGKEYKCKVSNKALAAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVMHEALHNHYTQKSLSLSPGK [symbol] [symbol]
NWVNVISDLKKIEDLIQSMHIDATLYTESDVHP
SCKVTAMKCFLLELQVISLESGDASIHDTVENLIILANNSLSSNGNV
TESGCKECEELEEKNIKEFLQSFVHIVQMFINTA [symbol] [symbol]
ITCPPPMSVEHADIWVKSYSLYSRERYICNSGFKRKAGTSSLTEC
VLNKATNVAHWTTPSLKCIRDPALVHQRPAPPS

Expected Cleavage Products from uPA or Matriptase Cleavage of P1336:

1) Fc-b-GPLGLAGR homodimer (product from
complete cleavage)
Chains 1 and 2
(SEQ ID NO: 99)
EPKSSDKTHTCPPCPAPEAAGGPSVFLFPPKPKDTLYITREPEVTCV
VVDVSHEDPEVKFNWYVDGVEVHNAKTKPREEQYNSTYRVVSVLTVL
HQDWLNGKEYKCKVSNKALAAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVMHEALHNHYTQKSLSLSPGK

[symbol]

2) SANAGS-IL-15-(GGS)5-IL-15Rα homodimer
(product from complete cleavage, active)
Chains 1 and 2
(SEQ ID NO: 100) [symbol]
NWVNVISDLKKIEDLIQSMHIDATLYTESDVHPSCKVTAMK
CFLLELQVISLESGDASIHDTVENLIILANNSLSSNGNVTESGCKECEEL
EEKNIKEFLQSFVHIVQMFINTA [symbol] [symbol] ITCPPP
MSVEHADIWVKSYSLYSRERYICNSGFKRKAGTSSLTECVLNKATNV
AHWTTPSLKCIRDPALVHQRPAPPS
3) Fc-b-GPLGLAGRSANAGS-IL-15(S162A)-(GGS)5-IL-
15Rα/Fc-b-GPLGLAGR/SANAGS-IL-15-(GGS)5-IL-15Rα
(product from partial cleavage, active)
Chain 1: Fc-b-GPLGLAGRSANAGS-IL-15(S162A)-
(GGS)5-IL-15Rα
(SEQ ID NO: 101)
EPKSSDKTHTCPPCPAPEAAGGPSVFLFPPKPKDTLYITREPEVTCV
VVDVSHEDPEVKFNWYVDGVEVHNAKTKPREEQYNSTYRVVSVLTVL
HQDWLNGKEYKCKVSNKALAAPIEKTISKAKGQPREPQVYTLPPSRE
EMTKNQVSLTCLVKGFYPSDIAVEWESNGQPENNYKTTPPVLDSDGSFFL
YSKLTVDKSRWQQGNVFSCSVMHEALHNHYTQKSLSLSPGK [symbol] [symbol]
NWVNVISDLKKIEDLIQSMHIDATLYTESDVHP
SCKVTAMKCFLLELQVISLESGDASIHDTVENLIILANNSLSSNGNV
TESGCKECEELEEKNIKEFLQSFVHIVQMFINTA [symbol] [symbol]
ITCPPPMSVEHADIWVKSYSLYSRERYIC
NSGFKRKAGTSSLTECVLNKATNVAHWTTPSLKCIRDPALVHQRPAPPS
Chain 2: Fc-b-GPLGLAGR
(SEQ ID NO: 102)
EPKSSDKTHTCPPCPAPEAAGGPSVFLFPPKPKDTLYITREPEVTCV
VVDVSHEDPEVKFNWYVDGVEVHNAKTKPREEQYNSTYRVVSVLTVL
HQDWLNGKEYKCKVSNKALAAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVMHEALHNHYTQKSLSLSPGK

[symbol]

Chain 3: SANAGS-IL-15-(GGS)5-IL-15Rα
(SEQ ID NO: 103) [symbol]
NWVNVISDLKKIEDLIQSMHIDATLYTESDVHPSCKVTAMK
CFLLELQVISLESGDASIHDTVENLIILANNSLSSNGNVTESGCKECEEL
EEKNIKEFLQSFVHIVQMFINTA [symbol] [symbol] ITCPPP
MSVEHADIWVKSYSLYSRERYICNSGFKRKAGTSSLTECVLNKATNV
AHWTTPSLKCIRDPALVHQRPAPPS

Expected Cleavage Products from Legumain Cleavage of P1336:

1) Fc-b-GPLGLAGRSAN homodimer (product from
complete cleavage)
Chains 1 and 2
(SEQ ID NO: 104)
EPKSSDKTHTCPPCPAPEAAGGPSVFLFPPKPKDTLYITREPEVTCV
VVDVSHEDPEVKFNWYVDGVEVHNAKTKPREEQYNSTYRVVSVLTVL
HQDWLNGKEYKCKVSNKALAAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVMHEALHNHYTQKSLSLSPGK

[symbol]

2) AGS-IL-15-(GGS)5-IL-15Rα homodimer (product
from complete cleavage, active)
Chains 1 and 2
(SEQ ID NO: 105) [symbol]
NWVNVISDLKKIEDLIQSMHIDATLYTESDVHPSCKVTAMKCFL
LELQVISLESGDASIHDTVENLIILANNSLSSNGNVTESGCKECEEL
EEKNIKEFLQSFVHIVQMFINTA [symbol] [symbol] ITCPPPMSV
EHADIWVKSYSLYSRERYICNSGFKRKAGTSSLTECVLNKATNVAHW
TTPSLKCIRDPALVHQRPAPPS
3) Fc-b-GPLGLAGRSANAGS-IL-15(S162A)-(GGS)5-IL-
15Rα/Fc-b-GPLGLAGRSAN/AGS-IL-15-(GGS)5-IL-15Rα
(product from partial cleavage, active)
Chain 1: Fc-b-GPLGLAGRSANAGS-IL-15(S162A)-
(GGS)5-IL-15Rα
(SEQ ID NO: 106)
EPKSSDKTHTCPPCPAPEAAGGPSVFLFPPKPKDTLYITREPEVTCV
VVDVSHEDPEVKFNWYVDGVEVHNAKTKPREEQYNSTYRVVSVLTVL
HQDWLNGKEYKCKVSNKALAAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVMHEALHNHYTQKSLSLSPGK [symbol] [symbol]
NWVNVISDLKKIEDLIQSMHIDATLYTESDVHP
SCKVTAMKCFLLELQVISLESGDASIHDTVENLIILANNSLSSNGNVTE
SGCKECEELEEKNIKEFLQSFVHIVQMFINTA [symbol] [symbol]
ITCPPPMSVEHADIWVKSYSLYSRERYICNSGFKRKAGTSSLTECVL
NKATNVAHWTTPSLKCIRDPALVHQRPAPPS
Chain 2: Fc-b-GPLGLAGRSAN
(SEQ ID NO: 107)
EPKSSDKTHTCPPCPAPEAAGGPSVFLFPPKPKDTLYITREPEVTCV
VVDVSHEDPEVKFNWYVDGVEVHNAKTKPREEQYNSTYRVVSVLTVL
HQDWLNGKEYKCKVSNKALAAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVMHEALHNHYTQKSLSLSPGK

[symbol]

Chain 3: AGS-IL-15-(GGS)5-IL-15Rα
(SEQ ID NO: 108) [symbol]
NWVNVISDLKKIEDLIQSMHIDATLYTESDVHPSCKVTAMKCFL
LELQVISLESGDASIHDTVENLIILANNSLSSNGNVTESGCKECEEL
EEKNIKEFLQSFVHIVQMFINTA [symbol] [symbol] ITCPPPMS
VEHADIWVKSYSLYSRERYICNSGFKRKAGTSSLTECVLNKATNVAH
WTTPSLKCIRDPALVHQRPAPPS

Expected Cleavage Products from MMP-2 or MMP-9 Cleavage of P1336:

1) Fc-b-GPLG homodimer (product from complete
cleavage)
Chains 1 and 2
(SEQ ID NO: 109)
EPKSSDKTHTCPPCPAPEAAGGPSVFLFPPKPKDTLYITREPEVTCV
VVDVSHEDPEVKFNWYVDGVEVHNAKTKPREEQYNSTYRVVSVLTVL
HQDWLNGKEYKCKVSNKALAAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVMHEALHNHYTQKSLSLSPGK [symbol]
2) LAGRSANAGS-IL-15-(GGS)5-IL-15Rα homodimer
(product from complete cleavage, active)
Chains 1 and 2
(SEQ ID NO: 110) [symbol]
NWVNVISDLKKIEDLIQSMHIDATLYTESDVHPSCKV
TAMKCFLLELQVISLESGDASIHDTVENLIILANNSLSSNGNVTESG
CKECEELEEKNIKEFLQSFVHIVQMFINTA [symbol] [symbol] IT
CPPPMSVEHADIWVKSYSLYSRERYICNSGFKRKAGTSSLTECVLNK
ATNVAHWTTPSLKCIRDPALVHQRPAPPS
3) Fc-b-GPLGLAGRSANAGS-IL-15(S162A)-(GGS)5-IL-
15Rα/Fc-b-GPLG/LAGRSANAGS-IL-15-(GGS)5-IL-15Rα
(product from partial cleavage, active)
Chain 1: Fc-b-GPLGLAGRSANAGS-IL-15(S162A)-
(GGS)5-IL-15Rα
(SEQ ID NO: 111)
EPKSSDKTHTCPPCPAPEAAGGPSVFLFPPKPKDTLYITREPEVTCV
VVDVSHEDPEVKFNWYVDGVEVHNAKTKPREEQYNSTYRVVSVLTVL
HQDWLNGKEYKCKVSNKALAAPIEKTISKAKGQPREPQVYTLPPSRE
EMTKNQVSLTCLVKGFYPSDIAVEWESNGQPENNYKTTPPVLDSDGSFFL
YSKLTVDKSRWQQGNVFSCSVMHEALHNHYTQKSLSLSPGK [symbol] [symbol]
NWVNVISDLKKIEDLIQSMHIDATLYTESDVHP
SCKVTAMKCFLLELQVISLESGDASIHDTVENLIILANNSLSSNGNV
TESGCKECEELEEKNIKEFLQSFVHIVQMFINTAGGSGGSGGSGGSG
GSITCPPPMSVEHADIWVKSYSLYSRERYICNSGFKRKAGTSSLTEC
VLNKATNVAHWTTPSLKCIRDPALVHQRPAPPS
Chain 2: Fc-b-GPLG
(SEQ ID NO: 112)
EPKSSDKTHTCPPCPAPEAAGGPSVFLFPPKPKDTLYITREPEVTCV
VVDVSHEDPEVKFNWYVDGVEVHNAKTKPREEQYNSTYRVVSVLTVL
HQDWLNGKEYKCKVSNKALAAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVMHEALHNHYTQKSLSLSPGK [symbol]
Chain 3: LAGRSANAGS-IL-15-(GGS)5-IL-15Rα
(SEQ ID NO: 113) [symbol]
NWVNVISDLKKIEDLIQSMHIDATLYTESDVHPSCKV
TAMKCFLLELQVISLESGDASIHDTVENLIILANNSLSSNGNVTESGCKE
CEELEEKNIKEFLQSFVHIVQMFINTA [symbol] [symbol]
ITCPPPMSVEHADIWVKSYSLYSRERYICNSGFKRKAGTSSLTECVLNK
ATNVAHWTTPSLKCIRDPALVHQRPAPPS
Fc-b-PLGLSGRSANAGPA-IL-15(S162A)-(GGS)5-IL-15Rα
>P1337_Fc-b-PS4-L15R-S162A chains 1 and 2
(SEQ ID NO: 114)
EPKSSDKTHTCPPCPAPEAAGGPSVFLFPPKPKDTLYITREPEVTCV
VVDVSHEDPEVKFNWYVDGVEVHNAKTKPREEQYNSTYRVVSVLTVL
HQDWLNGKEYKCKVSNKALAAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVMHEALHNHYTQKSLSLSPGK [symbol]
NWVNVISDLKKIEDLIQSMHIDATLYTESDV
HPSCKVTAMKCFLLELQVISLESGDASIHDTVENLIILANNSLSSNGNV
TESGCKECEELEEKNIKEFLQSFVHIVQMFINTA [symbol] [symbol]
ITCPPPMSVEHADIWVKSYSLYSRERYICNSGFKRKA
GTSSLTECVLNKATNVAHWTTPSLKCIRDPALVHQRPAPPS

Expected Cleavage Products from uPA or Matriptase Cleavage of P1337:

1) Fc-b-PLGLSGR homodimer (product from
complete cleavage)
Chains 1 and 2
(SEQ ID NO: 115)
EPKSSDKTHTCPPCPAPEAAGGPSVFLFPPKPKDTLYITREPEVTCV
VVDVSHEDPEVKFNWYVDGVEVHNAKTKPREEQYNSTYRVVSVLTVL
HQDWLNGKEYKCKVSNKALAAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVMHEALHNHYTQKSLSLSPGK

[symbol]

2) SANAGPA-IL-15-(GGS)5-IL-15Rα homodimer
(product from complete cleavage, active)
Chains 1 and 2
(SEQ ID NO: 116) [symbol]
NWVNVISDLKKIEDLIQSMHIDATLYTESDVHPSCKVTAM
KCFLLELQVISLESGDASIHDTVENLIILANNSLSSNGNVTESGCKE
CEELEEKNIKEFLQSFVHIVQMFINTA [symbol] [symbol] ITCPP
PMSVEHADIWVKSYSLYSRERYICNSGFKRKAGTSSLTECVLNKATN
VAHWTTPSLKCIRDPALVHQRPAPPS
3) Fc-b-PLGLSGRSANAGPA-IL-15(S162A)-(GGS)5-IL-
15Rα/Fc-b-PLGLSGR/SANAGPA-IL-15-(GGS)5-IL-15Rα
(product from partial cleavage, active)
Chain 1: Fc-b-PLGLSGRSANAGPA-IL-15(S162A)-
(GGS)5-IL-15Rα
(SEQ ID NO: 117)
EPKSSDKTHTCPPCPAPEAAGGPSVFLFPPKPKDTLYITREPEVTCV
VVDVSHEDPEVKFNWYVDGVEVHNAKTKPREEQYNSTYRVVSVLTVL
HQDWLNGKEYKCKVSNKALAAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVMHEALHNHYTQKSLSLSPGK [symbol] [symbol]
NWVNVISDLKKIEDLIQSMHIDATLYTESDVHP
SCKVTAMKCFLLELQVISLESGDASIHDTVENLIILANNSLSSNGNVTE
SGCKECEELEEKNIKEFLQSFVHIVQMFINTA [symbol] [symbol]
ITCPPPMSVEHADIWVKSYSLYSRERYICNSGFKRKAGTSSLTEC
VLNKATNVAHWTTPSLKCIRDPALVHQRPAPPS
Chain 2: Fc-b-PLGLSGR
(SEQ ID NO: 118)
EPKSSDKTHTCPPCPAPEAAGGPSVFLFPPKPKDTLYITREPEVTCV
VVDVSHEDPEVKFNWYVDGVEVHNAKTKPREEQYNSTYRVVSVLTVL
HQDWLNGKEYKCKVSNKALAAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVMHEALHNHYTQKSLSLSPGK

[symbol]

Chain 3: SANAGPA-IL-15-(GGS)5-IL-15Rα
(SEQ ID NO: 119) [symbol]
NWVNVISDLKKIEDLIQSMHIDATLYTESDVHPSCKVTAM
KCFLLELQVISLESGDASIHDTVENLIILANNSLSSNGNVTESGCKE
CEELEEKNIKEFLQSFVHIVQMFINTA [symbol] [symbol] ITCPP
PMSVEHADIWVKSYSLYSRERYICNSGFKRKAGTSSLTECVLNKATN
VAHWTTPSLKCIRDPALVHQRPAPPS

Expected Cleavage Products from Legumain Cleavage of P1337:

1) Fc-b-PLGLSGRSAN homodimer (product from complete cleavage)
Chains 1 and 2
(SEQ ID NO: 120)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) AGPA-IL-15-(GGS)5-IL-15Rα homodimer(product from complete
cleavage, active)
Chains 1 and 2
(SEQ ID NO: 121) [symbol]
NWVNVISDLKKIEDLIQSMHIDATLYTESDVHPSCKVTAMKCFLLELQVISLESGDASI
HDTVENLIILANNSLSSNGNVTESGCKECEELEEKNIKEFLQSFVHIVQMFINTA [symbol] [symbol] ITCPP
PMSVEHADIWVKSYSLYSRERYICNSGFKRKAGTSSLTECVLNKATNVAHWTTPSLKCIRDPALVHQRPAPPS
3) Fc-b-PLGLSGRSANAGPA-IL-15(S162A)-(GGS)5-IL-15Rα/Fc-b-
PLGLSGRSAN/AGPA-IL-15-(GGS)5-IL-15Rα (product from partial cleavage,
active)
Chain 1: Fc-b-PLGLSGRSANAGPA-IL-15(S162A)-(GGS)5-IL-15Rα
(SEQ ID NO: 122)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKVTAMKC
FLLELQVISLESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPSLKC
IRDPALVHQRPAPPS
Chain 2: Fc-b-PLGLSGRSAN
(SEQ ID NO: 123)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: AGPA-IL-15-(GGS)5-IL-15Rα
(SEQ ID NO: 124) [symbol]
NWVNVISDLKKIEDLIQSMHIDATLYTESDVHPSCKVTAMKCFLLELQVISLESGDASI
HDTVENLIILANNSLSSNGNVTESGCKECEELEEKNIKEFLQSFVHIVQMFINTA [symbol] [symbol] ITCPP
PMSVEHADIWVKSYSLYSRERYICNSGFKRKAGTSSLTECVLNKATNVAHWTTPSLKCIRDPALVHQRPAPPS

Expected Cleavage Products from MMP-2 or MMP-9 Cleavage of P1337:

1) Fc-b-PLG homodimer (product from complete cleavage)
Chains 1 and 2
(SEQ ID NO: 125)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) LSGRSANAGPA-IL-15-(GGS)5-IL-15Rα homodimer(product from
complete cleavage, active)
Chains 1 and 2
(SEQ ID NO: 126) [symbol]
NWVNVISDLKKIEDLIQSMHIDATLYTESDVHPSCKVTAMKCFLLELQVISL
ESGDASIHDTVENLIILANNSLSSNGNVTESGCKECEELEEKNIKEFLQSFVHIVQMFINTA [symbol] [symbol] [symbol]
ITCPPPMSVEHADIWVKSYSLYSRERYICNSGFKRKAGTSSLTECVLNKATNVAHWTTPSLKCIRDPALVHQR
PAPPS
3) Fc-b-PLGLSGRSANAGPA-IL-15(S162A)-(GGS)5-IL-15Rα/Fc-b-PLG/
LSGRSANAGPA-IL-15-(GGS)5-IL-15Rα (product from partial cleavage, active)
Chain 1: Fc-b-PLGLSGRSANAGPA-IL-15(S162A)-(GGS)5-IL-15Rα
(SEQ ID NO: 127)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKVTAMKC
FLLELQVISLESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPSLKC
IRDPALVHQRPAPPS
Chain 2: Fc-b-PLG
(SEQ ID NO: 128)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: LSGRSANAGPA-IL-15-(GGS)5-IL-15Rα
(SEQ ID NO: 129) [symbol]
NWVNVISDLKKIEDLIQSMHIDATLYTESDVHPSCKVTAMKCFLLELQVISL
ESGDASIHDTVENLIILANNSLSSNGNVTESGCKECEELEEKNIKEFLQSFVHIVQMFINTA [symbol] [symbol] [symbol]
ITCPPPMSVEHADIWVKSYSLYSRERYICNSGFKRKAGTSSLTECVLNKATNVAHWTTPSLKCIRDPALVHQR
PAPPS
Fc-b-PLGLAGRSANAGPA-IL-15(S162A)-(GGS)5-IL-15Rα
>P1338_Fc-b-PS5-L15R-S162A chains 1 and 2
(SEQ ID NO: 130)
EPKSSDKTHTCPPCPAPEAAGGPSVFLFPPKPKDTLYITREPEVTCVVVDVSHEDPEVKFNWYVDGVEVHNAKTK
PREEQYNSTYRVVSVLTVLHQDWLNGKEYKCKVSNKALAAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] [symbol] NWVNVISDLKKIEDLIQSMHIDATLYTESDVHPSCKVTAMKCFLLELQVISLES
GDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPSLKCIRDPALVHQRPA
PPS

Expected Cleavage Products from uPA or Matriptase Cleavage of P1338:

1) Fc-b-PLGLAGR homodimer (product from complete cleavage)
Chains 1 and 2
(SEQ ID NO: 131)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) SANAGPA-IL-15-(GGS)5-IL-15Rα homodimer (product from complete
cleavage, active)
Chains 1 and 2
(SEQ ID NO: 132) [symbol]
NWVNVISDLKKIEDLIQSMHIDATLYTESDVHPSCKVTAMKCFLLELQVISLESGD
ASIHDTVENLIILANNSLSSNGNVTESGCKECEELEEKNIKEFLQSFVHIVQMFINTA [symbol] [symbol] IT
CPPPMSVEHADIWVKSYSLYSRERYICNSGFKRKAGTSSLTECVLNKATNVAHWTTPSLKCIRDPALVHQRPAPP
S
3) Fc-b-PLGLAGRSANAGPA-IL-15(S162A)-(GGS)5-IL-15Rα/Fc-b-PLGLAGR/
SANAGPA-IL-15-(GGS)5-IL-15Rα (product from partial cleavage, active)
Chain 1: Fc-b-PLGLAGRSANAGPA-IL-15(S162A)-(GGS)5-IL-15Rα
(SEQ ID NO: 133)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKVTAMKC
FLLELQVISLESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPSLKC
IRDPALVHQRPAPPS
Chain 2: Fc-b-PLGLAGR
(SEQ ID NO: 134)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: SANAGPA-IL-15-(GGS)5-IL-15Rα
(SEQ ID NO: 135) [symbol]
NWVNVISDLKKIEDLIQSMHIDATLYTESDVHPSCKVTAMKCFLLELQVISLESGD
ASIHDTVENLIILANNSLSSNGNVTESGCKECEELEEKNIKEFLQSFVHIVQMFINTA [symbol] [symbol] IT
CPPPMSVEHADIWVKSYSLYSRERYICNSGFKRKAGTSSLTECVLNKATNVAHWTTPSLKCIRDPALVHQRPAPP
S

Expected Cleavage Products from Legumain Cleavage of P1338:

1) Fc-b-PLGLAGRSAN homodimer (product from complete cleavage)
Chains 1 and 2
(SEQ ID NO: 136)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) AGPA-IL-15-(GGS)5-IL-15Rα homodimer (product from complete
cleavage, active)
Chains 1 and 2
(SEQ ID NO: 137) [symbol]
NWVNVISDLKKIEDLIQSMHIDATLYTESDVHPSCKVTAMKCFLLELQVISLESGDASI
HDTVENLIILANNSLSSNGNVTESGCKECEELEEKNIKEFLQSFVHIVQMFINT [symbol] [symbol] ITCPP
PMSVEHADIWVKSYSLYSRERYICNSGFKRKAGTSSLTECVLNKATNVAHWTTPSLKCIRDPALVHQRPAPPS
3) Fc-b-PLGLAGRSANAGPA-IL-15(S162A)-(GGS)5-IL-15Rα/Fc-b-PLGLAGRSAN/
AGPA-IL-15-(GGS)5-IL-15Rα (product from partial cleavage, active)
Chain 1: Fc-b-PLGLAGRSANAGPA-IL-15(S162A)-(GGS)5-IL-15Rα
(SEQ ID NO: 138)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKVTAMKC
FLLELQVISLESGDASIHDTVENLIILANNSLSSNGNVTESGCKECEELEEKNIKEFLQSFVHIVQMFINT [symbol] [symbol]
ITCPPPMSVEHADIWVKSYSLYSRERYICNSGFKRKAGTSSLTECVLNKATNVAHWTTPSLKC
IRDPALVHQRPAPPS
Chain 2: Fc-b-PLGLAGRSAN
(SEQ ID NO: 139)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: AGPA-IL-15-(GGS)5-IL-15Rα
(SEQ ID NO: 140) [symbol]
NWVNVISDLKKIEDLIQSMHIDATLYTESDVHPSCKVTAMKCFLLELQVISLESGDASI
HDTVENLIILANNSLSSNGNVTESGCKECEELEEKNIKEFLQSFVHIVQMFINT [symbol] [symbol] ITCPP
PMSVEHADIWVKSYSLYSRERYICNSGFKRKAGTSSLTECVLNKATNVAHWTTPSLKCIRDPALVHQRPAPPS

Expected Cleavage Products from MMP-2 or MMP-9 Cleavage of P1338:

1) Fc-b-PLG homodimer (product from complete cleavage)
Chains 1 and 2
(SEQ ID NO: 141)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) LAGRSANAGPA-IL-15-(GGS)5-IL-15Rα homodimer (product from
complete cleavage, active)
Chains 1 and 2
(SEQ ID NO: 142) [symbol]
NWVNVISDLKKIEDLIQSMHIDATLYTESDVHPSCKVTAMKCFLLELQVISL
ESGDASIHDTVENLIILANNSLSSNGNVTESGCKECEELEEKNIKEFLQSFVHIVQMFINTA [symbol] [symbol] [symbol]
ITCPPPMSVEHADIWVKSYSLYSRERYICNSGFKRKAGTSSLTECVLNKATNVAHWTTPSLKCIRDPALVHQR
PAPPS
3) Fc-b-PLGLAGRSANAGPA-IL-15(S162A)-(GGS)5-IL-15Rα/Fc-b-PLG/
LAGRSANAGPA-IL-15-(GGS)5-IL-15Rα (product from partial cleavage, active)
Chain 1: Fc-b-PLGLAGRSANAGPA-IL-15(S162A)-(GGS)5-IL-15Rα
(SEQ ID NO: 143)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKVTAMKC
FLLELQVISLESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPSLKC
IRDPALVHQRPAPPS
Chain 2: Fc-b-PLG
(SEQ ID NO: 144)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: LAGRSANAGPA-IL-15-(GGS)5-IL-15Rα
(SEQ ID NO: 145) [symbol]
NWVNVISDLKKIEDLIQSMHIDATLYTESDVHPSCKVTAMKCFLLELQVISL
ESGDASIHDTVENLIILANNSLSSNGNVTESGCKECEELEEKNIKEFLQSFVHIVQMFINTA [symbol] [symbol] [symbol]
ITCPPPMSVEHADIWVKSYSLYSRERYICNSGFKRKAGTSSLTECVLNKATNVAHWTTPSLKCIRDPALVHQR
PAPPS
Fc-b-GPLGLSGRSANAGPASG-IL-15(S162A)-(GGS)5-IL-15Rα
>P1339_Fc-b-PS6-L15R-S162A chains 1 and 2
(SEQ ID NO: 146)
EPKSSDKTHTCPPCPAPEAAGGPSVFLFPPKPKDTLYITREPEVTCVVVDVSHEDPEVKFNWYVDGVEVHNAKTK
PREEQYNSTYRVVSVLTVLHQDWLNGKEYKCKVSNKALAAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] [symbol] NWVNVISDLKKIEDLIQSMHIDATLYTESDVHPSCKVTAMKCFLLELQVIS
LESGDASIHDTVENLIILANNSLSSNGNVTESGCKECEELEEKNIKEFLQSFVHIVQMFINTA [symbol] [symbol] [symbol]
ITCPPPMSVEHADIWVKSYSLYSRERYICNSGFKRKAGTSSLTECVLNKATNVAHWTTPSLKCIRDPALVHQ
RPAPPS

Expected Cleavage Products from uPA or Matriptase Cleavage of P1339:

1) Fc-b-GPLGLSGR homodimer (product from complete cleavage)
Chains 1 and 2
(SEQ ID NO: 147)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) SANAGPASG-IL-15-(GGS)5-IL-15Rα homodimer (product from
complete cleavage, active)
Chains 1 and 2
(SEQ ID NO: 148) [symbol]
NWVNVISDLKKIEDLIQSMHIDATLYTESDVHPSCKVTAMKCFLLELQVISLES
GDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPSLKCIRDPALVHQRPA
PPS
3) Fc-b-GPLGLSGRSANAGPASG-IL-15(S162A)-(GGS)5-IL-15Rα/Fc-b-GPLGLSGR/
SANAGPASG-IL-15-(GGS)5-IL-15Rα (product from partial cleavage, active)
Chain 1: Fc-b-PLGLAGRSANAGPA-IL-15(S162A)-(GGS)5-IL-15Rα
(SEQ ID NO: 149)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKVTA
MKCFLLELQVISLESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPS
LKCIRDPALVHQRPAPPS
Chain 2: Fc-b-GPLGLSGR
(SEQ ID NO: 150)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: SANAGPASG-IL-15-(GGS)5-IL-15Rα
(SEQ ID NO: 151) [symbol]
NWVNVISDLKKIEDLIQSMHIDATLYTESDVHPSCKVTAMKCFLLELQVISLES
GDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPSLKCIRDPALVHQRPA
PPS

Expected Cleavage Products from Legumain Cleavage of P1339:

1) Fc-b-GPLGLSGRSAN homodimer (product from complete cleavage)
Chains 1 and 2
(SEQ ID NO: 152)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) AGPASG-IL-15-(GGS)5-IL-15Rα homodimer (product from complete
cleavage, active)
Chains 1 and 2
(SEQ ID NO: 153) [symbol]
NWVNVISDLKKIEDLIQSMHIDATLYTESDVHPSCKVTAMKCFLLELQVISLESGDA
SIHDTVENLIILANNSLSSNGNVTESGCKECEELEEKNIKEFLQSFVHIVQMFINTA [symbol] [symbol] ITC
PPPMSVEHADIWVKSYSLYSRERYICNSGFKRKAGTSSLTECVLNKATNVAHWTTPSLKCIRDPALVHQRPAPPS
3) Fc-b-GPLGLSGRSANAGPASG-IL-15(S162A)-(GGS)5-IL-15Rα/
Fc-b-GPLGLSGRSAN/AGPA
Chain 1: Fc-b-PLGLAGRSANAGPA-IL-15(S162A)-(GGS)5-IL-15Rα
(SEQ ID NO: 154)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKVTA
MKCFLLELQVISLESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPS
LKCIRDPALVHQRPAPPS
Chain 2: Fc-b-GPLGLSGRSAN
(SEQ ID NO: 155)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: AGPASG-IL-15-(GGS)5-IL-15Rα
(SEQ ID NO: 156) [symbol]
NWVNVISDLKKIEDLIQSMHIDATLYTESDVHPSCKVTAMKCFLLELQVISLESGDA
SIHDTVENLIILANNSLSSNGNVTESGCKECEELEEKNIKEFLQSFVHIVQMFINTA [symbol] [symbol] ITC
PPPMSVEHADIWVKSYSLYSRERYICNSGFKRKAGTSSLTECVLNKATNVAHWTTPSLKCIRDPALVHQRPAPPS

Expected Cleavage Products from MMP-2 or MMP-9 Cleavage of P1339:

1) Fc-b-GPLG homodimer (product from complete cleavage)
Chains 1 and 2
(SEQ ID NO: 157)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) LSGRSANAGPASG-IL-15-(GGS)5-IL-15Rα homodimer (product from
complete cleavage, active)
Chains 1 and 2
(SEQ ID NO: 158) [symbol] [symbol]
NWVNVISDLKKIEDLIQSMHIDATLYTESDVHPSCKVTAMKCFLLELQVI
SLESGDASIHDTVENLIILANNSLSSNGNVTESGCKECEELEEKNIKEFLQSFVHIVQMFINT [symbol] [symbol]
ITCPPPMSVEHADIWVKSYSLYSRERYICNSGFKRKAGTSSLTECVLNKATNVAHWTTPSLKCIRDPALVH
QRPAPPS
3) Fc-b-GPLGLSGRSANAGPASG-IL-15(S162A)-(GGS)5-IL-15Rα/Fc-b-GPLG/
LSGRSANAGPASG-IL-15-(GGS)5-IL-15Rα (product from partial cleavage, active)
Chain 1: Fc-b-PLGLAGRSANAGPA-IL-15(S162A)-(GGS)5-IL-15Rα
(SEQ ID NO: 159)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKVTA
MKCFLLELQVISLESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPS
LKCIRDPALVHQRPAPPS
Chain 2: Fc-b-GPLG
(SEQ ID NO: 160)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: LSGRSANAGPASG-IL-15-(GGS)5-IL-15Rα
(SEQ ID NO: 161) [symbol] [symbol]
NWVNVISDLKKIEDLIQSMHIDATLYTESDVHPSCKVTAMKCFLLELQVI
SLESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPSLKCIRDPALVH
QRPAPPS
Fc-b-GPLGLAGRSANAGPASG-IL-15(S162A)-(GGS)5-IL-15Rα
>P1340_Fc-b-PS7-L15R-S162A chains 1 and 2
(SEQ ID NO: 162)
EPKSSDKTHTCPPCPAPEAAGGPSVFLFPPKPKDTLYITREPEVTCVVVDVSHEDPEVKFNWYVDGVEVHNAKTK
PREEQYNSTYRVVSVLTVLHQDWLNGKEYKCKVSNKALAAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] [symbol] NWVNVISDLKKIEDLIQSMHIDATLYTESDVHPSCKVTAMKCFLLELQVIS
LESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPSLKCIRDPALVHQ
RPAPPS

Expected Cleavage Products from uPA or Matriptase Cleavage of P1340:

1) Fc-b-GPLGLAGR homodimer (product from complete cleavage)
Chains 1 and 2
(SEQ ID NO: 163)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) SANAGPASG-IL-15-(GGS)5-IL-15Rα homodimer (product from
complete cleavage, active)
Chains 1 and 2
(SEQ ID NO: 164) [symbol]
NWVNVISDLKKIEDLIQSMHIDATLYTESDVHPSCKVTAMKCFLLELQVISLES
GDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPSLKCIRDPALVHQRPA
PPS
3) Fc-b-GPLGLAGRSANAGPASG-IL-15(S162A)-(GGS)5-IL-15Rα/Fc-b-GPLGLAGR/
SANAGPASG-IL-15-(GGS)5-IL-15Rα (product from partial cleavage, active)
Chain 1: Fc-b-GPLGLAGRSANAGPASG-IL-15(S162A)-(GGS)5-IL-15Rα
(SEQ ID NO: 165)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKVTA
MKCFLLELQVISLESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPS
LKCIRDPALVHQRPAPPS
Chain 2: Fc-b-GPLGLAGR
(SEQ ID NO: 166)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: SANAGPASG-IL-15-(GGS)5-IL-15Rα
(SEQ ID NO: 167) [symbol]
NWVNVISDLKKIEDLIQSMHIDATLYTESDVHPSCKVTAMKCFLLELQVISLES
GDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPSLKCIRDPALVHQRPA
PPS

Expected Cleavage Products from Legumain Cleavage of P1340:

1) Fc-b-GPLGLAGRSAN homodimer (product from complete cleavage)
Chains 1 and 2
(SEQ ID NO: 168)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) AGPASG-IL-15-(GGS)5-IL-15Rα homodimer (product from complete
cleavage, active)
Chains 1 and 2
(SEQ ID NO: 169) [symbol]
NWVNVISDLKKIEDLIQSMHIDATLYTESDVHPSCKVTAMKCFLLELQVISLESGDA
SIHDTVENLIILANNSLSSNGNVTESGCKECEELEEKNIKEFLQSFVHIVQMFINTA [symbol] [symbol] ITC
PPPMSVEHADIWVKSYSLYSRERYICNSGFKRKAGTSSLTECVLNKATNVAHWTTPSLKCIRDPALVHQRPAPPS
3) Fc-b-GPLGLAGRSANAGPASG-IL-15(S162A)-(GGS)5-IL-15Rα/Fc-b-GPLGLAGRSAN/
AGPASG-IL-15-(GGS)5-IL-15Rα (product from partial cleavage, active)
Chain 1: Fc-b-GPLGLAGRSANAGPASG-IL-15(S162A)-(GGS)5-IL-15Rα
(SEQ ID NO: 170)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKVTA
MKCFLLELQVISLESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPS
LKCIRDPALVHQRPAPPS
Chain 2: Fc-b-GPLGLAGRSAN
(SEQ ID NO: 171)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: AGPASG-IL-15-(GGS)5-IL-15Rα
(SEQ ID NO: 172) [symbol]
NWVNVISDLKKIEDLIQSMHIDATLYTESDVHPSCKVTAMKCFLLELQVISLESGDA
SIHDTVENLIILANNSLSSNGNVTESGCKECEELEEKNIKEFLQSFVHIVQMFINTA [symbol] [symbol] ITC
PPPMSVEHADIWVKSYSLYSRERYICNSGFKRKAGTSSLTECVLNKATNVAHWTTPSLKCIRDPALVHQRPAPPS

Expected Cleavage Products from MMP-2 or MMP-9 Cleavage of P1340:

1) Fc-b-GPLG homodimer (product from complete cleavage)
Chains 1 and 2
(SEQ ID NO: 173)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) LAGRSANAGPASG-IL-15-(GGS)5-IL-15Rα homodimer (product from
complete cleavage, active)
Chains 1 and 2
(SEQ ID NO: 174) [symbol] [symbol]
NWVNVISDLKKIEDLIQSMHIDATLYTESDVHPSCKVTAMKCFLLELQVI
SLESGDASIHDTVENLIILANNSLSSNGNVTESGCKECEELEEKNIKEFLQSFVHIVQMFINT [symbol] [symbol]
ITCPPPMSVEHADIWVKSYSLYSRERYICNSGFKRKAGTSSLTECVLNKATNVAHWTTPSLKCIRDPALVH
QRPAPPS
3) Fc-b-GPLGLAGRSANAGPASG-IL-15(S162A)-(GGS)5-IL-15Rα/Fc-b-GPLG/
LAGRSANAGPASG-IL-15-(GGS)5-IL-15Rα (product from partial cleavage, active)
Chain 1: Fc-b-GPLGLAGRSANAGPASG-IL-15(S162A)-(GGS)5-IL-15Rα
(SEQ ID NO: 175)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKVTA
MKCFLLELQVISLESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPS
LKCIRDPALVHQRPAPPS
Chain 2: Fc-b-GPLG
(SEQ ID NO: 176)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: LAGRSANAGPASG-IL-15-(GGS)5-IL-15Rα
(SEQ ID NO: 177) [symbol] [symbol]
NWVNVISDLKKIEDLIQSMHIDATLYTESDVHPSCKVTAMKCFLLELQVI
SLESGDASIHDTVENLIILANNSLSSNGNVTESGCKECEELEEKNIKEFLQSFVHIVQMFINTA [symbol] [symbol] [symbol]
ITCPPPMSVEHADIWVKSYSLYSRERYICNSGFKRKAGTSSLTECVLNKATNVAHWTTPSLKCIRDPALVH
QRPAPPS
Fc-b-SGPLGLAGRSANAGPAS-IL-15(S162A)-(GGS)5-IL-15Rα
>P1341_Fc-b-PS8-L15R-S162A) chains 1 and 2
(SEQ ID NO: 178)
EPKSSDKTHTCPPCPAPEAAGGPSVFLFPPKPKDTLYITREPEVTCVVVDVSHEDPEVKFNWYVDGVEVHNAKTK
PREEQYNSTYRVVSVLTVLHQDWLNGKEYKCKVSNKALAAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] [symbol] NWVNVISDLKKIEDLIQSMHIDATLYTESDVHPSCKVTAMKCFLLELQVIS
LESGDASIHDTVENLIILANNSLSSNGNVTESGCKECEELEEKNIKEFLQSFVHIVQMFINTA [symbol] [symbol] [symbol]
ITCPPPMSVEHADIWVKSYSLYSRERYICNSGFKRKAGTSSLTECVLNKATNVAHWTTPSLKCIRDPALVHQ
RPAPPS

Expected Cleavage Products from uPA or Matriptase Cleavage of P1341:

1) Fc-b-SGPLGLAGR homodimer (product from complete cleavage)
Chains 1 and 2
(SEQ ID NO: 179)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKSGPLGLAGR
2) SANAGPAS-IL-15-(GGS)5-IL-15Rα homodimer (product from
complete cleavage, active)
Chains 1 and 2
(SEQ ID NO: 180) [symbol]
NWVNVISDLKKIEDLIQSMHIDATLYTESDVHPSCKVTAMKCFLLELQVISLESG
DASIHDTVENLIILANNSLSSNGNVTESGCKECEELEEKNIKEFLQSFVHIVQMFINTA [symbol] [symbol] I
TCPPPMSVEHADIWVKSYSLYSRERYICNSGFKRKAGTSSLTECVLNKATNVAHWTTPSLKCIRDPALVHQRPAP
PS
3) Fc-b-SGPLGLAGRSANAGPAS-IL-15(S162A)-(GGS)5-IL-15Rα/Fc-b-SGPLGLAGR/
SANAGPAS-IL-15-(GGS)5-IL-15Rα (product from partial cleavage, active)
Chain 1: Fc-b-SGPLGLAGRSANAGPAS-IL-15(S162A)-(GGS)5-IL-15Rα
(SEQ ID NO: 181)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKVTA
MKCFLLELQVISLESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHW
TTPSLKCIRDPALVHQRPAPPS
Chain 2: Fc-b-SGPLGLAGR
(SEQ ID NO: 182)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: SANAGPAS-IL-15-(GGS)5-IL-15Rα
(SEQ ID NO: 183) [symbol]
NWVNVISDLKKIEDLIQSMHIDATLYTESDVHPSCKVTAMKCFLLELQVISLESG
DASIHDTVENLIILANNSLSSNGNVTESGCKECEELEEKNIKEFLQSFVHIVQMFINTA [symbol] [symbol] I
TCPPPMSVEHADIWVKSYSLYSRERYICNSGFKRKAGTSSLTECVLNKATNVAHWTTPSLKCIRDPALVHQRPAP
PS

Expected Cleavage Products from Legumain Cleavage of P1341:

1) Fc-b-SGPLGLAGRSAN homodimer (product from complete cleavage)
Chains 1 and 2
(SEQ ID NO: 184)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol]
2) AGPAS-IL-15-(GGS)5-IL-15Rα homodimer (product from complete
cleavage, active)
Chains 1 and 2
(SEQ ID NO: 185) [symbol]
NWVNVISDLKKIEDLIQSMHIDATLYTESDVHPSCKVTAMKCFLLELQVISLESGDAS
IHDTVENLIILANNSLSSNGNVTESGCKECEELEEKNIKEFLQSFVHIVQMFINTA [symbol] [symbol] ITCP
PPMSVEHADIWVKSYSLYSRERYICNSGFKRKAGTSSLTECVLNKATNVAHWTTPSLKCIRDPALVHQRPAPPS
3) Fc-b-SGPLGLAGRSANAGPAS-IL-15(S162A)-(GGS)5-IL-15Rα/Fc-b-SGPLGLAGRSAN/
AGPAS-IL-15-(GGS)5-IL-15Rα (product from partial cleavage, active)
Chain 1: Fc-b-SGPLGLAGRSANAGPAS-IL-15(S162A)-(GGS)5-IL-15Rα
(SEQ ID NO: 186)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKVTA
MKCFLLELQVISLESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPS
LKCIRDPALVHQRPAPPS
Chain 2: Fc-b-SGPLGLAGRSAN
(SEQ ID NO: 187)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: AGPAS-IL-15-(GGS)5-IL-15Rα
(SEQ ID NO: 188) [symbol]
NWVNVISDLKKIEDLIQSMHIDATLYTESDVHPSCKVTAMKCFLLELQVISLESGDAS
IHDTVENLIILANNSLSSNGNVTESGCKECEELEEKNIKEFLQSFVHIVQMFINTA [symbol] [symbol] ITCP
PPMSVEHADIWVKSYSLYSRERYICNSGFKRKAGTSSLTECVLNKATNVAHWTTPSLKCIRDPALVHQRPAPPS

Expected Cleavage Products from MMP-2 or MMP-9 Cleavage of P1341:

1) Fc-b-SGPLG homodimer (product from complete cleavage)
Chains 1 and 2
(SEQ ID NO: 189)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) LAGRSANAGPAS-IL-15-(GGS)5-IL-15Rα homodimer (product from
complete cleavage, active)
Chains 1 and 2
(SEQ ID NO: 190) [symbol]
NWVNVISDLKKIEDLIQSMHIDATLYTESDVHPSCKVTAMKCFLLELQVIS
LESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPSLKCIRDPALVHQ
RPAPPS
3) Fc-b-SGPLGLAGRSANAGPAS-IL-15(S162A)-(GGS)5-IL-15Rα/Fc-b-SGPLG/
LAGRSANAGPAS-IL-15-(GGS)5-IL-15Rα (product from partial cleavage, active)
Chain 1: Fc-b-SGPLGLAGRSANAGPAS-IL-15(S162A)-(GGS)5-IL-15Rα
(SEQ ID NO: 191)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKVTA
MKCFLLELQVISLESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPS
LKCIRDPALVHQRPAPPS
Chain 2: Fc-b-SGPLG
(SEQ ID NO: 192)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: LAGRSANAGPAS-IL-15-(GGS)5-IL-15Rα
(SEQ ID NO: 1953) [symbol]
NWVNVISDLKKIEDLIQSMHIDATLYTESDVHPSCKVTAMKCFLLELQVIS
LESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPSLKCIRDPALVHQ
RPAPPS
Fc-b-SGPASGRSANAPLGLAG-IL-15(S162A)-(GGS)5-IL-15Rα
>P1342_Fc-b-PS9-L15R-S162A chains 1 and 2
(SEQ ID NO: 194)
EPKSSDKTHTCPPCPAPEAAGGPSVFLFPPKPKDTLYITREPEVTCVVVDVSHEDPEVKFNWYVDGVEVHNAKTK
PREEQYNSTYRVVSVLTVLHQDWLNGKEYKCKVSNKALAAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] [symbol] NWVNVISDLKKIEDLIQSMHIDATLYTESDVHPSCKVTAMKCFLLELQVIS
LESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPSLKCIRDPALVHQ
RPAPPS

Expected Cleavage Products from uPA or Matriptase Cleavage of P1342:

```
1) Fc-b-SGPASGR homodimer (product from complete cleavage) (SEQ
ID NO: 195)
Chains 1 and 2
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) SANAPLGLAG-IL-15-(GGS)5-IL-15Rα homodimer(product from
complete cleavage, active) (SEQ ID NO: 196)
Chains 1 and 2 [symbol]
                  NWVNVISDLKKIEDLIQSMHIDATLYTESDVHPSCKVTAMKCELLELQVISLE
SGDASIHDTVENLIILANNSLSSNGNVTESGCKECEELEEKNIKEFLQSFVHIVQMFINTA [symbol] [symbol] [symbol]
ITCPPPMSVEHADIWVKSYSLYSRERYICNSGFKRKAGTSSLTECVLNKATNVAHWTTPSLKCIRDPALVHQRP
APPS
3) Fc-b-SGPASGRSANAPLGLAG-IL-15(S162A)-(GGS)5-IL-15Rα/Fc-b-
SGPASGR/SANAPLGLAG-IL-15-(GGS)5-IL-15Rα (product from partial cleavage,
active)
Chain 1: Fc-b-SGPASGRSANAPLGLAG-IL-15(S162A)-(GGS)5-IL-15Rα (SEQ
ID NO: 197)
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKVTA
MKCFLLELQVISLESGDASIHDTVENLIILANNSLSSNGNVTESGCKECEELEEKNIKEFLQSFVHIVQMFINTA [symbol] [symbol] [symbol]
ITCPPPMSVEHADIWVKSYSLYSRERYICNSGFKRKAGTSSLTECVLNKATNVAHWTTPS
LKCIRDPALVHQRPAPPS
Chain 2: Fc-b-SGPASGR (SEQ ID NO: 198)
                      EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: SANAPLGLAG-IL-15-(GGS)5-IL-15Rα (SEQ ID NO: 199) [symbol]
                        NWVNVISDLKKIEDLIQSMHIDATLYTESDVHPSCKVTAMKCELLELQVISLE
SGDASIHDTVENLIILANNSLSSNGNVTESGCKECEELEEKNIKEFLQSFVHIVQMFINTA [symbol] [symbol] [symbol]
ITCPPPMSVEHADIWVKSYSLYSRERYICNSGFKRKAGTSSLTECVLNKATNVAHWTTPSLKCIRDPALVHQRP
APPS
```

Expected Cleavage Products from Legumain Cleavage of P1342:

```
1) Fc-b-SGPASGRSAN homodimer (product from complete
cleavage) (SEQ ID NO: 200)
Chains 1 and 2
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) APLGLAG-IL-15-(GGS)5-IL-15Rα homodimer (product from complete
cleavage, active) (SEQ ID NO: 201)
Chains 1 and 2 [symbol]
                        NWVNVISDLKKIEDLIQSMHIDATLYTESDVHPSCKVTAMKCELLELQVISLESGD
ASIHDTVENLIILANNSLSSNGNVTESGCKECEELEEKNIKEFLQSFVHIVQMFINTA [symbol] [symbol] IT
CPPPMSVEHADIWVKSYSLYSRERYICNSGFKRKAGTSSLTECVLNKATNVAHWTTPSLKCIRDPALVHQRPAPP
S
3) Fc-b-SGPASGRSANAPLGLAG-IL-15(S162A)-(GGS)5-IL-15Rα/Fc-b-
SGPASGRSAN/APLGLAG-IL-15-(GGS)5-IL-15Rα (product from partial cleavage,
active)
Chain 1: Fc-b-SGPASGRSANAPLGLAG-IL-15(S162A)-(GGS)5-IL-15Rα (SEQ
ID NO: 202)
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKVTA
MKCFLLELQVISLESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPS
LKCIRDPALVHQRPAPPS
Chain 2: Fc-b-SGPASGRSAN (SEQ ID NO: 203)
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: APLGLAG-IL-15-(GGS)5-IL-15Rα (SEQ ID NO: 204) [symbol]
                        NWVNVISDLKKIEDLIQSMHIDATLYTESDVHPSCKVTAMKCELLELQVISLESGD
ASIHDTVENLIILANNSLSSNGNVTESGCKECEELEEKNIKEFLQSFVHIVQMFINTA [symbol] [symbol] IT
CPPPMSVEHADIWVKSYSLYSRERYICNSGFKRKAGTSSLTECVLNKATNVAHWTTPSLKCIRDPALVHQRPAPP
S
```

Expected Cleavage Products from MMP-2 or MMP-9 Cleavage of P1342:

```
1) Fc-b-SGPASGRSANAPLG homodimer (product from complete
cleavage) (SEQ ID NO: 205)
Chains 1 and 2
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol]
2) LAG-IL-15-(GGS)5-IL-15Rα homodimer (product from complete
cleavage, active) (SEQ ID NO: 206)
Chains 1 and 2 [symbol]
                        NWVNVISDLKKIEDLIQSMHIDATLYTESDVHPSCKVTAMKCELLELQVISLESGDASIH
DTVENLIILANNSLSSNGNVTESGCKECEELEEKNIKEFLQSFVHIVQMFINTA [symbol] [symbol] ITCPPP
MSVEHADIWVKSYSLYSRERYICNSGFKRKAGTSSLTECVLNKATNVAHWTTPSLKCIRDPALVHQRPAPPS
3) Fc-b-SGPASGRSANAPLGLAG-IL-15(S162A)-(GGS)5-IL-15Rα/Fc-b-
SGPASGRSANAPLG/LAG-IL-15-(GGS)5-IL-15Rα (product from partial cleavage,
active)
Chain 1: Fc-b-SGPASGRSANAPLGLAG-IL-15(S162A)-(GGS)5-IL-15Rα (SEQ
ID NO: 207)
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKVTA
MKCFLLELQVISLESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPS
LKCIRDPALVHQRPAPPS
Chain 2: Fc-b-SGPASGRSANAPLG (SEQ ID NO: 208)
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKSGPASGRSANAPLG
Chain 3: LAG-IL-15-(GGS)5-IL-15Rα (SEQ ID NO: 209) [symbol]
                        NWVNVISDLKKIEDLIQSMHIDATLYTESDVHPSCKVTAMKCELLELQVISLESGDASIH
DTVENLIILANNSLSSNGNVTESGCKECEELEEKNIKEFLQSFVHIVQMFINTA [symbol] [symbol] ITCPPP
MSVEHADIWVKSYSLYSRERYICNSGFKRKAGTSSLTECVLNKATNVAHWTTPSLKCIRDPALVHQRPAPPS
Fc-b-GSGPASGRSANAPLGLAGS-IL-15(S162A)-(GGS)5-IL-15Rα (SEQ ID NO:
210)
>P1343_Fc-b-PS10-L15R-S162A chains 1 and 2
EPKSSDKTHTCPPCPAPEAAGGPSVFLFPPKPKDTLYITREPEVTCVVVDVSHEDPEVKFNWYVDGVEVHNAKTK
PREEQYNSTYRVVSVLTVLHQDWLNGKEYKCKVSNKALAAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] [symbol] NWVNVISDLKKIEDLIQSMHIDATLYTESDVHPSCKVTAMKCELLELQV
ISLESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPSLKCIRDPALV
HQRPAPPS
```

Expected Cleavage Products from uPA or Matriptase Cleavage of P1343:

```
1) Fc-b-GSGPASGR homodimer (product from complete cleavage)(SEQ
ID NO: 211)
Chains 1 and 2
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) SANAPLGLAGS-IL-15-(GGS)5-IL-15Rα homodimer (product from
complete cleavage, active) (SEQ ID NO: 212)
Chains 1 and 2 [symbol]
                        NWVNVISDLKKIEDLIQSMHIDATLYTESDVHPSCKVTAMKCELLELQVISL
ESGDASIHDTVENLIILANNSLSSNGNVTESGCKECEELEEKNIKEFLQSFVHIVQMFINTA [symbol] [symbol] [symbol]
ITCPPPMSVEHADIWVKSYSLYSRERYICNSGFKRKAGTSSLTECVLNKATNVAHWTTPSLKCIRDPALVHQR
PAPPS
3) Fc-b-GSGPASGRSANAPLGLAGS-IL-15(S162A)-(GGS)5-IL-15Rα/Fc-b-
GSGPASGR/SANAPLGLAGS-IL-15-(GGS)5-IL-15Rα (product from partial cleavage,
active)
Chain 1: Fc-b-GSGPASGRSANAPLGLAGS-IL-15(S162A)-(GGS)5-IL-
15Rα (SEQ ID NO: 213)
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKV
TAMKCFLLELQVISLESGDASIHDTVENLIILANNSLSSNGNVTESGCKECEELEEKNIKEFLQSFVHIVQMFIN
TA [symbol] [symbol] ITCPPPMSVEHADIWVKSYSLYSRERYICNSGFKRKAGTSSLTECVLNKATNVAHWTT
PSLKCIRDPALVHQRPAPPS
Chain 2: Fc-b-GSGPASGR (SEQ ID NO: 214)
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: SANAPLGLAGS-IL-15-(GGS)5-IL-15Rα (SEQ ID NO: 215) [symbol]
                        SNWVNVISDLKKIEDLIQSMHIDATLYTESDVHPSCKVTAMKCELLELQVISL
ESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPSLKCIRDPALVHQR
PAPPS
```

Expected Cleavage Products from Legumain Cleavage of P1343:

```
1) Fc-b-GSGPASGRSAN homodimer (product from complete
cleavage) (SEQ ID NO: 216)
Chains 1 and 2
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) APLGLAGS-IL-15-(GGS)5-IL-15Rα homodimer (product from
complete cleavage, active) (SEQ ID NO: 217)
Chains 1 and 2 [symbol]
                  NWVNVISDLKKIEDLIQSMHIDATLYTESDVHPSCKVTAMKCELLELQVISLESG
DASIHDTVENLIILANNSLSSNGNVTESGCKECEELEEKNIKEFLQSFVHIVQMFINTA [symbol] [symbol] I
TCPPPMSVEHADIWVKSYSLYSRERYICNSGFKRKAGTSSLTECVLNKATNVAHWTTPSLKCIRDPALVHQRPAP
PS
3) Fc-b-GSGPASGRSANAPLGLAGS-IL-15(S162A)-(GGS)5-IL-15Rα/Fc-b-
GSGPASGRSAN/APLGLAGS-IL-15-(GGS)5-IL-15Rα (product from partial cleavage,
active)
Chain 1: Fc-b-GSGPASGRSANAPLGLAGS-IL-15(S162A)-(GGS)5-IL-
15Rα (SEQ ID NO: 218)
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKV
TAMKCFLLELQVISLESGDASIHDTVENLIILANNSLSSNGNVTESGCKECEELEEKNIKEFLQSFVHIVQMFIN
TA [symbol] [symbol] ITCPPPMSVEHADIWVKSYSLYSRERYICNSGFKRKAGTSSLTECVLNKATNVAHWTT
PSLKCIRDPALVHQRPAPPS
Chain 2: Fc-b-GSGPASGRSAN (SEQ ID NO: 219)
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: APLGLAGS-IL-15-(GGS)5-IL-15Ru(SEQ ID NO: 220) [symbol]
                      NWVNVISDLKKIEDLIQSMHIDATLYTESDVHPSCKVTAMKCELLELQVISLESG
DASIHDTVENLIILANNSLSSNGNVTESGCKECEELEEKNIKEFLQSFVHIVQMFINTA [symbol] [symbol] I
TCPPPMSVEHADIWVKSYSLYSRERYICNSGFKRKAGTSSLTECVLNKATNVAHWTTPSLKCIRDPALVHQRPAP
PS
```

Expected Cleavage Products from MMP-2 or MMP-9 Cleavage of P1343:

```
1) Fc-b-GSGPASGRSAMAPLG homodimer (product from complete
cleavage) (SEQ ID NO: 221)
Chains 1 and 2
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol]
2) LAGS-IL-15-(GGS)5-IL-15Rα homodimer (product from complete
cleavage, active) (SEQ ID NO: 222)
Chains 1 and 2 [symbol]
                        NWVNVISDLKKIEDLIQSMHIDATLYTESDVHPSCKVTAMKCELLELQVISLESGDASI
HDTVENLIILANNSLSSNGNVTESGCKECEELEEKNIKEFLQSFVHIVQMFINTA [symbol] [symbol] ITCPP
PMSVEHADIWVKSYSLYSRERYICNSGFKRKAGTSSLTECVLNKATNVAHWTTPSLKCIRDPALVHQRPAPPS
3) Fc-b-GSGPASGRSANAPLGLAGS-IL-15(S162A)-(GGS)5-IL-15Rα/Fc-b-
GSGPASGRSANAPLG/LAGS-IL-15-(GGS)5-IL-15Rα (product from partial cleavage,
active)
Chain 1: Fc-b-GSGPASGRSANAPLGLAGS-IL-15(S162A)-(GGS)5-IL-
15Rα (SEQ ID NO: 223)
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKV
TAMKCFLLELQVISLESGDASIHDTVENLIILANNSLSSNGNVTESGCKECEELEEKNIKEFLQSFVHIVQMFIN
TA [symbol] [symbol] ITCPPPMSVEHADIWVKSYSLYSRERYICNSGFKRKAGTSSLTECVLNKATNVAHWTT
PSLKCIRDPALVHQRPAPPS
Chain 2: Fc-b-GSGPASGRSANAPLG (SEQ ID NO: 224)
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol]
Chain 3: LAGS-IL-15-(GGS)5-IL-15Rα (SEQ ID NO: 225) [symbol]
                        NWVNVISDLKKIEDLIQSMHIDATLYTESDVHPSCKVTAMKCELLELQVISLESGDASI
HDTVENLIILANNSLSSNGNVTESGCKECEELEEKNIKEFLQSFVHIVQMFINTA [symbol] [symbol] ITCPP
PMSVEHADIWVKSYSLYSRERYICNSGFKRKAGTSSLTECVLNKATNVAHWTTPSLKCIRDPALVHQRPAPPS
Fc-GSENLYFQGGS-IL-15(L52C)-(GGS)5-IL-15Rα(S40C) (SEQ ID NO:
226)
>P1378_Fc-TEV-L15R_(L:L52C)_(R:S40C) chains 1 and 2
EPKSSDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] NWVNVISDLKKIEDLIQSMHIDATLYTESDVHPSCKVTAMKCFLLELQVISCESGDA
SIHDTVENLIILANNSLSSNGNVTESGCKECEELEEKNIKEFLQSFVHIVQMFINTS [symbol] [symbol] ITC
PPPMSVEHADIWVKSYSLYSRERYICNSGFKRKAGTCSLTECVLNKATNVAHWTTPSLKCIRDPALVHQRPAPPS
```

Expected Cleavage Products from TEV Cleavage of P1378:

```
1) Fc-GSENLYFQ homodimer (product from complete cleavage)(SEQ
ID NO: 227)
Chains 1 and 2
                        EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) GGS-IL-15(L52C)-(GGS)5-IL-15Rα(S40C) homodimer(product from
complete cleavage, active) (SEQ ID NO: 228)
Chains 1 and 2 [symbol]
                        NWVNVISDLKKIEDLIQSMHIDATLYTESDVHPSCKVTAMKCFLLELQVISCESGDASIH
DTVENLIILANNSLSSNGNVTESGCKECEELEEKNIKEFLQSFVHIVQMFINTS [symbol] [symbol] ITCPPP
MSVEHADIWVKSYSLYSRERYICNSGFKRKAGTCSLTECVLNKATNVAHWTTPSLKCIRDPALVHQRPAPPS
3) Fc-GSENLYFQGGS-IL-15(L52C)-(GGS)5-IL-15Rα(S40C)/Fc-GSENLYFQ/
GGS-IL-15(L52C)-(GGS)5-IL-15Rα(S40C) (product from partial cleavage,
active)
Chain 1: Fc-GSENLYFQGGS-IL-15(L52C)-(GGS)5-IL-15Rα(S40C) (SEQ
ID NO: 229)
                        EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCELL
ELQVISCESGDASIHDTVENLIILANNSLSSNGNVTESGCKECEELEEKNIKEFLQSFVHIVQMFINTS [symbol] [symbol]
ITCPPPMSVEHADIWVKSYSLYSRERYICNSGFKRKAGTCSLTECVLNKATNVAHWTTPSLKCIRD
PALVHQRPAPPS
Chain 2: Fc-GSENLYFQ (SEQ ID NO: 230)
                        EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: GGS-IL-15(L52C)-(GGS)5-IL-15Rα(S40C) (SEQ ID NO: 231) [symbol]
                        NWVNVISDLKKIEDLIQSMHIDATLYTESDVHPSCKVTAMKCFLLELQVISCESGDASIH
DTVENLIILANNSLSSNGNVTESGCKECEELEEKNIKEFLQSFVHIVQMFINTS [symbol] [symbol] ITCPPP
MSVEHADIWVKSYSLYSRERYICNSGFKRKAGTCSLTECVLNKATNVAHWTTPSLKCIRDPALVHQRPAPPS
SSFc-GSENLYFQGGS-IL-15(L52C)-(GGS)5-IL-15Rα(540C) (SEQ ID NO:
232)
>P1379_SSFc-TEV-L15R_(L:L52C)_(R:S40C) chains 1 and 2
                        EPKSSDKTHTSPPSPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCELL
ELQVISCESGDASIHDTVENLIILANNSLSSNGNVTESGCKECEELEEKNIKEFLQSFVHIVQMFINTS [symbol] [symbol]
ITCPPPMSVEHADIWVKSYSLYSRERYICNSGFKRKAGTCSLTECVLNKATNVAHWTTPSLKCIRD
PALVHQRPAPPS
```

Expected Cleavage Products from TEV Cleavage of P1379:

```
1) SSFc-GSENLYFQ homodimer (product from complete cleavage) (SEQ
ID NO: 233)
Chains 1 and 2
                        EPKSSDKTHTSPPSPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) GGS-IL-15(L52C)-(GGS)5-IL-15Rα(S40C) homodimer (product from
complete cleavage, active) (SEQ ID NO: 234)
Chains 1 and 2 [symbol]
                        NWVNVISDLKKIEDLIQSMHIDATLYTESDVHPSCKVTAMKCFLLELQVISCESGDASIH
DTVENLIILANNSLSSNGNVTESGCKECEELEEKNIKEFLQSFVHIVQMFINTS [symbol] [symbol] ITCPPP
MSVEHADIWVKSYSLYSRERYICNSGFKRKAGTCSLTECVLNKATNVAHWTTPSLKCIRDPALVHQRPAPPS
3) SSFc-GSENLYFQGGS-IL-15(L52C)-(GGS)5-IL-15Rα(S40C)/SSFc-
GSENLYFQ/GGS-IL-15(L52C)-(GGS)5-IL-15Rα(S40C) (product from partial
cleavage, active)
Chain 1: SSFc-GSENLYFQGGS-IL-15(L52C)-(GGS)5-IL-15Rα(540C) (SEQ
ID NO: 235)
                        EPKSSDKTHTSPPSPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCELL
ELQVISCESGDASIHDTVENLIILANNSLSSNGNVTESGCKECEELEEKNIKEFLQSFVHIVQMFINTS [symbol] [symbol]
ITCPPPMSVEHADIWVKSYSLYSRERYICNSGFKRKAGTCSLTECVLNKATNVAHWTTPSLKCIRD
PALVHQRPAPPS
Chain 2: SSFc-GSENLYFQ (SEQ ID NO: 236)
                        EPKSSDKTHTSPPSPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: GGS-IL-15(L52C)-(GGS)5-IL-15Rα(S40C) (SEQ ID NO: 237) [symbol]
                        NWVNVISDLKKIEDLIQSMHIDATLYTESDVHPSCKVTAMKCFLLELQVISCESGDASIH
DTVENLIILANNSLSSNGNVTESGCKECEELEEKNIKEFLQSFVHIVQMFINTS [symbol] [symbol] GGSITCPPP
MSVEHADIWVKSYSLYSRERYICNSGFKRKAGTCSLTECVLNKATNVAHWTTPSLKCIRDPALVHQRPAPPS
Fc-b-PLGLSGRSANAGPA-IL-15(S162A, L52C)-(GGS)5-IL-15Rα(S40C) (SEQ
ID NO: 238)
>P1380_Fc-b-PS4-L15R-S162A_(L:L52C)_(R:S40C) chains 1 and 2
EPKSSDKTHTCPPCPAPEAAGGPSVFLFPPKPKDTLYITREPEVTCVVVDVSHEDPEVKFNWYVDGVEVHNAKTK
PREEQYNSTYRVVSVLTVLHQDWLNGKEYKCKVSNKALAAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] [symbol] NWVNVISDLKKIEDLIQSMHIDATLYTESDVHPSCKVTAMKCELLELQVISCES
GDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTCSLTECVLNKATNVAHWTTPSLKCIRDPALVHQRPA
PPS
```

Expected Cleavage Products from uPA or Matriptase Cleavage of P1300;

```
1) Fc-b-PLGLSGR homodimer (product from complete cleavage)(SEQ
ID NO: 239)
Chains 1 and 2
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) SANAGPA-IL-15(S162A, L52C)-(GGS)5-IL-15Rα(540C)
homodimer (product from complete cleavage, active) (SEQ ID NO: 240)
Chains 1 and 2 [symbol]
                        NWVNVISDLKKIEDLIQSMHIDATLYTESDVHPSCKVTAMKCFLLELQVISCESGD
ASIHDTVENLIILANNSLSSNGNVTESGCKECEELEEKNIKEFLQSFVHIVQMFINTA [symbol] [symbol] IT
CPPPMSVEHADIWVKSYSLYSRERYICNSGFKRKAGTCSLTECVLNKATNVAHWTTPSLKCIRDPALVHQRPAPP
S
3) Fc-b-PLGLSGRSANAGPA-IL-15(S162A, L52C)-(GGS)5-IL-
15Rα(S40C)/Fc-b-PLGLSGR/SANAGPA-IL-15(S162A, L52C)-(GGS)5-IL-15Rα(S40C)
(product from partial cleavage, active)
Chain 1: Fc-b-PLGLSGRSANAGPA-IL-15(S162A, L52C)-(GGS)5-IL-
15Rα(S40C) (SEQ ID NO: 241)
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKVTAMKC
FLLELQVISCESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTCSLTECVLNKATNVAHWTTPSLKC
IRDPALVHQRPAPPS
Chain 2: Fc-b-PLGLSGR (SEQ ID NO: 242)
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: SANAGPA-IL-15(S162A, L52C)-(GGS)5-IL-15Rα(S40C) (SEQ ID
NO: 243) [symbol]
                        NWVNVISDLKKIEDLIQSMHIDATLYTESDVHPSCKVTAMKCFLLELQVISCESGD
ASIHDTVENLIILANNSLSSNGNVTESGCKECEELEEKNIKEFLQSFVHIVQMFINTA [symbol] [symbol] IT
CPPPMSVEHADIWVKSYSLYSRERYICNSGFKRKAGTCSLTECVLNKATNVAHWTTPSLKCIRDPALVHQRPAPP
S
```

Expected Cleavage Products from Legumain Cleavage of P1380:

```
1) Fc-b-PLGLSGRSAN homodimer (product from complete
cleavage) (SEQ ID NO: 244)
Chains 1 and 2
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) AGPA-IL-15(S162A, L52C)-(GGS)5-IL-15Rα(S40C)
homodimer (product from complete cleavage, active) (SEQ ID NO: 245)
Chains 1 and 2 [symbol]
                        NWVNVISDLKKIEDLIQSMHIDATLYTESDVHPSCKVTAMKCFLLELQVISCESGDASI
HDTVENLIILANNSLSSNGNVTESGCKECEELEEKNIKEFLQSFVHIVQMFINTA [symbol] [symbol] ITCPP
PMSVEHADIWVKSYSLYSRERYICNSGFKRKAGTCSLTECVLNKATNVAHWTTPSLKCIRDPALVHQRPAPPS
3) Fc-b-PLGLSGRSANAGPA-IL-15(S162A, L52C)-(GGS)5-IL-15Rα(S40C)/
Fc-b-PLGLSGRSAN/AGPA-IL-15(S162A, L52C)-(GGS)5-IL-15Rα(S40C) (product from
partial cleavage, active)
Chain 1: Fc-b-PLGLSGRSANAGPA-IL-15(S162A, L52C)-(GGS)5-IL-
15Rα(S40C) (SEQ ID NO: 246)
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKVTAMKC
FLLELQVISCESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTCSLTECVLNKATNVAHWTTPSLKC
IRDPALVHQRPAPPS
Chain 2: Fc-b-PLGLSGRSAN (SEQ ID NO: 247)
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: AGPA-IL-15(S162A, L52C)-(GGS)5-IL-15Rα(S40C) (SEQ ID
NO: 248) [symbol]
                        NWVNVISDLKKIEDLIQSMHIDATLYTESDVHPSCKVTAMKCELLELQVISCESGDASI
HDTVENLIILANNSLSSNGNVTESGCKECEELEEKNIKEFLQSFVHIVQMFINTA [symbol] [symbol] ITCPP
PMSVEHADIWVKSYSLYSRERYICNSGEKRKAGICSLTECVLNKATNVAHWTTPSLKCIRDPALVHQRPAPPS
```

Expected Cleavage Products from MMP-2 or MMP-9 Cleavage of P1380:

```
1) Fc-b-PLG homodimer (product from complete cleavage) (SEQ ID
NO: 249)
Chains 1 and 2
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) LSGRSANAGPA-IL-15(S162A, L52C)-(GGS)5-IL-15Rα(S40C)
homodimer (product from complete cleavage, active) (SEQ ID NO: 250)
Chains 1 and 2 [symbol]
                        NWVNVISDLKKIEDLIQSMHIDATLYTESDVHPSCKVTAMKCFLLELQVISC
ESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTCSLTECVLNKATNVAHWTTPSLKCIRDPALVHQR
PAPPS
3) Fc-b-PLGLSGRSANAGPA-IL-15(S162A, L52C)-(GGS)5-IL-
15Rα(S40C)/Fc-b-PLG/LSGRSANAGPA-IL-15(S162A, L52C)-(GGS)5-IL-15Rα(S40C)
(product from partial cleavage, active)
Chain 1: Fc-b-PLGLSGRSANAGPA-IL-15(S162A, L52C)-(GGS)5-IL-
15Rα(S40C) (SEQ ID NO: 251)
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKVTAMKC
FLLELQVISCESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTCSLTECVLNKATNVAHWTTPSLKC
IRDPALVHQRPAPPS
Chain 2: Fc-b-PLG (SEQ ID NO: 252)
                        EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: LSGRSANAGPA-IL-15(S162A, L52C)-(GGS)5-IL-15Rα(S40C)
(SEQ ID NO: 253) [symbol]
                        NWVNVISDLKKIEDLIQSMHIDATLYTESDVHPSCKVTAMKCELLELQVISC
ESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTCSLTECVLNKATNVAHWTTPSLKCIRDPALVHQR
PAPPS
SSFc-b-PLGLSGRSANAGPA-IL-15(S162A, L52C)-(GGS)5-IL-15Rα(S40C)
(SEQ ID NO: 254)
>P1381_SSFc-b-PS4-L15R-S162A_(L:L52C)_(R:S40C) chains 1 and 2
EPKSSDKTHTSPPSPAPEAAGGPSVFLFPPKPKDTLYITREPEVTCVVVDVSHEDPEVKFNWYVDGVEVHNAKTK
PREEQYNSTYRVVSVLTVLHQDWLNGKEYKCKVSNKALAAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] [symbol] NWVNVISDLKKIEDLIQSMHIDATLYTESDVHPSCKVTAMKCELLELQVISCES
GDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTCSLTECVLNKATNVAHWTTPSLKCIRDPALVHQRPA
PPS
```

Expected Cleavage Products from uPA or Matriptase Cleavage of P1381:

```
1) SSFc-b-PLGLSGR homodimer (product from complete
cleavage) (SEQ ID NO: 255)
Chains 1 and 2
                        EPKSSDKTHTSPPSPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) SANAGPA-IL-15(S162A, L52C)-(GGS)5-IL-15Rα(S40C)
homodimer (product from complete cleavage, active) (SEQ ID NO: 256)
Chains 1 and 2 [symbol]
                        NWVNVISDLKKIEDLIQSMHIDATLYTESDVHPSCKVTAMKCELLELQVISCESGD
ASIHDTVENLIILANNSLSSNGNVTESGCKECEELEEKNIKEFLQSFVHIVQMFINTA [symbol] [symbol] IT
CPPPMSVEHADIWVKSYSLYSRERYICNSGFKRKAGTCSLTECVLNKATNVAHWTTPSLKCIRDPALVHQRPAPP
S
3) SSFc-b-PLGLSGRSANAGPA-IL-15(S162A, L52C)-(GGS)5-IL-
15Rα(S40C)/SSFc-b-PLGLSGR/SANAGPA-IL-15(S162A, L52C)-(GGS)5-IL-15Rα(S40C)
(product from partial cleavage, active)
Chain 1: SSFc-b-PLGLSGRSANAGPA-IL-15(S162A, L52C)-(GGS)5-IL-
15Rα(S40C) (SEQ ID NO: 257)
                        EPKSSDKTHTSPPSPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKVTAMKC
FLLELQVISCESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTCSLTECVLNKATNVAHWTTPSLKC
IRDPALVHQRPAPPS
Chain 2: SSFc-b-PLGLSGR (SEQ ID NO: 26058)
                        EPKSSDKTHTSPPSPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: SANAGPA-IL-15(S162A, L52C)-(GGS)5-IL-15Rα(S40C) (SEQ
ID NO: 259) [symbol]
                        NWVNVISDLKKIEDLIQSMHIDATLYTESDVHPSCKVTAMKCELLELQVISCESGD
ASIHDTVENLIILANNSLSSNGNVTESGCKECEELEEKNIKEFLQSFVHIVQMFINTA [symbol] [symbol] IT
CPPPMSVEHADIWVKSYSLYSRERYICNSGFKRKAGTCSLTECVLNKATNVAHWTTPSLKCIRDPALVHQRPAPP
S
```

Expected Cleavage Products from Legumain Cleavage of P1381:

```
1) SSFc-b-PLGLSGRSAN homodimer (product from complete
cleavage) (SEQ ID NO: 260)
Chains 1 and 2
                        EPKSSDKTHTSPPSPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) AGPA-IL-15(S162A, L52C)-(GGS)5-IL-15Rα(S40C)
homodimer (product from complete cleavage, active) (SEQ ID NO: 261)
Chains 1 and 2 [symbol]
                        NWVNVISDLKKIEDLIQSMHIDATLYTESDVHPSCKVTAMKCFLLELQVISCESGDASI
HDTVENLIILANNSLSSNGNVTESGCKECEELEEKNIKEFLQSFVHIVQMFINTA [symbol] [symbol] ITCPP
PMSVEHADIWVKSYSLYSRERYICNSGFKRKAGTCSLTECVLNKATNVAHWTTPSLKCIRDPALVHQRPAPPS
3) SSFc-b-PLGLSGRSANAGPA-IL-15(S162A, L52C)-(GGS)5-IL-
15Rα(S40C)/SSFc-b-PLGLSGRSAN/AGPA-IL-15(S162A, L52C)-(GGS)5-IL-15Rα(S40C)
(product from partial cleavage, active)
Chain 1: SSFc-b-PLGLSGRSANAGPA-IL-15(S162A, L52C)-(GGS)5-IL-
15Rα(540C) (SEQ ID NO: 262)
                        EPKSSDKTHTSPPSPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKPLGLSGRSANAGPANWVNVISDLKKIEDLIQSMHIDATLYTESDVHPSCKVTAMKC
FLLELQVISCESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTCSLTECVLNKATNVAHWTTPSLKC
IRDPALVHQRPAPPS
Chain 2: SSFc-b-PLGLSGRSAN (SEQ ID NO: 263)
                        EPKSSDKTHTSPPSPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: AGPA-IL-15(S162A, L52C)-(GGS)5-IL-15Rα(S40C) (SEQ ID
NO: 264) [symbol]
                        NWVNVISDLKKIEDLIQSMHIDATLYTESDVHPSCKVTAMKCFLLELQVISCESGDASI
HDTVENLIILANNSLSSNGNVTESGCKECEELEEKNIKEFLQSFVHIVQMFINTA [symbol] [symbol] ITCPP
PMSVEHADIWVKSYSLYSRERYICNSGFKRKAGTCSLTECVLNKATNVAHWTTPSLKCIRDPALVHQRPAPPS
```

Expected Cleavage Products from MMP-2 or MMP-9 Cleavage of P1381:

1) SSFc-b-PLG homodimer (product from complete cleavage) (SEQ ID
NO: 265)
Chains 1 and 2
EPKSSDKTHTSPPSPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) LSGRSANAGPA-IL-15(S162A, L52C)-(GGS)5-IL-15Rα(S40C)
homodimer (product from complete cleavage, active) (SEQ ID NO: 266)
Chains 1 and 2 [symbol]
NWVNVISDLKKIEDLIQSMHIDATLYTESDVHPSCKVTAMKCELLELQVISC
ESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTCSLTECVLNKATNVAHWTTPSLKCIRDPALVHQR
PAPPS
3) SSFc-b-PLGLSGRSANAGPA-IL-15(S162A, L52C)-(GGS)5-IL-
15Rα(S40C)/SSFc-b-PLG/LSGRSANAGPA-IL-15(S162A, L52C)-(GGS)5-IL-15Rα(S40C)
(product from partial cleavage, active)
Chain 1: SSFc-b-PLGLSGRSANAGPA-IL-15(S162A, L52C)-(GGS)5-IL-
15Rα(S40C) (SEQ ID NO: 267)
EPKSSDKTHTSPPSPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] [symbol] NWVNVISDLKKIEDLIQSMHIDATLYTESDVHPSCKVTAMKC
FLLELQVISCESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTCSLTECVLNKATNVAHWTTPSLKC
IRDPALVHQRPAPPS
Chain 2: SSFc-b-PLG (SEQ ID NO: 268)
EPKSSDKTHTSPPSPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: LSGRSANAGPA-IL-15(S162A, L52C)-(GGS)5-IL-15Rα(S40C)
(SEQ ID NO: 269) [symbol]
NWVNVISDLKKIEDLIQSMHIDATLYTESDVHPSCKVTAMKCELLELQVISC
ESGDASIHDTVENLIILANNSLSSNGNVTESGCKECEELEEKNIKEFLQSFVHIVQMFINTA [symbol] [symbol] [symbol]
ITCPPPMSVEHADIWVKSYSLYSRERYICNSGFKRKAGTCSLTECVLNKATNVAHWTTPSLKCIRDPALVHQR
PAPPS
Fc-b-PLGLSGRSANAG-IL-15(S162A, L52C)-(GGS)5-IL-15Rα(S40C) (SEQ
ID NO: 270)
>P1382_Fc-b-PS4-L15R-S162A_(L:L52C)_(R:S40C) (PLGLSGRSANAGPA →
PLGLSGRSANAG) chains 1 and 2
EPKSSDKTHTCPPCPAPEAAGGPSVFLFPPKPKDTLYITREPEVTCVVVDVSHEDPEVKFNWYVDGVEVHNAKTK
PREEQYNSTYRVVSVLTVLHQDWLNGKEYKCKVSNKALAAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] NWVNVISDLKKIEDLIQSMHIDATLYTESDVHPSCKVTAMKCELLELQVISCESGD
ASIHDTVENLIILANNSLSSNGNVTESGCKECEELEEKNIKEFLQSFVHIVQMFINTA [symbol] [symbol] IT
CPPPMSVEHADIWVKSYSLYSRERYICNSGFKRKAGTCSLTECVLNKATNVAHWTTPSLKCIRDPALVHQRPAPP
S

Expected Cleavage Products from uPA or Matriptase Cleavage of P1382:

1) Fc-b-PLGLSGR homodimer (product from complete cleavage) (SEQ
ID NO: 271)
Chains 1 and 2
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) SANAG-IL-15(S162A, L52C)-(GGS)5-IL-15Rα(S40C)
homodimer(product from complete cleavage, active) (SEQ ID NO: 272)
Chains 1 and 2 [symbol]
NWVNVISDLKKIEDLIQSMHIDATLYTESDVHPSCKVTAMKCELLELQVISCESGDAS
IHDTVENLIILANNSLSSNGNVTESGCKECEELEEKNIKEFLQSFVHIVQMFINTA [symbol] [symbol] ITCP
PPMSVEHADIWVKSYSLYSRERYICNSGFKRKAGTCSLTECVLNKATNVAHWTTPSLKCIRDPALVHQRPAPPS
3) Fc-b-PLGLSGRSANAG-IL-15(S162A, L52C)-(GGS)5-IL-
15Rα(S40C)/Fc-b-PLGLSGR/SANAG-IL-15(S162A, L52C)-(GGS)5-IL-15Rα(S40C)
(product from partial cleavage, active)
Chain 1: Fc-b-PLGLSGRSANAG-IL-15(S162A, L52C)-(GGS)5-IL-
15Rα(S40C) (SEQ ID NO: 273)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCFL
LELQVISCESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTCSLTECVLNKATNVAHWTTPSLKCIR
DPALVHQRPAPPS
Chain 2: Fc-b-PLGLSGR (SEQ ID NO: 274)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: SANAG-IL-15(S162A, L52C)-(GGS)5-IL-15Rα(S40C) (SEQ ID
NO: 275) [symbol]
NWVNVISDLKKIEDLIQSMHIDATLYTESDVHPSCKVTAMKCELLELQVISCESGDAS
IHDTVENLIILANNSLSSNGNVTESGCKECEELEEKNIKEFLQSFVHIVQMFINTA [symbol] [symbol] ITCP
PPMSVEHADIWVKSYSLYSRERYICNSGFKRKAGTCSLTECVLNKATNVAHWTTPSLKCIRDPALVHQRPAPPS

Expected Cleavage Products from Legumain Cleavage of P1382:

1) Fc-b-PLGLSGRSAN homodimer (product from complete
cleavage) (SEQ ID NO: 276)
Chains 1 and 2
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) AG-IL-15(S162A, L52C)-(GGS)5-IL-15Rα(S40C) homodimer(product
from complete cleavage, active) (SEQ ID NO: 277)
Chains 1 and 2 [symbol]
NWVNVISDLKKIEDLIQSMHIDATLYTESDVHPSCKVTAMKCELLELQVISCESGDASIHD
TVENLIILANNSLSSNGNVTESGCKECEELEEKNIKEFLQSFVHIVQMFINTA [symbol] [symbol] ITCPPPM
SVEHADIWVKSYSLYSRERYICNSGFKRKAGTCSLTECVLNKATNVAHWTTPSLKCIRDPALVHQRPAPPS
3) Fc-b-PLGLSGRSANAG-IL-15(S162A, L52C)-(GGS)5-IL-
15Rα(S40C)/Fc-b-PLGLSGRSAN/AG-IL-15(S162A, L52C)-(GGS)5-IL-15Rα(S40C)
(product from partial cleavage, active)
Chain 1: Fc-b-PLGLSGRSANAG-IL-15(S162A, L52C)-(GGS)5-IL-
15Rα(S40C) (SEQ ID NO: 278)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCFL
LELQVISCESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTCSLTECVLNKATNVAHWTTPSLKCIR
DPALVHQRPAPPS
Chain 2: Fc-b-PLGLSGRSAN (SEQ ID NO: 279)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: AG-IL-15(S162A, L52C)-(GGS)5-IL-15Rα(S40C) (SEQ ID NO:
280) [symbol]
NWVNVISDLKKIEDLIQSMHIDATLYTESDVHPSCKVTAMKCELLELQVISCESGDASIHD
TVENLIILANNSLSSNGNVTESGCKECEELEEKNIKEFLQSFVHIVQMFINTA [symbol] [symbol] ITCPPPM
SVEHADIWVKSYSLYSRERYICNSGFKRKAGTCSLTECVLNKATNVAHWTTPSLKCIRDPALVHQRPAPPS

Expected Cleavage Products from MMP-2 or MMP-9 Cleavage of P1382:

1) Fc-b-PLG homodimer (product from complete cleavage) (SEQ ID
NO: 281)
Chains 1 and 2
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) LSGRSANAG-IL-15(S162A, L52C)-(GGS)5-IL-15Ru(S40C)
homodimer(product from complete cleavage, active) (SEQ ID NO: 282)
Chains 1 and 2 [symbol]
NWVNVISDLKKIEDLIQSMHIDATLYTESDVHPSCKVTAMKCELLELQVISCES
GDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTCSLTECVLNKATNVAHWTTPSLKCIRDPALVHQRPA
PPS
3) Fc-b-PLGLSGRSANAG-IL-15(S162A, L52C)-(GGS)5-IL-
15Rα(S40C)/Fc-b-PLG/LSGRSANAG-IL-15(S162A, L52C)-(GGS)5-IL-15Rα(S40C)
(product from partial cleavage, active)
Chain 1: Fc-b-PLGLSGRSANAG-IL-15(S162A, L52C)-(GGS)5-IL-
15Rα(S40C) (SEQ ID NO: 283)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCFL
LELQVISCESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTCSLTECVLNKATNVAHWTTPSLKCIR
DPALVHQRPAPPS
Chain 2: Fc-b-PLG (SEQ ID NO: 284)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: LSGRSANAG-IL-15(S162A, L52C)-(GGS)5-IL-15Rα(S40C) (SEQ
ID NO: 285) [symbol]
NWVNVISDLKKIEDLIQSMHIDATLYTESDVHPSCKVTAMKCELLELQVISCES
GDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTCSLTECVLNKATNVAHWTTPSLKCIRDPALVHQRPA
PPS
Fc-b-PLGLSGRSANA-IL-15(S162A, L52C)-(GGS)5-IL-15Rα(S40C) (SEQ ID
NO: 286)
>P1383_Fc-b-PS4-L15R-S162A_(L:L52C)_(R:S40C) (PLGLSGRSANAGPA →
PLGLSGRSANA) chains 1 and 2
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCFLL
ELQVISCESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTCSLTECVLNKATNVAHWTTPSLKCIRD
PALVHQRPAPPS

Expected Cleavage Products from uPA or Matriptase Cleavage of P1383:

1) Fc-b-PLGLSGR homodimer (product from complete cleavage) (SEQ
ID NO: 287)
Chains 1 and 2
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) SANA-IL-15(S162A, L52C)-(GGS)5-IL-15Rα(S40C)
homodimer (product from complete cleavage, active) (SEQ ID NO: 288)
Chains 1 and 2 [symbol]
NWVNVISDLKKIEDLIQSMHIDATLYTESDVHPSCKVTAMKCELLELQVISCESGDASI
HDTVENLIILANNSLSSNGNVTESGCKECEELEEKNIKEFLQSFVHIVQMFINTA [symbol] [symbol] ITCPP
PMSVEHADIWVKSYSLYSRERYICNSGFKRKAGTCSLTECVLNKATNVAHWTTPSLKCIRDPALVHQRPAPPS
3) Fc-b-PLGLSGRSANA-IL-15(S162A, L52C)-(GGS)5-IL-15Rα(S40C)/Fc-
b-PLGLSGR/SANA-IL-15(S162A, L52C)-(GGS)5-IL-15Rα(S40C) (product from partial
cleavage, active)
Chain 1: Fc-b-PLGLSGRSANA-IL-15(S162A, L52C)-(GGS)5-IL-
15Rα(S40C) (SEQ ID NO: 289)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCELL
ELQVISCESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTCSLTECVLNKATNVAHWTTPSLKCIRD
PALVHQRPAPPS
Chain 2: Fc-b-PLGLSGR (SEQ ID NO: 290)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: SANA-IL-15(S162A, L52C)-(GGS)5-IL-15Rα(S40C) (SEQ ID
NO: 291) [symbol]
NWVNVISDLKKIEDLIQSMHIDATLYTESDVHPSCKVTAMKCELLELQVISCESGDASI
HDTVENLIILANNSLSSNGNVTESGCKECEELEEKNIKEFLQSFVHIVQMFINTA [symbol] [symbol] ITCPP
PMSVEHADIWVKSYSLYSRERYICNSGFKRKAGTCSLTECVLNKATNVAHWTTPSLKCIRDPALVHQRPAPPS

Expected Cleavage Products from Legumain Cleavage of P1383:

1) Fc-b-PLGLSGRSAN homodimer (product from complete
cleavage) (SEQ ID NO: 292)
Chains 1 and 2
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) A-IL-15(S162A, L52C)-(GGS)5-IL-15Rα(S40C) homodimer(product
from complete cleavage, active) (SEQ ID NO: 293)
Chains 1 and 2
ANWVNVISDLKKIEDLIQSMHIDATLYTESDVHPSCKVTAMKCFLLELQVISCESGDASIHDT
VENLIILANNSLSSNGNVTESGCKECEELEEKNIKEFLQSFVHIVQMFINTA [symbol] [symbol] ITCPPPMS
VEHADIWVKSYSLYSRERYICNSGFKRKAGTCSLTECVLNKATNVAHWTTPSLKCIRDPALVHQRPAPPS
3) Fc-b-PLGLSGRSANA-IL-15(S162A, L52C)-(GGS)5-IL-15Rα(S40C)/Fc-
b-PLGLSGRSAN/A-IL-15(S162A, L52C)-(GGS)5-IL-15Rα(S40C) (product from partial
cleavage, active)
Chain 1: Fc-b-PLGLSGRSANA-IL-15(S162A, L52C)-(GGS)5-IL-
15Rα(S40C) (SEQ ID NO: 294)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCELL
ELQVISCESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTCSLTECVLNKATNVAHWTTPSLKCIRD
PALVHQRPAPPS
Chain 2: Fc-b-PLGLSGRSAN (SEQ ID NO: 295)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: A-IL-15(S162A, L52C)-(GGS)5-IL-15Rα(S40C) (SEQ ID NO:
296)
ANWVNVISDLKKIEDLIQSMHIDATLYTESDVHPSCKVTAMKCFLLELQVISCESGDASIHDT
VENLIILANNSLSSNGNVTESGCKECEELEEKNIKEFLQSFVHIVQMFINTA [symbol] [symbol] ITCPPPMS
VEHADIWVKSYSLYSRERYICNSGFKRKAGTCSLTECVLNKATNVAHWTTPSLKCIRDPALVHQRPAPPS

Expected Cleavage Products from MMP-2 or MMP-9 Cleavage of P1383:

1) Fc-b-PLG homodimer (product from complete cleavage)(SEQ ID
NO: 297)
Chains 1 and 2
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
2) LSGRSANA-IL-15(S162A, L52C)-(GGS)5-IL-15Rα(540C)
homodimer(product from complete cleavage, active) (SEQ ID NO: 298)
Chains 1 and 2 [symbol]
NWVNVISDLKKIEDLIQSMHIDATLYTESDVHPSCKVTAMKCELLELQVISCESG
DASIHDTVENLIILANNSLSSNGNVTESGCKECEELEEKNIKEFLQSFVHIVQMFINTA [symbol] [symbol] I
TCPPPMSVEHADIWVKSYSLYSRERYICNSGFKRKAGTCSLTECVLNKATNVAHWTTPSLKCIRDPALVHQRPAP
PS
3) Fc-b-PLGLSGRSANA-IL-15(S162A, L52C)-(GGS)5-IL-15Rα(S40C)/Fc-
b-PLG/LSGRSANA-IL-15(S162A, L52C)-(GGS)5-IL-15Rα(S40C) (product from partial
cleavage, active)
Chain 1: Fc-b-PLGLSGRSANA-IL-15(S162A, L52C)-(GGS)5-IL-
15Rα(S40C) (SEQ ID NO: 299)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCELL
ELQVISCESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTCSLTECVLNKATNVAHWTTPSLKCIRD
PALVHQRPAPPS
Chain 2: Fc-b-PLG (SEQ ID NO: 300)
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol]
Chain 3: LSGRSANA-IL-15(S162A, L52C)-(GGS)5-IL-15Rα(S40C) (SEQ
ID NO: 301) [symbol]
NWVNVISDLKKIEDLIQSMHIDATLYTESDVHPSCKVTAMKCELLELQVISCESG
DASIHDTVENLIILANNSLSSNGNVTESGCKECEELEEKNIKEFLQSFVHIVQMFINTA [symbol] [symbol] I
TCPPPMSVEHADIWVKSYSLYSRERYICNSGFKRKAGTCSLTECVLNKATNVAHWTTPSLKCIRDPALVHQRPAP
PS
Fc-G-IL-15(S162A)-GGSGGSEPKENLYFQGGGSGGSGGGS-IL-15Rα (SEQ ID NO: 302)
>P1393_Fc-1-L26-PS-R (IL15_S162A) chains 1 and 2
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCELLELQVISLESG
DASIHDTVENLIILANNSLSSNGNVTESGCKECEELEEKNIKEFLQSFVHIVQMFINTA [symbol] [symbol] [symbol]
ITCPPPMSVEHADIWVKSYSLYSRERYICNSGFKRKAGTSSLTECVLNKATNVAHWTTPSLKCIR
DPALVHQRPAPPS

Expected Cleavage Products from TEV Cleavage of P1393:

1) Fc-G-IL-15(S162A)-GGSGGSEPKENLYFQ homodimer (product from
complete cleavage) (SEQ ID NO: 303)
Chains 1 and 2
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCELLELQVISLESG
DASIHDTVENLIILANNSLSSNGNVTESGCKECEELEEKNIKEFLQSFVHIVQMFINTA [symbol] [symbol]
2) GGGSGGSGGGS-IL-15Rα homodimer(product from complete
cleavage, active) (SEQ ID NO: 304)
Chains 1 and 2 [symbol]
ITCPPPMSVEHADIWVKSYSLYSRERYICNSGFKRKAGTSSLTECVLNKATN
VAHWTTPSLKCIRDPALVHQRPAPPS
3) Fc-G-IL-15(S162A)-GGSGGSEPKENLYFQGGGSGGSGGGS-IL-15Rα/Fc-G-
IL-15(S162A)-GGSGGSEPKENLYFQ/GGGSGGSGGGS-IL-15Rα (product from partial
cleavage, active)
Chain 1: Fc-G-IL-15(S162A)-GGSGGSEPKENLYFQGGGSGGSGGGS-IL-
15Rα (SEQ ID NO: 305)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCELLELQVISLESG
DASIHDTVENLIILANNSLSSNGNVTESGCKECEELEEKNIKEFLQSFVHIVQMFINTA [symbol] [symbol] [symbol]
ITCPPPMSVEHADIWVKSYSLYSRERYICNSGFKRKAGTSSLTECVLNKATNVAHWTTPSLKCIR
DPALVHQRPAPPS
Chain 2: Fc-G-IL-15(S162A)-GGSGGSEPKENLYFQ (SEQ ID NO: 306)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCELLELQVISLESG
DASIHDTVENLIILANNSLSSNGNVTESGCKECEELEEKNIKEFLQSFVHIVQMFINTA [symbol] [symbol]
Chain 3: GGGSGGSGGGS-IL-15Rα (SEQ ID NO: 307) [symbol]
ITCPPPMSVEHADIWVKSYSLYSRERYICNSGFKRKAGTSSLTECVLNKATN
VAHWTTPSLKCIRDPALVHQRPAPPS
Fc-GGSGGS-IL-15(S162A)-GGSGGSEPKENLYFQGGGSGGSGGGS-IL-15Rα (SEQ
ID NO: 308)
>P1394_Fc-6-L26-PS-R (IL15_S162A) chains 1 and 2
EPKSSDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] NWVNVISDLKKIEDLIQSMHIDATLYTESDVHPSCKVTAMKCFLLELQVISLESGDASIHDT
VENLIILANNSLSSNGNVTESGCKECEELEEKNIKEFLQSFVHIVQMFINTA [symbol] [symbol] [symbol]
ITCPPPMSVEHADIWVKSYSLYSRERYICNSGFKRKAGTSSLTECVLNKATNVAHWTTPSLKCIRDPALVHQ
RPAPPS

Expected Cleavage Products from TEV Cleavage of P1394:

1) Fc-GGSGGS-IL-15(S162A)-GGSGGSEPKENLYFQ homodimer (product
from complete cleavage) (SEQ ID NO: 309)
Chains 1 and 2
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCFLLELQVI
SLESGDASIHDTVENLIILANNSLSSNGNVTESGCKECEELEEKNIKEFLQSFVHIVQMFINTA

[symbol]

[symbol]

2) GGGSGGSGGGS-IL-15Rα homodimer (product from complete
cleavage, active) (SEQ ID NO: 310)
Chains 1 and 2 [symbol]
ITCPPPMSVEHADIWVKSYSLYSRERYICNSGFKRKAGTSSLTECVLNKATN
VAHWTTPSLKCIRDPALVHQRPAPPS
3) Fc-GGSGGS-IL-15(S162A)-GGSGGSEPKENLYFQGGGSGGSGGGS-IL-
15Rα/Fc-GGSGGS-IL-15(S162A)-GGSGGSEPKENLYFQ/GGGSGGSGGGS-IL-15Rα (product
from partial cleavage, active)
Chain 1: Fc-GGSGGS-IL-15(S162A)-GGSGGSEPKENLYFQGGGSGGSGGGS-IL-
15Rα (SEQ ID NO: 311)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCELLELQVI
SLESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPS
LKCIRDPALVHQRPAPPS
Chain 2: Fc-GGSGGS-IL-15(S162A)-GGSGGSEPKENLYFQ (SEQ ID NO: 312)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCELLELQVI
SLESGDASIHDTVENLIILANNSLSSNGNVTESGCKECEELEEKNIKEFLQSFVHIVQMFINTA

[symbol]

[symbol]

Chain 3: GGGSGGSGGGS-IL-15Rα (SEQ ID NO: 313) [symbol]
ITCPPPMSVEHADIWVKSYSLYSRERYICNSGFKRKAGTSSLTECVLNKATN
VAHWTTPSLKCIRDPALVHQRPAPPS
Fc-GGSGGSGGSGGS-IL-15(S162A)-GGSGGSEPKENLYFQGGGSGGSGGGS-IL-
15Rα (SEQ ID NO: 314)
>P1395_Fc-12-L26-PS-R (IL15_S162A) chains 1 and 2
EPKSSDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] NWVNVISDLKKIEDLIQSMHIDATLYTESDVHPSCKVTAMKCFLLELQVISLESGD
ASIHDTVENLIILANNSLSSNGNVTESGCKECEELEEKNIKEFLQSFVHIVQMFINTA [symbol] [symbol] [symbol]
ITCPPPMSVEHADIWVKSYSLYSRERYICNSGFKRKAGTSSLTECVLNKATNVAHWTTPSLKCIRD
PALVHQRPAPPS

Expected Cleavage Products from TEV Cleavage of P1395:

1) Fc-GGSGGSGGSGGS-IL-15(S162A)-GGSGGSEPKENLYFQ homodimer
(product from complete cleavage) (SEQ ID NO: 315)
Chains 1 and 2
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTA

[symbol]

[symbol]

2) GGGSGGSGGGS-IL-15Rα homodimer(product from complete
cleavage, active) (SEQ ID NO: 316)
Chains 1 and 2 [symbol]
ITCPPPMSVEHADIWVKSYSLYSRERYICNSGFKRKAGTSSLTECVLNKATN
VAHWTTPSLKCIRDPALVHQRPAPPS
3) Fc-GGSGGSGGSGGS-IL-15(S162A)-GGSGGSEPKENLYFQGGGSGGSGGGS-IL-
15Rα/Fc-GGSGGSGGSGGS-IL-15(S162A)-GGSGGSEPKENLYFQ/GGGSGGSGGGS-IL-15Rα
(product from partial cleavage, active)
Chain 1: Fc-GGSGGSGGSGGS-IL-15(S162A)-
GGSGGSEPKENLYFQGGGSGGSGGGS-IL-15Rα (SEQ ID NO: 317)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTA [symbol] [symbol] [symbol]
ITCPPPMSVEHADIWVKSYSLYSRERYICNSGFKRKAGTSSLTECVLNKATNVA
HWTTPSLKCIRDPALVHQRPAPPS
Chain 2: Fc-GGSGGSGGSGGS-IL-15(S162A)-GGSGGSEPKENLYFQ (SEQ ID
NO: 318)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTA

[symbol]

[symbol]

Chain 3: GGGSGGSGGGS-IL-15Rα (SEQ ID NO: 319) [symbol]
ITCPPPMSVEHADIWVKSYSLYSRERYICNSGFKRKAGTSSLTECVLNKATN
VAHWTTPSLKCIRDPALVHQRPAPPS
Fc-G-IL-15(S162A, L52C)-GGSGGSEPKENLYFQGGGSGGSGGGS-IL-
15Rα(S40C) (SEQ ID NO: 320)
>P1396_Fc-1-L26-PS-R (IL15:S162A, L52C_IL15R:S40C) chains 1 and 2
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCELLELQVISCESG
DASIHDTVENLIILANNSLSSNGNVTESGCKECEELEEKNIKEFLQSFVHIVQMFINTA [symbol] [symbol] [symbol]
ITCPPPMSVEHADIWVKSYSLYSRERYICNSGFKRKAGTCSLTECVLNKATNVAHWTTPSLKCIR
DPALVHQRPAPPS

Expected Cleavage Products from TEV Cleavage of P1396:

1) Fc-G-IL-15(S162A, L52C)-GGSGGSEPKENLYFQ homodimer (product
from complete cleavage) (SEQ ID NO: 321)
Chains 1 and 2
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCELLELQVISCESG
DASIHDTVENLIILANNSLSSNGNVTESGCKECEELEEKNIKEFLQSFVHIVQMFINTA [symbol] [symbol]
2) GGGSGGSGGGS-IL-15Rα(S40C) homodimer(product from complete
cleavage, active) (SEQ ID NO: 322)
Chains 1 and 2 [symbol]
ITCPPPMSVEHADIWVKSYSLYSRERYICNSGFKRKAGTCSLTECVLNKATN
VAHWTTPSLKCIRDPALVHQRPAPPS
3) Fc-G-IL-15(S162A, L52C)-GGSGGSEPKENLYFQGGGSGGSGGGS-IL-
15Rα(S40C)/Fc-G-IL-15(S162A, L52C)-GGSGGSEPKENLYFQ/GGGSGGSGGGS-IL-
15Rα(540C) (product from partial cleavage, active)
Chain 1: Fc-G-IL-15(S162A, L52C)-GGSGGSEPKENLYFQGGGSGGSGGGS-IL-
15Rα(540C) (SEQ ID NO: 323)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCELLELQVISCESG
DASIHDTVENLIILANNSLSSNGNVTESGCKECEELEEKNIKEFLQSFVHIVQMFINTA [symbol] [symbol] [symbol]
ITCPPPMSVEHADIWVKSYSLYSRERYICNSGFKRKAGTCSLTECVLNKATNVAHWTTPSLKCIR
DPALVHQRPAPPS
Chain 2: Fc-G-IL-15(S162A, L52C)-GGSGGSEPKENLYFQ (SEQ ID NO:
324)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCELLELQVISCESG
DASIHDTVENLIILANNSLSSNGNVTESGCKECEELEEKNIKEFLQSFVHIVQMFINTA [symbol] [symbol]
Chain 3: GGGSGGSGGGS-IL-15Rα(S40C) (SEQ ID NO: 325) [symbol]
ITCPPPMSVEHADIWVKSYSLYSRERYICNSGFKRKAGTCSLTECVLNKATN
VAHWTTPSLKCIRDPALVHQRPAPPS
Fc-GGSGGS-IL-15(S162A, L52C)-GGSGGSEPKENLYFQGGGSGGSGGGS-IL-
15Rα(S40C) (SEQ ID NO: 326)
>P1397_Fc-6-L26-PS-R (IL15:S162A, L52C_IL15R:S40C) chains 1 and 2
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCELLELQVI
SCESGDASIHDTVENLIILANNSLSSNGNVTESGCKECEELEEKNIKEFLQSFVHIVQMFINTA [symbol] [symbol] [symbol]
ITCPPPMSVEHADIWVKSYSLYSRERYICNSGFKRKAGTCSLTECVLNKATNVAHWTTPS
LKCIRDPALVHQRPAPPS

Expected Cleavage Products from TEV Cleavage of P1397:

1) Fc-GGSGGS-IL-15(S162A, L52C)-GGSGGSEPKENLYFQ homodimer
(product from complete cleavage) (SEQ ID NO: 327)
Chains 1 and 2
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCFLLELQVI
SCESGDASIHDTVENLIILANNSLSSNGNVTESGCKECEELEEKNIKEFLQSFVHIVQMFINTA

[symbol]

[symbol]

2) GGGSGGSGGGS-IL-15Rα(540C) homodimer(product from complete
cleavage, active) (SEQ ID NO: 328)
Chains 1 and 2 [symbol]
ITCPPPMSVEHADIWVKSYSLYSRERYICNSGFKRKAGTCSLTECVLNKATN
VAHWTTPSLKCIRDPALVHQRPAPPS
3) Fc-GGSGGS-IL-15(S162A, L52C)-GGSGGSEPKENLYFQGGGSGGSGGGS-IL-
15Rα(S40C)/Fc-GGSGGS-IL-15(S162A, L52C)-GGSGGSEPKENLYFQ/GGGSGGSGGGS-IL-
15Rα(S40C) (product from partial cleavage, active)
Chain 1: Fc-GGSGGS-IL-15(S162A, L52C)-
GGSGGSEPKENLYFQGGGSGGSGGGS-IL-15Rα(S40C) (SEQ ID NO: 329)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCFLLELQVI
SCESGDASIHDTVENLIILANNSLSSNGNVTESGCKECEELEEKNIKEFLQSFVHIVQMFINTA [symbol] [symbol] [symbol]
ITCPPPMSVEHADIWVKSYSLYSRERYICNSGFKRKAGTCSLTECVLNKATNVAHWTTPS
LKCIRDPALVHQRPAPPS
Chain 2: Fc-GGSGGS-IL-15(S162A, L52C)-GGSGGSEPKENLYFQ(SEQ ID
NO: 330)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCFLLELQVI
SCESGDASIHDTVENLIILANNSLSSNGNVTESGCKECEELEEKNIKEFLQSFVHIVQMFINTA

[symbol]

[symbol]

Chain 3: GGGSGGSGGGS-IL-15Rα(S40C) (SEQ ID NO: 331) [symbol]
ITCPPPMSVEHADIWVKSYSLYSRERYICNSGFKRKAGTCSLTECVLNKATN
VAHWTTPSLKCIRDPALVHQRPAPPS
Fc-GGSGGSGGSGGS-IL-15(S162A, L52C)-GGSGGSEPKENLYFQGGGSGGSGGGS-
IL-15Rα(S40C) (SEQ ID NO: 332)
>P1398_Fc-12-L26-PS-R (IL15:S162A, L52C IL15R:S40C) chains 1 and 2
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCFL
LELQVISCESGDASIHDTVENLIILANNSLSSNGNVTESGCKECEELEEKNIKEFLQSFVHIVQMFINTA [symbol] [symbol] [symbol]
ITCPPPMSVEHADIWVKSYSLYSRERYICNSGFKRKAGTCSLTECVLNKATNVA
HWTTPSLKCIRDPALVHQRPAPPS

Expected Cleavage Products from TEV Cleavage of P1398:

1)_Fc-GGSGGSGGSGGS-IL-15(S162A, L52C)-GGSGGSEPKENLYFQ homodimer
(product from complete cleavage) (SEQ ID NO: 333)
Chains 1 and 2
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCFL
LELQVISCESGDASIHDTVENLIILANNSLSSNGNVTESGCKECEELEEKNIKEFLQSFVHIVQMFINTA

[symbol]

[symbol]

2) GGGSGGSGGGS-IL-15Rα(S40C) homodimer (product from complete
cleavage, active) (SEQ ID NO: 334)
Chains 1 and 2 [symbol]
ITCPPPMSVEHADIWVKSYSLYSRERYICNSGFKRKAGTCSLTECVLNKATN
VAHWTTPSLKCIRDPALVHQRPAPPS
3)_Fc-GGSGGSGGSGGS-IL-15(S162A, L52C)-
GGSGGSEPKENLYFQGGGSGGSGGGS-IL-15Rα(540C)/Fc-GGSGGSGGSGGS-IL-15(S162A,
L52C)-GGSGGSEPKENLYFQ/GGGSGGSGGGS-IL-15Rα(S40C) (product from partial
cleavage, active)(
Chain 1:_Fc-GGSGGSGGSGGS-IL-15(S162A, L52C)-
GGSGGSEPKENLYFQGGGSGGSGGGS-IL-15Rα(S40C) (SEQ ID NO: 335)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCFL
LELQVISCESGDASIHDTVENLIILANNSLSSNGNVTESGCKECEELEEKNIKEFLQSFVHIVQMFINTA [symbol] [symbol] [symbol]
ITCPPPMSVEHADIWVKSYSLYSRERYICNSGFKRKAGTCSLTECVLNKATNVA
HWTTPSLKCIRDPALVHQRPAPPS
Chain 2:_Fc-GGSGGSGGSGGS-IL-15(S162A, L52C)-GGSGGSEPKENLYFQ (SEQ
ID NO: 336)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGK [symbol] NWVNVISDLKKIEDLIQSMHIDATLYTESDVHPSCKVTAMKCFL
LELQVISCESGDASIHDTVENLIILANNSLSSNGNVTESGCKECEELEEKNIKEFLQSFVHIVQMFINTAGGSGG
SEPKENLYFQ
Chain 3: GGGSGGSGGGS-IL-15Rα(S40C) (SEQ ID NO: 337) [symbol]
ITCPPPMSVEHADIWVKSYSLYSRERYICNSGFKRKAGTCSLTECVLNKATN
VAHWTTPSLKCIRDPALVHQRPAPPS
Fc-b-GPLGLAGRSANPGPASG-IL-15(S162A)-(GGS)5-IL-15Rα (SEQ ID NO:
3438)
>P1423_Fc-b-PS11-L15R-S162A chains 1 and 2
EPKSSDKTHTCPPCPAPEAAGGPSVFLFPPKPKDTLYITREPEVTCVVVDVSHEDPEVKFNWYVDGVEVHNAKTK
PREEQYNSTYRVVSVLTVLHQDWLNGKEYKCKVSNKALAAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] [symbol] NWVNVISDLKKIEDLIQSMHIDATLYTESDVHPSCKVTAMKCELLELQVIS
LESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPSLKCIRDPALVHQ
RPAPPS
Fc-b-GPLGLAGRSDNHGPASG-IL-15(S162A)-(GGS)5-IL-15Rα (SEQ ID NO:
339)
>P1424_Fc-b-PS12-L15R-S162A chains 1 and 2
EPKSSDKTHTCPPCPAPEAAGGPSVFLFPPKPKDTLYITREPEVTCVVVDVSHEDPEVKFNWYVDGVEVHNAKTK
PREEQYNSTYRVVSVLTVLHQDWLNGKEYKCKVSNKALAAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] [symbol] NWVNVISDLKKIEDLIQSMHIDATLYTESDVHPSCKVTAMKCELLELQVIS
LESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPSLKCIRDPALVHQ
RPAPPS
Fc-b-GPLGLAGRSDNPGPASG-IL-15(S162A)-(GGS)5-IL-15Rα (SEQ ID NO:
340)
>P1425_Fc-b-PS13-L15R-S162A chains 1 and 2
EPKSSDKTHTCPPCPAPEAAGGPSVFLFPPKPKDTLYITREPEVTCVVVDVSHEDPEVKFNWYVDGVEVHNAKTK
PREEQYNSTYRVVSVLTVLHQDWLNGKEYKCKVSNKALAAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] [symbol] NWVNVISDLKKIEDLIQSMHIDATLYTESDVHPSCKVTAMKCELLELQVIS
LESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPSLKCIRDPALVHQ
RPAPPS
Fc-b-GPLGLAGRSENPGPASG-IL-15(S162A)-(GGS)5-IL-15Rα (SEQ ID NO:
341)
>P1426_Fc-b-PS14-L15R-S162A chains 1 and 2
EPKSSDKTHTCPPCPAPEAAGGPSVFLFPPKPKDTLYITREPEVTCVVVDVSHEDPEVKFNWYVDGVEVHNAKTK
PREEQYNSTYRVVSVLTVLHQDWLNGKEYKCKVSNKALAAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] [symbol] NWVNVISDLKKIEDLIQSMHIDATLYTESDVHPSCKVTAMKCELLELQVIS
LESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPSLKCIRDPALVHQ
RPAPPS
Fc-b-GPLGLAGRSDNLGPASG-IL-15(S162A)-(GGS)5-IL-15Rα (SEQ ID NO:
342)
>P1427_Fc-b-PS15-L15R-S162A chains 1 and 2
EPKSSDKTHTCPPCPAPEAAGGPSVFLFPPKPKDTLYITREPEVTCVVVDVSHEDPEVKFNWYVDGVEVHNAKTK
PREEQYNSTYRVVSVLTVLHQDWLNGKEYKCKVSNKALAAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] [symbol] NWVNVISDLKKIEDLIQSMHIDATLYTESDVHPSCKVTAMKCELLELQVIS
LESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPSLKCIRDPALVHQ
RPAPPS
Fc-b-GPLGLAGRNAQVGPASG-IL-15(S162A)-(GGS)5-IL-15Rα (SEQ ID NO:
343)
>P1428_Fc-b-PS16-L15R-S162A chains 1 and 2
EPKSSDKTHTCPPCPAPEAAGGPSVFLFPPKPKDTLYITREPEVTCVVVDVSHEDPEVKFNWYVDGVEVHNAKTK
PREEQYNSTYRVVSVLTVLHQDWLNGKEYKCKVSNKALAAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKS
LSLSPGK [symbol] [symbol] NWVNVISDLKKIEDLIQSMHIDATLYTESDVHPSCKVTAMKCELLELQVIS
LESGDASIHDTVENLIILANNSLSSNGNVTESGCKECEELEEKNIKEFLQSFVHIVQMFINTA [symbol] [symbol]
ITCPPPMSVEHADIWVKSYSLYSRERYICNSGFKRKAGTSSLTECVLNKATNVAHWTTPSLKCIRDPALVHQ
RPAPPS
TEV cleavage site (SEQ ID NO: 344)
ENLYFQG
Linker sequence (SEQ ID NO: 345)
GGSGGSGGSGGSGGS
Exemplary cleavable linker (SEQ ID NO: 346)
GSENLYFQGGS
Fc-GSLSGRSDNAGS-IL-15-(GGS)3-IL-15Rα (SEQ ID NO: 347)
>P1471_Fc-PS17-L15R chains 1 and 2
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSDNAGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSDNDGS-IL-15-(GGS)3-IL-15Rα (SEQ ID NO: 348)
>P1472_Fc-PS18-L15R Chains 1 and 2
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSDNDGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSDNEGS-IL-15-(GGS)3-IL-15Rα (SEQ ID NO: 349)
>P1473_Fc-PS19-L15R Chains 1 and 2
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSDNEGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSDNEGS-IL-15-(GGS)3-IL-15Rα (SEQ ID NO: 350)
>P1474_Fc-PS20-L15R Chains 1 and 2
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSDNEGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSDNGGS-IL-15-(GGS)3-IL-15Rα (SEQ ID NO: 351)
>P1475_Fc-PS21-L15R Chains 1 and 2
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSDNGGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
c-GSLSGRSDNIGS-IL-15-(GGS)3-IL-15Rα (SEQ ID NO: 352)
>P1476_Fc-PS22-L15R Chains 1 and 2
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSDNIGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSDNKGS-IL-15-(GGS)3-IL-15Rα (SEQ ID NO: 353)
>P1477_Fc-PS23-L15R Chains 1 and 2
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSDNKGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSDNLGS-IL-15-(GGS)3-IL-15Rα (SEQ ID NO: 354)
>P1478_Fc-PS24-L15R Chains 1 and 2
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSDNLGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSDNMGS-IL-15-(GGS)3-IL-15Rα (SEQ ID NO: 355)
>P1479_Fc-PS25-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSDNMGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSDNNGS-IL-15-(GGS)3-IL-15Rα (SEQ ID NO: 356)
>P1480_Fc-PS26-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSDNNGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSDNPGS-IL-15-(GGS)3-IL-15Rα (SEQ ID NO: 357)
>P1481_Fc-PS27-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSDNPGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSDNQGS-IL-15-(GGS)3-IL-15Rα (SEQ ID NO: 358)
>P1482_Fc-PS28-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSDNQGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSDNRGS-IL-15-(GGS)3-IL-15Rα (SEQ ID NO: 359)
>P1483_Fc-PS29-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSDNRGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSDNSGS-IL-15-(GGS)3-IL-15Rα (SEQ ID NO: 360)
>P1484_Fc-PS30-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSDNSGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSDNTGS-IL-15-(GGS)3-IL-15Rα (SEQ ID NO: 361)
>P1485_Fc-PS31-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSDNTGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSDNVGS-IL-15-(GGS)3-IL-15Rα (SEQ ID NO: 362)
>P1486_Fc-32-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSDNVGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSDNWGS-IL-15-(GGS)3-IL-15Rα (SEQ ID NO: 363)
>P1487_Fc-PS33-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSDNWGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSDNYGS-IL-15-(GGS)3-IL-15Rα (SEQ ID NO: 364)
22P1488_Fc-PS34-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSDNYGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSANDGS-IL-15-(GGS)3-IL-15Rα (SEQ ID NO: 365)
>P1489_Fc-PS35-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSANDGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSANEGS-IL-15-(GGS)3-IL-15Rα (SEQ ID NO: 366)
>P1490_Fc-PS36-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSANEGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSANFGS-IL-15-(GGS)3-IL-15Rα (SEQ ID NO: 367)
>P1491_Fc-PS37-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSANFGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSANGGS-IL-15-(GGS)3-IL-15Rα (SEQ ID NO: 368)
>P1492_Fc-PS38-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSANGGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSANHGS-IL-15-(GGS)3-IL-15Rα (SEQ ID NO: 369)
>P1493_Fc-PS39-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSANHGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSANIGS-IL-15-(GGS)3-IL-15Rα (SEQ ID NO: 370)
>P1494_Fc-PS40-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSANIGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSANKGS-IL-15-(GGS)3-IL-15Rα (SEQ ID NO: 371)
>P1495_Fc-PS41-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSANKGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSANLGS-IL-15-(GGS)3-IL-15Rα (SEQ ID NO: 372)
>P1496_Fc-PS42-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSANLGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSANMGS-IL-15-(GGS)3-IL-15Rα (SEQ ID NO: 373)
>P1497_Fc-PS43-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSANMGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSANNGS-IL-15-(GGS)3-IL-15Rα (SEQ ID NO: 374)
>P1498_Fc-PS44-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSANNGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSANPGS-IL-15-(GGS)3-IL-15Rα (SEQ ID NO: 375)
>P1499_Fc-PS45-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSANPGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSANQGS-IL-15-(GGS)3-IL-15Rα (SEQ ID NO: 376)
>P1500_Fc-PS46-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSANQGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSANRGS-IL-15-(GGS)3-IL-15Rα (SEQ ID NO: 377)
>P1501_Fc-PS47-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSANRGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSANSGS-IL-15-(GGS)3-IL-15Rα (SEQ ID NO: 378)
>P1502_Fc-PS48-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSANSGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSANTGS-IL-15-(GGS)3-IL-15Rα (SEQ ID NO: 379)
>P1503_Fc-PS49-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSANTGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSANVGS-IL-15-(GGS)3-IL-15Rα (SEQ ID NO: 380)
>P1504_Fc-PS50-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSANVGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSANWGS-IL-15-(GGS)3-IL-15Rα (SEQ ID NO: 381)
>P1505_Fc-PS51-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSANWGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-GSLSGRSANYGS-IL-15-(GGS)3-IL-15Rα (SEQ ID NO: 382)
>P1506_Fc-PS52-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSANYGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-b-GPLGLAGRSDNHSG-IL-15(S162A)-(GGS)3-IL-15Rα (SEQ ID NO: 383)
>P1511_Fc-b-PS12b-L15R-S162A
EPKSSDKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALAAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGPLGLAGRSDNHSGNWVNVISDLKKIEDLIQSMHIDATLYTESDVHPSCKVTAMKC
FLLELQVISLESGDASIHDTVENLIILANNSLSSNGNVTESGCKECEELEEKNIKEFLQSFVHIVQMFINTAGGS
GGSGGSGGSGGSITCPPPMSVEHADIWVKSYSLYSRERYICNSGFKRKAGTSSLTECVLNKATNVAHWTTPSLKC
IRDPALVHQRPAPPS
Fc-PLGLAGSGRSDNR-IL-15(S162A)-(GGS)3-IL-15Rα (SEQ ID NO: 384)
>P1540_Fc-PS53-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKPLGLAGSGRSDNRNWVNVISDLKKIEDLIQSMHIDATLYTESDVHPSCKVTAMKCF
LLELQVISLESGDASIHDTVENLIILANNSLSSNGNVTESGCKECEELEEKNIKEFLQSFVHIVQMFINTAGGSG
GSGGSGGSGGSITCPPPMSVEHADIWVKSYSLYSRERYICNSGFKRKAGTSSLTECVLNKATNVAHWTTPSLKCI
RDPALVHQRPAPPS
Fc-PLGLAGSGRSDNRGS-IL-15(S162A)-(GGS)3-IL-15Rα (SEQ ID NO: 385)
>P1541_Fc-PS54-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKPLGLAGSGRSDNRGSNWVNVISDLKKIEDLIQSMHIDATLYTESDVHPSCKVTAMK
CFLLELQVISLESGDASIHDTVENLIILANNSLSSNGNVTESGCKECEELEEKNIKEFLQSFVHIVQMFINTAGG
SGGSGGSGGSGGSITCPPPMSVEHADIWVKSYSLYSRERYICNSGFKRKAGTSSLTECVLNKATNVAHWTTPSLK
CIRDPALVHQRPAPPS
Fc-GGGSKTHTGGGS-IL-15(S162A)-GSPLGLAGSGRSDNRGS-IL-15Rα (SEQ ID
NO: 386)
>P1542_Fc-StableS-L-PS55-R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGGGSKTHTGGGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTAGSPLG
LAGSGRSDNRGSITCPPPMSVEHADIWVKSYSLYSRERYICNSGFKRKAGTSSLTECVLNKATNVAHWTTPSLKC
IRDPALVHQRPAPPS
Fc-delK-GGGSKTHTGGGS-IL-15(S162A)-GSPLGLAGSGRSDNRGS-IL-15Rα
(SEQ ID NO: 387)
>P1636_(Fc)SPG-stable linker-L-PS55-R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGGGGSKTHTGGGSNWVNVISDLKKIEDLIQSMHIDATLYTESDVHPSCKVTAMKCELL
ELQVISLESGDASIHDTVENLIILANNSLSSNGNVTESGCKECEELEEKNIKEFLQSFVHIVQMFINTAGSPLGL
AGSGRSDNRGSITCPPPMSVEHADIWVKSYSLYSRERYICNSGFKRKAGTSSLTECVLNKATNVAHWTTPSLKCI
RDPALVHQRPAPPS
Fc-GGGSAPTSSGSLSGRSDNHGS-IL-15-(GGS)3-IL-15Rα (SEQ ID NO: 388)
>P1637_(Fc)SPGK-GGGSAPTSS-GSLSGRSDNHGS-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGGGSAPTSSGSLSGRSDNHGSNWVNVISDLKKIEDLIQSMHIDATLYTESDVHPSC
KVTAMKCFLLELQVISLESGDASIHDTVENLIILANNSLSSNGNVTESGCKECEELEEKNIKEFLQSFVHIVQMF
INTSGGSGGSGGSGGSGGSITCPPPMSVEHADIWVKSYSLYSRERYICNSGFKRKAGTSSLTECVLNKATNVAHW
TTPSLKCIRDPALVHQRPAPPS
Fc-GSLSGRSDNRGS-IL-15-(GGS)3-IL-15Rα (SEQ ID NO: 389)
>P1638_(Fc)SPGK-GSLSGRSDNRGS-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGSLSGRSDNRGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTSGGSGG
SGGSGGSGGSITCPPPMSVEHADIWVKSYSLYSRERYICNSGFKRKAGTSSLTECVLNKATNVAHWTTPSLKCIR
DPALVHQRPAPPS
Fc-delK-GSLSGRSDNHGS-IL-15-(GGS)3-IL-15Rα (SEQ ID NO: 390)
>P1639_(Fc)SPG-GSLSGRSDNHGS-L15R
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGGSLSGRSDNHGSNWVNVISDLKKIEDLIQSMHIDATLYTESDVHPSCKVTAMKCELL
ELQVISLESGDASIHDTVENLIILANNSLSSNGNVTESGCKECEELEEKNIKEFLQSFVHIVQMFINTSGGSGGS
GGSGGSGGSITCPPPMSVEHADIWVKSYSLYSRERYICNSGFKRKAGTSSLTECVLNKATNVAHWTTPSLKCIRD
PALVHQRPAPPS
Fc-delK-PLGLAGSGRSDNRGA-IL-15(D8N, S162A)-(GGS)3-IL-15Rα (SEQ
ID NO: 391)
>P1652_Fc_delK_PS103_L15R(IL15-D8N-S162A)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGPLGLAGSGRSDNRGANWVNVISNLKKIEDLIQSMHIDATLYTESDVHPSCKVTAMKC
FLLELQVISLESGDASIHDTVENLIILANNSLSSNGNVTESGCKECEELEEKNIKEFLQSFVHIVQMFINTAGGS
GGSGGSGGSGGSITCPPPMSVEHADIWVKSYSLYSRERYICNSGFKRKAGTSSLTECVLNKATNVAHWTTPSLKC
IRDPALVHQRPAPPS
Fc-delK-PLGLAGSGRSDNRGA-IL-15(E64Q, S162A)-(GGS)3-IL-15Rα (SEQ
ID NO: 392)
>P1653_Fc_delK_PS103_L15R(IL15-E64Q-S162A)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGPLGLAGSGRSDNRGANWVNVISDLKKIEDLIQSMHIDATLYTESDVHPSCKVTAMKC
FLLELQVISLESGDASIHDTVQNLIILANNSLSSNGNVTESGCKECEELEEKNIKEFLQSFVHIVQMFINTAGGS
GGSGGSGGSGGSITCPPPMSVEHADIWVKSYSLYSRERYICNSGFKRKAGTSSLTECVLNKATNVAHWTTPSLKC
IRDPALVHQRPAPPS
Fc-delK-PLGLAGSGRSDNRGA-IL-15(E64K, S162A)-(GGS)3-IL-15Rα (SEQ
ID NO: 393)
>P1654_Fc_delK_PS103_L15R(IL15-E64K-S162A)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGPLGLAGSGRSDNRGANWVNVISDLKKIEDLIQSMHIDATLYTESDVHPSCKVTAMKC
FLLELQVISLESGDASIHDTVKNLIILANNSLSSNGNVTESGCKECEELEEKNIKEFLQSFVHIVQMFINTAGGS
GGSGGSGGSGGSITCPPPMSVEHADIWVKSYSLYSRERYICNSGFKRKAGTSSLTECVLNKATNVAHWTTPSLKC
IRDPALVHQRPAPPS
Fc-delK-PLGLAGSGRSDNRGA-IL-15(N65A, S162A)-(GGS)3-IL-15Rα (SEQ
ID NO: 394)
>P1655_Fc_delK_PS103_L15R(IL15-N65A-S162A)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGPLGLAGSGRSDNRGANWVNVISDLKKIEDLIQSMHIDATLYTESDVHPSCKVTAMKC
FLLELQVISLESGDASIHDTVEALIILANNSLSSNGNVTESGCKECEELEEKNIKEFLQSFVHIVQMFINTAGGS
GGSGGSGGSGGSITCPPPMSVEHADIWVKSYSLYSRERYICNSGFKRKAGTSSLTECVLNKATNVAHWTTPSLKC
IRDPALVHQRPAPPS
Fc-delK-PLGLAGSGRSDNRGA-IL-15(N65D, S162A)-(GGS)3-IL-15Rα (SEQ
ID NO: 395)
>P1656_Fc_delK_PS103_L15R(IL15-N65D-S162A)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGPLGLAGSGRSDNRGANWVNVISDLKKIEDLIQSMHIDATLYTESDVHPSCKVTAMKC
FLLELQVISLESGDASIHDTVEDLIILANNSLSSNGNVTESGCKECEELEEKNIKEFLQSFVHIVQMFINTAGGS
GGSGGSGGSGGSITCPPPMSVEHADIWVKSYSLYSRERYICNSGFKRKAGTSSLTECVLNKATNVAHWTTPSLKC
IRDPALVHQRPAPPS
Fc-delK-PLGLAGSGRSDNRGA-IL-15(I68D, S162A)-(GGS)3-IL-15Rα (SEQ
ID NO: 396)
>P1657_Fc_delK_PS103_L15R(IL15-168D-S162A)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGPLGLAGSGRSDNRGANWVNVISDLKKIEDLIQSMHIDATLYTESDVHPSCKVTAMKC
FLLELQVISLESGDASIHDTVENLIDLANNSLSSNGNVTESGCKECEELEEKNIKEFLQSFVHIVQMFINTAGGS
GGSGGSGGSGGSITCPPPMSVEHADIWVKSYSLYSRERYICNSGFKRKAGTSSLTECVLNKATNVAHWTTPSLKC
IRDPALVHQRPAPPS
Fc-delK-PLGLAGSGRSDNQGA-IL-15(S162A)-(GGS)3-IL-15Rα (SEQ ID
NO: 397)
>P1658_Fc_delK_PS104_L15R(IL15-S162A)
EPKSSDKTHTCPPCPAPELLGGPSVFLEPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFELYSKLTVDKSRWQQGNVESCSVM
HEALHNHYTQKSLSLSPGPLGLAGSGRSDNQGANWVNVISDLKKIEDLIQSMHIDATLYTESDVHPSCKVTAMKC
FLLELQVISLESGDASIHDTVENLIILANNSLSSNGNVTESGCKECEELEEKNIKEFLQSFVHIVQMFINTAGGS
GGSGGSGGSGGSITCPPPMSVEHADIWVKSYSLYSRERYICNSGFKRKAGTSSLTECVLNKATNVAHWTTPSLKC
IRDPALVHQRPAPPS
Fc-delK-PLGLAGSGRSDNYGA-IL-15(S162A)-(GGS)3-IL-15Rα (SEQ ID
NO: 398)
>P1659_Fc_delK_PS105_L15R(IL15-S162A)
EPKSSDKTHTCPPCPAPELLGGPSVFLEPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFELYSKLTVDKSRWQQGNVESCSVM
HEALHNHYTQKSLSLSPGPLGLAGSGRSDNYGANWVNVISDLKKIEDLIQSMHIDATLYTESDVHPSCKVTAMKC
FLLELQVISLESGDASIHDTVENLIILANNSLSSNGNVTESGCKECEELEEKNIKEFLQSFVHIVQMFINTAGGS
GGSGGSGGSGGSITCPPPMSVEHADIWVKSYSLYSRERYICNSGFKRKAGTSSLTECVLNKATNVAHWTTPSLKC
IRDPALVHQRPAPPS
Fc-delK-PLGLAGSGRSDNYGA-IL-15(S162A)-(GGS)3-IL-15Rα (IL-15opt1,
IL-15Ropt) (SEQ ID NO: 399)
>P1660_1659a_Fc_delK-P5105-L15R (IL-15-S162A, IL-15opt1, IL-15Ropt)
EPKSSDKTHTCPPCPAPELLGGPSVFLEPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFELYSKLTVDKSRWQQGNVESCSVM
HEALHNHYTQKSLSLSPGPLGLAGSGRSDNYGANWVNVISDLKKIEDLIQSMHIDATLYTESDVHPSCKVTAMKC
FLLELQVISLESGDASIHDTVENLIILANNSLSSNGNVTESGCKECEELEEKNIKEFLQSFVHIVQMFINTAGGS
GGSGGSGGSGGSITCPPPMSVEHADIWVKSYSLYSRERYICNSGFKRKAGTSSLTECVLNKATNVAHWTTPSLKC
IRDPALVHQRPAPPS
GAGCCCAAATCTTCTGACAAAACTCACACATGCCCACCGTGCCCAGCACCTGAACTCCTGGGG
GGACCGTCAGTCTTCCTCTTCCCCCCAAAACCCAAGGACACCCTCATGATCTCCCGGACCCCTGAGGTCACATGC
GTGGTGGTGGACGTGAGCCACGAAGACCCTGAGGTCAAGTTCAACTGGTACGTGGACGGCGTGGAGGTGCATAAT
GCCAAGACAAAGCCGCGGGAGGAGCAGTACAACAGCACGTACCGTGTGGTCAGCGTCCTCACCGTCCTGCACCAG
GACTGGCTGAATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCACGGGAGGAGATGACCAAGAAC
CAGGTCAGCCTGACCTGCCTGGTCAAAGGCTTCTATCCCAGCGACATCGCCGTGGAGTGGGAGAGCAATGGGCAG
CCGGAGAACAACTACAAGACCACGCCTCCCGTGCTGGACTCCGACGGCTCCTTCTTCCTCTATAGCAAGCTCACC
GTGGACAAGAGCAGGTGGCAGCAGGGGAACGTCTTCTCATGCTCCGTGATGCATGAGGCTCTGCACAACCACTAC
ACGCAGAAGAGCCTCTCCCTGTCCCCGGGTCCATTGGGACTGGCTGGGAGCGGTAGATCCGACAATTACGGAGCC
AACTGGGTGAACGTGATCTCGGACCTGAAGAAGATCGAGGACCTCATCCAGTCGATGCACATCGACGCGACGCTG
TACACGGAGTCGGACGTCCACCCGTCGTGCAAGGTCACGGCGATGAAGTGCTTCCTCCTGGAGCTCCAAGTCATC
TCGCTCGAGTCGGGGGACGCGTCGATCCACGACACGGTGGAGAACCTGATCATCCTGGCGAACAACTCGCTGTCG
TCGAACGGGAACGTCACGGAGTCGGGCTGCAAGGAGTGCGAGGAGCTGGAGGAGAAGAACATCAAGGAGTTCCTG
CAGTCGTTCGTGCACATCGTCCAGATGTTCATCAACACGGCCGGTGGCTCCGGCGGAAGCGGAGGTTCCGGCGGT
AGCGGTGGATCCATCACGTGCCCGCCCCCCATGTCCGTGGAGCACGCAGACATCTGGGTCAAGAGCTACAGCTTG
TACTCCCGGGAGCGGTACATCTGCAACTCGGGTTTCAAGCGGAAGGCCGGCACGTCCAGCCTGACGGAGTGCGTG
TTGAACAAGGCCACGAATGTCGCCCACTGGACGACCCCCTCGCTCAAGTGCATCCGCGACCCGGCCCTGGTTCAC
CAGCGGCCCGCGCCACCCTCC (SEQ ID NO: 400)
Fc-delK-PLGLAGSGRSDNYGA-IL-15(S162A)-(GGS)3-IL-15Rα (IL-150pt2,
IL-15Ropt) (SEQ ID NO: 401)
>P1661_1659b_Fc_delK-PS105-L15R (IL-15-S162A, IL-15opt2, IL-15Ropt)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGPLGLAGSGRSDNYGANWVNVISDLKKIEDLIQSMHIDATLYTESDVHPSCKVTAMKC
FLLELQVISLESGDASIHDTVENLIILANNSLSSNGNVTESGCKECEELEEKNIKEFLQSFVHIVQMFINTAGGS
GGSGGSGGSGGSITCPPPMSVEHADIWVKSYSLYSRERYICNSGFKRKAGTSSLTECVLNKATNVAHWTTPSLKC
IRDPALVHQRPAPPS
GAGCCCAAATCTTCTGACAAAACTCACACATGCCCACCGTGCCCAGCACCTGAACTCCTGGGG
GGACCGTCAGTCTTCCTCTTCCCCCCAAAACCCAAGGACACCCTCATGATCTCCCGGACCCCTGAGGTCACATGC
GTGGTGGTGGACGTGAGCCACGAAGACCCTGAGGTCAAGTTCAACTGGTACGTGGACGGCGTGGAGGTGCATAAT
GCCAAGACAAAGCCGCGGGAGGAGCAGTACAACAGCACGTACCGTGTGGTCAGCGTCCTCACCGTCCTGCACCAG
GACTGGCTGAATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCACGGGAGGAGATGACCAAGAAC
CAGGTCAGCCTGACCTGCCTGGTCAAAGGCTTCTATCCCAGCGACATCGCCGTGGAGTGGGAGAGCAATGGGCAG
CCGGAGAACAACTACAAGACCACGCCTCCCGTGCTGGACTCCGACGGCTCCTTCTTCCTCTATAGCAAGCTCACC
GTGGACAAGAGCAGGTGGCAGCAGGGGAACGTCTTCTCATGCTCCGTGATGCATGAGGCTCTGCACAACCACTAC
ACGCAGAAGAGCCTCTCCCTGTCCCCGGGTCCATTGGGACTGGCTGGGAGCGGTAGATCCGACAATTACGGAGCC
AATTGGGTGAACGTGATCAGCGACCTGAAGAAGATCGAGGACCTCATCCAGAGCATGCACATCGACGCCACCCTG
TACACCGAGAGCGATGTGCACCCCAGCTGTAAGGTGACCGCCATGAAGTGCTTTCTGCTGGAGCTGCAAGTGATC
AGCCTGGAGAGCGGCGACGCCAGCATCCACGACACCGTGGAGAACCTGATCATCCTGGCCAACAACAGCCTGAGC
AGCAACGGCAATGTGACCGAGAGCGGCTGTAAGGAGTGTGAGGAGCTGGAGGAGAAGAACATCAAGGAGTTTCTG
CAGAGCTTCGTGCACATCGTGCAGATGTTCATCAACACCGCCGGTGGCTCCGGCGGAAGCGGAGGTTCCGGCGGT
AGCGGTGGATCCATCACGTGCCCGCCCCCCATGTCCGTGGAGCACGCAGACATCTGGGTCAAGAGCTACAGCTTG
TACTCCCGGGAGCGGTACATCTGCAACTCGGGTTTCAAGCGGAAGGCCGGCACGTCCAGCCTGACGGAGTGCGTG
TTGAACAAGGCCACGAATGTCGCCCACTGGACGACCCCCTCGCTCAAGTGCATCCGCGACCCGGCCCTGGTTCAC
CAGCGGCCCGCGCCACCCTCC (SEQ ID NO: 402)
Fc-delK-PLGLAGSGRSDNYGA-IL-15(S162A)-(GGS)3-IL-15Rα (IL-15co,
IL-15aco) (SEQ ID NO: 403)
>P1663_1659c_Fc_delK-PS105-L15R (IL-15-S162A, IL15co, IL15Raco)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGPLGLAGSGRSDNYGANWVNVISDLKKIEDLIQSMHIDATLYTESDVHPSCKVTAMKC
FLLELQVISLESGDASIHDTVENLIILANNSLSSNGNVTESGCKECEELEEKNIKEFLQSFVHIVQMFINTAGGS
GGSGGSGGSGGSITCPPPMSVEHADIWVKSYSLYSRERYICNSGFKRKAGTSSLTECVLNKATNVAHWTTPSLKC
IRDPALVHQRPAPPS
GAGCCCAAATCTTCTGACAAAACTCACACATGCCCACCGTGCCCAGCACCTGAACTCCTGGGG
GGACCGTCAGTCTTCCTCTTCCCCCCAAAACCCAAGGACACCCTCATGATCTCCCGGACCCCTGAGGTCACATGC
GTGGTGGTGGACGTGAGCCACGAAGACCCTGAGGTCAAGTTCAACTGGTACGTGGACGGCGTGGAGGTGCATAAT
GCCAAGACAAAGCCGCGGGAGGAGCAGTACAACAGCACGTACCGTGTGGTCAGCGTCCTCACCGTCCTGCACCAG
GACTGGCTGAATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCACGGGAGGAGATGACCAAGAAC
CAGGTCAGCCTGACCTGCCTGGTCAAAGGCTTCTATCCCAGCGACATCGCCGTGGAGTGGGAGAGCAATGGGCAG
CCGGAGAACAACTACAAGACCACGCCTCCCGTGCTGGACTCCGACGGCTCCTTCTTCCTCTATAGCAAGCTCACC
GTGGACAAGAGCAGGTGGCAGCAGGGGAACGTCTTCTCATGCTCCGTGATGCATGAGGCTCTGCACAACCACTAC
ACGCAGAAGAGCCTCTCCCTGTCCCCGGGTCCATTGGGACTGGCTGGGAGCGGTAGATCCGACAATTACGGAGCC
AACTGGGTGAACGTGATCAGCGACCTGAAGAAGATCGAGGACCTGATCCAGAGCATGCACATCGACGCCACCCTG
TACACCGAGAGCGACGTGCACCCCAGCTGCAAGGTGACCGCCATGAAGTGCTTCCTGCTGGAGCTGCAGGTGATC
AGCCTGGAGAGCGGCGACGCCAGCATCCACGACACCGTGGAGAACCTGATCATCCTGGCCAACAACAGCCTGAGC
AGCAACGGCAACGTGACCGAGAGCGGCTGCAAGGAGTGCGAGGAGCTGGAGGAGAAGAACATCAAGGAGTTCCTG
CAGAGCTTCGTGCACATCGTGCAGATGTTCATCAACACCGCCGGTGGCTCCGGCGGAAGCGGAGGTTCCGGCGGT
AGCGGTGGATCCATCACCTGCCCTCCTCCAATGAGCGTGGAGCACGCCGACATCTGGGTGAAGAGCTACAGCCTG
TACAGCCGGGAGCGGTACATCTGCAACAGCGGCTTCAAGCGGAAGGCTGGCACCAGCAGCCTGACCGAGTGCGTG
CTGAACAAGGCCACCAACGTGGCCCACTGGACCACCCCCAGCCTGAAGTGCATCCGGGACCCTGCCCTGGTGCAT
CAACGGCCTGCTCCTCCTAGC (SEQ ID NO: 404)
Fc-delK-PLGLAGSGRSDNYGA-IL-15(S162A)-(GGS)3-IL-15Rα-sushi(T2A)
(SEQ ID NO: 405)
>P1682_Fc_delK-PS105-L15Rsushi (IL15_S162A-IL15Rsushi_T2A)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGPLGLAGSGRSDNYGANWVNVISDLKKIEDLIQSMHIDATLYTESDVHPSCKVTAMKC
FLLELQVISLESGDASIHDTVENLIILANNSLSSNGNVTESGCKECEELEEKNIKEFLQSFVHIVQMFINTAGGS
GGSGGSGGSGGSIACPPPMSVEHADIWVKSYSLYSRERYICNSGFKRKAGTSSLTECVLNKATNVAHWTTPSLKC
IR
Fc-delK-PLGLAGSGRSDNYGA-IL-15(L52C, S162A)-(GGS)3-IL-15Rα-
sushi(T2A, S40C) (SEQ ID NO: 406)
>P1683_Fc_delK-PS105-L15Rsushi (IL15_L52C_S162A-IL15Rsushi_T2A_S40C)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGPLGLAGSGRSDNYGANWVNVISDLKKIEDLIQSMHIDATLYTESDVHPSCKVTAMKC
FLLELQVISCESGDASIHDTVENLIILANNSLSSNGNVTESGCKECEELEEKNIKEFLQSFVHIVQMFINTAGGS
GGSGGSGGSGGSIACPPPMSVEHADIWVKSYSLYSRERYICNSGFKRKAGTCSLTECVLNKATNVAHWTTPSLKC
IR
Fc-delK-GG-IL-15(S162A)-GPLGLAGSGRSDNQG-IL-15Rα(T2A) (SEQ ID
NO: 407)
>P1696_Fc_delK-GG-L_S162A-GPLGLAGSGRSDNQG-R_T2A (2/15Q)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGGGNWVNVISDLKKIEDLIQSMHIDATLYTESDVHPSCKVTAMKCELLELQVISLESG
DASIHDTVENLIILANNSLSSNGNVTESGCKECEELEEKNIKEFLQSFVHIVQMFINTAGPLGLAGSGRSDNQGI
ACPPPMSVEHADIWVKSYSLYSRERYICNSGFKRKAGTSSLTECVLNKATNVAHWTTPSLKCIRDPALVHQRPAP
PS
Fc-delK-GG-IL-15(S162A)-GSPLGLAGSGRSDNQGA-IL-15Rα(T2A) (SEQ ID
NO: 408)
>P1697_Fc_delK-GG-L_S162A-GSPLGLAGSGRSDNQGA-R_T2A (2/17Q)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGGGNWVNVISDLKKIEDLIQSMHIDATLYTESDVHPSCKVTAMKCELLELQVISLESG
DASIHDTVENLIILANNSLSSNGNVTESGCKECEELEEKNIKEFLQSFVHIVQMFINTAGSPLGLAGSGRSDNQG
AIACPPPMSVEHADIWVKSYSLYSRERYICNSGFKRKAGTSSLTECVLNKATNVAHWTTPSLKCIRDPALVHQRP
APPS
Fc-delK-GG-IL-15(S162A)-GGSPLGLAGSGRSDNQGGA-IL-15Rα(T2A) (SEQ
ID NO: 409)
>P1698_Fc_delK-GG-L_S162A-GGSPLGLAGSGRSDNQGGA-R_T2A (2/19Q)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGGGNWVNVISDLKKIEDLIQSMHIDATLYTESDVHPSCKVTAMKCELLELQVISLESG
DASIHDTVENLIILANNSLSSNGNVTESGCKECEELEEKNIKEFLQSFVHIVQMFINTAGGSPLGLAGSGRSDNQ
GGAIACPPPMSVEHADIWVKSYSLYSRERYICNSGFKRKAGTSSLTECVLNKATNVAHWTTPSLKCIRDPALVHQ
RPAPPS
Fc-delK-GG-IL-15(S162A)-GGGSPLGLAGSGRSDNQGGGA-IL-15Rα(T2A) (SEQ
ID NO: 410)
>P1699_15LR-119_Fc_delK-GG-L_S162A-GGGSPLGLAGSGRSDNQGGGA-R_T2A
(2/21Q)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGGGNWVNVISDLKKIEDLIQSMHIDATLYTESDVHPSCKVTAMKCELLELQVISLESG
DASIHDTVENLIILANNSLSSNGNVTESGCKECEELEEKNIKEFLQSFVHIVQMFINTAGGGSPLGLAGSGRSDN
QGGGAIACPPPMSVEHADIWVKSYSLYSRERYICNSGFKRKAGTSSLTECVLNKATNVAHWTTPSLKCIRDPALV
HQRPAPPS
Fc-delK-GG-IL-15(S162A)-GGSGSPLGLAGSGRSDNQGGGGA-IL-15Rα(T2A)
(SEQ ID NO: 411)
>P1700_Fc_delK-GG-L_S162A-GGSGSPLGLAGSGRSDNQGGGGA-R_T2A (2/23Q)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGGGNWVNVISDLKKIEDLIQSMHIDATLYTESDVHPSCKVTAMKCELLELQVISLESG
DASIHDTVENLIILANNSLSSNGNVTESGCKECEELEEKNIKEFLQSFVHIVQMFINTAGGSGSPLGLAGSGRSD
NQGGGGAIACPPPMSVEHADIWVKSYSLYSRERYICNSGFKRKAGTSSLTECVLNKATNVAHWTTPSLKCIRDPA
LVHQRPAPPS
Fc-delK-GG-IL-15(S162A)-GGSGGSPLGLAGSGRSDNQGGSGGA-IL-15Rα(T2A)
(SEQ ID NO: 412)
>P1701_Fc_delK-GG-L_S162A-GGSGGSPLGLAGSGRSDNQGGSGGA-R_T2A (2/25Q)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGGGNWVNVISDLKKIEDLIQSMHIDATLYTESDVHPSCKVTAMKCELLELQVISLESG
DASIHDTVENLIILANNSLSSNGNVTESGCKECEELEEKNIKEFLQSFVHIVQMFINTAGGSGGSPLGLAGSGRS
DNQGGSGGAIACPPPMSVEHADIWVKSYSLYSRERYICNSGFKRKAGTSSLTECVLNKATNVAHWTTPSLKCIRD
PALVHQRPAPPS
Fc-delK-GG-IL-15(S162A)-GPLGLAGSGRSDNRG-IL-15Rα(T2A) (SEQ ID
NO: 413)
>P1702_Fc_delK-GG-L_S162A-GPLGLAGSGRSDNRG-R_T2A (2/15R)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGGGNWVNVISDLKKIEDLIQSMHIDATLYTESDVHPSCKVTAMKCELLELQVISLESG
DASIHDTVENLIILANNSLSSNGNVTESGCKECEELEEKNIKEFLQSFVHIVQMFINTAGPLGLAGSGRSDNRGI
ACPPPMSVEHADIWVKSYSLYSRERYICNSGFKRKAGTSSLTECVLNKATNVAHWTTPSLKCIRDPALVHQRPAP
PS
Fc-delK-GG-IL-15(S162A)-GSPLGLAGSGRSDNRGA-IL-15Rα(T2A) (SEQ ID
NO: 414)
>P1703_Fc_delK-GG-L_S162A-GSPLGLAGSGRSDNRGA-R_T2A (2/17R)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGGGNWVNVISDLKKIEDLIQSMHIDATLYTESDVHPSCKVTAMKCELLELQVISLESG
DASIHDTVENLIILANNSLSSNGNVTESGCKECEELEEKNIKEFLQSFVHIVQMFINTAGSPLGLAGSGRSDNRG
AIACPPPMSVEHADIWVKSYSLYSRERYICNSGFKRKAGTSSLTECVLNKATNVAHWTTPSLKCIRDPALVHQRP
APPS
Fc-delK-GG-IL-15(S162A)-GGSPLGLAGSGRSDNRGGA-IL-15Rα(T2A) (SEQ
ID NO: 415)
>P1704_Fc_delK-GG-L_S162A-GGSPLGLAGSGRSDNRGGA-R_T2A (2/19R)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGGGNWVNVISDLKKIEDLIQSMHIDATLYTESDVHPSCKVTAMKCELLELQVISLESG
DASIHDTVENLIILANNSLSSNGNVTESGCKECEELEEKNIKEFLQSFVHIVQMFINTAGGSPLGLAGSGRSDNR
GGAIACPPPMSVEHADIWVKSYSLYSRERYICNSGFKRKAGTSSLTECVLNKATNVAHWTTPSLKCIRDPALVHQ
RPAPPS
Fc-delK-GG-IL-15(S162A)-GGGSPLGLAGSGRSDNRGGGA-IL-15Rα(T2A) (SEQ
ID NO: 416)
>P1705_Fc_delK-GG-L_S162A-GGGSPLGLAGSGRSDNRGGGA-R_T2A (2/21R)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGGGNWVNVISDLKKIEDLIQSMHIDATLYTESDVHPSCKVTAMKCELLELQVISLESG
DASIHDTVENLIILANNSLSSNGNVTESGCKECEELEEKNIKEFLQSFVHIVQMFINTAGGGSPLGLAGSGRSDN
RGGGAIACPPPMSVEHADIWVKSYSLYSRERYICNSGFKRKAGTSSLTECVLNKATNVAHWTTPSLKCIRDPALV
HQRPAPPS
Fc-delK-GG-IL-15(S162A)-GGSGSPLGLAGSGRSDNRGGGGA-IL-15Rα(T2A)
(SEQ ID NO: 417)
>P1706_Fc_delK-GG-L_S162A-GGSGSPLGLAGSGRSDNRGGGGA-R_T2A (2/23R)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGGGNWVNVISDLKKIEDLIQSMHIDATLYTESDVHPSCKVTAMKCFLLELQVISLESG
DASIHDTVENLIILANNSLSSNGNVTESGCKECEELEEKNIKEFLQSFVHIVQMFINTAGGSGSPLGLAGSGRSD
NRGGGGAIACPPPMSVEHADIWVKSYSLYSRERYICNSGFKRKAGTSSLTECVLNKATNVAHWTTPSLKCIRDPA
LVHQRPAPPS
Fc-delK-GG-IL-15(S162A)-GGSGGSPLGLAGSGRSDNRGGSGGA-IL-15Rα(T2A)
(SEQ ID NO: 418)
>P1707_Fc_delK-GG-L_S162A-GGSGGSPLGLAGSGRSDNRGGSGGA-R_T2A (2/25R)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGGGNWVNVISDLKKIEDLIQSMHIDATLYTESDVHPSCKVTAMKCFLLELQVISLESG
DASIHDTVENLIILANNSLSSNGNVTESGCKECEELEEKNIKEFLQSFVHIVQMFINTAGGSGGSPLGLAGSGRS
DNRGGSGGAIACPPPMSVEHADIWVKSYSLYSRERYICNSGFKRKAGTSSLTECVLNKATNVAHWTTPSLKCIRD
PALVHQRPAP
Fc-G-IL-15(S162A)-GGSGGSEPKENLYFQGGGSGGSGGGS-IL-15Rα(A50C,
T58C) (SEQ ID NO: 419)
>P1973_Fc-1-L26-PS-R (IL15_S162A, IL15Rα_A50C_T58C)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGNWVNVISDLKKIEDLIQSMHIDATLYTESDVHPSCKVTAMKCFLLELQVISLESG
DASIHDTVENLIILANNSLSSNGNVTESGCKECEELEEKNIKEFLQSFVHIVQMFINTAGGSGGSEPKENLYFQG
GGSGGSGGGSITCPPPMSVEHADIWVKSYSLYSRERYICNSGFKRKAGTSSLTECVLNKCTNVAHWTCPSLKCIR
DPALVHQRPAPPS
Fc-GGSGGS-IL-15(S162A)-GGSGGSEPKENLYFQGGGSGGSGGGS-IL-15Rα(A50C,
T58C) (SEQ ID NO: 420)
>P1974_Fc-6-L26-PS-R (IL15_S162A, IL15Rα_A50C_T58C)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGGSGGSNWVNVISDLKKIEDLIQSMHIDATLYTESDVHPSCKVTAMKCFLLELQVI
SLESGDASIHDTVENLIILANNSLSSNGNVTESGCKECEELEEKNIKEFLQSFVHIVQMFINTAGGSGGSEPKEN
LYFQGGGSGGSGGGSITCPPPMSVEHADIWVKSYSLYSRERYICNSGFKRKAGTSSLTECVLNKCTNVAHWTCPS
LKCIRDPALVHQRPAPPS
Fc-GGSGGSGGSGGS-IL-15(S162A)-GGSGGSEPKENLYFQGGGSGGSGGGS-IL-
15Rα(A50C, T58C) (SEQ ID NO: 421)
>P1975_Fc-12-L26-PS-R (IL15_S162A, IL15Rα_A50C_T58C)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGKGGSGGSGGSGGSNWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHIVQMFINTAGGSGG
SEPKENLYFQGGGSGGSGGGSITCPPPMSVEHADIWVKSYSLYSRERYICNSGFKRKAGTSSLTECVLNKCTNVA
HWTCPSLKCIRDPALVHQRPAPPS
Fc-delK-PLGLAGSGRSDNQGA-IL-15(S162A)-GGSGGSGGSGGS-IL-15Rα (SEQ
ID NO: 422)
>P2162_Fc_delK-PS104-L12R (IL15-S162A)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGPLGLAGSGRSDNQGANWVNVISDLKKIEDLIQSMHIDATLYTESDVHPSCKVTAMKC
FLLELQVISLESGDASIHDTVENLIILANNSLSSNGNVTESGCKECEELEEKNIKEFLQSFVHIVQMFINTAGGS
GGSGGSGGSITCPPPMSVEHADIWVKSYSLYSRERYICNSGFKRKAGTSSLTECVLNKATNVAHWTTPSLKCIRD
PALVHQRPAPPS
Fc-delK-PLGLAGSGRSDNQGA-IL-15(S162A)-GGSGGSGGS-IL-15Rα (SEQ ID
NO: 423)
>P2163_Fc_delK-PS104-L9R (IL15-S162A)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGPLGLAGSGRSDNQGANWVNVISDLKKIEDLIQSMHIDATLYTESDVHPSCKVTAMKC
FLLELQVISLESGDASIHDTVENLIILANNSLSSNGNVTESGCKECEELEEKNIKEFLQSFVHIVQMFINTAGGS
GGSGGSITCPPPMSVEHADIWVKSYSLYSRERYICNSGFKRKAGTSSLTECVLNKATNVAHWTTPSLKCIRDPAL
VHQRPAPPS
Fc-delK-PLGLAGSGRSDNQGA-IL-15(S162A)-GGSGGS-IL-15Rα (SEQ ID
NO: 424)
>P2164_Fc_delK-PS104-L6R (IL15-S162A)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGPLGLAGSGRSDNQGANWVNVISDLKKIEDLIQSMHIDATLYTESDVHPSCKVTAMKC
FLLELQVISLESGDASIHDTVENLIILANNSLSSNGNVTESGCKECEELEEKNIKEFLQSFVHIVQMFINTAGGS
GGSITCPPPMSVEHADIWVKSYSLYSRERYICNSGFKRKAGTSSLTECVLNKATNVAHWTTPSLKCIRDPALVHQ
RPAPPS
Fc-delK-PLGLAGSGRSDNQGA-IL-15(S162A)-GGS-IL-15Rα (SEQ ID
NO: 425)
>P2165_Fc_delK-PS104-L3R (IL15-S162A)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGPLGLAGSGRSDNQGANWVNVISDLKKIEDLIQSMHIDATLYTESDVHPSCKVTAMKC
FLLELQVISLESGDASIHDTVENLIILANNSLSSNGNVTESGCKECEELEEKNIKEFLQSFVHIVQMFINTAGGS
ITCPPPMSVEHADIWVKSYSLYSRERYICNSGFKRKAGTSSLTECVLNKATNVAHWTTPSLKCIRDPALVHQRPA
PPS
Fc-delK-GSPLGLAGSGRSDNQGSGA-IL-15(S162A)-GGSGGSGGSGGS-IL-15Rα
(SEQ ID NO: 426)
>P2166_Fc_delK-PS106-L12R (IL15-S162A)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGGSPLGLAGSGRSDNQGSGANWVNVISDLKKIEDLIQSMHIDATLYTESDVHPSCKVT
AMKCFLLELQVISLESGDASIHDTVENLIILANNSLSSNGNVTESGCKECEELEEKNIKEFLQSFVHIVQMFINT
AGGSGGSGGSGGSITCPPPMSVEHADIWVKSYSLYSRERYICNSGFKRKAGTSSLTECVLNKATNVAHWTTPSLK
CIRDPALVHQRPAPPS
Fc-delK-GSPLGLAGSGRSDNQGSGA-IL-15(S162A)-GGSGGSGGS-IL-15Rα (SEQ
ID NO: 427)
>P2167_Fc_delK-PS106-L9R (IL15-S162A)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGGSPLGLAGSGRSDNQGSGANWVNVISDLKKIEDLIQSMHIDATLYTESDVHPSCKVT
AMKCFLLELQVISLESGDASIHDTVENLIILANNSLSSNGNVTESGCKECEELEEKNIKEFLQSFVHIVQMFINT
AGGSGGSGGSITCPPPMSVEHADIWVKSYSLYSRERYICNSGFKRKAGTSSLTECVLNKATNVAHWTTPSLKCIR
DPALVHQRPAPPS
Fc-delK-GSPLGLAGSGRSDNQGSGA-IL-15(S162A)-GGSGGS-IL-15Rα (SEQ ID
NO: 428)
>P2168_Fc_delK-PS106-L6R (IL15-S162A)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGGSPLGLAGSGRSDNQGSGANWVNVISDLKKIEDLIQSMHIDATLYTESDVHPSCKVT
AMKCFLLELQVISLESGDASIHDTVENLIILANNSLSSNGNVTESGCKECEELEEKNIKEFLQSFVHIVQMFINT
AGGSGGSITCPPPMSVEHADIWVKSYSLYSRERYICNSGFKRKAGTSSLTECVLNKATNVAHWTTPSLKCIRDPA
LVHQRPAPPS
Fc-delK-GSPLGLAGSGRSDNQGSGA-IL-15(S162A)-GGS-IL-15Rα (SEQ ID
NO: 429)
>P2169_Fc_delK-PS106-L3R (IL15-S162A)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGGSPLGLAGSGRSDNQGSGANWVNVISDLKKIEDLIQSMHIDATLYTESDVHPSCKVT
AMKCFLLELQVISLESGDASIHDTVENLIILANNSLSSNGNVTESGCKECEELEEKNIKEFLQSFVHIVQMFINT
AGGSITCPPPMSVEHADIWVKSYSLYSRERYICNSGFKRKAGTSSLTECVLNKATNVAHWTTPSLKCIRDPALVH
QRPAPPS

Examples of Protease Cleavage Products

Expected cleavage products from uPA/Matriptase
cleavage of P1482:
1) Fc-GSLSGR (product from complete cleavage)
Chains 1 and 2
(SEQ ID NO: 430)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGKGSLSGR
2) SDNQGS-IL-15-(GGS)3-IL-15Rα (product from
complete cleavage, active)
Chains 1 and 2
(SEQ ID NO: 431)
SDNQGSNWVNVISDLKKIEDLIQSMHIDATLYTESDVHPSCKVTAMKCEL
LELQVISLESGDASIHDTVENLIILANNSLSSNGNVTESGCKECEELEEK
NIKEFLQSFVHIVQMFINTSGGSGGSGGSGGSGGSITCPPPMSVEHADIW
VKSYSLYSRERYICNSGFKRKAGTSSLTECVLNKATNVAHWTTPSLKCIR
DPALVHQRPAPPS
3) Fc-GSLSGRSDNQGS-IL-15-(GGS)3-IL-15Rα/Fc-
GSLSGR/SDNQGS-IL-15-(GGS)3-IL-15Rα (product
from partial cleavage, active)
Chain 1: Fc-GSLSGRSDNQGS-IL-15-(GGS)3-IL-15Rα
(SEQ ID NO: 432)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGKGSLSGRSDNQGSNWVNVI
SDLKKIEDLIQSMHIDATLYTESDVHPSCKVTAMKCFLLELQVISLESGD
ASIHDTVENLIILANNSLSSNGNVTESGCKECEELEEKNIKEFLQSFVHI
VQMFINTSGGSGGSGGSGGSGGSITCPPPMSVEHADIWVKSYSLYSRERY
ICNSGFKRKAGTSSLTECVLNKATNVAHWTTPSLKCIRDPALVHQRPAPP
S
Chain 2: Fc-GSLSGR
(SEQ ID NO: 433)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGKGSLSGR
Chain 3: SDNQGS-IL-15-(GGS)3-IL-15Rα
(SEQ ID NO: 434)
SDNQGSNWVNVISDLKKIEDLIQSMHIDATLYTESDVHPSCKVTAMKCEL
LELQVISLESGDASIHDTVENLIILANNSLSSNGNVTESGCKECEELEEK
NIKEFLQSFVHIVQMFINTSGGSGGSGGSGGSGGSITCPPPMSVEHADIW
VKSYSLYSRERYICNSGFKRKAGTSSLTECVLNKATNVAHWTTPSLKCIR
DPALVHQRPAPPS
Expected cleavage products from Legumain cleavage
of P1482:
1) Fc-GSLSGRSDN (product from complete cleavage)
Chains 1 and 2
(SEQ ID NO: 435)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGKGSLSGRSDN
2) QGS-IL-15-(GGS)3-IL-15Rα (product from
complete cleavage, active)
Chains 1 and 2
(SEQ ID NO: 436)
QGSNWVNVISDLKKIEDLIQSMHIDATLYTESDVHPSCKVTAMKCFLLEL
QVISLESGDASIHDTVENLIILANNSLSSNGNVTESGCKECEELEEKNIK
EFLQSFVHIVQMFINTSGGSGGSGGSGGSGGSITCPPPMSVEHADIWVKS
YSLYSRERYICNSGFKRKAGTSSLTECVLNKATNVAHWTTPSLKCIRDPA
LVHQRPAPPS
3) Fc-GSLSGRSDNQGS-IL-15-(GGS)3-IL-15Rα/Fc-
GSLSGRSDN/QGS-IL-15-(GGS)3-IL-15Rα (product
from partial cleavage, active)
Chain 1: Fc-GSLSGRSDNQGS-IL-15-(GGS)3-IL-15Rα
(SEQ ID NO: 437)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGKGSLSGRSDNQGSNWVNVI
SDLKKIEDLIQSMHIDATLYTESDVHPSCKVTAMKCFLLELQVISLESGD
ASIHDTVENLIILANNSLSSNGNVTESGCKECEELEEKNIKEFLQSFVHI
VQMFINTSGGSGGSGGSGGSGGSITCPPPMSVEHADIWVKSYSLYSRERY
ICNSGFKRKAGTSSLTECVLNKATNVAHWTTPSLKCIRDPALVHQRPAPP
S
Chain 2: Fc-GSLSGRSDN
(SEQ ID NO: 438)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGKGSLSGRSDN
Chain 3: QGS-IL-15-(GGS)3-IL-15Rα
(SEQ ID NO: 439)
QGSNWVNVISDLKKIEDLIQSMHIDATLYTESDVHPSCKVTAMKCFLLEL
QVISLESGDASIHDTVENLIILANNSLSSNGNVTESGCKECEELEEKNIK
EFLQSFVHIVQMFINTSGGSGGSGGSGGSGGSITCPPPMSVEHADIWVKS
YSLYSRERYICNSGFKRKAGTSSLTECVLNKATNVAHWTTPSLKCIRDPA
LVHQRPAPPS

Expected Cleavage Products from uPA/Matriptase Cleavage of P1540:

1) Fc-PLGLAGSGR (product from complete cleavage)
Chains 1 and 2
(SEQ ID NO: 440)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGKPLGLAGSGR
2) SDNR-IL-15(S162A)-(GGS)3-IL-15Rα (product
from complete cleavage, active)
Chains 1 and 2
(SEQ ID NO: 441)
SDNRNWVNVISDLKKIEDLIQSMHIDATLYTESDVHPSCKVTAMKCFLLE
LQVISLESGDASIHDTVENLIILANNSLSSNGNVTESGCKECEELEEKNI
KEFLQSFVHIVQMFINTAGGSGGSGGSGGSGGSITCPPPMSVEHADIWVK
SYSLYSRERYICNSGFKRKAGTSSLTECVLNKATNVAHWTTPSLKCIRDP
ALVHQRPAPPS
3) Fc-PLGLAGSGRSDNR-IL-15(S162A)-(GGS)3-IL-
15Rα/Fc-PLGLAGSGR/SDNR-IL-15(S162A)-(GGS)3-IL-
15Rα (product from partial cleavage, active)
Chain 1: Fc-PLGLAGSGRSDNR-IL-15(S162A)-(GGS)3-IL-
15Rα
(SEQ ID NO: 442)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGKPLGLAGSGRSDNRNWVNV
ISDLKKIEDLIQSMHIDATLYTESDVHPSCKVTAMKCFLLELQVISLESG
DASIHDTVENLIILANNSLSSNGNVTESGCKECEELEEKNIKEFLQSFVH
IVQMFINTAGGSGGSGGSGGSGGSITCPPPMSVEHADIWVKSYSLYSRER
YICNSGFKRKAGTSSLTECVLNKATNVAHWTTPSLKCIRDPALVHQRPAP
PS
Chain 2: Fc-PLGLAGSGR
(SEQ ID NO: 443)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGKPLGLAGSGR
Chain 3: SDNR-IL-15(S162A)-(GGS)3-IL-15Rα
(SEQ ID NO: 44)
SDNRNWVNVISDLKKIEDLIQSMHIDATLYTESDVHPSCKVTAMKCFLLE
LQVISLESGDASIHDTVENLIILANNSLSSNGNVIESGCKECEELEEKNI
KEFLQSFVHIVQMFINTAGGSGGSGGSGGSGGSITCPPPMSVEHADIWVK
SYSLYSRERYICNSGFKRKAGTSSLTECVLNKAINVAHWTTPSLKCIRDP
ALVHQRPAPPS

Expected Cleavage Products from MMP-2/MMP-9 Cleavage of P1540:

1) Fc-PLG (product from complete cleavage)
Chains 1 and 2
(SEQ ID NO: 445)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGKPLG
2) LAGSGRSDNR-IL-15(S162A)-(GGS)3-IL-15Rα
(product from complete cleavage, active)
Chains 1 and 2
(SEQ ID NO: 446)
LAGSGRSDNRNWVNVISDLKKIEDLIQSMHIDATLYTESDVHPSCKVTAM
KCFLLELQVISLESGDASIHDTVENLIILANNSLSSNGNVTESGCKECEE
LEEKNIKEFLQSFVHIVQMFINTAGGSGGSGGSGGSGGSITCPPPMSVEH
ADIWVKSYSLYSRERYICNSGFKRKAGTSSLTECVLNKATNVAHWTTPSL
KCIRDPALVHQRPAPPS
3) Fc-PLGLAGSGRSDNR-IL-15(S162A)-(GGS)3-IL-15Rα/
Fc-PLG/LAGSGRSDNR-IL-15(S162A)-(GGS)3-IL-15Rα
(product from partial cleavage, active)
Chain 1: Fc-PLGLAGSGRSDNR-IL-15(S162A)-(GGS)3-IL-
15Rα
(SEQ ID NO: 447)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGKPLGLAGSGRSDNRNWVNV
ISDLKKIEDLIQSMHIDATLYTESDVHPSCKVTAMKCFLLELQVISLESG
DASIHDTVENLIILANNSLSSNGNVTESGCKECEELEEKNIKEFLQSFVH
IVQMFINTAGGSGGSGGSGGSGGSITCPPPMSVEHADIWVKSYSLYSRER
YICNSGFKRKAGTSSLTECVLNKATNVAHWTTPSLKCIRDPALVHQRPAP
PS
Chain 2: Fc-PLG
(SEQ ID NO: 448)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGKPLG
Chain 3: LAGSGRSDNR-IL-15(S162A)-(GGS)3-IL-15Rα
(SEQ ID NO: 449)
LAGSGRSDNRNWVNVISDLKKIEDLIQSMHIDATLYTESDVHPSCKVTAM
KCFLLELQVISLESGDASIHDTVENLIILANNSLSSNGNVTESGCKECEE
LEEKNIKEFLQSFVHIVQMFINTAGGSGGSGGSGGSGGSITCPPPMSVEH
ADIWVKSYSLYSRERYICNSGFKRKAGTSSLTECVLNKATNVAHWTTPSL
KCIRDPALVHQRPAPPS

Expected Cleavage Products from uPA/Matriptase Cleavage of P1636:

1) Fc-delK-GGGSKTHTGGGS-IL-15(S162A)-GSPLGLAGSGR
(product from complete cleavage)
Chains 1 and 2
(SEQ ID NO: 450)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGGGGSKTHTGGGSNWVNVIS
DLKKIEDLIQSMHIDATLYTESDVHPSCKVTAMKCFLLELQVISLESGDA
SIHDTVENLIILANNSLSSNGNVTESGCKECEELEEKNIKEFLQSFVHIV
QMFINTAGSPLGLAGSGR
2) SDNRGS-IL-15Rα (product from complete cleavage,
active)
Chains 1 and 2
(SEQ ID NO: 451)
SDNRGSITCPPPMSVEHADIWVKSYSLYSRERYICNSGFKRKAGTSSLTE
CVLNKATNVAHWTTPSLKCIRDPALVHQRPAPPS
3) Fc-delK-GGGSKTHTGGGS-IL-15(S162A)-GSPLGLAGSGRSD
NRGS-IL-15Rα/Fc-delK-GGGSKTHTGGGS-IL-15(S162A)-
GSPLGLAGSGR/DNRGS-IL-15Rα (product from partial
cleavage, active)
Chain 1: Fc-delK-GGGSKTHTGGGS-IL-15(S162A)-
GSPLGLAGSGRSDNRGS-IL-15Rα
(SEQ ID NO: 452)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGGGGSKTHTGGGSNWVNVIS
DLKKIEDLIQSMHIDATLYTESDVHPSCKVTAMKCFLLELQVISLESGDA
SIHDTVENLIILANNSLSSNGNVTESGCKECEELEEKNIKEFLQSFVHIV
QMFINTAGSPLGLAGSGRSDNRGSITCPPPMSVEHADIWVKSYSLYSRER
YICNSGFKRKAGTSSLTECVLNKATNVAHWTTPSLKCIRDPALVHQRPAP
PS
Chain 2: Fc-delK-GGGSKTHTGGGS-IL-15(S162A)-
GSPLGLAGSGR
(SEQ ID NO: 453)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGGGGSKTHTGGGSNWVNVIS
DLKKIEDLIQSMHIDATLYTESDVHPSCKVTAMKCFLLELQVISLESGDA
SIHDTVENLIILANNSLSSNGNVIESGCKECEELEEKNIKEFLQSFVHIV
QMFINTAGSPLGLAGSGR
Chain 3: SDNRGS-IL-15Rα
(SEQ ID NO: 454)
SDNRGSITCPPPMSVEHADIWVKSYSLYSRERYICNSGFKRKAGTSSLTE
CVLNKAINVAHWTTPSLKCIRDPALVHQRPAPPS

Expected Cleavage Products from MMP-2/MMP-9 Cleavage of P1636:

1) Fc-delK-GGGSKTHTGGGS-IL-15(S162A)-GSPLG
(product from complete cleavage)
Chains 1 and 2
(SEQ ID NO: 455)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGGGGSKTHTGGGSNWVNVIS
DLKKIEDLIQSMHIDATLYTESDVHPSCKVTAMKCFLLELQVISLESGDA
SIHDTVENLIILANNSLSSNGNVTESGCKECEELEEKNIKEFLQSFVHIV
QMFINTAGSPLG
2) LAGSGRSDNRGS-IL-15Rα (product from complete
cleavage, active)
Chains 1 and 2
(SEQ ID NO: 456)
LAGSGRSDNRGSITCPPPMSVEHADIWVKSYSLYSRERYICNSGFKRKAG
TSSLTECVLNKATNVAHWTTPSLKCIRDPALVHQRPAPPS
3) Fc-delK-GGGSKTHTGGGS-IL-15(S162A)-
GSPLGLAGSGRSDNRGS-IL-15Rα/Fc-delK-GGGSKTHTGGGS-
IL-15(S162A)-GSPLG/LAGSGRSDNRGS-IL-15Rα
(product from partial cleavage, active)
Chain 1: Fc-delK-GGGSKTHTGGGS-IL-15(S162A)-
GSPLGLAGSGRSDNRGS-IL-15Rα
(SEQ ID NO: 457)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGGGGSKTHTGGGSNWVNVIS
DLKKIEDLIQSMHIDATLYTESDVHPSCKVTAMKCFLLELQVISLESGDA
SIHDTVENLIILANNSLSSNGNVTESGCKECEELEEKNIKEFLQSFVHIV
QMFINTAGSPLGLAGSGRSDNRGSITCPPPMSVEHADIWVKSYSLYSRER
YICNSGFKRKAGTSSLTECVLNKATNVAHWTTPSLKCIRDPALVHQRPAP
PS
Chain 2: Fc-delK-GGGSKTHTGGGS-IL-15(S162A)-GSPLG
(SEQ ID NO: 458)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGGGGSKTHIGGGSNWVNVIS
DLKKIEDLIQSMHIDATLYTESDVHPSCKVTAMKCELLELQVISLESGDA
SIHDTVENLIILANNSLSSNGNVIESGCKECEELEEKNIKEFLQSFVHIV
QMFINTAGSPLG
Chain 3: LAGSGRSDNRGS-IL-15Rα
(SEQ ID NO: 459)
LAGSGRSDNRGSITCPPPMSVEHADIWVKSYSLYSRERYICNSGFKRKAG
TSSLTECVLNKATNVAHWTTPSLKCIRDPALVHQRPAPPS

Expected Cleavage Products from uPA/Matriptase Cleavage of P1658:

1) Fc-delK-PLGLAGSGR (product from complete
cleavage)
Chains 1 and 2
(SEQ ID NO: 460)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGPLGLAGSGR
2) SDNQGA-IL-15(S162A)-(GGS)3-IL-15Rα
(product from complete cleavage, active)
Chains 1 and 2
(SEQ ID NO: 461)
SDNQGANWVNVISDLKKIEDLIQSMHIDATLYTESDVHPSCKVTAMKCFL
LELQVISLESGDASIHDTVENLIILANNSLSSNGNVTESGCKECEELEEK
NIKEFLQSFVHIVQMFINTAGGSGGSGGSGGSGGSITCPPPMSVEHADIW
VKSYSLYSRERYICNSGFKRKAGTSSLTECVLNKATNVAHWTTPSLKCIR
DPALVHQRPAPPS
3) Fc-delK-PLGLAGSGRSDNQGA-IL-15(S162A)-(GGS)3-IL-
15Rα/Fc-delK-PLGLAGSGR/SDNQGA-IL-15(S162A)-
(GGS)3-IL-15Rα (product from partial cleavage,
active)
Chain 1: Fc-delK-PLGLAGSGRSDNQGA-IL-15(S162A)-
(GGS)3-IL-15Rα
(SEQ ID NO: 462)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGPLGLAGSGRSDNQGANWVN
VISDLKKIEDLIQSMHIDATLYTESDVHPSCKVTAMKCFLLELQVISLES
GDASIHDTVENLIILANNSLSSNGNVTESGCKECEELEEKNIKEFLQSFV
HIVQMFINTAGGSGGSGGSGGSGGSITCPPPMSVEHADIWVKSYSLYSRE
RYICNSGFKRKAGTSSLTECVLNKATNVAHWTTPSLKCIRDPALVHQRPA
PPS
Chain 2: Fc-delK-PLGLAGSGR
(SEQ ID NO: 463)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGPLGLAGSGR
Chain 3: SDNQGA-IL-15(S162A)-(GGS)3-IL-15Rα
(SEQ ID NO: 464)
SDNQGANWVNVISDLKKIEDLIQSMHIDATLYTESDVHPSCKVTAMKCFL
LELQVISLESGDASIHDTVENLIILANNSLSSNGNVIESGCKECEELEEK
NIKEFLQSFVHIVQMFINTAGGSGGSGGSGGSGGSITCPPPMSVEHADIW
VKSYSLYSRERYICNSGFKRKAGTSSLTECVLNKAINVAHWTTPSLKCIR
DPALVHQRPAPPS

Expected Cleavage Products from MMP-2/MMP-9 Cleavage of P1658:

1) Fc-delK-PLG (product from complete cleavage)
Chains 1 and 2
(SEQ ID NO: 465)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGPLG
2) LAGSGRSDNQGA-IL-15(S162A)-(GGS)3-IL-15Rα
(product from complete cleavage, active)
Chains 1 and 2
(SEQ ID NO: 466)
LAGSGRSDNQGANWVNVISDLKKIEDLIQSMHIDATLYTESDVHPSCKVT
AMKCFLLELQVISLESGDASIHDTVENLIILANNSLSSNGNVTESGCKEC
EELEEKNIKEFLQSFVHIVQMFINTAGGSGGSGGSGGSGGSITCPPPMSV
EHADIWVKSYSLYSRERYICNSGFKRKAGTSSLTECVLNKATNVAHWTTP
SLKCIRDPALVHQRPAPPS
3) Fc-delK-PLGLAGSGRSDNQGA-IL-15(S162A)-(GGS)3-IL-
15Rα/Fc-delK-PLG/LAGSGRSDNQGA-IL-15(S162A)-
(GGS)3-IL-15Rα (product from partial cleavage,
active)
Chain 1: Fc-delK-PLGLAGSGRSDNQGA-IL-15(S162A)-
(GGS)3-IL-15Rα
(SEQ ID NO: 467)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGPLGLAGSGRSDNQGANWVN
VISDLKKIEDLIQSMHIDATLYTESDVHPSCKVTAMKCFLLELQVISLES
GDASIHDTVENLIILANNSLSSNGNVTESGCKECEELEEKNIKEFLQSFV
HIVQMFINTAGGSGGSGGSGGSGGSITCPPPMSVEHADIWVKSYSLYSRE
RYICNSGFKRKAGTSSLTECVLNKATNVAHWTTPSLKCIRDPALVHQRPA
PPS
Chain 2: Fc-delK-PLG
(SEQ ID NO: 468)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGPLG
Chain 3: LAGSGRSDNQGA-IL-15(S162A)-(GGS)3-IL-15Rα
(SEQ ID NO: 469)
LAGSGRSDNQGANWVNVISDLKKIEDLIQSMHIDATLYTESDVHPSCKVT
AMKCFLLELQVISLESGDASIHDTVENLIILANNSLSSNGNVTESGCKEC
EELEEKNIKEFLQSFVHIVQMFINTAGGSGGSGGSGGSGGSITCPPPMSV
EHADIWVKSYSLYSRERYICNSGFKRKAGTSSLTECVLNKATNVAHWTTP
SLKCIRDPALVHQRPAPPS

Expected Cleavage Products from uPA/Matriptase Cleavage of P1701:

1) Fc-delK-GG-IL-15(S162A)-GGSGGSPLGLAGSGR
(product from complete cleavage)
Chains 1 and 2
(SEQ ID NO: 470)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGGGNWVNVISDLKKIEDLIQ
SMHIDATLYTESDVHPSCKVTAMKCFLLELQVISLESGDASIHDTVENLI
ILANNSLSSNGNVTESGCKECEELEEKNIKEFLQSFVHIVQMFINTAGGS
GGSPLGLAGSGR
2) SDNQGGSGGA-IL-15Rα(T2A) (product from complete
cleavage, active)
Chains 1 and 2
(SEQ ID NO: 471)
SDNQGGSGGAIACPPPMSVEHADIWVKSYSLYSRERYICNSGFKRKAGTS
SLTECVLNKATNVAHWTTPSLKCIRDPALVHQRPAPPS
3) Fc-delK-GG-IL-15(S162A)-
GGSGGSPLGLAGSGRSDNQGGSGGA-IL-15Rα(T2A)/Fc-delK-
GG-IL-15(S162A)-GGSGGSPLGLAGSGR/SDNQGGSGGA-IL-
15Rα(T2A) (product from partial cleavage,
active)
Chain 1: Fc-delK-GG-IL-15(S162A)-
GGSGGSPLGLAGSGRSDNQGGSGGA-IL-15Rα(T2A)
(SEQ ID NO: 472)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGGGNWVNVISDLKKIEDLIQ
SMHIDATLYTESDVHPSCKVTAMKCFLLELQVISLESGDASIHDTVENLI
ILANNSLSSNGNVTESGCKECEELEEKNIKEFLQSFVHIVQMFINTAGGS
GGSPLGLAGSGRSDNQGGSGGAIACPPPMSVEHADIWVKSYSLYSRERYI
CNSGFKRKAGTSSLTECVLNKATNVAHWTTPSLKCIRDPALVHQRPAPPS
Chain 2: Fc-delK-GG-IL-15(S162A)-GGSGGSPLGLAGSGR
(SEQ ID NO: 473)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGGGNWVNVISDLKKIEDLIQ
SMHIDATLYTESDVHPSCKVTAMKCFLLELQVISLESGDASIHDTVENLI
ILANNSLSSNGNVTESGCKECEELEEKNIKEFLQSFVHIVQMFINTAGGS
GGSPLGLAGSGR
Chain 3: SDNQGGSGGA-IL-15Rα(T2A)
(SEQ ID NO: 474)
SDNQGGSGGAIACPPPMSVEHADIWVKSYSLYSRERYICNSGFKRKAGTS
SLTECVLNKATNVAHWTTPSLKCIRDPALVHQRPAPPS

Expected Cleavage Products from MMP-2/MMP-9 Cleavage of P1701:

1) Fc-delK-GG-IL-15(S162A)-GGSGGSPLG (product from
complete cleavage)
Chains 1 and 2
(SEQ ID NO: 475)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGGGNWVNVISDLKKIEDLIQ
SMHIDATLYTESDVHPSCKVTAMKCFLLELQVISLESGDASIHDTVENLI
ILANNSLSSNGNVTESGCKECEELEEKNIKEFLQSFVHIVQMFINTAGGS
GGSPLG
2) LAGSGRSDNQGGSGGA-IL-15Rα(T2A) (product from
complete cleavage, active)
Chains 1 and 2
(SEQ ID NO: 476)
LAGSGRSDNQGGSGGAIACPPPMSVEHADIWVKSYSLYSRERYICNSGFK
RKAGTSSLTECVLNKATNVAHWTTPSLKCIRDPALVHQRPAPPS
3) Fc-delK-GG-IL-15(S162A)-
GGSGGSPLGLAGSGRSDNQGGSGGA-IL-15Rα(T2A)/Fc-delK-
GG-IL-15(S162A)-GGSGGSPLG/LAGSGRSDNQGGSGGA-IL-15Rα
(T2A) (product from partial cleavage, active)
Chain 1: Fc-delK-GG-IL-15(S162A)-
GGSGGSPLGLAGSGRSDNQGGSGGA-IL-15Rα(T2A)
(SEQ ID NO: 477)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGGGNWVNVISDLKKIEDLIQ
SMHIDATLYTESDVHPSCKVTAMKCFLLELQVISLESGDASIHDTVENLI
ILANNSLSSNGNVTESGCKECEELEEKNIKEFLQSFVHIVQMFINTAGGS
GGSPLGLAGSGRSDNQGGSGGAIACPPPMSVEHADIWVKSYSLYSRERYI
CNSGFKRKAGTSSLTECVLNKATNVAHWTTPSLKCIRDPALVHQRPAPPS
Chain 2: Fc-delK-GG-IL-15(S162A)-GGSGGSPLG
(SEQ ID NO: 478)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGGGNWVNVISDLKKIEDLIQ
SMHIDATLYTESDVHPSCKVTAMKCFLLELQVISLESGDASIHDTVENLI
ILANNSLSSNGNVTESGCKECEELEEKNIKEFLQSFVHIVQMFINTAGGS
GGSPLG
Chain 3: LAGSGRSDNQGGSGGA-IL-15Rα(T2A)
(SEQ ID NO: 479)
LAGSGRSDNQGGSGGAIACPPPMSVEHADIWVKSYSLYSRERYICNSGFK
RKAGTSSLTECVLNKATNVAHWTTPSLKCIRDPALVHQRPAPPS

Expected Cleavage Products from TEV Cleavage of P1973:

1) Fc-G-IL-15(S162A)-GGSGGSEPKENLYFQ (product from
complete cleavage)
Chains 1 and 2
(SEQ ID NO: 480)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGKGNWVNVISDLKKIEDLIQ
SMHIDATLYTESDVHPSCKVTAMKCFLLELQVISLESGDASIHDTVENLI
ILANNSLSSNGNVTESGCKECEELEEKNIKEFLQSFVHIVQMFINTAGGS
GGSEPKENLYFQ
2) GGGSGGSGGGS-IL-15Rα(A50C, T58C) (product
from complete cleavage, active)
Chains 1 and 2
(SEQ ID NO: 481)
GGGSGGSGGGSITCPPPMSVEHADIWVKSYSLYSRERYICNSGFKRKAGT
SSLTECVLNKCTNVAHWTCPSLKCIRDPALVHQRPAPPS
3) Fc-G-IL-15(S162A)-GGSGGSEPKENLYFQGGGSGGSGGGS-
IL-15Rα(A50C, T58C)/Fc-G-IL-15(S162A)-GGSGGSEPK
ENLYFQ/GGGSGGSGGGS-IL-15Rα(A50C, T58C)
(product from partial cleavage, active)
Chain 1: Fc-G-IL-15(S162A)-
GGSGGSEPKENLYFQGGGSGGSGGGS-IL-15Rα(A50C, T58C)
(SEQ ID NO: 482)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGKGNWVNVISDLKKIEDLIQ
SMHIDATLYTESDVHPSCKVTAMKCFLLELQVISLESGDASIHDTVENLI
ILANNSLSSNGNVTESGCKECEELEEKNIKEFLQSFVHIVQMFINTAGGS
GGSEPKENLYFQGGGSGGSGGGSITCPPPMSVEHADIWVKSYSLYSRERY
ICNSGFKRKAGTSSLTECVLNKCTNVAHWTCPSLKCIRDPALVHQRPAPP
S
Chain 2: Fc-G-IL-15(S162A)-GGSGGSEPKENLYFQ
(SEQ ID NO: 483)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGKGNWVNVISDLKKIEDLIQ
SMHIDATLYTESDVHPSCKVTAMKCFLLELQVISLESGDASIHDTVENLI
ILANNSLSSNGNVTESGCKECEELEEKNIKEFLQSFVHIVQMFINTAGGS
GGSEPKENLYFQ
Chain 3: GGGSGGSGGGS-IL-15Rα(A50C, T58C)
(SEQ ID NO: 484)
GGGSGGSGGGSITCPPPMSVEHADIWVKSYSLYSRERYICNSGFKRKAGT
SSLTECVLNKCTNVAHWTCPSLKCIRDPALVHQRPAPPS

Expected Cleavage Products from uPA/Matriptase Cleavage of P2169:

1) Fc-delK-GSPLGLAGSGR (product from complete
cleavage)
Chains 1 and 2
(SEQ ID NO: 485)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGGSPLGLAGSGR
2) SDNQGSGA-IL-15(S162A)-GGS-IL-15Rα (product
from complete cleavage, active)
Chains 1 and 2
(SEQ ID NO: 486)
SDNQGSGANWVNVISDLKKIEDLIQSMHIDATLYTESDVHPSCKVTAMKC
FLLELQVISLESGDASIHDTVENLIILANNSLSSNGNVTESGCKECEELE
EKNIKEFLQSFVHIVQMFINTAGGSITCPPPMSVEHADIWVKSYSLYSRE
RYICNSGFKRKAGTSSLTECVLNKATNVAHWTTPSLKCIRDPALVHQRPA
PPS
3) Fc-delK-GSPLGLAGSGRSDNQGSGA-IL-15(S162A)-GGS-IL-
15Rα/Fc-delK-GSPLGLAGSGR/SDNQGSGA-IL-15(S162A)-
GGS-IL-15Rα (product from partial cleavage,
active)
Chain 1: Fc-delK-GSPLGLAGSGRSDNQGSGA-IL-15(S162A)-
GGS-IL-15Rα
(SEQ ID NO: 487)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGGSPLGLAGSGRSDNQGSGA
NWVNVISDLKKIEDLIQSMHIDATLYTESDVHPSCKVTAMKCFLLELQVI
SLESGDASIHDTVENLIILANNSLSSNGNVTESGCKECEELEEKNIKEFL
QSFVHIVQMFINTAGGSITCPPPMSVEHADIWVKSYSLYSRERYICNSGF
KRKAGTSSLTECVLNKATNVAHWTTPSLKCIRDPALVHQRPAPPS
Chain 2: Fc-delK-GSPLGLAGSGR
(SEQ ID NO: 488)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGGSPLGLAGSGR
Chain 3: SDNQGSGA-IL-15(S162A)-GGS-IL-15Rα
(SEQ ID NO: 489)
SDNQGSGANWVNVISDLKKIEDLIQSMHIDATLYTESDVHPSCKVTAMKC
FLLELQVISLESGDASIHDTVENLIILANNSLSSNGNVTESGCKECEELE
EKNIKEFLQSFVHIVQMFINTAGGSITCPPPMSVEHADIWVKSYSLYSRE
RYICNSGFKRKAGTSSLTECVLNKATNVAHWTTPSLKCIRDPALVHQRPA
PPS

Expected Cleavage Products from MMP-2/MMP-9 Cleavage of P2169:

1) Fc-delK-GSPLG (product from complete cleavage)
Chains 1 and 2
(SEQ ID NO: 490)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGGSPLG
2) LAGSGRSDNQGSGA-IL-15(S162A)-GGS-IL-15Rα (product from
complete cleavage, active)
Chains 1 and 2
(SEQ ID NO: 491)
LAGSGRSDNQGSGANWVNVISDLKKIEDLIQSMHIDATLYTESDVHPSCKVTAMKCFLLELQV
ISLESGDASIHDTVENLIILANNSLSSNGNVTESGCKECEELEEKNIKEFLQSFVHIVQMFINTAGGSITCPPPM
SVEHADIWVKSYSLYSRERYICNSGFKRKAGTSSLTECVLNKATNVAHWTTPSLKCIRDPALVHQRPAPPS
3) Fc-delK-GSPLGLAGSGRSDNQGSGA-IL-15(S162A)-GGS-IL-15Rα/Fc-
delK-GSPLG/LAGSGRSDNQGSGA-IL-15(S162A)-GGS-IL-15Rα (product from partial
cleavage, active)
Chain 1: Fc-delK-GSPLGLAGSGRSDNQGSGA-IL-15(S162A)-GGS-IL-15Rα
(SEQ ID NO: 492)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGGSPLGLAGSGRSDNQGSGANWVNVISDLKKIEDLIQSMHIDATLYTESDVHPSCKVT
AMKCFLLELQVISLESGDASIHDTVENLIILANNSLSSNGNVTESGCKECEELEEKNIKEFLQSFVHIVQMFINT
AGGSITCPPPMSVEHADIWVKSYSLYSRERYICNSGFKRKAGTSSLTECVLNKATNVAHWTTPSLKCIRDPALVH
QRPAPPS
Chain 2: Fc-delK-GSPLG
(SEQ ID NO: 493)
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVFSCSVM
HEALHNHYTQKSLSLSPGGSPLG
Chain 3: LAGSGRSDNQGSGA-IL-15(S162A)-GGS-IL-15Rα
(SEQ ID NO: 494)
LAGSGRSDNQGSGANWVNVISDLKKIEDLIQSMHIDATLYTESDVHPSCKVTAMKCFLLELQV
ISLESGDASIHDTVENLIILANNSLSSNGNVTESGCKECEELEEKNIKEFLQSFVHIVQMFINTAGGSITCPPPM
SVEHADIWVKSYSLYSRERYICNSGFKRKAGTSSLTECVLNKATNVAHWTTPSLKCIRDPALVHQRPAPPS
P15431450: FAP-H1L1-huIgG1-PLGLAGSGRSDNR-IL-15(S162A)-
GGSGGSGGSGGSGGS-IL-15Rα
>Chains 1 and 2: FAP-H1-hG1-PS53-L15R
(SEQ ID NO: 495)
EVQLVQSGAEVKKPGESLKISCKGSGYTFTENIIHWVRQMPGKGLEWIGWFHPGSGSIKYNEKEKDQ
VTISADKSISTAYLQWSSLKASDTAMYFCARHGGTGRGAMDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKK
VEPKSCDKTHTCPPCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCSVMHEALHNHYTQK
SLSLSPGKPLGLAGSGRSDNRNwvnvisdlkkiedliqsmhidatlytesdvhpsckvtamkcfllelqvisles
gdasihdtvenliilannslssngnvtesgckeceeleeknikeflqsfvhivqmfintAGGSGGSGGSGGSGGS
ITCPPPMSVEHADIWVKSYSLYSRERYICNSGFKRKAGTSSLTECVLNKATNVAHWTTPSLKCIRDPALVHQRPA
PPS
>Chains 3 and 4: FAP-L1-huIgKLC
(SEQ ID NO: 496)
DIVMTQTPLSLSVTPGQPASISCRASKSVSTSAYSYMHWYLQKPGQSPQLLIYLASNLESGVP
DRFSGSGSGTDFTLKISRVEAEDVGVYYCQHSRELPYTEGQGTKLEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSSPVTKSF
NRGEC
P16401450: FAP-H1L1-huIgG1-delK-PLGLAGSGRSDNR-IL-15(S162A)-
GGSGGSGGSGGSGGS-IL-15Rα
>Chains 1 and 2: FAP-H1-hG1-delK-PS53-L15R
(SEQ ID NO: 497)
EVQLVQSGAEVKKPGESLKISCKGSGYTFTENIIHWVRQMPGKGLEWIGWFHPGSGSIKYNEKEKDQ
VTISADKSISTAYLQWSSLKASDTAMYFCARHGGTGRGAMDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKK
VEPKSCDKTHTCPPCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCSVMHEALHNHYTQK
SLSLSPGPLGLAGSGRSDNRNwvnvisdlkkiedliqsmhidatlytesdvhpsckvtamkcfllelqvislesg
dasihdtvenliilannslssngnvtesgckeceeleeknikeflqsfvhivqmfintAGGSGGSGGSGGSGGSI
TCPPPMSVEHADIWVKSYSLYSRERYICNSGFKRKAGTSSLTECVLNKATNVAHWTTPSLKCIRDPALVHQRPAP
PS
>Chains 3 and 4: FAP-L1-huIgKLC
(SEQ ID NO: 498)
DIVMTQTPLSLSVTPGQPASISCRASKSVSTSAYSYMHWYLQKPGQSPQLLIYLASNLESGVPDRFS
GSGSGTDFTLKISRVEAEDVGVYYCQHSRELPYTEGQGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTKSFNRGE
C
P18121450: FAP-H1L1-huIgG1(L234A, L235A, P329A)-delK-GG-IL-
15(S162A)-GGSGGSPLGLAGSGRSDNQGGSGGA-IL-15Rα(T2A)
>Chains 1 and 2: FAP-H1-huIgG1(AAA)_delK-GG-L_(S162A)-GGSGGSP
LGLAGSGRSDNQGGSGGA-R_T2A
(SEQ ID NO: 499)
EVQLVQSGAEVKKPGESLKISCKGSGYTFTENIIHWVRQMPGKGLEWIGWFHPGSGSIKYNEK
FKDQVTISADKSISTAYLQWSSLKASDTAMYFCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGNWVNVISDLKKIEDLIQSMHIDATLYTESDVHPSCKVTAMKCFLLELQVISLESGDASIHDT
VENLIILANNSLSSNGNVTESGCKECEELEEKNIKEFLQSFVHIVQMFINTAGGSGGSPLGLAGSGRSDNQGGSG
GAIACPPPMSVEHADIWVKSYSLYSRERYICNSGFKRKAGTSSLTECVLNKATNVAHWTTPSLKCIRDPALVHQR
PAPPS
>Chains 3 and 4: FAP-L1-huIgKLC
(SEQ ID NO: 500)
DIVMTQTPLSLSVTPGQPASISCRASKSVSTSAYSYMHWYLQKPGQSPQLLIYLASNLESGVP
DRFSGSGSGTDFTLKISRVEAEDVGVYYCQHSRELPYTFGQGTKLEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSSPVTKSF
NRGEC
P18131453: FAP-H2L2-huIgG1(L234A, L235A, P329A)-delK-GG-IL-
15(S162A)-GGSGGSPLGLAGSGRSDNQGGSGGA-IL-15Rα(T2A)
>Chains 1 and 2: FAP-H2-huIgG1(AAA)_delK-GG-L_(S162A)-GGSGGSPL
GLAGSGRSDNQGGSGGA-R_T2A
(SEQ ID NO: 501)
QVQLVQSGAEVKKPGASVKVSCKASGYTFTENIIHWVRQAPGQGLEWMGWFHPGSGSIKYNEK
FKDRVTMTADTSTSTVYMELSSLRSEDTAVYYCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGNWVNVISDLKKIEDLIQSMHIDATLYTESDVHPSCKVTAMKCFLLELQVISLESGDASIHDT
VENLIILANNSLSSNGNVTESGCKECEELEEKNIKEFLQSFVHIVQMFINTAGGSGGSPLGLAGSGRSDNQGGSG
GAIACPPPMSVEHADIWVKSYSLYSRERYICNSGFKRKAGTSSLTECVLNKATNVAHWTTPSLKCIRDPALVHQR
PAPPS
>Chains 3 and 4: FAP-L2-huIgKLC
(SEQ ID NO: 502)
DIQMTQSPSSLSASVGDRVTITCRASKSVSTSAYSYMHWYQQKPGKAPKLLIYLASNLESGVP
SRFSGSGSGTDFTLTISSLQPEDFATYYCQHSRELPYTEGQGTKLEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSSPVTKSF
NRGEC
P18141563: FAP-H4L4-huIgG1(L234A, L235A, P329A)-delK-GG-IL-
15(S162A)-GGSGGSPLGLAGSGRSDNQGGSGGA-IL-15Rα(T2A)
>Chains 1 and 2: FAP-H4-huIgG1(AAA)_delK-GG-L_(S162A)-GGSGGSPL
GLAGSGRSDNQGGSGGA-R_T2A
(SEQ ID NO: 503)
EVQLLESGGGLVQPGGSLRLSCAASGFTFSSYAMSWVRQAPGKGLEWVSAIIGSGASTYYADS
VKGRFTISRDNSKNTLYLQMNSLRAEDTAVYYCAKGWEGGFNYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSNTKVDK
KVEPKSCDKTHTCPPCPAPEAAGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALAAPIEKTISKAKGQPREPQVYTLPPSRDELTKNQV
SLTCLVKGFYPSDIAVEWESNGQPENNYKTTPPVLDSDGSFFLYSKLTVDKSRWQQGNVFSCSVMHEALHNHYTQ
KSLSLSPGGGNWVNVISDLKKIEDLIQSMHIDATLYTESDVHPSCKVTAMKCFLLELQVISLESGDASIHDTVEN
LIILANNSLSSNGNVTESGCKECEELEEKNIKEFLQSFVHIVQMFINTAGGSGGSPLGLAGSGRSDNQGGSGGAI
ACPPPMSVEHADIWVKSYSLYSRERYICNSGFKRKAGTSSLTECVLNKATNVAHWTTPSLKCIRDPALVHQRPAP
PS
>Chains 3 and 4: FAP-L4-huIgKLC
(SEQ ID NO: 504)
EIVLTQSPGTLSLSPGERATLSCRASQSVTSSYLAWYQQKPGQAPRLLINVGSRRATGIPDRF
SGSGSGTDFTLTISRLEPEDFAVYYCQQGIMLPPTEGQGTKVEIKRTVAAPSVFIFPPSDEQLKSGTASVVCLLN
NFYPREAKVQWKVDNALQSGNSQESVTEQDSKDSTYSLSSTLTLSKADYEKHKVYACEVTHQGLSSPVTKSFNRG
EC
P18151450: FAP-H1L1-huIgG1(L234A, L235A, P329A)-delK-GG-IL-
15(S162A)-GGSGGSPLGLAGSGRSDNHGGSGGA-IL-15Rα(T2A)
>Chains 1 and 2: FAP-H1-huIgG1(AAA)_delK-GG-L_(S162A)-GGSGGSP
LGLAGSGRSDNHGGSGGA-R_T2A
(SEQ ID NO: 505)
EVQLVQSGAEVKKPGESLKISCKGSGYTFTENIIHWVRQMPGKGLEWIGWFHPGSGSIKYNEK
FKDQVTISADKSISTAYLQWSSLKASDTAMYFCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGNWVNVISDLKKIEDLIQSMHIDATLYTESDVHPSCKVTAMKCFLLELQVISLESGDASIHDT
VENLIILANNSLSSNGNVTESGCKECEELEEKNIKEFLQSFVHIVQMFINTAGGSGGSPLGLAGSGRSDNHGGSG
GAIACPPPMSVEHADIWVKSYSLYSRERYICNSGFKRKAGTSSLTECVLNKATNVAHWTTPSLKCIRDPALVHQR
PAPPS
>Chains 3 and 4: FAP-L1-huIgKLC
(SEQ ID NO: 506)
DIVMTQTPLSLSVTPGQPASISCRASKSVSTSAYSYMHWYLQKPGQSPQLLIYLASNLESGVP
DRFSGSGSGTDFTLKISRVEAEDVGVYYCQHSRELPYTFGQGTKLEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSSPVTKSF
NRGEC
P18161453: FAP-H2L2-huIgG1(L234A, L235A, P329A)-delK-GG-IL-
15(S162A)-GGSGGSPLGLAGSGRSDNHGGSGGA-IL-15Rα(T2A)
>Chains 1 and 2: FAP-H2-huIgG1(AAA)_delK-GG-L_(S162A)-GGSGGSP
LGLAGSGRSDNHGGSGGA-R_T2A
(SEQ ID NO: 507)
QVQLVQSGAEVKKPGASVKVSCKASGYTFTENIIHWVRQAPGQGLEWMGWFHPGSGSIKYNEK
FKDRVTMTADTSTSTVYMELSSLRSEDTAVYYCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGNWVNVISDLKKIEDLIQSMHIDATLYTESDVHPSCKVTAMKCFLLELQVISLESGDASIHDT
VENLIILANNSLSSNGNVTESGCKECEELEEKNIKEFLQSFVHIVQMFINTAGGSGGSPLGLAGSGRSDNHGGSG
GAIACPPPMSVEHADIWVKSYSLYSRERYICNSGFKRKAGTSSLTECVLNKATNVAHWTTPSLKCIRDPALVHQR
PAPPS
>Chains 3 and 4: FAP-L1-huIgKLC
(SEQ ID NO: 508)
DIQMTQSPSSLSASVGDRVTITCRASKSVSTSAYSYMHWYQQKPGKAPKLLIYLASNLESGVP
SRFSGSGSGTDFTLTISSLQPEDFATYYCQHSRELPYTEGQGTKLEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSSPVTKSF
NRGEC
P18171563: FAP-H4L4-huIgG1(L234A, L235A, P329A)-delK-GG-IL-
15(S162A)-GGSGGSPLGLAGSGRSDNHGGSGGA-IL-15Rα(T2A)
>Chains 1 and 2: FAP-H4-huIgG1(AAA)_delK-GG-L_(S162A)-GGSGGSP
LGLAGSGRSDNHGGSGGA-R_T2A
(SEQ ID NO: 509)
EVQLLESGGGLVQPGGSLRLSCAASGFTFSSYAMSWVRQAPGKGLEWVSAIIGSGASTYYADS
VKGRFTISRDNSKNTLYLQMNSLRAEDTAVYYCAKGWEGGFNYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSNTKVDK
KVEPKSCDKTHTCPPCPAPEAAGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALAAPIEKTISKAKGQPREPQVYTLPPSRDELTKNQV
SLTCLVKGFYPSDIAVEWESNGQPENNYKTTPPVLDSDGSFFLYSKLTVDKSRWQQGNVFSCSVMHEALHNHYTQ
KSLSLSPGGGNWVNVISDLKKIEDLIQSMHIDATLYTESDVHPSCKVTAMKCFLLELQVISLESGDASIHDTVEN
LIILANNSLSSNGNVTESGCKECEELEEKNIKEFLQSFVHIVQMFINTAGGSGGSPLGLAGSGRSDNHGGSGGAI
ACPPPMSVEHADIWVKSYSLYSRERYICNSGFKRKAGTSSLTECVLNKATNVAHWTTPSLKCIRDPALVHQRPAP
PS
>Chains 3 and 4: FAP-L4-huIgKLC
(SEQ ID NO: 510)
EIVLTQSPGTLSLSPGERATLSCRASQSVTSSYLAWYQQKPGQAPRLLINVGSRRATGIPDRF
SGSGSGTDFTLTISRLEPEDFAVYYCQQGIMLPPTEGQGTKVEIKRTVAAPSVFIFPPSDEQLKSGTASVVCLLN
NFYPREAKVQWKVDNALQSGNSQESVTEQDSKDSTYSLSSTLTLSKADYEKHKVYACEVTHQGLSSPVTKSFNRG
EC
P19681450: FAP-H1L1-huIgG1(L234A, L235A, P329A)-delK-GG-IL-
15(K86A, S162A)-GGSGGSPLGLAGSGRSDNQGGSGGA-IL-15Rα(T2A)
>Chains 1 and 2: FAP-H1-huIgG1(AAA)_delK-GG-L_(K86A, S162A)-
GGSGGS PLGLAGSGRSDNQGGSGGA-R_T2A
(SEQ ID NO: 511)
EVQLVQSGAEVKKPGESLKISCKGSGYTFTENIIHWVRQMPGKGLEWIGWFHPGSGSIKYNEK
FKDQVTISADKSISTAYLQWSSLKASDTAMYFCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGNWVNVISDLKKIEDLIQSMHIDATLYTESDVHPSCKVTAMKCFLLELQVISLESGDASIHDT
VENLIILANNSLSSNGNVTESGCAECEELEEKNIKEFLQSFVHIVQMFINTAGGSGGSPLGLAGSGRSDNQGGSG
GAIACPPPMSVEHADIWVKSYSLYSRERYICNSGFKRKAGTSSLTECVLNKATNVAHWTTPSLKCIRDPALVHQR
PAPPS
>Chains 3 and 4: FAP-L1-huIgKLC
(SEQ ID NO: 512)
DIVMTQTPLSLSVTPGQPASISCRASKSVSTSAYSYMHWYLQKPGQSPQLLIYLASNLESGVP
DRFSGSGSGTDFTLKISRVEAEDVGVYYCQHSRELPYTFGQGTKLEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSSPVTKSF
NRGEC
P19691450: FAP-H1L1-huIgG1(L234A, L235A, P329A)-delK-GG-IL-
15(K86R, S162A)-GGSGGSPLGLAGSGRSDNQGGSGGA-IL-15Rα(T2A)
>Chains 1 and 2: FAP-H1-huIgG1(AAA)_delK-GG-L_(K86R, S162A)-
GGSGGS PLGLAGSGRSDNQGGSGGA-R_T2A
(SEQ ID NO: 513)
EVQLVQSGAEVKKPGESLKISCKGSGYTFTENIIHWVRQMPGKGLEWIGWFHPGSGSIKYNEK
FKDQVTISADKSISTAYLQWSSLKASDTAMYFCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGNWVNVISDLKKIEDLIQSMHIDATLYTESDVHPSCKVTAMKCFLLELQVISLESGDASIHDT
VENLIILANNSLSSNGNVTESGCRECEELEEKNIKEFLQSFVHIVQMFINTAGGSGGSPLGLAGSGRSDNQGGSG
GAIACPPPMSVEHADIWVKSYSLYSRERYICNSGFKRKAGTSSLTECVLNKATNVAHWTTPSLKCIRDPALVHQR
PAPPS
>Chains 3 and 4: FAP-L1-huIgKLC
(SEQ ID NO: 514)
DIVMTQTPLSLSVTPGQPASISCRASKSVSTSAYSYMHWYLQKPGQSPQLLIYLASNLESGVP
DRFSGSGSGTDFTLKISRVEAEDVGVYYCQHSRELPYTFGQGTKLEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSSPVTKSF
NRGEC
P24872158: FAP-H6L7-huIgG1(L234A, L235A, P329A)-delK-GGSPLGLAGGGS-
IL-15(S162A)-GGGSGGGSGRSDNQGGGSG-IL-15Rα(T2A)
>Chains 1 and 2: FAP-H6_huIgG1(AAA)_delK-GGS-PLGLAG-GGS-
L_(S162A)-GGGSGGG-SGRSDNQ-GGGSG-R_T2A
(SEQ ID NO: 515)
QVQLVQSGAEVKKPGASVKVSCKASGYTFTENIIHWVRQAPGQGLEWMGWIHPGSGSIKYAQK
LQGRVTMTTDTSTSTAYMELRSLRSDDTAVYYCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGSPLGLAGGGSNWVNVISDLKKIEDLIQSMHIDATLYTESDVHPSCKVTAMKCFLLELQVISL
ESGDASIHDTVENLIILANNSLSSNGNVTESGCKECEELEEKNIKEFLQSFVHIVQMFINTAGGGSGGGSGRSDN
QGGGSGIACPPPMSVEHADIWVKSYSLYSRERYICNSGFKRKAGTSSLTECVLNKATNVAHWTTPSLKCIRDPAL
VHQRPAPPS
>Chains 3 and 4: FAP-L7-huIgKLC
(SEQ ID NO: 516)
EIVLTQSPGTLSLSPGERATLSCRASQSVSTSAYSYMHWYQQKPGQAPRLLIYLASNLESGIP
DRFSGSGSGTDFTLTISRLEPEDFAVYYCQQSRELPYTEGQGTKLEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSSPVTKSF
NRGEC
P24882158: FAP-H6L7-huIgG1(L234A, L235A, P329A)-delK-GGSPLGLAGGGS-
IL-15(S162A)-GGGSGGGSGRSDNQGGGSGGG-IL-15Rα(T2A)
>Chains 1 and 2: FAP-H6_huIgG1(AAA)_delK-GGS-PLGLAG-GGS-
L_(S162A)-GGGSGGG-SGRSDNQ-GGGSGGG-R_T2A
(SEQ ID NO: 517)
QVQLVQSGAEVKKPGASVKVSCKASGYTFTENIIHWVRQAPGQGLEWMGWIHPGSGSIKYAQK
LQGRVTMTTDTSTSTAYMELRSLRSDDTAVYYCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGSPLGLAGGGSNWVNVISDLKKIEDLIQSMHIDATLYTESDVHPSCKVTAMKCFLLELQVISL
ESGDASIHDTVENLIILANNSLSSNGNVTESGCKECEELEEKNIKEFLQSFVHIVQMFINTAGGGSGGGSGRSDN
QGGGSGGGIACPPPMSVEHADIWVKSYSLYSRERYICNSGFKRKAGTSSLTECVLNKATNVAHWTTPSLKCIRDP
ALVHQRPAPPS*
>Chains 3 and 4: FAP-L7-huIgKLC
(SEQ ID NO: 518)
EIVLTQSPGTLSLSPGERATLSCRASQSVSTSAYSYMHWYQQKPGQAPRLLIYLASNLESGIP
DRFSGSGSGTDFTLTISRLEPEDFAVYYCQQSRELPYTEGQGTKLEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSSPVTKSF
NRGEC*
P24892158: FAP-H6L7-huIgG1(L234A, L235A, P329A)-delK-GGSPLGLAGGGS-
IL-15(S162A)-GGGSGGGSGRSDNQGGGSGGGSG-IL-15Rα(T2A)
>Chains 1 and 2: FAP-H6_huIgG1(AAA)_delK-GGS-PLGLAG-GGS-
L_(S162A)-GGGSGGG-SGRSDNQ-GGGSGGGSG-R_T2A
(SEQ ID NO: 519)
QVQLVQSGAEVKKPGASVKVSCKASGYTFTENIIHWVRQAPGQGLEWMGWIHPGSGSIKYAQK
LQGRVTMTTDTSTSTAYMELRSLRSDDTAVYYCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGSPLGLAGGGSNWVNVISDLKKIEDLIQSMHIDATLYTESDVHPSCKVTAMKCFLLELQVISL
ESGDASIHDTVENLIILANNSLSSNGNVTESGCKECEELEEKNIKEFLQSFVHIVQMFINTAGGGSGGGSGRSDN
QGGGSGGGSGIACPPPMSVEHADIWVKSYSLYSRERYICNSGFKRKAGTSSLTECVLNKATNVAHWTTPSLKCIR
DPALVHQRPAPPS*
>Chains 3 and 4: FAP-L7-huIgKLC
(SEQ ID NO: 520)
EIVLTQSPGTLSLSPGERATLSCRASQSVSTSAYSYMHWYQQKPGQAPRLLIYLASNLESGIP
DRFSGSGSGTDFTLTISRLEPEDFAVYYCQQSRELPYTEGQGTKLEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSSPVTKSF
NRGEC*
P24902158: FAP-H6L7-huIgG1(L234A, L235A, P329A)-delK-GGPLGLAGG-IL-
15(S162A)-GGGSGGGSGRSDNQGGGSGGGSG-IL-15Rα(T2A)
>Chains 1 and 2: FAP-H6_huIgG1(AAA)_delK-GG-PLGLAG-G-L_(S162A)-
GGGSGGG-SGRSDNQ-GGGSGGGSG-R_T2A
(SEQ ID NO: 521)
QVQLVQSGAEVKKPGASVKVSCKASGYTFTENIIHWVRQAPGQGLEWMGWIHPGSGSIKYAQK
LQGRVTMTTDTSTSTAYMELRSLRSDDTAVYYCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGPLGLAGGNWVNVISDLKKIEDLIQSMHIDATLYTESDVHPSCKVTAMKCFLLELQVISLESG
DASIHDTVENLIILANNSLSSNGNVTESGCKECEELEEKNIKEFLQSFVHIVQMFINTAGGGSGGGSGRSDNQGG
GSGGGSGIACPPPMSVEHADIWVKSYSLYSRERYICNSGFKRKAGTSSLTECVLNKATNVAHWTTPSLKCIRDPA
LVHQRPAPPS
>Chains 3 and 4: FAP-L7-huIgKLC
(SEQ ID NO: 522)
EIVLTQSPGTLSLSPGERATLSCRASQSVSTSAYSYMHWYQQKPGQAPRLLIYLASNLESGIP
DRFSGSGSGTDFTLTISRLEPEDFAVYYCQQSRELPYTEGQGTKLEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSSPVTKSF
NRGEC
P24912158: FAP-H6L7-huIgG1(L234A, L235A, P329A)-delK-GGSGGSGGSGGS-
IL-15(S162A)-GGGSGGGSGRSDNQGGGSGGGSG-IL-15Rα (T2A)
>Chains 1 and 2: FAP-H6_huIgG1(AAA)_delK-GGS-GGSGGS-GGS-
L_(S162A)-GGGSGGG-SGRSDNQ-GGGSGGGSG-R_T2A
(SEQ ID NO: 523)
QVQLVQSGAEVKKPGASVKVSCKASGYTFTENIIHWVRQAPGQGLEWMGWIHPGSGSIKYAQK
LQGRVTMTTDTSTSTAYMELRSLRSDDTAVYYCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGSGGSGGSGGSNWVNVISDLKKIEDLIQSMHIDATLYTESDVHPSCKVTAMKCFLLELQVISL
ESGDASIHDTVENLIILANNSLSSNGNVTESGCKECEELEEKNIKEFLQSFVHIVQMFINTAGGGSGGGSGRSDN
QGGGSGGGSGIACPPPMSVEHADIWVKSYSLYSRERYICNSGFKRKAGTSSLTECVLNKATNVAHWTTPSLKCIR
DPALVHQRPAPPS
>Chains 3 and 4: FAP-L7-huIgKLC
(SEQ ID NO: 524)
EIVLTQSPGTLSLSPGERATLSCRASQSVSTSAYSYMHWYQQKPGQAPRLLIYLASNLESGIP
DRFSGSGSGTDFTLTISRLEPEDFAVYYCQQSRELPYTEGQGTKLEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSSPVTKSF
NRGEC

Examples of Protease Cleavage Products

Expected Cleavage Products from uPA/Matriptase Cleavage of P15431450:

1) FAP-H1L1-huIgG1-PLGLAGSGR/SDNR-IL-15(S162A)-
GGSGGSGGSGGSGGS-IL-15Rα/FAP-L1-huIgKLC (product
from complete cleavage)
Chains 1 and 2: FAP-H1L1-huIgG1-PLGLAGSGR
(SEQ ID NO: 525)
EVQLVQSGAEVKKPGESLKISCKGSGYTFTENIIHWVRQMPGKGLEWIGW
FHPGSGSIKYNEKFKDQVTISADKSISTAYLQWSSLKASDTAMYFCARHG
GTGRGAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
PLGLAGSGR
Chains 3 and 4: SDNR-IL-15(S162A)-GGSGGSGGSGGSGGS-
IL-15Rα
(SEQ ID NO: 526)
SDNRNWVNVISDLKKIEDLIQSMHIDATLYTESDVHPSCKVTAMKCFLLE
LQVISLESGDASIHDTVENLIILANNSLSSNGNVTESGCKECEELEEKNI
KEFLQSFVHIVQMFINTAGGSGGSGGSGGSGGSITCPPPMSVEHADIWVK
SYSLYSRERYICNSGFKRKAGTSSLTECVLNKATNVAHWTTPSLKCIRDP
ALVHQRPAPPS
Chains 5 and 6: FAP-L1-huIgKLC
(SEQ ID NO: 527)
DIVMTQTPLSLSVTPGQPASISCRASKSVSTSAYSYMHWYLQKPGQSPQL
LIYLASNLESGVPDRFSGSGSGTDFTLKISRVEAEDVGVYYCQHSRELPY
TFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
2) FAP-H1L1-huIgG1-PLGLAGSGRSDNR-IL-15(S162A)-
GGSGGSGGSGGSGGS-IL-15Rα/FAP-H1L1-huIgG1-
PLGLAGSGR/SDNR-IL-15(S162A)-GGSGGSGGSGGSGGS-IL-
15Rα/FAP-L1-huIgKLC (product from partial
cleavage, active)
Chain 1: FAP-H1L1-huIgG1-PLGLAGSGRSDNR-IL-
15(S162A)-GGSGGSGGSGGSGGS-IL-15Rα
(SEQ ID NO: 528)
EVQLVQSGAEVKKPGESLKISCKGSGYTFTENIIHWVRQMPGKGLEWIGW
FHPGSGSIKYNEKEKDQVTISADKSISTAYLQWSSLKASDTAMYFCARHG
GTGRGAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
PLGLAGSGRSDNRNWVNVISDLKKIEDLIQSMHIDATLYTESDVHPSCKV
TAMKCELLELQVISLESGDASIHDTVENLIILANNSLSSNGNVTESGCKE
CEELEEKNIKEFLQSFVHIVQMFINTAGGSGGSGGSGGSGGSITCPPPMS
VEHADIWVKSYSLYSRERYICNSGFKRKAGTSSLTECVLNKATNVAHWTT
PSLKCIRDPALVHQRPAPPS
Chain 2: FAP-H1L1-huIgG1-PLGLAGSGR
(SEQ ID NO: 529)
EVQLVQSGAEVKKPGESLKISCKGSGYTFTENIIHWVRQMPGKGLEWIGW
FHPGSGSIKYNEKFKDQVTISADKSISTAYLQWSSLKASDTAMYFCARHG
GTGRGAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
PLGLAGSGR
Chain 3: SDNR-IL-15(S162A)-GGSGGSGGSGGSGGS-IL-15Rα
(SEQ ID NO: 530)
SDNRNWVNVISDLKKIEDLIQSMHIDATLYTESDVHPSCKVTAMKCFLLE
LQVISLESGDASIHDTVENLIILANNSLSSNGNVTESGCKECEELEEKNI
KEFLQSFVHIVQMFINTAGGSGGSGGSGGSGGSITCPPPMSVEHADIWVK
SYSLYSRERYICNSGFKRKAGTSSLTECVLNKATNVAHWTTPSLKCIRDP
ALVHQRPAPPS*
Chain 4 and 5: FAP-L1-huIgKLC
(SEQ ID NO: 531)
DIVMTQTPLSLSVTPGQPASISCRASKSVSTSAYSYMHWYLQKPGQSPQL
LIYLASNLESGVPDRFSGSGSGTDFTLKISRVEAEDVGVYYCQHSRELPY
TFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

Expected Cleavage Products from MMP-2/MMP-9 Cleavage of P15431450:

1) FAP-H1L1-huIgG1-PLG/LAGSGRSDNR-IL-15(S162A)-
GGSGGSGGSGGSGGS-IL-15Rα/FAP-L1-huIgKLC (product
from complete cleavage)
Chains 1 and 2: FAP-H1L1-huIgG1-PLG
(SEQ ID NO: 532)
EVQLVQSGAEVKKPGESLKISCKGSGYTFTENIIHWVRQMPGKGLEWIGW
FHPGSGSIKYNEKFKDQVTISADKSISTAYLQWSSLKASDTAMYFCARHG
GTGRGAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
PLG
Chains 3 and 4: LAGSGRSDNR-IL-15(S162A)-
GGSGGSGGSGGSGGS-IL-15Rα
(SEQ ID NO: 533)
LAGSGRSDNRNWVNVISDLKKIEDLIQSMHIDATLYTESDVHPSCKVTAM
KCFLLELQVISLESGDASIHDTVENLIILANNSLSSNGNVTESGCKECEE
LEEKNIKEFLQSFVHIVQMFINTAGGSGGSGGSGGSGGSITCPPPMSVEH
ADIWVKSYSLYSRERYICNSGFKRKAGTSSLTECVLNKATNVAHWTTPSL
KCIRDPALVHQRPAPPS
Chains 5 and 6: FAP-L1-huIgKLC
(SEQ ID NO: 534)
DIVMTQTPLSLSVTPGQPASISCRASKSVSTSAYSYMHWYLQKPGQSPQL
LIYLASNLESGVPDRFSGSGSGTDFTLKISRVEAEDVGVYYCQHSRELPY
TFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
2) FAP-H1L1-huIgG1-PLGLAGSGRSDNR-IL-15(S162A)-
GGSGGSGGSGGSGGS-IL-15Rα/FAP-H1L1-huIgG1-PLG/
LAGSGRSDNR-IL-15(S162A)-GGSGGSGGSGGSGGS-IL-
15Rα/FAP-L1-huIgKLC (product from partial
cleavage, active)
Chain 1: FAP-H1L1-huIgG1-PLGLAGSGRSDNR-IL-
15(S162A)-GGSGGSGGSGGSGGS-IL-15Rα
(SEQ ID NO: 535)
EVQLVQSGAEVKKPGESLKISCKGSGYTFTENIIHWVRQMPGKGLEWIGW
FHPGSGSIKYNEKEKDQVTISADKSISTAYLQWSSLKASDTAMYFCARHG
GTGRGAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
PLGLAGSGRSDNRNWVNVISDLKKIEDLIQSMHIDATLYTESDVHPSCKV
TAMKCELLELQVISLESGDASIHDTVENLIILANNSLSSNGNVTESGCKE
CEELEEKNIKEFLQSFVHIVQMFINTAGGSGGSGGSGGSGGSITCPPPMS
VEHADIWVKSYSLYSRERYICNSGFKRKAGTSSLTECVLNKATNVAHWTT
PSLKCIRDPALVHQRPAPPS
Chain 2: FAP-H1L1-huIgG1-PLG
(SEQ ID NO: 536)
EVQLVQSGAEVKKPGESLKISCKGSGYTFTENIIHWVRQMPGKGLEWIGW
FHPGSGSIKYNEKFKDQVTISADKSISTAYLQWSSLKASDTAMYFCARHG
GTGRGAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
PLG
Chain 3: LAGSGRSDNR-IL-15(S162A)-GGSGGSGGSGGSGGS-
IL-15Rα
(SEQ ID NO: 537)
LAGSGRSDNRNWVNVISDLKKIEDLIQSMHIDATLYTESDVHPSCKVTAM
KCFLLELQVISLESGDASIHDTVENLIILANNSLSSNGNVTESGCKECEE
LEEKNIKEFLQSFVHIVQMFINTAGGSGGSGGSGGSGGSITCPPPMSVEH
ADIWVKSYSLYSRERYICNSGFKRKAGTSSLTECVLNKATNVAHWTTPSL
KCIRDPALVHQRPAPPS
Chain 4 and 5: FAP-L1-huIgKLC
(SEQ ID NO: 538)
DIVMTQTPLSLSVTPGQPASISCRASKSVSTSAYSYMHWYLQKPGQSPQL
LIYLASNLESGVPDRFSGSGSGTDFTLKISRVEAEDVGVYYCQHSRELPY
TFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

Expected Cleavage Products from uPA/Matriptase Cleavage of P18121450:

1) FAP-H1-huIgG1(AAA) delK-GG-L (S162A)-
GGSGGSPLGLAGSGR/SDNQGGSGGA-IL-15Rα(T2A)/FAP-L1-
huIgKLC (product from complete cleavage)
Chains 1 and 2: FAP-H1-huIgG1(AAA)_delK-GG-
L_(S162A)-GGSGGSPLGLAGSGR
(SEQ ID NO: 539)
EVQLVQSGAEVKKPGESLKISCKGSGYTFTENIIHWVRQMPGKGLEWIGW
FHPGSGSIKYNEKFKDQVTISADKSISTAYLQWSSLKASDTAMYFCARHG
GTGRGAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALAAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGG
GNWVNVISDLKKIEDLIQSMHIDATLYTESDVHPSCKVTAMKCFLLELQV
ISLESGDASIHDTVENLIILANNSLSSNGNVTESGCKECEELEEKNIKEF
LQSFVHIVQMFINTAGGSGGSPLGLAGSGR
Chains 3 and 4: SDNQGGSGGA-IL-15Rα(T2A)
(SEQ ID NO: 540)
SDNQGGSGGAIACPPPMSVEHADIWVKSYSLYSRERYICNSGFKRKAGTS
SLTECVLNKATNVAHWTTPSLKCIRDPALVHQRPAPPS
Chains 5 and 6: FAP-L1-huIgKLC
(SEQ ID NO: 541)
DIVMTQTPLSLSVTPGQPASISCRASKSVSTSAYSYMHWYLQKPGQSPQL
LIYLASNLESGVPDRFSGSGSGTDFTLKISRVEAEDVGVYYCQHSRELPY
TFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
2) FAP-H1-huIgG1(AAA) delK-GG-L (S162A)-
GGSGGSPLGLAGSGRSDNQGGSGGA-IL-15Rα(T2A)/FAP-H1-
huIgG1(AAA) delK-GG-L (S162A)-GGSGGSPLGLAGSGR/
SDNQGGSGGA-IL-15Rα(T2A)/FAP-L1-huIgKLC (product
from partial cleavage, active)
Chain 1: FAP-H1-huIgG1(AAA)_delK-GG-L_(S162A)-
GGSGGSPLGLAGSGRSDNQGGSGGA-IL-15Rα(T2A)
(SEQ ID NO: 542)
EVQLVQSGAEVKKPGESLKISCKGSGYTFTENIIHWVRQMPGKGLEWIGW
FHPGSGSIKYNEKFKDQVTISADKSISTAYLQWSSLKASDTAMYFCARHG
GTGRGAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALAAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGG
GNWVNVISDLKKIEDLIQSMHIDATLYTESDVHPSCKVTAMKCFLLELQV
ISLESGDASIHDTVENLIILANNSLSSNGNVTESGCKECEELEEKNIKEF
LQSFVHIVQMFINTAGGSGGSPLGLAGSGRSDNQGGSGGAIACPPPMSVE
HADIWVKSYSLYSRERYICNSGFKRKAGTSSLTECVLNKATNVAHWTTPS
LKCIRDPALVHQRPAPPS
Chain 2: FAP-H1-huIgG1(AAA)_delK-GG-L_(S162A)-
GGSGGSPLGLAGSGR
(SEQ ID NO: 543)
EVQLVQSGAEVKKPGESLKISCKGSGYTFTENIIHWVRQMPGKGLEWIGW
FHPGSGSIKYNEKFKDQVTISADKSISTAYLQWSSLKASDTAMYFCARHG
GTGRGAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALAAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGG
GNWVNVISDLKKIEDLIQSMHIDATLYTESDVHPSCKVTAMKCFLLELQV
ISLESGDASIHDTVENLIILANNSLSSNGNVTESGCKECEELEEKNIKEF
LQSFVHIVQMFINTAGGSGGSPLGLAGSGR
Chain 3: SDNQGGSGGA-IL-15Rα(T2A)
(SEQ ID NO: 544)
SDNQGGSGGAIACPPPMSVEHADIWVKSYSLYSRERYICNSGFKRKAGTS
SLTECVLNKATNVAHWTTPSLKCIRDPALVHQRPAPPS
Chain 4 and 5: FAP-L1-huIgKLC
(SEQ ID NO: 545)
DIVMTQTPLSLSVTPGQPASISCRASKSVSTSAYSYMHWYLQKPGQSPQL
LIYLASNLESGVPDRFSGSGSGTDFTLKISRVEAEDVGVYYCQHSRELPY
TFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

Expected Cleavage Products from MMP-2/MMP-9 Cleavage of P18121450:

1) FAP-H1-huIgG1(AAA) delK-GG-L (S162A)-
GGSGGSPLG/LAGSGRSDNQGGSGGA-IL-15Rα(T2A)/FAP-L1-
huIgKLC (product from complete cleavage)
Chains 1 and 2: FAP-H1-huIgG1(AAA)_delK-GG-
L_(S162A)-GGSGGSP LG
(SEQ ID NO: 546)
EVQLVQSGAEVKKPGESLKISCKGSGYTFTENIIHWVRQMPGKGLEWIGW
FHPGSGSIKYNEKFKDQVTISADKSISTAYLQWSSLKASDTAMYFCARHG
GTGRGAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALAAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGG
GNWVNVISDLKKIEDLIQSMHIDATLYTESDVHPSCKVTAMKCFLLELQV
ISLESGDASIHDTVENLIILANNSLSSNGNVTESGCKECEELEEKNIKEF
LQSFVHIVQMFINTAGGSGGSPLG
Chains 3 and 4: LAGSGRSDNQGGSGGA-IL-15Rα(T2A)
(SEQ ID NO: 547)
LAGSGRSDNQGGSGGAIACPPPMSVEHADIWVKSYSLYSRERYICNSGFK
RKAGTSSLTECVLNKATNVAHWTTPSLKCIRDPALVHQRPAPPS
Chains 5 and 6: FAP-L1-huIgKLC
(SEQ ID NO: 548)
DIVMTQTPLSLSVTPGQPASISCRASKSVSTSAYSYMHWYLQKPGQSPQL
LIYLASNLESGVPDRFSGSGSGTDFTLKISRVEAEDVGVYYCQHSRELPY
TFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
2) FAP-H1-huIgG1(AAA) delK-GG-L (S162A)-
GGSGGSPLGLAGSGRSDNQGGSGGA-IL-15Rα(T2A)/FAP-H1-
huIgG1(AAA) delK-GG-L (S162A)-GGSGGSPLG/
LAGSGRSDNQGGSGGA-IL-15Rα(T2A)/FAP-L1-huIgKLC
(product from partial cleavage, active)
Chain 1: FAP-H1-huIgG1(AAA)_delK-GG-L_(S162A)-
GGSGGSPLGLAGSGRSDNQGGSGGA-IL-15Rα(T2A)
(SEQ ID NO: 549)
EVQLVQSGAEVKKPGESLKISCKGSGYTFTENIIHWVRQMPGKGLEWIGW
FHPGSGSIKYNEKFKDQVTISADKSISTAYLQWSSLKASDTAMYFCARHG
GTGRGAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALAAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGG
GNWVNVISDLKKIEDLIQSMHIDATLYTESDVHPSCKVTAMKCFLLELQV
ISLESGDASIHDTVENLIILANNSLSSNGNVTESGCKECEELEEKNIKEF
LQSFVHIVQMFINTAGGSGGSPLGLAGSGRSDNQGGSGGAIACPPPMSVE
HADIWVKSYSLYSRERYICNSGFKRKAGTSSLTECVLNKATNVAHWTTPS
LKCIRDPALVHQRPAPPS
Chain 2: FAP-H1-huIgG1(AAA)_delK-GG-L_(S162A)-
GGSGGSPLG
(SEQ ID NO: 550)
EVQLVQSGAEVKKPGESLKISCKGSGYTFTENIIHWVRQMPGKGLEWIGW
FHPGSGSIKYNEKFKDQVTISADKSISTAYLQWSSLKASDTAMYFCARHG
GTGRGAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALAAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGG
GNWVNVISDLKKIEDLIQSMHIDATLYTESDVHPSCKVTAMKCFLLELQV
ISLESGDASIHDTVENLIILANNSLSSNGNVTESGCKECEELEEKNIKEF
LQSFVHIVQMFINTAGGSGGSPLG
Chain 3: LAGSGRSDNQGGSGGA-IL-15Rα(T2A)
(SEQ ID NO: 551)
LAGSGRSDNQGGSGGAIACPPPMSVEHADIWVKSYSLYSRERYICNSGFK
RKAGTSSLTECVLNKATNVAHWTTPSLKCIRDPALVHQRPAPPS
Chain 4 and 5: FAP-L1-huIgKLC
(SEQ ID NO: 552)
DIVMTQTPLSLSVTPGQPASISCRASKSVSTSAYSYMHWYLQKPGQSPQL
LIYLASNLESGVPDRFSGSGSGTDFTLKISRVEAEDVGVYYCQHSRELPY
TFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

Expected Cleavage Products from uPA/Matriptase Cleavage of P24872158:

1) FAP-H6L7-huIgG1(L234A, L235A, P329A)-delK-
GGSPLGLAGGGS-IL-15(S162A)-GGGSGGGSGR/SDNQGGGSG-IL-
15Rα(T2A)/FAP-L7-huIgKLC (product from complete
cleavage)
Chains 1 and 2: FAP-H6L7-huIgG1(L234A, L235A,
P329A)-delK-GGSPLGLAGGGS-IL-15(S162A)-GGGSGGGSGR
(SEQ ID NO: 553)
QVQLVQSGAEVKKPGASVKVSCKASGYTFTENIIHWVRQAPGQGLEWMGW
IHPGSGSIKYAQKLQGRVTMTTDTSTSTAYMELRSLRSDDTAVYYCARHG
GTGRGAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALAAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGG
GSPLGLAGGGSNWVNVISDLKKIEDLIQSMHIDATLYTESDVHPSCKVTA
MKCFLLELQVISLESGDASIHDTVENLIILANNSLSSNGNVTESGCKECE
ELEEKNIKEFLQSFVHIVQMFINTAGGGSGGGSGR
Chains 3 and 4: SDNQGGGSG-IL-15Rα(T2A)
(SEQ ID NO: 554)
SDNQGGGSGIACPPPMSVEHADIWVKSYSLYSRERYICNSGFKRKAGTSS
LTECVLNKATNVAHWTTPSLKCIRDPALVHQRPAPPS
Chains 5 and 6: FAP-L7-huIgKLC
(SEQ ID NO: 555)
EIVLTQSPGTLSLSPGERATLSCRASQSVSTSAYSYMHWYQQKPGQAPRL
LIYLASNLESGIPDRFSGSGSGTDFTLTISRLEPEDFAVYYCQQSRELPY
TEGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
2) FAP-H6L7-huIgG1(L234A, L235A, P329A)-delK-
GGSPLGLAGGGS-IL-15(S162A)-GGGSGGGSGRSDNQGGGSG-IL-
15Rα(T2A)/FAP-H6L7-huIgG1(L234A, L235A, P329A)-
delK-GGSPLGLAGGGS-IL-15(S162A)-GGGSGGGSGR/
SDNQGGGSG-IL-15Rα(T2A)/FAP-L7-huIgKLC (product
from partial cleavage, active)
Chain 1: FAP-H6L7-huIgG1(L234A, L235A, P329A)-
delK-GGSPLGLAGGGS-IL-15(S162A)-
GGGSGGGSGRSDNQGGGSG-IL-15Rα(T2A)
(SEQ ID NO: 556)
QVQLVQSGAEVKKPGASVKVSCKASGYTFTENIIHWVRQAPGQGLEWMGW
IHPGSGSIKYAQKLQGRVTMTTDTSTSTAYMELRSLRSDDTAVYYCARHG
GTGRGAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALAAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGG
GSPLGLAGGGSNWVNVISDLKKIEDLIQSMHIDATLYTESDVHPSCKVTA
MKCFLLELQVISLESGDASIHDTVENLIILANNSLSSNGNVTESGCKECE
ELEEKNIKEFLQSFVHIVQMFINTAGGGSGGGSGRSDNQGGGSGIACPPP
MSVEHADIWVKSYSLYSRERYICNSGFKRKAGTSSLTECVLNKATNVAHW
TTPSLKCIRDPALVHQRPAPPS
Chain 2: FAP-H6L7-huIgG1(L234A, L235A, P329A)-
delK-GGSPLGLAGGGS-IL-15(S162A)-GGGSGGGSGR
(SEQ ID NO: 557)
QVQLVQSGAEVKKPGASVKVSCKASGYTFTENIIHWVRQAPGQGLEWMGW
IHPGSGSIKYAQKLQGRVTMTTDTSTSTAYMELRSLRSDDTAVYYCARHG
GTGRGAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALAAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGG
GSPLGLAGGGSNWVNVISDLKKIEDLIQSMHIDATLYTESDVHPSCKVTA
MKCFLLELQVISLESGDASIHDTVENLIILANNSLSSNGNVTESGCKECE
ELEEKNIKEFLQSFVHIVQMFINTAGGGSGGGSGR
Chain 3: SDNQGGGSG-IL-15Rα(T2A)
(SEQ ID NO: 558)
SDNQGGGSGIACPPPMSVEHADIWVKSYSLYSRERYICNSGFKRKAGTSS
LTECVLNKATNVAHWTTPSLKCIRDPALVHQRPAPPS
Chain 4 and 5: FAP-L7-huIgKLC
(SEQ ID NO: 559)
EIVLTQSPGTLSLSPGERATLSCRASQSVSTSAYSYMHWYQQKPGQAPRL
LIYLASNLESGIPDRFSGSGSGTDFTLTISRLEPEDFAVYYCQQSRELPY
TEGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

Expected Cleavage Products from MMP-2/MMP-9 Cleavage of P24872158:

1) FAP-H6L7-huIgG1(L234A, L235A, P329A)-delK-
GGSPLG/LAGGGS-IL-15(S162A)-GGGSGGGSGRSDNQGGGSG-IL-
15Rα(T2A)/FAP-L7-huIgKLC (product from complete
cleavage)
Chains 1 and 2: FAP-H6L7-huIgG1(L234A, L235A,
P329A)-delK-GGSPLG
(SEQ ID NO: 560)
QVQLVQSGAEVKKPGASVKVSCKASGYTFTENIIHWVRQAPGQGLEWMGW
IHPGSGSIKYAQKLQGRVTMTTDTSTSTAYMELRSLRSDDTAVYYCARHG
GTGRGAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALAAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGG
GSPLG
Chains 3 and 4: LAGGGS-IL-15(S162A)-
GGGSGGGSGRSDNQGGGSG-IL-15Rα(T2A)
(SEQ ID NO: 561)
LAGGGSNWVNVISDLKKIEDLIQSMHIDATLYTESDVHPSCKVTAMKCFL
LELQVISLESGDASIHDTVENLIILANNSLSSNGNVTESGCKECEELEEK
NIKEFLQSFVHIVQMFINTAGGGSGGGSGRSDNQGGGSGIACPPPMSVEH
ADIWVKSYSLYSRERYICNSGFKRKAGTSSLTECVLNKATNVAHWTTPSL
KCIRDPALVHQRPAPPS
Chains 5 and 6: FAP-L7-huIgKLC
(SEQ ID NO: 562)
EIVLTQSPGTLSLSPGERATLSCRASQSVSTSAYSYMHWYQQKPGQAPRL
LIYLASNLESGIPDRFSGSGSGTDFTLTISRLEPEDFAVYYCQQSRELPY
TEGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
2) FAP-H6L7-huIgG1(L234A, L235A, P329A)-delK-
GGSPLGLAGGGS-IL-15(S162A)-GGGSGGGSGRSDNQGGGSG-IL-
15Rα(T2A)/FAP-H6L7-huIgG1(L234A, L235A, P329A)-
delK-GGSPLG/LAGGGS-IL-15(S162A)-
GGGSGGGSGRSDNQGGGSG-IL-15Rα(T2A)/FAP-L7-huIgKLC
(product from partial cleavage, active)
Chain 1: FAP-H6L7-huIgG1(L234A, L235A, P329A)-
delK-GGSPLGLAGGGS-IL-15(S162A)-
GGGSGGGSGRSDNQGGGSG-IL-15Rα(T2A)
(SEQ ID NO: 563)
QVQLVQSGAEVKKPGASVKVSCKASGYTFTENIIHWVRQAPGQGLEWMGW
IHPGSGSIKYAQKLQGRVTMTTDTSTSTAYMELRSLRSDDTAVYYCARHG
GTGRGAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALAAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGG
GSPLGLAGGGSNWVNVISDLKKIEDLIQSMHIDATLYTESDVHPSCKVTA
MKCFLLELQVISLESGDASIHDTVENLIILANNSLSSNGNVTESGCKECE
ELEEKNIKEFLQSFVHIVQMFINTAGGGSGGGSGRSDNQGGGSGIACPPP
MSVEHADIWVKSYSLYSRERYICNSGFKRKAGTSSLTECVLNKATNVAHW
TTPSLKCIRDPALVHQRPAPPS
Chain 2: FAP-H6L7-huIgG1(L234A, L235A, P329A)-
delK-GGSPLG
(SEQ ID NO: 564)
QVQLVQSGAEVKKPGASVKVSCKASGYTFTENIIHWVRQAPGQGLEWMGW
IHPGSGSIKYAQKLQGRVTMTTDTSTSTAYMELRSLRSDDTAVYYCARHG
GTGRGAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALAAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGG
GSPLG
Chain 3: LAGGGS-IL-15(S162A)-GGGSGGGSGRSDNQGGGSG-
IL-15Rα(T2A)
(SEQ ID NO: 565)
LAGGGSNWVNVISDLKKIEDLIQSMHIDATLYTESDVHPSCKVTAMKCFL
LELQVISLESGDASIHDTVENLIILANNSLSSNGNVTESGCKECEELEEK
NIKEFLQSFVHIVQMFINTAGGGSGGGSGRSDNQGGGSGIACPPPMSVEH
ADIWVKSYSLYSRERYICNSGFKRKAGTSSLTECVLNKATNVAHWTTPSL
KCIRDPALVHQRPAPPS
Chain 4 and 5: FAP-L7-huIgKLC
(SEQ ID NO: 566)
EIVLTQSPGTLSLSPGERATLSCRASQSVSTSAYSYMHWYQQKPGQAPRL
LIYLASNLESGIPDRFSGSGSGTDFTLTISRLEPEDFAVYYCQQSRELPY
TEGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

Expected Cleavage Products from uPA/Matriptase and MMP-2/MMP-9 Cleavage of P24872158:

1) FAP-H6L7-huIgG1(L234A, L235A, P329A)-delK-GGSPLG/LAGGGS-IL-
15(S162A)-GGGSGGGSGR/SDNQGGGSG-IL-15Rα(T2A)/FAP-L7-huIgKLC (product
from complete cleavage)
Chains 1 and 2: FAP-H6L7-huIgG1(L234A, L235A, P329A)-delK-GGSPLG
(SEQ ID NO: 567)
QVQLVQSGAEVKKPGASVKVSCKASGYTFTENIIHWVRQAPGQGLEWMGWIHPGSGSIKYAQK
LQGRVTMTTDTSTSTAYMELRSLRSDDTAVYYCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGSPLG
Chains 3 and 4: LAGGGS-IL-15(S162A)-GGGSGGGSGRSDNQGGGSG-IL-
15Rα(T2A)
(SEQ ID NO: 568)
LAGGGSNWVNVISDLKKIEDLIQSMHIDATLYTESDVHPSCKVTAMKCFLLELQVISLESGDA
SIHDTVENLIILANNSLSSNGNVTESGCKECEELEEKNIKEFLQSFVHIVQMFINTAGGGSGGGSGR
Chains 5 and 6: SDNQGGGSG-IL-15Rα(T2A)
(SEQ ID NO: 569)
SDNQGGGSGIACPPPMSVEHADIWVKSYSLYSRERYICNSGFKRKAGTSSLTECVLNKATNVA
HWTTPSLKCIRDPALVHQRPAPPS
Chains 7 and 8: FAP-L7-huIgKLC
(SEQ ID NO: 570)
EIVLTQSPGTLSLSPGERATLSCRASQSVSTSAYSYMHWYQQKPGQAPRLLIYLASNLESGIP
DRFSGSGSGTDFTLTISRLEPEDFAVYYCQQSRELPYTEGQGTKLEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSSPVTKSF
NRGEC
2) FAP-H6L7-huIgG1(L234A, L235A, P329A)-delK-GGSPLGLAGGGS-IL-
15(S162A)-GGGSGGGSGRSDNQGGGSG-IL-15Rα(T2A)/FAP-H6L7-
huIgG1(L234A, L235A, P329A)-delK-GGSPLG/LAGGGS-IL-15(S162A)-
GGGSGGGSGR/SDNQGGGSG-IL-15Rα(T2A)/FAP-L7-huIgKLC (product from partial
cleavage, active)
Chain 1: FAP-H6L7-huIgG1(L234A, L235A, P329A)-delK-GGSPLGLAGGGS-
IL-15(S162A)-GGGSGGGSGRSDNQGGGSG-IL-15Rα(T2A)
(SEQ ID NO: 571)
QVQLVQSGAEVKKPGASVKVSCKASGYTFTENIIHWVRQAPGQGLEWMGWIHPGSGSIKYAQK
LQGRVTMTTDTSTSTAYMELRSLRSDDTAVYYCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGSPLGLAGGGSNWVNVISDLKKIEDLIQSMHIDATLYTESDVHPSCKVTAMKCFLLELQVISL
ESGDASIHDTVENLIILANNSLSSNGNVTESGCKECEELEEKNIKEFLQSFVHIVQMFINTAGGGSGGGSGRSDN
QGGGSGIACPPPMSVEHADIWVKSYSLYSRERYICNSGFKRKAGTSSLTECVLNKATNVAHWTTPSLKCIRDPAL
VHQRPAPPS
Chain 2: FAP-H6L7-huIgG1(L234A, L235A, P329A)-delK-GGSPLG
(SEQ ID NO: 572)
QVQLVQSGAEVKKPGASVKVSCKASGYTFTENIIHWVRQAPGQGLEWMGWIHPGSGSIKYAQK
LQGRVTMTTDTSTSTAYMELRSLRSDDTAVYYCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGSPLG
Chain 3: LAGGGS-IL-15(S162A)-GGGSGGGSGR
(SEQ ID NO: 573)
LAGGGSNWVNVISDLKKIEDLIQSMHIDATLYTESDVHPSCKVTAMKCFLLELQVISLESGDA
SIHDTVENLIILANNSLSSNGNVTESGCKECEELEEKNIKEFLQSFVHIVQMFINTAGGGSGGGSGR
Chain 4: SDNQGGGSG-IL-15Rα(T2A)
(SEQ ID NO: 574)
SDNQGGGSGIACPPPMSVEHADIWVKSYSLYSRERYICNSGFKRKAGTSSLTECVLNKATNVA
HWTTPSLKCIRDPALVHQRPAPPS
Chain 5 and 6: FAP-L7-huIgKLC
(SEQ ID NO: 575)
EIVLTQSPGTLSLSPGERATLSCRASQSVSTSAYSYMHWYQQKPGQAPRLLIYLASNLESGIP
DRFSGSGSGTDFTLTISRLEPEDFAVYYCQQSRELPYTEGQGTKLEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSSPVTKSF
NRGEC
3) FAP-H6L7-huIgG1(L234A, L235A, P329A)-delK-GGSPLGLAGGGS-IL-
15(S162A)-GGGSGGGSGRSDNQGGGSG-IL-15Rα(T2A)/FAP-H6L7-
huIgG1(L234A, L235A, P329A)-delK-GGSPLGLAGGGS-IL-15(S162A)-
GGGSGGGSGR/SDNQGGGSG-IL-15Rα(T2A)/FAP-L7-huIgKLC (product from partial
cleavage, active)
Chain 1: FAP-H6L7-huIgG1(L234A, L235A, P329A)-delK-GGSPLGLAGGGS-
IL-15(S162A)-GGGSGGGSGRSDNQGGGSG-IL-15Rα(T2A)
(SEQ ID NO: 576)
QVQLVQSGAEVKKPGASVKVSCKASGYTFTENIIHWVRQAPGQGLEWMGWIHPGSGSIKYAQK
LQGRVTMTTDTSTSTAYMELRSLRSDDTAVYYCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGSPLGLAGGGSNWVNVISDLKKIEDLIQSMHIDATLYTESDVHPSCKVTAMKCFLLELQVISL
ESGDASIHDTVENLIILANNSLSSNGNVTESGCKECEELEEKNIKEFLQSFVHIVQMFINTAGGGSGGGSGRSDN
QGGGSGIACPPPMSVEHADIWVKSYSLYSRERYICNSGFKRKAGTSSLTECVLNKATNVAHWTTPSLKCIRDPAL
VHQRPAPPS
Chain 2: FAP-H6L7-huIgG1(L234A, L235A, P329A)-delK-GGSPLGLAGGGS-
IL-15(S162A)-GGGSGGGSGR
(SEQ ID NO: 577)
QVQLVQSGAEVKKPGASVKVSCKASGYTFTENIIHWVRQAPGQGLEWMGWIHPGSGSIKYAQK
LQGRVTMTTDTSTSTAYMELRSLRSDDTAVYYCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGSPLGLAGGGSNWVNVISDLKKIEDLIQSMHIDATLYTESDVHPSCKVTAMKCFLLELQVISL
ESGDASIHDTVENLIILANNSLSSNGNVTESGCKECEELEEKNIKEFLQSFVHIVQMFINTAGGGSGGGSGR
Chain 3: SDNQGGGSG-IL-15Rα(T2A)
(SEQ ID NO: 578)
SDNQGGGSGIACPPPMSVEHADIWVKSYSLYSRERYICNSGFKRKAGTSSLTECVLNKATNVA
HWTTPSLKCIRDPALVHQRPAPPS
Chain 4 and 5: FAP-L7-huIgKLC
(SEQ ID NO: 579)
EIVLTQSPGTLSLSPGERATLSCRASQSVSTSAYSYMHWYQQKPGQAPRLLIYLASNLESGIP
DRFSGSGSGTDFTLTISRLEPEDFAVYYCQQSRELPYTEGQGTKLEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSSPVTKSF
NRGEC
4) FAP-H6L7-huIgG1(L234A, L235A, P329A)-delK-GGSPLGLAGGGS-IL-
15(S162A)-GGGSGGGSGRSDNQGGGSG-IL-15Rα(T2A)/FAP-H6L7-
huIgG1(L234A, L235A, P329A)-delK-GGSPLG/LAGGGS-IL-15(S162A)-
GGGSGGGSGRSDNQGGGSG-IL-15Rα(T2A)/FAP-L7-huIgKLC (product from partial
cleavage, active)
Chain 1: FAP-H6L7-huIgG1(L234A, L235A, P329A)-delK-GGSPLGLAGGGS-
IL-15(S162A)-GGGSGGGSGRSDNQGGGSG-IL-15Rα(T2A)
(SEQ ID NO: 580)
QVQLVQSGAEVKKPGASVKVSCKASGYTFTENIIHWVRQAPGQGLEWMGWIHPGSGSIKYAQK
LQGRVTMTTDTSTSTAYMELRSLRSDDTAVYYCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGSPLGLAGGGSNWVNVISDLKKIEDLIQSMHIDATLYTESDVHPSCKVTAMKCFLLELQVISL
ESGDASIHDTVENLIILANNSLSSNGNVTESGCKECEELEEKNIKEFLQSFVHIVQMFINTAGGGSGGGSGRSDN
QGGGSGIACPPPMSVEHADIWVKSYSLYSRERYICNSGFKRKAGTSSLTECVLNKATNVAHWTTPSLKCIRDPAL
VHQRPAPPS
Chain 2: FAP-H6L7-huIgG1(L234A, L235A, P329A)-delK-GGSPLG
(SEQ ID NO: 581)
QVQLVQSGAEVKKPGASVKVSCKASGYTFTENIIHWVRQAPGQGLEWMGWIHPGSGSIKYAQK
LQGRVTMTTDTSTSTAYMELRSLRSDDTAVYYCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGSPLG
Chain 3: LAGGGS-IL-15(S162A)-GGGSGGGSGRSDNQGGGSG-IL-15Rα(T2A)
(SEQ ID NO: 582)
LAGGGSNWVNVISDLKKIEDLIQSMHIDATLYTESDVHPSCKVTAMKCFLLELQVISLESGDA
SIHDTVENLIILANNSLSSNGNVTESGCKECEELEEKNIKEFLQSFVHIVQMFINTAGGGSGGGSGRSDNQGGGS
GIACPPPMSVEHADIWVKSYSLYSRERYICNSGFKRKAGTSSLTECVLNKATNVAHWTTPSLKCIRDPALVHQRP
APPS
Chain 4 and 5: FAP-L7-huIgKLC
(SEQ ID NO: 583)
EIVLTQSPGTLSLSPGERATLSCRASQSVSTSAYSYMHWYQQKPGQAPRLLIYLASNLESGIP
DRFSGSGSGTDFTLTISRLEPEDFAVYYCQQSRELPYTEGQGTKLEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSSPVTKSF
NRGEC
5) FAP-H6L7-huIgG1(L234A, L235A, P329A)-delK-GGSPLG/LAGGGS-IL-
15(S162A)-GGGSGGGSGRSDNQGGGSG-IL-15Rα(T2A)/FAP-H6L7-
huIgG1(L234A, L235A, P329A)-delK-GGSPLGLAGGGS-IL-15(S162A)-
GGGSGGGSGR/SDNQGGGSG-IL-15Rα(T2A)/FAP-L7-huIgKLC (product from partial
cleavage, active)
Chain 1: FAP-H6L7-huIgG1(L234A, L235A, P329A)-delK-GGSPLG
(SEQ ID NO: 584)
QVQLVQSGAEVKKPGASVKVSCKASGYTFTENIIHWVRQAPGQGLEWMGWIHPGSGSIKYAQK
LQGRVTMTTDTSTSTAYMELRSLRSDDTAVYYCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGSPLG
Chain 2: LAGGGS-IL-15(S162A)-GGGSGGGSGRSDNQGGGSG-IL-15Rα(T2A)
(SEQ ID NO: 585)
LAGGGSNWVNVISDLKKIEDLIQSMHIDATLYTESDVHPSCKVTAMKCFLLELQVISLESGDA
SIHDTVENLIILANNSLSSNGNVTESGCKECEELEEKNIKEFLQSFVHIVQMFINTAGGGSGGGSGRSDNQGGGS
GIACPPPMSVEHADIWVKSYSLYSRERYICNSGFKRKAGTSSLTECVLNKATNVAHWTTPSLKCIRDPALVHQRP
APPS
Chain 3: FAP-H6L7-huIgG1(L234A, L235A, P329A)-delK-GGSPLGLAGGGS-
IL-15(S162A)-GGGSGGGSGR
(SEQ ID NO: 586)
QVQLVQSGAEVKKPGASVKVSCKASGYTFTENIIHWVRQAPGQGLEWMGWIHPGSGSIKYAQK
LQGRVTMTTDTSTSTAYMELRSLRSDDTAVYYCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGSPLGLAGGGSNWVNVISDLKKIEDLIQSMHIDATLYTESDVHPSCKVTAMKCFLLELQVISL
ESGDASIHDTVENLIILANNSLSSNGNVTESGCKECEELEEKNIKEFLQSFVHIVQMFINTAGGGSGGGSGR
Chain 4: SDNQGGGSG-IL-15Rα(T2A)
(SEQ ID NO: 587)
SDNQGGGSGIACPPPMSVEHADIWVKSYSLYSRERYICNSGFKRKAGTSSLTECVLNKATNVA
HWTTPSLKCIRDPALVHQRPAPPS
Chain 5 and 6: FAP-L7-huIgKLC
(SEQ ID NO: 588)
EIVLTQSPGTLSLSPGERATLSCRASQSVSTSAYSYMHWYQQKPGQAPRLLIYLASNLESGIP
DRFSGSGSGTDFTLTISRLEPEDFAVYYCQQSRELPYTEGQGTKLEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSSPVTKSF
NRGEC
6) FAP-H6L7-huIgG1(L234A, L235A, P329A)-delK-GGSPLG/LAGGGS-IL-
15(S162A)-GGGSGGGSGRSDNQGGGSG-IL-15Rα(T2A)/FAP-H6L7-
huIgG1(L234A, L235A, P329A)-delK-GGSPLG/LAGGGS-IL-15(S162A)-
GGGSGGGSGR/SDNQGGGSG-IL-15Rα(T2A)/FAP-L7-huIgKLC (product from partial
cleavage, active)
Chain 1: FAP-H6L7-huIgG1(L234A, L235A, P329A)-delK-GGSPLG
(SEQ ID NO: 589)
QVQLVQSGAEVKKPGASVKVSCKASGYTFTENIIHWVRQAPGQGLEWMGWIHPGSGSIKYAQK
LQGRVTMTTDTSTSTAYMELRSLRSDDTAVYYCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGSPLG
Chain 2: LAGGGS-IL-15(S162A)-GGGSGGGSGRSDNQGGGSG-IL-15Rα(T2A)
(SEQ ID NO: 590)
LAGGGSNWVNVISDLKKIEDLIQSMHIDATLYTESDVHPSCKVTAMKCFLLELQVISLESGDA
SIHDTVENLIILANNSLSSNGNVTESGCKECEELEEKNIKEFLQSFVHIVQMFINTAGGGSGGGSGRSDNQGGGS
GIACPPPMSVEHADIWVKSYSLYSRERYICNSGFKRKAGTSSLTECVLNKATNVAHWTTPSLKCIRDPALVHQRP
APPS
Chain 3: FAP-H6L7-huIgG1(L234A, L235A, P329A)-delK-GGSPLGLAGGGS-
IL-15(S162A)-GGGSGGGSGR
(SEQ ID NO: 591)
QVQLVQSGAEVKKPGASVKVSCKASGYTFTENIIHWVRQAPGQGLEWMGWIHPGSGSIKYAQK
LQGRVTMTTDTSTSTAYMELRSLRSDDTAVYYCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGSPLG
Chain 4: LAGGGS-IL-15(S162A)-GGGSGGGSGR
(SEQ ID NO: 592)
LAGGGSNWVNVISDLKKIEDLIQSMHIDATLYTESDVHPSCKVTAMKCFLLELQVISLESGDA
SIHDTVENLIILANNSLSSNGNVTESGCKECEELEEKNIKEFLQSFVHIVQMFINTAGGGSGGGSGR
Chain 5: SDNQGGGSG-IL-15Rα(T2A)
(SEQ ID NO: 593)
SDNQGGGSGIACPPPMSVEHADIWVKSYSLYSRERYICNSGFKRKAGTSSLTECVLNKATNVA
HWTTPSLKCIRDPALVHQRPAPPS
Chain 6 and 7: FAP-L7-huIgKLC
(SEQ ID NO: 594)
EIVLTQSPGTLSLSPGERATLSCRASQSVSTSAYSYMHWYQQKPGQAPRLLIYLASNLESGIP
DRFSGSGSGTDFTLTISRLEPEDFAVYYCQQSRELPYTEGQGTKLEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSSPVTKSF
NRGEC
7) FAP-H6L7-huIgG1(L234A, L235A, P329A)-delK-GGSPLGLAGGGS-IL-
15(S162A)-GGGSGGGSGR/SDNQGGGSG-IL-15Rα(T2A)/FAP-H6L7-
huIgG1(L234A, L235A, P329A)-delK-GGSPLG/LAGGGS-IL-15(S162A)-
GGGSGGGSGR/SDNQGGGSG-IL-15Rα(T2A)/FAP-L7-huIgKLC (product from partial
cleavage, active)
Chain 1: FAP-H6L7-huIgG1(L234A, L235A, P329A)-delK-GGSPLGLAGGGS-
IL-15(S162A)-GGGSGGGSGR
(SEQ ID NO: 595)
QVQLVQSGAEVKKPGASVKVSCKASGYTFTENIIHWVRQAPGQGLEWMGWIHPGSGSIKYAQK
LQGRVTMTTDTSTSTAYMELRSLRSDDTAVYYCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGSPLGLAGGGSNWVNVISDLKKIEDLIQSMHIDATLYTESDVHPSCKVTAMKCFLLELQVISL
ESGDASIHDTVENLIILANNSLSSNGNVTESGCKECEELEEKNIKEFLQSFVHIVQMFINTAGGGSGGGSGR
Chain 2: SDNQGGGSG-IL-15Rα(T2A)
(SEQ ID NO: 596)
SDNQGGGSGIACPPPMSVEHADIWVKSYSLYSRERYICNSGFKRKAGTSSLTECVLNKATNVA
HWTTPSLKCIRDPALVHQRPAPPS
Chain 3: FAP-H6L7-huIgG1(L234A, L235A, P329A)-delK-GGSPLGLAGGGS-
IL-15(S162A)-GGGSGGGSGR
(SEQ ID NO: 597)
QVQLVQSGAEVKKPGASVKVSCKASGYTFTENIIHWVRQAPGQGLEWMGWIHPGSGSIKYAQK
LQGRVTMTTDTSTSTAYMELRSLRSDDTAVYYCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGSPLG
Chain 4: LAGGGS-IL-15(S162A)-GGGSGGGSGR
(SEQ ID NO: 598)
LAGGGSNWVNVISDLKKIEDLIQSMHIDATLYTESDVHPSCKVTAMKCFLLELQVISLESGDA
SIHDTVENLIILANNSLSSNGNVTESGCKECEELEEKNIKEFLQSFVHIVQMFINTAGGGSGGGSGR
Chain 5: SDNQGGGSG-IL-15Rα(T2A)
(SEQ ID NO: 599)
SDNQGGGSGIACPPPMSVEHADIWVKSYSLYSRERYICNSGFKRKAGTSSLTECVLNKATNVA
HWTTPSLKCIRDPALVHQRPAPPS
Chain 6 and 7: FAP-L7-huIgKLC
(SEQ ID NO: 600)
EIVLTQSPGTLSLSPGERATLSCRASQSVSTSAYSYMHWYQQKPGQAPRLLIYLASNLESGIP
DRFSGSGSGTDFTLTISRLEPEDFAVYYCQQSRELPYTEGQGTKLEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSSPVTKSF
NRGEC
8) FAP-H6L7-huIgG1(L234A, L235A, P329A)-delK-GGSPLGLAGGGS-IL-
15(S162A)-GGGSGGGSGR/SDNQGGGSG-IL-15Rα(T2A)/FAP-L7-huIgKLC (product from
partial cleavage, active)
Chains 1 and 2: FAP-H6L7-huIgG1(L234A, L235A, P329A)-delK-
GGSPLGLAGGGS-IL-15(S162A)-GGGSGGGSGR
(SEQ ID NO: 601)
QVQLVQSGAEVKKPGASVKVSCKASGYTFTENIIHWVRQAPGQGLEWMGWIHPGSGSIKYAQK
LQGRVTMTTDTSTSTAYMELRSLRSDDTAVYYCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGSPLGLAGGGSNWVNVISDLKKIEDLIQSMHIDATLYTESDVHPSCKVTAMKCFLLELQVISL
ESGDASIHDTVENLIILANNSLSSNGNVTESGCKECEELEEKNIKEFLQSFVHIVQMFINTAGGGSGGGSGR
Chains 3 and 4: SDNQGGGSG-IL-15Rα(T2A)
(SEQ ID NO: 602)
SDNQGGGSGIACPPPMSVEHADIWVKSYSLYSRERYICNSGFKRKAGTSSLTECVLNKATNVA
HWTTPSLKCIRDPALVHQRPAPPS
Chain 5 and 6: FAP-L7-huIgKLC
(SEQ ID NO: 603)
EIVLTQSPGTLSLSPGERATLSCRASQSVSTSAYSYMHWYQQKPGQAPRLLIYLASNLESGIP
DRFSGSGSGTDFTLTISRLEPEDFAVYYCQQSRELPYTEGQGTKLEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSSPVTKSF
NRGEC
9) FAP-H6L7-huIgG1(L234A, L235A, P329A)-delK-GGSPLG/LAGGGS-IL-
15(S162A)-GGGSGGGSGRSDNQGGGSG-IL-15Rα(T2A)/FAP-L7-huIgKLC (product from
partial cleavage, active)
Chains 1 and 2: FAP-H6L7-huIgG1(L234A, L235A, P329A)-delK-GGSPLG
(SEQ ID NO: 604)
QVQLVQSGAEVKKPGASVKVSCKASGYTFTENIIHWVRQAPGQGLEWMGWIHPGSGSIKYAQK
LQGRVTMTTDTSTSTAYMELRSLRSDDTAVYYCARHGGTGRGAMDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALAAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGGGSPLG
Chains 3 and 4: LAGGGS-IL-15(S162A)-GGGSGGGSGRSDNQGGGSG-IL-
15Rα(T2A)
(SEQ ID NO: 605)
LAGGGSNWVNVISDLKKIEDLIQSMHIDATLYTESDVHPSCKVTAMKCFLLELQVISLESGDA
SIHDTVENLIILANNSLSSNGNVTESGCKECEELEEKNIKEFLQSFVHIVQMFINTAGGGSGGGSGRSDNQGGGS
GIACPPPMSVEHADIWVKSYSLYSRERYICNSGFKRKAGTSSLTECVLNKATNVAHWTTPSLKCIRDPALVHQRP
APPS
Chain 5 and 6: FAP-L7-huIgKLC
(SEQ ID NO: 606)
EIVLTQSPGTLSLSPGERATLSCRASQSVSTSAYSYMHWYQQKPGQAPRLLTYLASNLESGIP
DRFSGSGSGTDFTLTISRLEPEDFAVYYCQQSRELPYTEGQGTKLEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSSPVTKSF
NRGEC

Expected Cleavage Products from uPA/Matriptase Cleavage of P24912158:

1) FAP-H6L7-huIgG1(L234A, L235A, P329A)-delK-
GGSGGSGGSGGS-IL-15(S162A)-GGGSGGGSGR/
SDNQGGGSGGGSG-IL-15Rα(T2A)/FAP-L7-huIgKLC
(product from complete cleavage)
Chains 1 and 2: FAP-H6L7-huIgG1(L234A, L235A,
P329A)-delK-GGSGGSGGSGGS-IL-15(S162A)-GGGSGGGSGR
(SEQ ID NO: 607)
QVQLVQSGAEVKKPGASVKVSCKASGYTFTENIIHWVRQAPGQGLEWMGW
IHPGSGSIKYAQKLQGRVTMTTDTSTSTAYMELRSLRSDDTAVYYCARHG
GTGRGAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALAAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGG
GSGGSGGSGGSNWVNVISDLKKIEDLIQSMHIDATLYTESDVHPSCKVTA
MKCELLELQVISLESGDASIHDTVENLIILANNSLSSNGNVTESGCKECE
ELEEKNIKEFLQSFVHIVQMFINTAGGGSGGGSGR
Chains 3 and 4: SDNQGGGSGGGSG-IL-15Rα(T2A)
(SEQ ID NO: 608)
SDNQGGGSGGGSGIACPPPMSVEHADIWVKSYSLYSRERYICNSGFKRKA
GTSSLTECVLNKATNVAHWTTPSLKCIRDPALVHQRPAPPS
Chains 5 and 6: FAP-L7-huIgKLC
(SEQ ID NO: 609)
EIVLTQSPGTLSLSPGERATLSCRASQSVSTSAYSYMHWYQQKPGQAPRL
LIYLASNLESGIPDRFSGSGSGTDFTLTISRLEPEDFAVYYCQQSRELPY
TEGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
2) FAP-H6L7-huIgG1(L234A, L235A, P329A)-delK-
GGSGGSGGSGGS-IL-15(S162A)-GGGSGGGSGRSDNQGGGSGGGSG-
IL-15Rα(T2A)/FAP-H6L7-huIgG1(L234A, L235A,
P329A)-delK-GGSGGSGGSGGS-IL-15(S162A)-GGGSGGGSGR/
SDNQGGGSGGGSG-IL-15Rα(T2A)/FAP-L7-huIgKLC
(product from partial cleavage, active)
Chain 1: FAP-H6L7-huIgG1(L234A, L235A, P329A)-
delK-GGSGGSGGSGGS-IL-15(S162A)-
GGGSGGGSGRSDNQGGGSGGGSG-IL-15Rα(T2A)
(SEQ ID NO: 610)
QVQLVQSGAEVKKPGASVKVSCKASGYTFTENIIHWVRQAPGQGLEWMGW
IHPGSGSIKYAQKLQGRVTMTTDTSTSTAYMELRSLRSDDTAVYYCARHG
GTGRGAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALAAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGG
GSGGSGGSGGSNWVNVISDLKKIEDLIQSMHIDATLYTESDVHPSCKVTA
MKCFLLELQVISLESGDASIHDTVENLIILANNSLSSNGNVTESGCKECE
ELEEKNIKEFLQSFVHIVQMFINTAGGGSGGGSGRSDNQGGGSGGGSGIA
CPPPMSVEHADIWVKSYSLYSRERYICNSGFKRKAGTSSLTECVLNKATN
VAHWTTPSLKCIRDPALVHQRPAPPS
Chain 2: FAP-H6L7-huIgG1(L234A, L235A, P329A)-
delK-GGSGGSGGSGGS-IL-15(S162A)-GGGSGGGSGR
(SEQ ID NO: 611)
QVQLVQSGAEVKKPGASVKVSCKASGYTFTENIIHWVRQAPGQGLEWMGW
IHPGSGSIKYAQKLQGRVTMTTDTSTSTAYMELRSLRSDDTAVYYCARHG
GTGRGAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALAAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGG
GSGGSGGSGGSNWVNVISDLKKIEDLIQSMHIDATLYTESDVHPSCKVTA
MKCFLLELQVISLESGDASIHDTVENLIILANNSLSSNGNVTESGCKECE
ELEEKNIKEFLQSFVHIVQMFINTAGGGSGGGSGR
Chain 3: SDNQGGGSGGGSG-IL-15Rα(T2A)
(SEQ ID NO: 612)
SDNQGGGSGGGSGIACPPPMSVEHADIWVKSYSLYSRERYICNSGFKRKA
GTSSLTECVLNKATNVAHWTTPSLKCIRDPALVHQRPAPPS
Chain 4 and 5: FAP-L7-huIgKLC
(SEQ ID NO: 613)
EIVLTQSPGTLSLSPGERATLSCRASQSVSTSAYSYMHWYQQKPGQAPRL
LIYLASNLESGIPDRFSGSGSGTDFTLTISRLEPEDFAVYYCQQSRELPY
TEGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

Having described embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. An activatable proprotein comprising a first polypeptide and a second polypeptide, wherein the first and second polypeptide each comprise a masking moiety operably linked via a first linker on the C-terminus to an IL-15 or variant thereof, wherein the IL-15 or variant thereof is linked via a second linker on the C-terminus to an IL-15Rα or variant thereof, and wherein the masking moiety masks the active portion of the proprotein.

2. The activatable proprotein of claim 1, wherein the first linker on the first or second polypeptide, or on both the first and the second polypeptide is a cleavable linker.

3. The activatable proprotein of any one of claims 1-2, wherein the second linker on the first or second polypeptide, or on both the first and the second polypeptide is a cleavable linker.

4. The activatable proprotein of any one of claims 1-3, wherein the IL-15 or variant thereof and the IL-15Rα or variant thereof in the first polypeptide and the IL-15 or variant thereof and the IL-15Rα or variant thereof in the second polypeptide comprise one or more Cys substitution mutations.

5. The activatable proprotein of claim 4, wherein the IL-15 or variant thereof in the first polypeptide comprising one or more Cys substitution mutations forms a disulfide bond with the IL-15Rα or variant thereof comprising one or more Cys substitution mutations in the second polypeptide, or wherein the IL-15 or variant thereof in the second polypeptide comprising one or more Cys substitution mutations forms a disulfide bond with the IL-15Rα or variant thereof comprising one or more Cys substitution mutations in the first polypeptide.

6. The activatable proprotein of any one of claims 1-5, wherein the proprotein is a dimeric proprotein.

7. The activatable proprotein of claim 6, wherein the dimeric proprotein comprises a first polypeptide and a second polypeptide each comprising from N-terminus to C-terminus the masking moiety linked via the first linker to the IL-15 or variant thereof, wherein the IL-15 or variant thereof is linked via the second linker to the IL-15Rα or variant thereof, and wherein the masking moiety of the first polypeptide forms one or more covalent disulfide bonds or one or more non-covalent bonds with the masking moiety of the second polypeptide.

8. The activatable proprotein of any one of claims 1-7, wherein the masking moiety in the first polypeptide comprises one or more protein domains.

9. The activatable proprotein of any one of claims 1-8, wherein the masking moiety in the second polypeptide comprises one or more protein domains.

10. The activatable proprotein of any one of claims 1-9, wherein the masking moiety in the first polypeptide does not bind to an antigen.

11. The activatable proprotein of any one of claims 1-9, wherein the masking moiety in the first polypeptide binds to an antigen.

12. The activatable proprotein of any one of claims 1-9, wherein the masking moiety in the second polypeptide does not bind to an antigen.

13. The activatable proprotein of any one of claims 1-9, wherein the masking moiety in the second polypeptide binds to an antigen.

14. The activatable proprotein of any one of claims 1-13, wherein the masking moiety in the first polypeptide comprises a CH1, CH2, CH3, CH2CH3, or CH1CH2CH3 domain of an antibody constant (Fc) region.

15. The activatable proprotein of any one of claims 1-14, wherein the masking moiety in the first polypeptide comprises one or more heterodimers of at least two different CH3 variant domains of an antibody constant (Fc) region.

16. The activatable proprotein of any one of claims 1-9 and 11, wherein the masking moiety in the first polypeptide comprises a fusion of an antigen binding domain with a CH1, CH2, CH3, CH2CH3, or CH1CH2CH3 domain of an antibody constant (Fc) region.

17. The activatable proprotein of any one of claims 1-13, wherein the masking moiety in the second polypeptide comprises a CH1, CH2, CH3, CH2CH3, or CH1CH2CH3 domain of an antibody constant (Fc) region.

18. The activatable proprotein of any one of claims 1-13 or 17, wherein the masking moiety in the second polypeptide comprises one or more heterodimers of at least two different CH3 variant domains of an antibody constant (Fc) region.

19. The activatable proprotein of any one of claims 1-9 and 13, wherein the masking moiety in the second polypeptide comprises a fusion of an antigen binding domain with a CH1, CH2, CH3, CH2CH3, or CH1CH2CH3 domain of an antibody constant (Fc) region.

20. The activatable proprotein of any one of claims 14-19, wherein the antibody Fc region is from a immunoglobulin class selected from IgG1, IgG2, IgG3, IgG4, IgD, IgA, or IgM.

21. The activatable proprotein of any one of claims 10 or 12, wherein the masking moiety comprises a constant domain of an antibody light chain (CL), and wherein the light chain is a lambda or kappa chain.

22. The activatable proprotein of claim 11, wherein the masking moiety in the first polypeptide comprises an antigen binding domain.

23. The activatable proprotein of claim 13, wherein the masking moiety in the second polypeptide comprises an antigen binding domain.

24. The activatable proprotein of any one of claims 22-23, wherein the antigen binding domain is a variable heavy chain domain (VH), a variable domain from a heavy chain antibody (VHH), or an antigen specific peptide.

25. The activatable proprotein of any one of claims 22-23, wherein the antigen binding domain comprises an antibody light chain (LC) and an antibody heavy chain (HC).

26. The activatable proprotein of any one of claims 2-25, wherein the cleavable linker(s) comprises a cleavage site for a protease.

27. The activatable proprotein of claim 26, wherein the protease is selected from a metalloprotease, a serine protease, a cysteine protease or an aspartic acid protease or any combination thereof.

28. The activatable proprotein of any one of claims 26-27, wherein the protease cleavage site is cleavable by a MMP1, MMP2, MMP3, MMP4, MMP5, MMP6, MMP7, MMP8, MMP9, MMP10, MMP11, MMP12, MMP13, MMP14, TEV, matriptase, uPA, FAP, Legumain, PSA, Kallikrein, Cathepsin A, or a Cathepsin B protease.

29. The activatable proprotein of any one of claims 1-28, wherein the masking moiety in the first and/or second polypeptide prevents binding of a complex of IL-15/IL-15Rα in the first and/or second polypeptide to IL-15Rβ/γC present on the surface of a lymphocyte or a blood cell in vitro or in vivo.

30. The activatable proprotein of any one of claims 2-29, wherein cleavage of the masking moieties in any one of the first and/or second polypeptides in the activatable proprotein leads to partial activation of the activatable proprotein.

31. The activatable proprotein of any one of claims 2-30, wherein cleavage of the masking moiety in both of the first and second polypeptides in the activatable proprotein leads to complete activation of the activatable proprotein into an active complex of IL-15/IL-15Rα or a variant thereof.

32. The activatable proprotein of any one of claims 1-30, wherein the IL-15 or variant thereof is a human IL-15 or an amino acid mutant derived therefrom.

33. The activatable proprotein of any one of claims 1-30, wherein the IL-15Rα or variant thereof comprises a human IL-15Rα, a truncated human IL-15Rα or an amino acid mutant derived from human IL-15Rα.

34. The activatable proprotein of any one of claims 1-31, wherein the IL-15/IL-15Rα or variant thereof comprises a human IL-15 or an amino acid mutant derived therefrom, and a human IL-15Rα, a truncated human IL-15Rα or an amino acid mutant derived from human IL-15Rα.

35. The activatable proprotein of any one of claims 1-34, wherein said activatable proprotein comprises an amino acid sequence set forth in SEQ ID NO: 26 or SEQ ID NO: 228, or a sequence in the Sequence Listing, including variants thereof having at least 80, 85, 90, 95, 97, 98, 99% identity to a sequence in the Sequence Listing.

36. A recombinant nucleic acid molecule encoding the activatable proprotein of any one of claims 1-35.

37. A vector comprising the recombinant nucleic acid molecule of claim 36.

38. A pharmaceutical composition comprising the activatable proprotein of any one of claims 1-35, and a pharmaceutically acceptable carrier.

39. An activatable proprotein comprising a fusion of a first polypeptide and a second polypeptide, wherein the first and second polypeptide each comprise an IL-15 or variant thereof operably linked via a first linker on the C-terminus to an IL-15Rα or variant thereof, wherein the IL-15Rα or variant thereof is linked via a second linker on the C-terminus to a masking moiety present on each of the first and second polypeptides, and wherein the masking moiety masks the active portion of the proprotein.

40. The activatable proprotein of claim 39, wherein the first linker on the first or second polypeptide, or on both the first and the second polypeptide is a cleavable linker.

41. The activatable proprotein of any one of claims 39-40, wherein the second linker on the first or second polypeptide, or on both the first and the second polypeptide is a cleavable linker.

42. The activatable proprotein of any one of claims 40-41, wherein the IL-15 or variant thereof and the IL-15Rα or variant thereof in the first polypeptide and the IL-15 or variant thereof and the IL-15Rα or variant thereof in the second polypeptide comprise one or more Cys substitution mutations.

43. The activatable proprotein of claim 42, wherein the IL-15 or variant thereof in the first polypeptide comprising one or more Cys substitution mutations forms a disulfide bond with the IL-15Rα or variant thereof comprising one or more Cys substitution mutations in the second polypeptide, or wherein the IL-15 or variant thereof in the second polypeptide comprising one or more Cys substitution mutations forms a disulfide bond with the IL-15Rα or variant thereof comprising one or more Cys substitution mutations in the first polypeptide.

44. The activatable proprotein of any one of claims 39-43, wherein the proprotein is a dimeric proprotein.

45. The activatable proprotein of claim 44, wherein the dimeric proprotein comprises a first polypeptide and a second polypeptide each comprising from N-terminus to C-terminus the IL-15 or variant thereof linked via the first linker on the C-terminus to the IL-15Rα or variant thereof, wherein the IL-15Rα or variant thereof is linked via the second linker on the C-terminus to the masking moiety, and wherein the masking moiety of the first polypeptide forms one or more covalent disulfide bonds or non-covalent bonds with the masking moiety of the second polypeptide.

46. The activatable proprotein of any one of claims 39-45, wherein the masking moiety in the first polypeptide comprises one or more protein domains.

47. The activatable proprotein of any one of claims 39-45, wherein the masking moiety in the second polypeptide comprises one or more protein domains.

48. The activatable proprotein of any one of claims 39-47, wherein the masking moiety in the first polypeptide does not bind to an antigen.

49. The activatable proprotein of any one of claims 39-47, wherein the masking moiety in the first polypeptide binds to an antigen.

50. The activatable proprotein of any one of claims 39-47, wherein the masking moiety in the second polypeptide does not bind to an antigen.

51. The activatable proprotein of any one of claims 39-47, wherein the masking moiety in the second polypeptide binds to an antigen.

52. The activatable proprotein of any one of claims 40-52, wherein the masking moiety in the first polypeptide comprises a CH1, CH2, CH3, CH2CH3, or CH1CH2CH3 domain of an antibody constant (Fc) region.

53. The activatable proprotein of any one of claims 39-52, wherein the masking moiety in the first polypeptide comprises a one or more heterodimers of at least two different CH3 variant domains of an antibody constant (Fc) region.

54. The activatable proprotein of any one of claims 39-47 and 49, wherein the masking moiety in the first polypeptide comprises a fusion of an antigen binding domain with a CH1, CH2, CH3, CH2CH3, or CH1CH2CH3 domain of an antibody constant (Fc) region.

55. The activatable proprotein of any one of claims 39-51, wherein the masking moiety in the second polypeptide comprises a CH1, CH2, CH3, CH2CH3, or CH1CH2CH3 domain of an antibody constant (Fc) region.

56. The activatable proprotein of any one of claims 39-51 or 55, wherein the masking moiety in the first polypeptide comprises one or more heterodimers of at least two different CH3 variant domains of an antibody constant (Fc) region.

57. The activatable proprotein of any one of claims 39-51 and 51, wherein the masking moiety in the second polypeptide comprises a fusion of an antigen binding domain with a CH1, CH2, CH3, CH2CH3, or CH1CH2CH3 domain of an antibody constant (Fc) region.

58. The activatable proprotein of any one of claims 52-57, wherein the antibody Fc region is from a immunoglobulin class selected from IgG1, IgG2, IgG3, IgG4, IgD, IgA, or IgM.

59. The activatable proprotein of any one of claims 48 or 50, wherein the masking moiety comprises a constant domain of an antibody light chain (CL), wherein the light chain is a lambda or kappa chain.

60. The activatable proprotein of claim 54, wherein the masking moiety in the first polypeptide comprises an antigen binding domain.

61. The activatable proprotein of claim 57, wherein the masking moiety in the second polypeptide comprises an antigen binding domain.

62. The activatable proprotein of any one of claims 60-61, wherein the antigen binding domain is a variable heavy chain domain (VH), a variable domain from a heavy chain antibody (VHH), or an antigen specific peptide.

63. The activatable proprotein of any one of claims 60-61, wherein the antigen binding domain comprises an antibody light chain (LC) and an antibody heavy chain (HC).

64. The activatable proprotein of any one of claims 40-63, wherein the first linker comprises 26 amino acids.

65. The activatable proprotein of any one of claims 40-64, wherein the cleavable linker(s) comprises a cleavage site for a protease.

66. The activatable proprotein of claim 64, wherein the protease is selected from a metalloprotease, a serine protease, a cysteine protease or an aspartic acid protease or any combination thereof.

67. The activatable proprotein of any one of claims 65-66, wherein the protease cleavage site is cleavable by a MMP1, MMP2, MMP3, MMP4, MMP5, MMP6, MMP7, MMP8, MMP9, MMP10, MMP11, MMP12, MMP13, MMP14, TEV, matriptase, uPA, FAP, Legumain, PSA, Kallikrein, Cathepsin A, or a Cathepsin B protease.

68. The activatable proprotein of any one of claims 39-67, wherein the masking moiety in the first and/or second polypeptide prevents binding of a complex of IL-15/IL-15Rα in the first and/or second polypeptide to IL-15Rβ/γC present on the surface of a lymphocyte or a blood cell in vitro or in vivo.

69. The activatable proprotein of any one of claims 40-68, wherein cleavage of the masking moiety in any one of the first or second polypeptides in the activatable proprotein leads to partial activation of the activatable proprotein.

70. The activatable proprotein of any one of claims 40-69, wherein cleavage of the masking moieties in both of the first and second polypeptides in the activatable proprotein leads to complete activation of the activatable proprotein into an active complex of IL-15/IL-15Rα or a variant thereof.

71. The activatable proprotein of any one of claims 39-69, wherein the IL-15 or variant thereof is a human IL-15 or an amino acid mutant derived therefrom.

72. The activatable proprotein of any one of claims 39-69, wherein the IL-15Rα or variant thereof comprises a human IL-15Rα, a truncated human IL-15Rα or an amino acid mutant derived from human IL-15Rα.

73. The activatable proprotein of any one of claims 39-70, wherein the IL-15/IL-15Rα or variant thereof comprises a human IL-15 or an amino acid mutant derived therefrom, and a human IL-15Rα, a truncated human IL-15Rα or an amino acid mutant derived from human IL-15Rα.

74. The activatable proprotein of any one of claims 39-73, wherein said activatable proprotein comprises an amino acid sequence set forth in SEQ ID NO: 17, or SEQ ID NO: 73, or a sequence in the Sequence Listing, including variants thereof having at least 80, 85, 90, 95, 97, 98, 99% identity to a sequence in the Sequence Listing.

75. A recombinant nucleic acid molecule encoding the activatable proprotein of any one of claims 39-74.

76. A vector comprising the recombinant nucleic acid molecule of claim 75.

77. A pharmaceutical composition comprising the activatable proprotein of any one of claims 39-74, and a pharmaceutically acceptable carrier.

78. A method comprising administering the activatable proprotein of any one of claims 1-35, 39-74 or the pharmaceutical composition of any one of claims 39 or 79 to a subject having a cancer to treat the cancer in the subject, wherein following administration, the activatable proprotein is activated through protease cleavage in a cancerous tissue.

79. A method comprising administering the activatable proprotein of any one of claims 1-35, 39-74 or the pharmaceutical composition of any one of claims 39 or 77 to a subject in need thereof, to elicit or enhance an anti-tumor immune response in the subject, wherein following administration, the activatable proprotein is activated through protease cleavage in the tumor.

80. Use of the activatable proprotein of any one of claims 1-35, 39-74 or the pharmaceutical composition of any one of claims 39 or 77 for treating a cancer in a subject, comprising the step of administering the activatable proprotein or pharmaceutical composition, and wherein following administration, the activatable proprotein is activated through protease cleavage in a cancerous tissue.

81. Use of the activatable proprotein of any one of claims 1-35, 39-74 or the pharmaceutical composition of any one of claims 39 or 77 for eliciting or enhancing an anti-tumor immune response in a subject, comprising the step of administering the activatable proprotein or pharmaceutical composition, and wherein following administration, the activatable proprotein is activated through protease cleavage in a tumor.

82. The method of any one of claims 78-79 or the use of any one of claims 80-81, wherein protease cleavage partially or completely removes the masking moiety in the activatable proprotein such that the IL-15/IL-IL-15Rα complex in the first and the second polypeptide can bind IL-15Rβ/γC present on the surface of a lymphocyte or a blood cell in vitro or in vivo.

83. The method of any one of claims 78-79 or the use of any one of claims 80-81, wherein the cancer is selected from prostate cancer, colon cancer, renal carcinoma, melanoma, lung cancer, breast cancer, thyroid cancer, bladder cancer, gastric and esophageal cancer, pancreatic cancer, liver cancer, brain cancer, head and neck cancer, neuroblastoma, soft tissue carcinoma, lymphoma, leukemia, multiple myeloma, or any metastases therefrom.

84. The activatable proprotein of any one of claims 29 or 68, or the method or use of claim 84, wherein the lymphocyte or a blood cell is a CD4⁺ T cell, a CD8⁺ T cell, a natural killer (NK) cell or B cells.
